(19) United States
(12) Patent Application Publication
(10) Pub. No.: US 2026/0007683 A1
(43) Pub. Date: Jan. 8, 2026

(54) PHARMACEUTICAL COMPOSITIONS COMPRISING HIV INTEGRASE INHIBITORS

(71) Applicant: Gilead Sciences, Inc., Foster City, CA (US)

(72) Inventor: Scott Victor Jermain, San Mateo, CA (US)

(21) Appl. No.: 19/238,056

(22) Filed: Jun. 13, 2025

Related U.S. Application Data
(60) Provisional application No. 63/660,007, filed on Jun. 14, 2024.

(51) Int. Cl. A61K 31/55; A61K 9/00; A61K 9/14; A61K 47/02; A61K 47/26; A61K 47/44

(52) U.S. Cl. CPC A61K 31/55; A61K 9/0019; A61K 9/145; A61K 47/02; A61K 47/26; A61K 47/44

(57) ABSTRACT

The present disclosure relates to pharmaceutical compositions (e.g., suspension formulations) including a compound of Formula I:

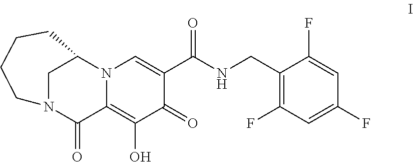 [SMILES: O=C(NCc1c(F)cc(F)cc1F)c1cn2c(c(O)c1=O)C(=O)N1CCCC[C@H]2C1]

or a pharmaceutically acceptable salt thereof, and a pharmaceutically acceptable liquid vehicle.

Also disclosed are methods of treating or preventing human immunodeficiency virus (HIV) in a human, including administering to the human a pharmaceutical composition disclosed herein.

### CROSS REFERENCE TO RELATED APPLICATIONS

This application claims the benefit of U.S. Provisional Application No. 63/660,007, filed on Jun. 14, 2024, the entire contents of which is hereby incorporated by reference in its entirety.

### TECHNICAL FIELD

The present invention provides pharmaceutical compositions comprising the HIV integrase inhibitor (7S)-12-hydroxy-1,11-dioxo-N-(2,4,6-trifluorobenzyl)-1,4,5,6,7,11-hexahydro-3H-2,7-methanopyrido[1,2-a][1,4]diazonine-10-carboxamide, or a pharmaceutically acceptable salt thereof, and methods of treating or preventing HIV in a subject comprising administering to the subject a therapeutically effective amount of said pharmaceutical composition by injection, optionally in combination with one or more other therapeutic agents.

### BACKGROUND

There is an ongoing need for antiviral agents and methods for treating HIV viral infections. There is also a constant need to develop methods for preparation and purification of the antiviral agents, as well as prepare improved pharmaceutical formulations of the same. The solid forms disclosed herein help meet these and other needs. HIV-1 infection is a life threatening and serious disease of major public health significance, with approximately 38 million people infected worldwide and approximately 22 million taking antiretroviral (ARV) treatment (Joint United Nations Programme on HIV/AIDS (UNAIDS). UNAIDS Data. Geneva, Switzerland. 2018). Standard of care for the treatment of HIV-1 infection uses combination ARV therapy (ART) to suppress viral replication to below detectable limits, allow cluster determinant 4 (CD4) cell counts to increase, and stop disease progression. For ART-naive, HIV-infected patients, current treatment guidelines suggest that initial therapy consist of 2 nucleos(t)ide reverse transcriptase inhibitors (N[t]RTIs) and preferably an integrase strand-transfer inhibitor (INSTI) (Department of Health and Human Services (DHHS), Panel on Antiretroviral Therapy and Medical Management of HIV-Infected Children. www.aidsinfo.nih.gov/guidelines. May 22, 2018; Gunthard H F, et al. Antiretroviral Drugs for Treatment and Prevention of HIV Infection in Adults: 2016 Recommendations of the International Antiviral Society-USA Panel. JAMA 2016; 316 (2):191-210; The Department of Health and Human Services (DHHS) Panel on Antiretroviral Guidelines for Adults and Adolescents; www.aidsinfo.nih.gov/ContentFiles/AdultandAdolescentGL 25 Oct. 2018).

Virologically suppressed, HIV-infected patients can benefit by switching from their current regimen to improve safety or tolerability or to simplify the regimen (European AIDS Clinical Society (EACS). The EACS Guidelines Version 10. November. 2019; The Department of Health and Human Services (DHHS) Panel on Antiretroviral Guidelines for Adults and Adolescents).

While current combination ARV therapy for the treatment of HIV-1 infection is efficacious and well tolerated, these agents need to be taken every day and require adherence to minimize the emergence of drug-resistant variants. As a result, “treatment fatigue” can occur, defined as “decreased desire and motivation to maintain vigilance in adhering to a treatment regimen” among people living with HIV (PLWH) prescribed chronic or life-long treatment (Claborn K R, et al., A Systematic Review Of Treatment Fatigue Among HIV-Infected Patients Prescribed Antiretroviral Therapy. Psychology, health & medicine 2015; 20 (3):1-11), which can lead to nonadherence and treatment failure. As such, there remains a medical need for ARVs that can be administered less frequently (i.e., long-acting drug products), thereby providing an alternative treatment option for PLWH.

### SUMMARY

The present disclosure provides pharmaceutical compositions comprising a compound of Formula I:

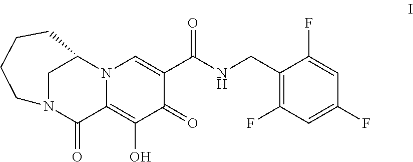 [SMILES: O=C(NCc1c(F)cc(F)cc1F)c1cn2c(c(O)c1=O)C(=O)N1CCCC[C@H]2C1]

or a pharmaceutically acceptable salt thereof, and a pharmaceutically acceptable liquid vehicle;
wherein the pharmaceutical composition is a suspension formulation that is suitable for injection. The present disclosure also provides methods of treating or preventing HIV in a subject comprising administering to the subject a therapeutically effective amount of said pharmaceutical composition by injection, optionally in combination with one or more other therapeutic agents.

The pharmaceutical compositions described herein include long acting formulations comprising the compound of Formula I, or a pharmaceutically acceptable salt thereof.

Also described herein are methods for the preparation of long acting formulations of the compound of Formula I, or a pharmaceutically acceptable salt thereof, and uses of the long acting formulations as therapeutic or prophylactic agents.

### BRIEF DESCRIPTION OF THE DRAWINGS

FIG. 1. Shows the XRPD pattern for the compound of Formula I, Form I

FIG. 2. Shows the DSC curve for the compound of Formula I, Form I

FIG. 3. Shows the TGA curve for the compound of Formula I, Form I.

FIG. 4. Shows the DVS curve for the compound of Formula I, Form I.

FIG. 5. Shows the atomic displacement ellipsoid drawing of the compound of Formula I, Form I.

FIG. 6. Shows the XRPD pattern for the compound of Formula I, Form II.

FIG. 7. Shows the XRPD pattern for the compound of Formula I, Form III.

FIG. 8. Shows the DSC curve for the compound of Formula I, Form III.

FIG. 9. Shows the TGA curve for the compound of Formula I, Form III.

FIG. 10. Shows the DVS curve for the compound of Formula I, Form III.

FIG. 11. Shows the XRPD pattern for the compound of Formula I, Form IV.

FIG. 12. Shows the DSC curve for the compound of Formula I, Form IV.

FIG. 13. Shows the TGA curve for the compound of Formula I, Form IV.

FIG. 14. Shows the DVS curve for the compound of Formula I, Form IV.

FIG. 15. Shows the XRPD pattern for the compound of Formula I, Amorphous.

FIG. 16. Shows the DSC curve for the compound of Formula I, Amorphous.

FIG. 17. Shows the XRPD pattern for the compound of Formula I, Sodium Salt, Form I.

FIG. 18. Shows the DSC curve for the compound of Formula I, Sodium Salt, Form I.

FIG. 19. Shows the TGA curve for the compound of Formula I, Sodium Salt, Form I.

FIG. 20. Shows the DVS curve for the compound of Formula I, Sodium Salt, Form I.

FIG. 21. Shows the XRPD pattern for the compound of Formula I, Calcium Salt, Form I.

FIG. 22. Shows the DSC curve for the compound of Formula I, Calcium Salt, Form I.

FIG. 23. Shows the TGA curve for the compound of Formula I, Calcium Salt, Form I.

FIG. 24. Shows the DVS curve for the compound of Formula I, Calcium Salt, Form I.

FIG. 25. Shows the XRPD pattern for the compound of Formula I, Calcium Salt, Form II.

FIG. 26. Shows the DSC curve for the compound of Formula I, Calcium Salt, Form II.

FIG. 27. Shows the TGA curve for the compound of Formula I, Calcium Salt, Form II.

FIG. 28. Shows the XRPD pattern for the compound of Formula I, Calcium Salt, Form III.

FIG. 29. Shows the DSC curve for the compound of Formula I, Calcium Salt, Form III.

FIG. 30. Shows the TGA curve for the compound of Formula I, Calcium Salt, Form III.

FIG. 31. Shows the DVS curve for the compound of Formula I, Calcium Salt, Form III.

FIG. 32. Shows the XRPD pattern for the compound of Formula I, Calcium Salt, Form IV.

FIG. 33. Shows the DSC curve for the compound of Formula I, Calcium Salt, Form IV.

FIG. 34. Shows the TGA curve for the compound of Formula I, Calcium Salt, Form IV.

FIG. 35. Shows the DVS curve for the compound of Formula I, Calcium Salt, Form IV.

FIG. 36. Shows the XRPD pattern for the compound of Formula I, Calcium Salt, Form V.

FIG. 37. Shows the DSC curve for the compound of Formula I, Calcium Salt, Form V.

FIG. 38. Shows the TGA curve for the compound of Formula I, Calcium Salt, Form V.

FIG. 39. Shows the DVS curve for the compound of Formula I, Calcium Salt, Form V.

FIG. 40. Shows the XRPD pattern for the compound of Formula I, Calcium Salt, Form VI.

FIG. 41. Shows the DSC curve for the compound of Formula I, Calcium Salt, Form VI.

FIG. 42. Shows the TGA curve for the compound of Formula I, Calcium Salt, Form VI.

FIG. 43. Shows the DVS curve for the compound of Formula I, Calcium Salt, Form VI.

FIG. 44. Shows the XRPD pattern for the compound of Formula I, Calcium Salt, Amorphous.

FIG. 45. Shows the DSC curve for the compound of Formula I, Calcium Salt, Amorphous.

FIG. 46. Shows a schematic of example microsuspension formulations provided as one component or two components (e.g., a compound of Formula I, Form I and a vehicle).

FIG. 47. Shows a schematic of an example manufacturing process for the Compound of Formula I, Form I.

FIG. 48. Shows a schematic of an example manufacturing process for a liquid vehicle comprising a mixture of PEG 300 and water.

FIG. 49. Shows a schematic of an example compounding process for a sesame oil vehicle.

FIG. 50. Shows a schematic of an example process for preparing a nanosuspension comprising a compound of Formula I, or a pharmaceutically acceptable salt thereof.

FIG. 51. Shows a schematic of an example process for preparing a nanosuspension comprising a compound of Formula I, or a pharmaceutically acceptable salt thereof.

FIG. 52. Shows a schematic of an example process for preparing a nanosuspension comprising a compound of Formula I, or a pharmaceutically acceptable salt thereof.

### DETAILED DESCRIPTION

The present disclosure relates to pharmaceutical compositions comprising the HIV integrase inhibitor (7S)-12-hydroxy-1,11-dioxo-N-(2,4,6-trifluorobenzyl)-1,4,5,6,7,11-hexahydro-3H-2,7-methanopyrido[1,2-a][1,4]diazonine-10-carboxamide, or a pharmaceutically acceptable salt thereof (Compound of Formula I, see below), which was disclosed in WO2020/197991.

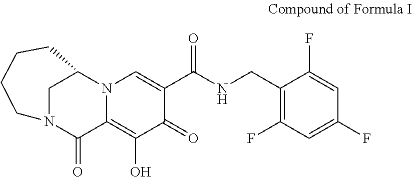 [SMILES: O=C(NCc1c(F)cc(F)cc1F)c1cn2c(c(O)c1=O)C(=O)N1CCCC[C@H]2C1]

The pharmaceutical compositions disclosed herein can be useful for treating or preventing an HIV infection (e.g., HIV-1 and/or HIV-2) in a subject (e.g., a human) by administering a therapeutically effective amount of the compound of Formula I, or pharmaceutically acceptable salt thereof. In some embodiments, the compound of Formula I, or a pharmaceutically acceptable salt thereof, is administered as a monotherapy (i.e., in the absence of an additional therapeutic agent). In some embodiments, the compound of Formula I, or a pharmaceutically acceptable salt thereof, is administered in combination with one or more other therapeutic agents, such as anti-HIV agents. In some embodiments, the compound of Formula I, or a pharmaceutically acceptable salt thereof, and is administered as a long acting injectable. In some embodiments, the pharmaceutical compositions provided herein provide good tolerability and improved, manageable, minimal or no injection site reactions. In some embodiments, the pharmaceutical compositions provided herein provide enhanced plasma concentrations, pharmacokinetic profiles, and/or drug release profiles.

### L Definitions

Unless the context requires otherwise, throughout the present description and claims, the word “comprise” and variations thereof, such as, “comprises” and “comprising” are to be construed in an open, inclusive sense, that is as “including, but not limited to”.

Reference throughout this specification to “one embodiment” or “an embodiment” means that a particular feature, structure or characteristic described in connection with the embodiment is included in at least one embodiment described herein. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used herein, “crystalline form” is meant to refer to a certain lattice configuration of a crystalline substance (e.g., a salt or a cocrystal). Different crystalline forms of the same substance typically have different crystalline lattices (e.g., unit cells) which are attributed to different physical properties that are characteristic of each of the crystalline forms. In some instances, different lattice configurations have different water or solvent content giving rise to solvated or hydrated crystalline forms. The term “solvated,” as used herein, is meant to refer to a crystalline form that includes solvent molecules in the crystalline lattice. The term “hydrated,” as used herein, is meant to refer to a crystalline form that is solvated, where the solvent is water and water molecules are included in the crystalline lattice. Example “hydrated” crystalline forms include hemihydrates, monohydrates, dihydrates, and the like. Other hydrated forms such as channel hydrates and the like are also included within the meaning of the term. The term “fully hydrated” is meant to refer to where the water content of the hydrate is present in the expected stoichiometric amounts. The term “partially hydrated” is meant to refer to where the water content of the hydrate is present in less than the expected stoichiometric amounts (e.g., where some of the water of a monohydrate has been removed). Similarly, the term “unsolvated” or “anhydrous” refers to a crystalline form being substantially free of solvent or water, respectively, although some residual solvent or water may be present, for example, left over from the processes used to prepare the crystalline form.

The different crystalline forms can be identified by solid state characterization methods such as by X-ray powder diffraction (XRPD). Other characterization methods such as differential scanning calorimetry (DSC), thermogravimetric analysis (TGA), and dynamic vapor sorption (DVS) further help identify the form as well as help determine stability and solvent/water content.

An XRPD pattern of reflections (peaks) is typically considered a fingerprint of a particular crystalline form. Unless otherwise stated, XRPD patterns referred to herein were conducted on a diffractometer (PANanalytical XPERT-PRO, PANanalytical B.V., Almelo, Netherlands) using copper radiation (Cu Kα, λ=1.5418 Å). Samples were prepared for analysis by depositing the powdered sample in the center of an aluminum holder equipped with a zero background plate. The generator was operated at a voltage of 45 kV and amperage of 40 mA. Slits used were Soller 0.02 rad., antiscatter 1.0°, and divergence. The sample rotation speed was 2 sec. Scans were performed from 2 to 40° 2θ during 15 min with a step size of 0.0167° 2θ. Data analysis was performed by X'Pert Highscore version 2.2c (PANalytical B.V., Almelo, Netherlands) and X'Pert data viewer version 1.2d (PANalytical B.V., Almelo, Netherlands).

It is well known that the relative intensities of the XRPD peaks can widely vary depending on, inter alia, the sample preparation technique, crystal size distribution, various filters used, the sample mounting procedure, and the particular instrument employed. In some instances, new peaks may be observed or existing peaks may disappear, depending on the type of the instrument or the settings. As used herein, the term “peak” refers to a reflection having a relative height/intensity of at least about 5% of the maximum peak height/intensity. Moreover, instrument variation and other factors can affect the 2-theta values. Thus, peak assignments, such as those reported herein, can vary by plus or minus about 0.2° (2-theta), and the term “substantially” and “about” as used in the context of XRPD herein is meant to encompass the above-mentioned variations.

In the same way, temperature readings in connection with DSC can vary about ±3° C. depending on the instrument, particular settings, sample preparation, etc. Accordingly, a crystalline form reported herein having a DSC thermogram “substantially” as shown in any of the Figures or the term “about” is understood to accommodate such variation.

The d₉₀ values referred herein describe the size where ninety percent of particles in a sample have a smaller particle size than the specified d₉₀ value. For example, a d₉₀ of about 4 μm, means that 90% of the particles in the sample are smaller than 4 μm. Likewise, any d₅₀ values specified herein describe the size such that that 50% of particles in the sample are smaller than the specified d₅₀ value. Similarly, any d₁₀ values listed herein are the size at which 10% of the particles in the sample have a size smaller than this value.

“Pharmaceutically acceptable” refers to compounds, salts, compositions, dosage forms and other materials which are useful in preparing a pharmaceutical composition that is suitable for veterinary or human pharmaceutical use.

“Pharmaceutically acceptable excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

“Pharmaceutically acceptable salt” refers to a salt of a compound that is pharmaceutically acceptable and that possesses (or can be converted to a form that possesses) the desired pharmacological activity of the parent compound. Such salts include acid addition salts formed with inorganic acids, and salts formed when an acidic proton present in the parent compound is replaced by either a metal ion, e.g., an alkali metal ion, an alkaline earth ion, or an aluminum ion; or coordinates with an organic base such as diethanolamine, triethanolamine, N-methylglucamine and the like. Also included in this definition are ammonium and substituted or quaternized ammonium salts. Representative non-limiting lists of pharmaceutically acceptable salts can be found in S. M. Berge et al., J. Pharma Sci., 66(1), 1-19 (1977), and Remington: The Science and Practice of Pharmacy, R. Hendrickson, ed., 21st edition, Lippincott, Williams & Wilkins, Philadelphia, PA, (2005), at p. 732, Table 38-5, both of which are hereby incorporated by reference herein.

“Treatment” or “treating” is an approach for obtaining beneficial or desired results including clinical results. Beneficial or desired clinical results may include one or more of the following: a) inhibiting the disease or condition (e.g., decreasing one or more symptoms resulting from the disease or condition, and/or diminishing the extent of the disease or condition); b) slowing or arresting the development of one or more clinical symptoms associated with the disease or condition (e.g., stabilizing the disease or condition, preventing or delaying the worsening or progression of the disease or condition, and/or preventing or delaying the spread (e.g., metastasis) of the disease or condition); and/or c) relieving the disease, that is, causing the regression of clinical symptoms (e.g., ameliorating the disease state, providing partial or total remission of the disease or condition, enhancing effect of another medication, delaying the progression of the disease, increasing the quality of life, and/or prolonging survival).

As used herein, the terms “prevention” or “preventing” refers to the administration of a compound, composition, or pharmaceutically salt according to the present disclosure pre- or post-exposure of the human to the virus but before the appearance of symptoms of the disease, and/or prior to the detection of the virus in the blood. The terms also refer to prevention of the appearance of symptoms of the disease and/or to prevent the virus from reaching detectible levels in the blood. The term includes both pre-exposure prophylaxis (PrEP), as well as post-exposure prophylaxis (PEP) and event driven or “on demand” prophylaxis. The term also refers to prevention of perinatal transmission of HIV from mother to baby, by administration to the mother before giving birth and to the child within the first days of life. The term also refers to prevention of transmission of HIV through blood transfusion.

“Subject” refers to an animal, such as a mammal (e.g., a human), that has been or will be the object of treatment, observation or experiment. The methods described herein may be useful in human therapy and/or veterinary applications. In some embodiments, the subject is a mammal. In one embodiment, the subject is a human.

The term “therapeutically effective amount” or “effective amount” of a composition or a compound or pharmaceutically acceptable salts, isomer, or a mixture thereof, described herein means an amount sufficient to effect treatment when administered to a subject, to provide a therapeutic benefit such as amelioration of symptoms or slowing of disease progression. For example, a therapeutically effective amount may be an amount sufficient to decrease a symptom of a disease or condition responsive to HIV activity. The therapeutically effective amount may vary depending on the subject, and the disease or condition being treated, the weight and age of the subject, the severity of the disease or condition, and the manner of administering, which can readily be determined by one of ordinary skill in the art.

Reference to “about” a value or parameter herein includes (and describes) embodiments that are directed to that value or parameter per se. In certain embodiments, the term “about” includes the indicated amount ±10%. In other embodiments, the term “about” includes the indicated amount ±5%. In certain other embodiments, the term “about” includes the indicated amount ±1%. Also, the term “about X” includes description of “X”.

The invention herein is also meant to encompass all pharmaceutically acceptable salts and/or co-crystals of the compound of Formula I being isotopically-labeled by having one or more atoms replaced by an atom having a different atomic mass or mass number. Examples of isotopes that can be incorporated into the described compounds include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. These radiolabeled compounds could be useful to help determine or measure the effectiveness of the compounds, by characterizing, for example, the site or mode of action, or binding affinity to pharmacologically important site of action. Certain isotopically-labeled salts and/or co-crystals of tenofovir alafenamide, for example, those incorporating a radioactive isotope, are useful in drug and/or substrate tissue distribution studies. The radioactive isotopes tritium, i.e., ³H, and carbon-14, i.e., ⁴C, are particularly useful for this purpose in view of their ease of incorporation and ready means of detection.

Substitution with heavier isotopes such as deuterium, i.e., ²H, may afford certain therapeutic advantages resulting from greater metabolic stability. For example, in vivo half-life may increase or dosage requirements may be reduced. Thus, heavier isotopes may be preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy. Isotopically-labeled salts and/or co-crystals of the compound of Formula I can generally be prepared by conventional techniques known to those skilled in the art.

### I. Compound of Formula I

The methods and pharmaceutical compositions described herein utilize the compound of Formula I, wherein the compound of Formula I is in the form of a free acid and/or a pharmaceutically acceptable salt thereof. In some embodiments, the pharmaceutical composition comprises the compound of Formula I. In some embodiments, the pharmaceutical composition comprises a pharmaceutically acceptable salt of the compound of Formula I. The compound of Formula I, or pharmaceutically acceptable salt thereof, can be crystalline, amorphous, or a combination thereof. In some embodiments, the compound of Formula I, or pharmaceutically acceptable salt thereof, is crystalline. In some embodiments, the compound of Formula I, or pharmaceutically acceptable salt thereof, is amorphous. The compound of Formula I, or pharmaceutically acceptable salt thereof, may exhibit different properties in the solid form vs. in a solvent or solution as is used in the pharmaceutical compositions provided herein. For example, a pharmaceutical composition or formulation may be prepared with a salt of the compound of Formula I, however, depending on the composition or formulation, the salt may disproportionate to a free acid form.

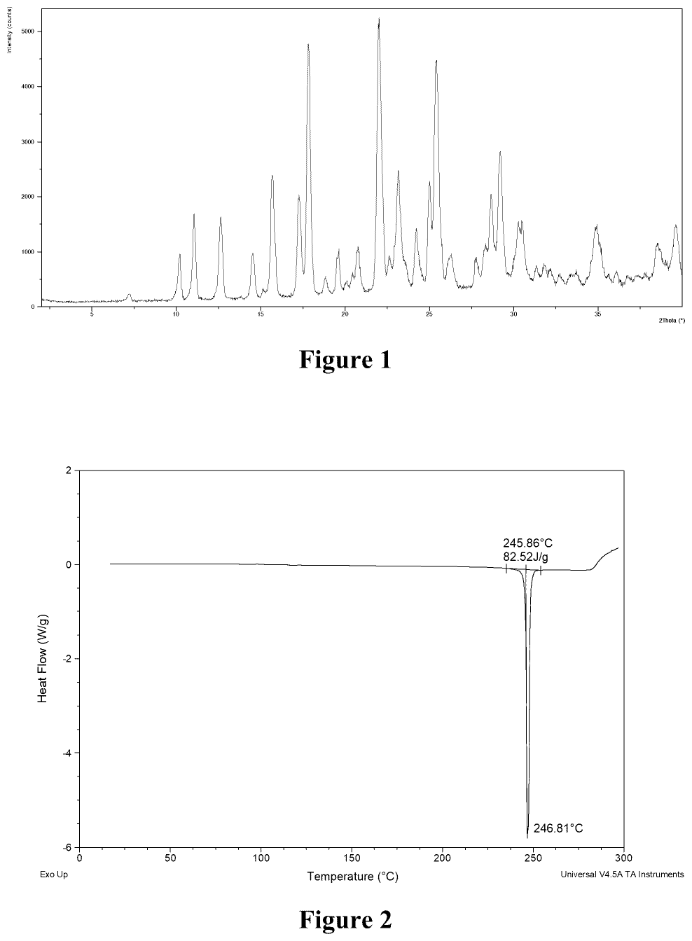

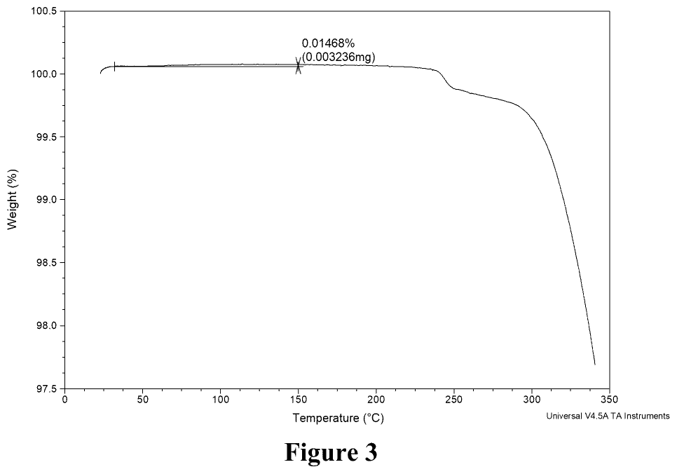

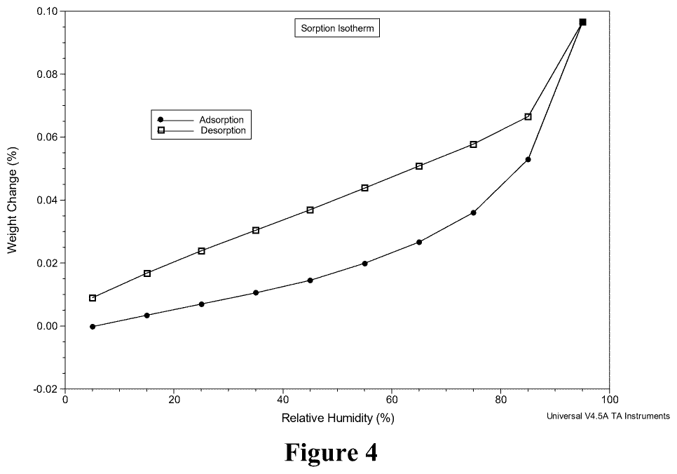

In some embodiments, the compound of Formula I is crystalline. In some embodiments, the compound of Formula I is crystalline Form I (Formula I, Form I), wherein the crystal structure exhibits an X-ray powder diffraction (XRPD) pattern substantially as shown in FIG. 1. Formula I, Form I may exhibit a differential scanning calorimetry (DSC) thermogram substantially as shown in FIG. 2. Formula I, Form I may exhibit a thermogravimetric analysis (TGA) graph substantially as shown in FIG. 3. Formula I, Form I may exhibit a dynamic vapor sorption (DVS) curve substantially as shown in FIG. 4.

The term “substantially as shown in” when referring, for example, to an XRPD pattern, a DSC thermogram, or a TGA graph includes a pattern, thermogram or graph that is not necessarily identical to those depicted herein, but that falls within the limits of experimental error or deviations when considered by one of ordinary skill in the art.

In some embodiments, Formula I, Form I has an XRPD pattern displaying at least two, at least three, at least four, at least five, or at least six of the degree 20-reflections with the greatest intensity as the XRPD pattern substantially as shown in FIG. 1.

In some embodiments, Formula I, Form I has an XRPD pattern comprising degree 20-reflections (+/−0.2 degrees 2θ) at 17.8°, 22.0°, and 25.4°. In some embodiments, Formula I, Form I has an XRPD pattern comprising degree 20-reflections (+/−0.2 degrees 2θ) at 17.8°, 22.0°, and 25.4°, and one, two or three of the degree 20-reflections (+/−0.2 degrees 2θ) at 15.6°, 23.10, and 29.2°. In some embodiments, Formula I, Form I has an XRPD pattern comprising degree 20-reflections (+/−0.2 degrees 2θ) at 17.8°, 22.0°, and 25.4°, and one or two of the degree 20-reflections (+/−0.2 degrees 2θ) at 15.6°, 23.1°, and 29.2°. In some embodiments, Formula I, Form I has an XRPD pattern comprising degree 20-reflections (+/−0.2 degrees 2θ) at 17.8°, 22.0°, and 25.4°, and one of the degree 20-reflections (+/−0.2 degrees 2θ) at 15.6°, 23.1°, and 29.2°±0.2°. In some embodiments, Formula I, Form I has an XRPD pattern comprising degree 20-reflections (+/−0.2 degrees 2θ) at 17.8°, 22.0°, and 25.4°, and two of the degree 20-reflections (+/−0.2 degrees 2θ) at 15.6°, 23.1°, and 29.2°. In some embodiments, Formula I, Form I has an XRPD pattern comprising degree 20-reflections (+/−0.2 degrees 2θ) at 15.6°, 17.8°, 22.0°, 23.1°, 25.4°, and 29.2°. In some embodiments, Formula I, Form I has an XRPD pattern comprising any three of the degree 20-reflections (+/−0.2 degrees 2θ) selected from the group consisting of 15.6°, 17.8°, 22.0°, 23.1°, 25.4°, and 29.2°.

In some embodiments, Formula I, Form I has an XRPD pattern comprising degree 20-reflections (+/−0.2 degrees 2θ) at 15.6°, 17.8°, 22.0°, 23.1°, 25.4°, and 29.2°, and one, two, or three of the degree 20-reflections (+/−0.2 degrees 2θ) at 11.1°, 12.6°, and 17.3°. In some embodiments, Formula I, Form I has an XRPD pattern comprising degree 2θ-reflections (+/−0.2 degrees 2θ) at 15.6°, 17.8°, 22.0°, 23.1°, 25.4°, and 29.2°, and one or two of the degree 2θ-reflections (+/−0.2 degrees 2θ) at 11.1°, 12.6°, and 17.3°. In some embodiments, Formula I, Form I has an XRPD pattern comprising degree 2θ-reflections (+/−0.2 degrees 2θ) at 15.6°, 17.8°, 22.0°, 23.1°, 25.4°, and 29.2°, and one of the degree 2θ-reflections (+/−0.2 degrees 2θ) at 11.1°, 12.6°, and 17.3°. In some embodiments, Formula I, Form I has an XRPD pattern comprising degree 2θ-reflections (+/−0.2 degrees 2θ) at 15.6°, 17.8°, 22.0°, 23.1°, 25.4°, and 29.2°, and two of the degree 2θ-reflections (+/−0.2 degrees 2θ) at 11.1°, 12.6°, and 17.3°. In some embodiments, Formula I, Form I has an XRPD pattern comprising degree 2θ-reflections (+/−0.2 degrees 2θ) at 11.1°, 12.6°, 15.6°, 17.3°, 17.8°, 22.0°, 23.1°, 25.4°, and 29.2°. In some embodiments, Formula I, Form I has an XRPD pattern comprising three of the degree 2θ-reflections (+/−0.2 degrees 2θ) at 11.1°, 12.6°, 15.6°, 17.3°, 17.8°, 22.0°, 23.1°, 25.4°, and 29.2°. In some embodiments, Formula I, Form I has an XRPD pattern comprising at least three of the degree 2θ-reflections (+/−0.2 degrees 2θ) at 10.2°, 11.1°, 12.6°, 14.5°, 15.6°, 17.3°, 17.8°, 22.0°, 23.1°, 25.4°, and 29.2°. In some embodiments, Formula I, Form I has an XRPD pattern comprising at least four of the degree 2θ-reflections (+/−0.2 degrees 2θ) at 10.2°, 11.1°, 12.6°, 14.5°, 15.6°, 17.3°, 17.8°, 22.0°, 23.1°, 25.4°, and 29.2°. In some embodiments, Formula I, Form I has an XRPD pattern comprising at least five of the degree 2θ-reflections (+/−0.2 degrees 2θ) at 10.2°, 11.1°, 12.6°, 14.5°, 15.6°, 17.3°, 17.8°, 22.0°, 23.1°, 25.4°, and 29.2°.

In some embodiments, Formula I, Form I has an XRPD pattern comprising peaks at:

| Pos. | |
|---|---|
| [°2Th. ± | Rel. Int. |
| 0.2] | [%] |
| 10.2051 | 17.91 |
| 11.0588 | 34.41 |
| 12.6118 | 31.59 |
| 14.5193 | 17.32 |
| 15.6463 | 41.62 |
| 17.2528 | 38.69 |
| 17.8322 | 100.00 |
| 19.6384 | 14.92 |
| 20.7623 | 16.29 |
| 21.9574 | 96.32 |
| 23.1489 | 44.24 |
| 24.2050 | 21.41 |
| 25.0185 | 40.07 |
| 25.3612 | 84.53 |
| 26.2356 | 11.77 |
| 27.7314 | 10.03 |
| 28.6533 | 34.12 |
| 29.1593 | 49.06 |
| 30.5358 | 20.39 |
| 34.8604 | 22.44 |
| 38.5013 | 12.61 |

In some embodiments, Formula I, Form I is unsolvated. In some embodiments, Formula I, Form I is anhydrous.

In some embodiments, Formula I, Form I, is characterized by a differential scanning calorimetry (DSC) curve comprising an endothermic transition with an onset at about 246° C.

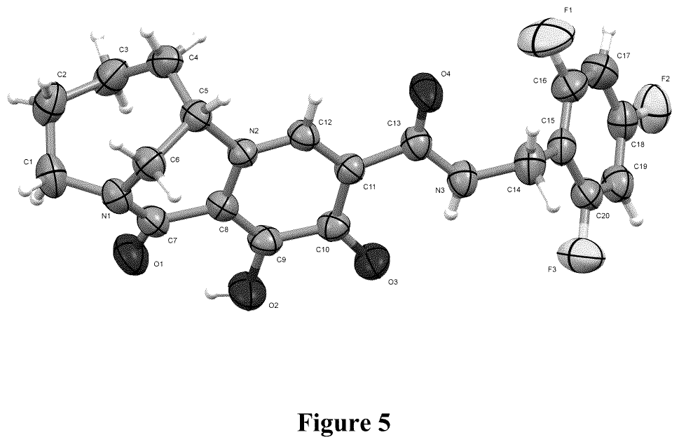

The single crystal data collected on Form I are summarized in Table 1 below and also shown in FIG. 5. The crystal system of Formula I, Form I is monoclinic and the space group is P2₁. The cell parameters and calculated volume are: a=8.94841(12) Å, b=8.58582(16) Å, c=12.59306(18) Å, α=90°, β=103.2526(13°), γ=90°, V=941.75(3) Å³. The molecular weight is 421.37 g mol⁻¹ with Z=2, resulting in a calculated density of 1.486 g cm⁻³. In some embodiments, Formula I, Form I has space group P2₁ having cell parameters a=8.94 Å, b=8.59 Å, c=12.59 Å, a=90°, β=103.3°, and γ=90°.

TABLE 1
Crystal Data and Data Collection Parameters for Formula I, Form I

| | |
|---|---|
| Empirical formula | C₂₀H₁₈F₃N₃O₄ |
| Formula weight (g mol⁻¹) | 421.37 |
| Temperature (K) | 300.1(2) |
| Wavelength (Å) | 1.54184 |
| Crystal system | monoclinic |
| Space group | P2₁ |
| Unit cell parameters | |
| a = 8.94841(12) Å | α = 90° |
| b = 8.58582(16) Å | β = 103.2526(13)° |
| c = 12.59306(18) Å | γ = 90° |
| Unit cell volume (Å³) | 941.75(3) |
| Cell formula units, Z | 2 |
| Calculated density (g cm⁻³) | 1.486 |
| Absorption coefficient (mm⁻¹) | 1.070 |
| F(000) | 436 |
| Crystal size (mm³) | 0.21 × 0.08 × 0.02 |
| Reflections used for cell measurement | 6168 |
| 0 range for cell measurement | 5.0740°-77.2490° |
| Total reflections collected | 9769 |
| Index ranges | −10 ≤ h ≤ 11; −10 ≤ k ≤ 9; −15 ≤ / ≤ 15 |
| θ range for data collection | θₘᵢₙ = 5.078°, θₘₐₓ = 77.513° |
| Completeness to θₘₐₓ | 97.8% |
| Completeness to θ_{full} = 67.684° | 99.8% |
| Absorption correction | multi-scan |
| Transmission coefficient range | 0.891-1.000 |
| Refinement method | full matrix least-squares on F² |
| Independent reflections | 3339 [Rᵢₙₜ = 0.0233, R_{σ} = 0.0230] |
| Reflections [I > 2σ(I)] | 3062 |
| Reflections/restraints/parameters | 3339/1/343 |
| Goodness-of-fit on F² | S = 1.05 |
| Final residuals [I > 2σ(I)] | R = 0.0303, R_{w} = 0.0811 |
| Final residuals [all reflections] | R = 0.0331, R_{w} = 0.0835 |
| Largest diff. peak and hole (e Å⁻³) | 0.104, −0.149 |
| Max/mean shift/standard uncertainty | 0.000/0.000 |
| Absolute structure determination | Flack |
| | parameter: 0.00(8) |
| | Hooft parameter: |
| | 0.03(7) |
| | Friedel coverage: 66.4% |

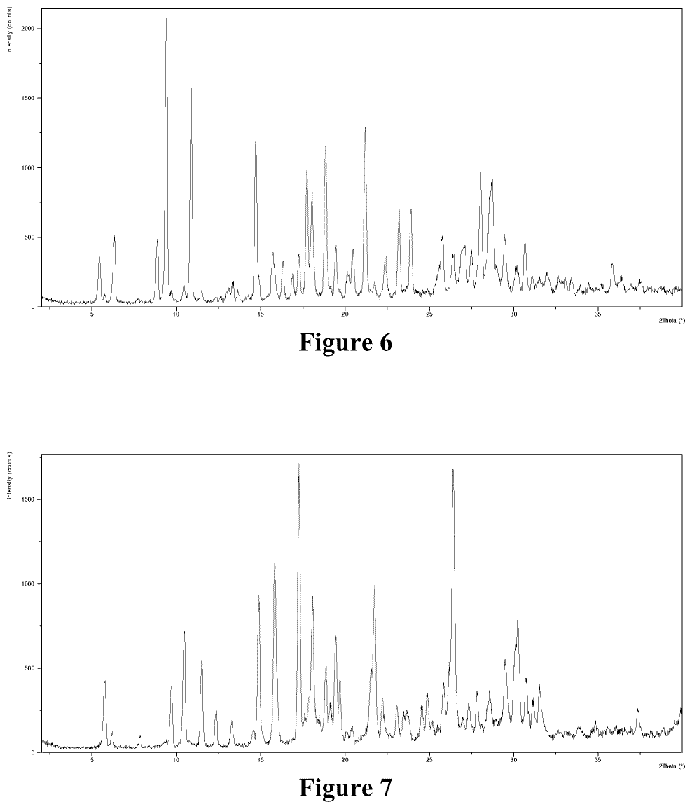

In some embodiments, the compound of Formula I is crystalline Form II (Formula I, Form II), wherein the crystal structure exhibits an X-ray powder diffraction (XRPD) pattern substantially as shown in FIG. 6.

In some embodiments, Formula I, Form II has an XRPD pattern displaying at least two, at least three, at least four, at least five, or at least six of the degree 2θ-reflections with the greatest intensity as the XRPD pattern substantially as shown in Figure. 6.

In some embodiments, Formula I, Form II has an XRPD pattern comprising degree 2θ-reflections (+/−0.2 degrees 2θ) at 9.5°, 10.9°, and 21.2°. In some embodiments, crystalline Formula I, Form II has an XRPD pattern comprising degree 2θ-reflections (+/−0.2 degrees 2θ) at 9.5°, 10.9°, and 21.2°, and one, two or three of the degree 2θ-reflections (+/−0.2 degrees 2θ) at 14.7°, 17.8°, and 18.9°. In some embodiments, crystalline Formula I, Form II has an XRPD pattern comprising degree 2θ-reflections (+/−0.2 degrees 2θ) at 9.5°, 10.9°, and 21.2°, and one or two of the degree 2θ-reflections (+/−0.2 degrees 2θ) at 14.7°, 17.8°, and 18.9°. In some embodiments, crystalline Formula I, Form II has an XRPD pattern comprising degree 2θ-reflections (+/−0.2 degrees 2θ) at 9.5°, 10.9°, and 21.2°, and one of the degree 2θ-reflections (+/−0.2 degrees 2θ) at 14.7°, 17.8°, and 18.9°. In some embodiments, crystalline Formula I, Form II has an XRPD pattern comprising degree 2θ-reflections (+/−0.2 degrees 2θ) at 9.5°, 10.9°, and 21.2°, and two of the degree 2θ-reflections (+/−0.2 degrees 2θ) at 14.7°, 17.8°, and 18.9°. In some embodiments, crystalline Formula I, Form II has an XRPD pattern comprising degree 2θ-reflections (+/−0.2 degrees 2θ) at 9.5°, 10.9°, 14.7°, 17.8°, 18.9°, and 21.2°. In some embodiments, crystalline Formula I, Form II has an XRPD pattern comprising any three of the degree 2θ-reflections (+/−0.2 degrees 2θ) selected from the group consisting of 9.5°, 10.9°, 14.7°, 17.8°, 18.9°, and 21.2°.

In some embodiments, crystalline Formula I, Form II has an XRPD pattern comprising 20-reflections (+/−0.2 degrees 2θ) at 9.5°, 10.9°, 14.7°, 17.8°, 18.9°, and 21.200, and one, two or three of the degree 2θ-reflections (+/−0.2 degrees 2θ) at 6.3°, 28.0°, and 28.7°. In some embodiments, crystalline Formula I, Form II has an XRPD pattern comprising 20-reflections (+/−0.2 degrees 2θ) at 9.5°, 10.9°, 14.7°, 17.8°, 18.9°, and 21.200, and one or two of the degree 2θ-reflections (+/−0.2 degrees 2θ) at 6.3°, 28.0°, and 28.7°. In some embodiments, crystalline Formula I, Form II has an XRPD pattern comprising 20-reflections (+/−0.2 degrees 2θ) at 9.5°, 10.9°, 14.7°, 17.8°, 18.9°, and 21.200, and one of the degree 2θ-reflections (+/−0.2 degrees 2θ) at 6.3°, 28.0°, and 28.7°. In some embodiments, crystalline Formula I, Form II has an XRPD pattern comprising 20-reflections (+/−0.2 degrees 2θ) at 9.5°, 10.9°, 14.7°, 17.8°, 18.9°, and 21.200, and two of the degree 2θ-reflections (+/−0.2 degrees 2θ) at 6.3°, 28.0°, and 28.7°. In some embodiments, crystalline Formula I, Form II has an XRPD pattern comprising any three of the degree 2θ-reflections (+/−0.2 degrees 2θ) selected from the group consisting of 6.3°, 9.5°, 10.9°, 14.7°, 17.8°, 18.9°, 21.2°, 28.0°, and 28.7°. In some embodiments, crystalline Formula I, Form II has an XRPD pattern comprising 20-reflections (+/−0.2 degrees 2θ) at 6.3°, 9.5°, 10.9°, 14.7°, 17.8°, 18.9°, 21.2°, 28.0°, and 28.7°. In some embodiments, crystalline Formula I, Form II has an XRPD pattern comprising at least three 20-reflections (+/−0.2 degrees 2θ) at 5.5, 6.3°, 8.9°, 9.5°, 10.9°, 14.7°, 15.7°, 16.3°, 17.3°, 17.8°, 18.0°, 18.9°, 19.5°, 21.2°, 28.0°, and 28.7°. In some embodiments, crystalline Formula I, Form II has an XRPD pattern comprising at least four 2θ-reflections (+/−0.2 degrees 2θ) at 5.5, 6.3°, 8.9°, 9.5°, 10.9°, 14.7°, 15.7°, 16.3°, 17.3°, 17.8°, 18.0°, 18.9°, 19.5°, 21.2°, 28.0°, and 28.7°. In some embodiments, crystalline Formula I, Form II has an XRPD pattern comprising at least five 2θ-reflections (+/−0.2 degrees 2θ) at 5.5, 6.3°, 8.9°, 9.5°, 10.9°, 14.7°, 15.7°, 16.3°, 17.3°, 17.8°, 18.0°, 18.9°, 19.5°, 21.2°, 28.0°, and 28.7°.

In some embodiments, Formula I, Form II has an XRPD pattern comprising peaks at:

| Pos. | |
|---|---|
| [°2Th. ± | Rel. Int. |
| 0.2] | [%] |
| 5.4610 | 17.91 |
| 6.3322 | 25.41 |
| 8.9092 | 23.95 |
| 9.4542 | 100.00 |
| 10.9298 | 69.05 |
| 13.2035 | 4.15 |
| 14.7383 | 62.27 |
| 15.6982 | 15.85 |
| 16.3292 | 15.33 |
| 17.2677 | 16.89 |
| 17.7594 | 48.53 |
| 18.0435 | 39.97 |
| 18.8815 | 55.12 |
| 19.4545 | 19.10 |
| 21.2189 | 64.10 |
| 23.1999 | 32.03 |
| 23.9297 | 32.13 |
| 25.7520 | 21.42 |
| 27.0226 | 14.90 |
| 27.4848 | 12.72 |
| 28.0375 | 43.04 |
| 28.6660 | 40.58 |
| 29.4663 | 20.64 |
| 30.1798 | 8.24 |
| 30.6728 | 18.29 |

In some embodiments, Formula I, Form II is solvated. In some embodiments, Formula I, Form II is partially or fully solvated. In some embodiments, Formula I, Form II is solvated by methanol.

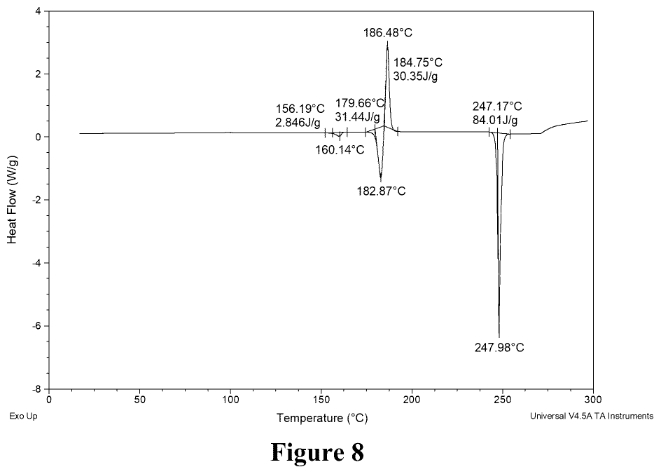

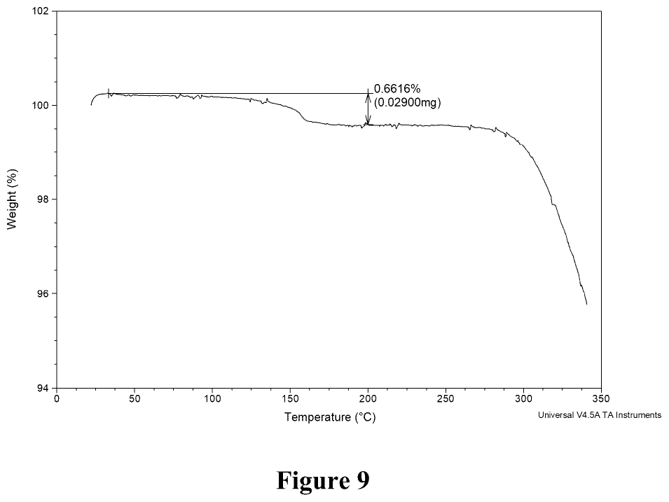

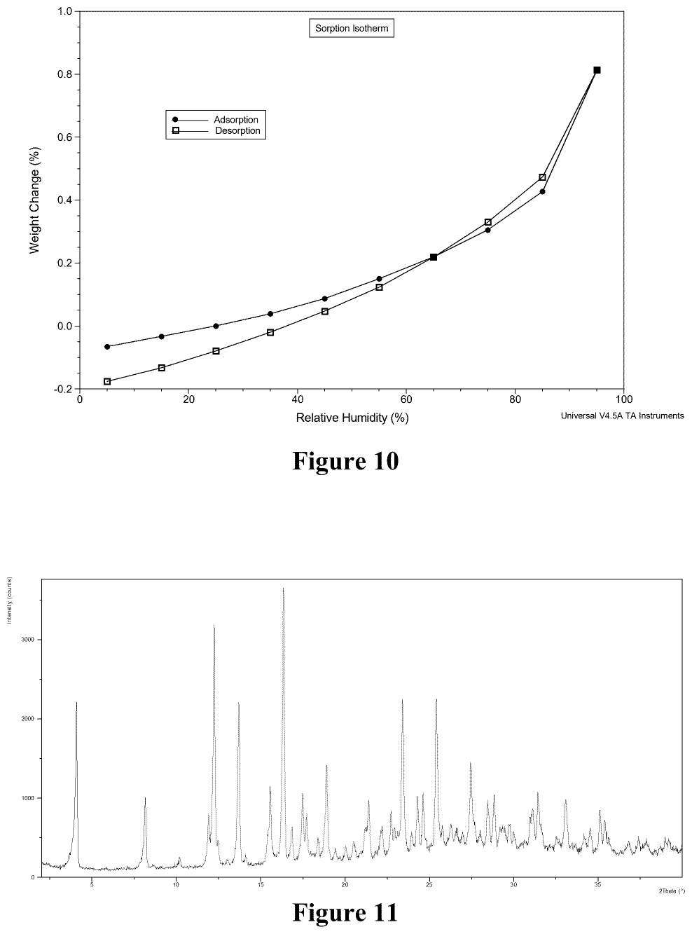

In some embodiments, the compound of Formula I is crystalline Form III (Formula I, Form III). In some embodiments, Formula I, Form III is solvated. In some embodiments, Formula I, Form III is methanol solvated. In some embodiments, Formula I, Form III exhibits an XRPD pattern substantially as shown in FIG. 7. In some embodiments, Formula I, Form III exhibits a DSC thermogram substantially as shown in FIG. 8. In some embodiments, Formula I, Form III exhibits a TGA curve substantially as shown in FIG. 9. In some embodiments, Formula I, Form III exhibits a DVS graph substantially as shown in FIG. 10.

In some embodiments, crystalline Formula I, Form III has an XRPD pattern displaying at least two, at least three, at least four, at least five, or at least six of the degree 2θ-reflections with the greatest intensity as the XRPD pattern substantially as shown in FIG. 7.

In some embodiments, crystalline Formula I, Form III has an XRPD pattern comprising degree 2θ-reflections (+/−0.2 degrees 2θ) at 15.8°, 17.3°, and 26.4°. In some embodiments, crystalline Formula I, Form III has an XRPD pattern comprising degree 2θ-reflections (+/−0.2 degrees 2θ) at 15.8°, 17.3°, and 26.4°, and one, two or three of the degree 2θ-reflections (+/−0.2 degrees 2θ) at 14.9°, 18.1°, and 21.8°. In some embodiments, crystalline Formula I, Form III has an XRPD pattern comprising degree 2θ-reflections (+/−0.2 degrees 2θ) at 15.8°, 17.3°, and 26.4°, and one or two of the degree 2θ-reflections (+/−0.2 degrees 2θ) at 14.9°, 18.1°, and 21.8°. In some embodiments, crystalline Formula I, Form III has an XRPD pattern comprising degree 2θ-reflections (+/−0.2 degrees 2θ) at 15.8°, 17.3°, and 26.4°, and one of the degree 2θ-reflections (+/−0.2 degrees 2θ) at 14.9°, 18.10, and 21.8°. In some embodiments, crystalline Formula I, Form III has an XRPD pattern comprising degree 2θ-reflections (+/−0.2 degrees 2θ) at 15.8°, 17.3°, and 26.4°, and two of the degree 2θ-reflections (+/−0.2 degrees 2θ) at 14.9°, 18.10, and 21.8°. In some embodiments, crystalline Formula I, Form III has an XRPD pattern comprising degree 2θ-reflections (+/−0.2 degrees 2θ) at 14.9°, 15.8°, 17.3°, 18.1°, 21.8°, and 26.4°. In some embodiments, crystalline Formula I, Form III has an XRPD pattern comprising any three of the degree 2θ-reflections (+/−0.2 degrees 2θ) at 14.9°, 15.8°, 17.3°, 18.1°, 21.8°, and 26.4°.

In some embodiments, crystalline Formula I, Form III has an XRPD pattern comprising degree 2θ-reflections (+/−0.2 degrees 2θ) at 14.9°, 15.8°, 17.3°, 18.1°, 21.8°, and 26.4°, and one, two or three of the degree 2θ-reflections (+/−0.2 degrees 2θ) at 5.8°, 9.7°, and 10.5°. In some embodiments, crystalline Formula I, Form III has an XRPD pattern comprising degree 2θ-reflections (+/−0.2 degrees 2θ) at 14.9°, 15.8°, 17.3°, 18.1°, 21.8°, and 26.4°, and one or two of the degree 2θ-reflections (+/−0.2 degrees 2θ) at 5.8°, 9.7°, and 10.5°. In some embodiments, crystalline Formula I, Form III has an XRPD pattern comprising degree 2θ-reflections (+/−0.2 degrees 2θ) at 14.9°, 15.8°, 17.3°, 18.1°, 21.8°, and 26.4°, and one of the degree 2θ-reflections (+/−0.2 degrees 2θ) at 5.8°, 9.7°, and 10.5°. In some embodiments, crystalline Formula I, Form III has an XRPD pattern comprising degree 2θ-reflections (+/−0.2 degrees 2θ) at 14.9°, 15.8°, 17.3°, 18.1°, 21.8°, and 26.4°, and two of the degree 2θ-reflections (+/−0.2 degrees 2θ) at 5.8°, 9.7°, and 10.5°. In some embodiments, crystalline Formula I, Form III has an XRPD pattern comprising degree 2θ-reflections (+/−0.2 degrees 2θ) at 5.8°, 9.7°, 10.5°, 14.9°, 15.8°, 17.3°, 18.1°, 21.8°, and 26.4°. In some embodiments, crystalline Formula I, Form III has an XRPD pattern comprising any three of the degree 2θ-reflections (+/−0.2 degrees 2θ) at 5.8°, 9.7°, 10.5°, 14.9°, 15.8°, 17.3°, 18.1°, 21.8°, and 26.4°. In some embodiments, Formula I, Form III has an XRPD pattern comprising at least three of the degree 2θ-reflections (+/−0.2 degrees 2θ) at 5.8°, 9.7°, 10.5°, 11.5°, 14.9°, 15.8°, 17.3°, 18.1°, 21.8°, 26.4°, and 30.2°. In some embodiments, Formula I, Form III has an XRPD pattern comprising at least four of the degree 2θ-reflections (+/−0.2 degrees 2θ) at 5.8°, 9.7°, 10.5°, 11.5°, 14.9°, 15.8°, 17.3°, 18.1°, 21.8°, 26.4°, and 30.2°. In some embodiments, Formula I, Form III has an XRPD pattern comprising at least five of the degree 2θ-reflections (+/−0.2 degrees 2θ) at 5.8°, 9.7°, 10.5°, 11.5°, 14.9°, 15.8°, 17.3°, 18.1°, 21.8°, 26.4°, and 30.2°.

In some embodiments, Formula I, Form III has an XRPD pattern comprising peaks at:

| Pos. | |
|---|---|
| [°2Th. ± | Rel. Int. |
| 0.2] | [%] |
| 5.7681 | 24.93 |
| 9.7205 | 22.56 |
| 10.5053 | 40.02 |
| 11.5440 | 29.19 |
| 14.9183 | 53.73 |
| 15.8074 | 64.25 |
| 17.2791 | 100.00 |
| 18.0835 | 51.41 |
| 19.4465 | 36.71 |
| 21.7763 | 55.41 |
| 23.7336 | 8.60 |
| 24.7317 | 5.43 |
| 26.3864 | 88.10 |
| 29.4755 | 23.36 |
| 30.2145 | 37.76 |
| 30.7309 | 18.01 |

In some embodiments, Formula I, Form III is characterized by a DSC thermogram substantially as shown in FIG. 8.

In some embodiments, Formula I, Form III is characterized by a DSC thermogram having one, two, three, or all of (i) an endothermic transition at about 156° C., (ii) an endothermic transition at about 180° C., (iii) an exothermic transition at about 186° C., and (iv) an endothermic transition at about 247° C.

In some embodiments, Formula I, Form III is characterized by a TGA curve substantially as shown in FIG. 9.

In some embodiments, Formula I, Form III is characterized by a DVS curve substantially as shown in shown in FIG. 10.

In some embodiments, Formula I, Form III is solvated. In some embodiments, Formula I, Form III is partially or fully solvated. In some embodiments, Formula I, Form III is solvated by methanol.

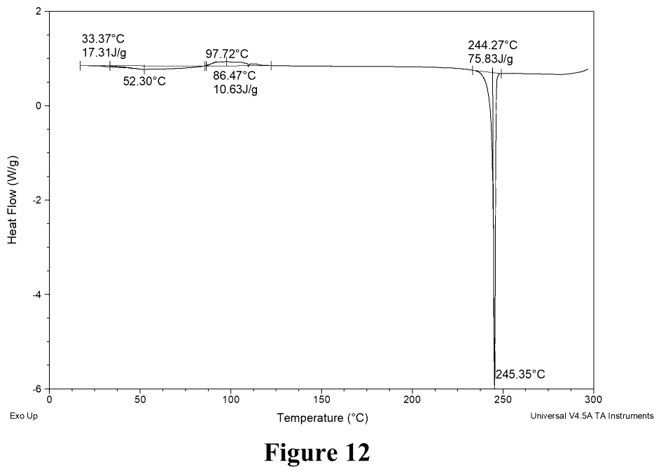

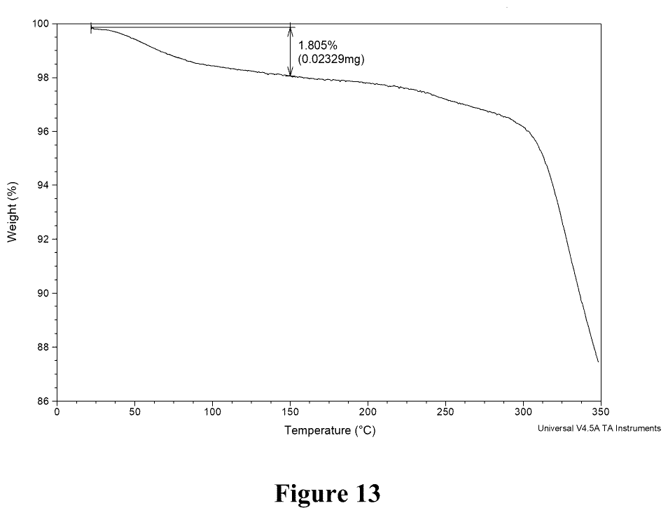

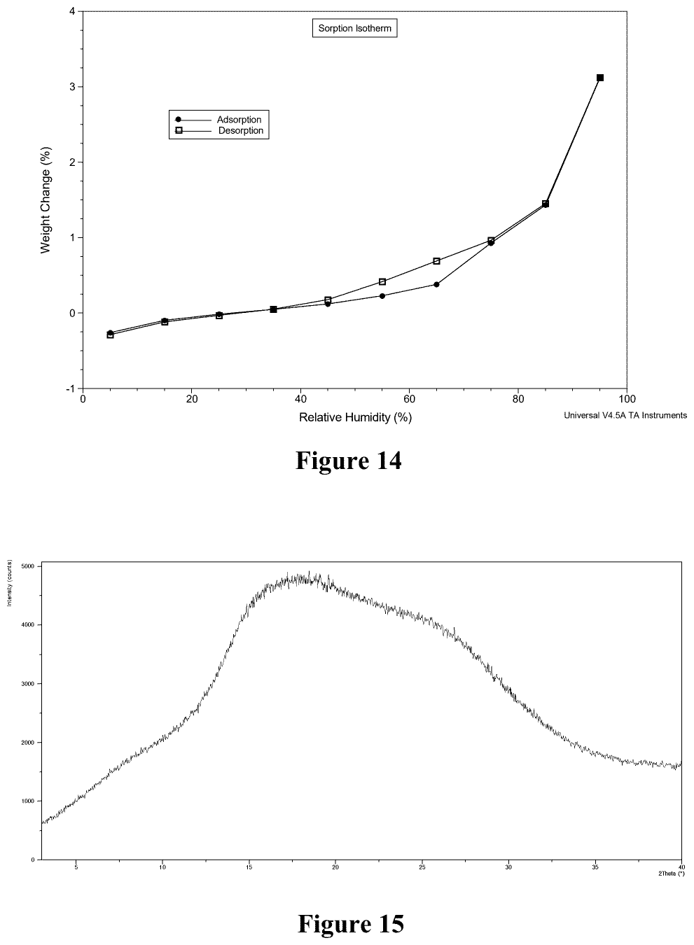

In some embodiments, the compound of Formula I is crystalline Form IV (Formula I, Form IV). In some embodiments, Formula I, Form IV is solvated. In some embodiments, Formula I, Form IV is hydrated. In some embodiments, Formula I, Form IV exhibits an XRPD pattern substantially as shown in FIG. 11. In some embodiments, Formula I, Form IV exhibits a DSC thermogram substantially as shown in FIG. 12. In some embodiments, Formula I, Form IV exhibits a TGA curve substantially as shown in FIG. 13. In some embodiments, Formula I, Form IV exhibits a DVS graph substantially as shown in FIG. 14.

In some embodiments, crystalline Formula I, Form IV has an XRPD pattern displaying at least two, at least three, at least four, at least five, or at least six of the degree 2θ-reflections with the greatest intensity as the XRPD pattern substantially as shown in FIG. 11.

In some embodiments, crystalline Formula I, Form IV has an XRPD pattern comprising degree 2θ-reflections (+/−0.2 degrees 2θ) at 4.1°, 12.3°, and 16.4°. In some embodiments, crystalline Formula I, Form IV has an XRPD pattern comprising degree 2θ-reflections (+/−0.2 degrees 2θ) at 4.1°, 12.3°, and 16.4°, and one, two or three of the degree 2θ-reflections (+/−0.2 degrees 2θ) at 13.7°, 23.4°, and 25.4°. In some embodiments, crystalline Formula I, Form IV has an XRPD pattern comprising degree 2θ-reflections (+/−0.2 degrees 2θ) at 4.1°, 12.3°, and 16.4°, and one or two of the degree 2θ-reflections (+/−0.2 degrees 2θ) at 13.7°, 23.4°, and 25.4°. In some embodiments, crystalline Formula I, Form IV has an XRPD pattern comprising degree 2θ-reflections (+/−0.2 degrees 2θ) at 4.1°, 12.3°, and 16.4°, and one of the degree 2θ-reflections (+/−0.2 degrees 2θ) at 13.7°, 23.4°, and 25.4°. In some embodiments, crystalline Formula I, Form IV has an XRPD pattern comprising degree 2θ-reflections (+/−0.2 degrees 2θ) at 4.1°, 12.3°, and 16.4°, and two of the degree 2θ-reflections (+/−0.2 degrees 2θ) at 13.7°, 23.4°, and 25.4°. In some embodiments, crystalline Formula I, Form IV has an XRPD pattern comprising degree 2θ-reflections (+/−0.2 degrees 2θ) at 4.1°, 12.3°, 13.7°, 16.4°, 23.4°, and 25.4°. In some embodiments, crystalline Formula I, Form IV has an XRPD pattern comprising any three of the degree 2θ-reflections (+/−0.2 degrees 2θ) at 4.1°, 12.3°, 13.7°, 16.4°, 23.4°, and 25.4°.

In some embodiments, crystalline Formula I, Form IV has an XRPD pattern comprising degree 2θ-reflections (+/−0.2 degrees 2θ) at 4.1°, 12.3°, 13.7°, 16.4°, 23.4°, and 25.4°, and one, two or three of the degree 2θ-reflections (+/−0.2 degrees 2θ) at 8.1°, 18.9°, and 27.4°. In some embodiments, crystalline Formula I, Form IV has an XRPD pattern comprising degree 2θ-reflections (+/−0.2 degrees 2θ) at 4.1°, 12.3°, 13.7°, 16.4°, 23.4°, and 25.4°, and one or two of the degree 2θ-reflections (+/−0.2 degrees 2θ) at 8.1°, 18.9°, and 27.4°. In some embodiments, crystalline Formula I, Form IV has an XRPD pattern comprising degree 2θ-reflections (+/−0.2 degrees 2θ) at 4.1°, 12.3°, 13.7°, 16.4°, 23.4°, and 25.4°, and one of the degree 2θ-reflections (+/−0.2 degrees 2θ) at 8.1°, 18.9°, and 27.4°. In some embodiments, crystalline Formula I, Form IV has an XRPD pattern comprising degree 2θ-reflections (+/−0.2 degrees 2θ) at 4.1°, 12.3°, 13.7°, 16.4°, 23.4°, and 25.4°, and two of the degree 2θ-reflections (+/−0.2 degrees 2θ) at 8.1°, 18.9°, and 27.4°. In some embodiments, crystalline Formula I, Form IV has an XRPD pattern comprising degree 2θ-reflections (+/−0.2 degrees 2θ) at 4.1°, 8.1°, 12.3°, 13.7°, 16.4°, 18.9°, 23.4°, 25.4°, and 27.4°. In some embodiments, crystalline Formula I, Form IV has an XRPD pattern comprising any three of the degree 2θ-reflections (+/−0.2 degrees 2θ) at 4.1°, 8.1°, 12.3°, 13.7°, 16.4°, 18.9°, 23.4°, 25.4°, and 27.4°. In some embodiments, Formula I, Form IV has an XRPD pattern comprising at least three of the degree 2θ-reflections (+/−0.2 degrees 2θ) at 4.10, 8.1°, 12.3°, 13.7°, 15.6°, 16.4°, 18.9°, 23.4°, 25.4°, and 27.4°. In some embodiments, Formula I, Form IV has an XRPD pattern comprising at least four of the degree 2θ-reflections (+/−0.2 degrees 2θ) at 4.1°, 8.1°, 12.3°, 13.7°, 15.6°, 16.4°, 18.9°, 23.4°, 25.4°, and 27.4°. In some embodiments, Formula I, Form IV has an XRPD pattern comprising at least five of the degree 2θ-reflections (+/−0.2 degrees 2θ) at 4.1°, 8.1°, 12.3°, 13.7°, 15.6°, 16.4°, 18.9°, 23.4°, 25.4°, and 27.4°.

In some embodiments, Formula I, Form IV has an XRPD pattern comprising peaks at:

| Pos. | |
|---|---|
| [°2Th. ± | Rel. Int. |
| 0.2] | [%] |
| 4.0905 | 61.21 |
| 8.1684 | 25.79 |
| 12.2546 | 85.48 |
| 13.7183 | 59.27 |
| 15.5882 | 25.92 |
| 16.3620 | 100.00 |
| 17.4922 | 23.39 |
| 18.4025 | 6.79 |
| 18.9112 | 34.07 |
| 21.3775 | 18.03 |
| 22.1915 | 9.88 |
| 23.3990 | 54.83 |
| 24.6191 | 18.60 |
| 25.4026 | 52.81 |
| 27.4476 | 29.32 |
| 28.4581 | 15.08 |
| 28.8361 | 16.20 |
| 31.4865 | 14.90 |
| 33.0815 | 17.71 |
| 35.1201 | 13.63 |

In some embodiments, Formula I, Form IV is characterized by a DSC thermogram substantially as shown in FIG. 12.

In some embodiments, Formula I, Form IV is characterized by a DSC thermogram having one, two or all of (i) an endothermic transition at about 33° C., (ii) an exothermic transition at about 86° C., and (iii) an endothermic transition at about 244° C.

In some embodiments, Formula I, Form IV is characterized by a TGA curve substantially as shown in FIG. 13.

In some embodiments, Formula I, Form IV is characterized by a DVS curve substantially as shown in shown in FIG. 14.

In some embodiments, Formula I, Form IV is hydrated. In some embodiments, Formula I, Form IV is partially or fully hydrated. In some embodiments, Formula I, Form IV is partially hydrated.

In some embodiments, the compound of Formula I is amorphous (Formula I, amorphous). In some embodiments, Formula I, amorphous has an XRPD profile substantially as shown in FIG. 15.

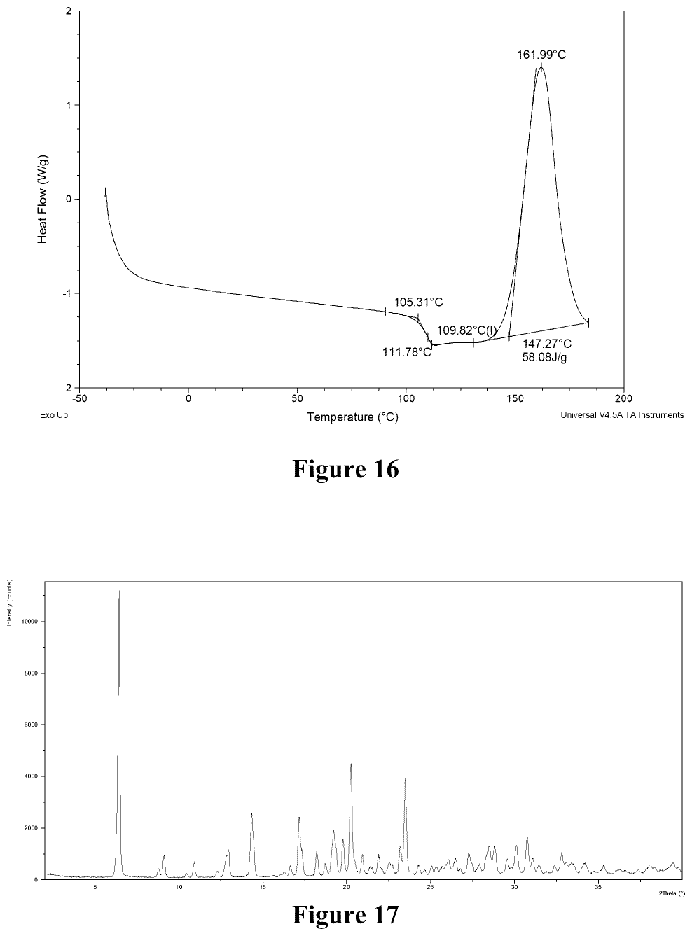

In some embodiments, Formula I, amorphous is characterized by a DSC thermogram substantially as shown in FIG. 16.

In some embodiments, Formula I, amorphous is characterized by a DSC thermogram having a glass transition about 110° C.

In some embodiments, the compound of Formula I, or a pharmaceutically acceptable salt thereof, is a sodium salt of the compound of Formula I. The sodium salt of the compound of Formula I can be amorphous or crystalline. In some embodiments, the sodium salt of the compound of Formula I is crystalline.

In some embodiments, the sodium salt of the compound of Formula I is the crystalline Form I (Formula I, sodium salt, Form I). In some embodiments, Formula I, sodium salt, Form I has an XRPD profile substantially as shown in FIG. 17.

In some embodiments, the Formula I, sodium salt, Form I has an XRPD pattern comprising degree 2θ-reflections (+/−0.2 degrees 2θ) at 6.5°, 20.2°, and 23.5°. In some embodiments, Formula I, sodium salt, Form I has an XRPD pattern comprising degree 2θ-reflections (+/−0.2 degrees 2θ) at 6.5°, 20.2°, and 23.5°, and one, two or three of the degree 2θ-reflections (+/−0.2 degrees 2θ) at 14.3°, 17.2°, and 19.2°. In some embodiments, Formula I, sodium salt, Form I has an XRPD pattern comprising degree 2θ-reflections (+/−0.2 degrees 2θ) at 6.5°, 20.2°, and 23.5°, and one or two of the degree 2θ-reflections (+/−0.2 degrees 2θ) at 14.3°, 17.2°, and 19.2°. In some embodiments, Formula I, sodium salt, Form I has an XRPD pattern comprising degree 2θ-reflections (+/−0.2 degrees 2θ) at 6.5°, 20.2°, and 23.5°, and one of the degree 2θ-reflections (+/−0.2 degrees 2θ) at 14.3°, 17.2°, and 19.2°. In some embodiments, Formula I, sodium salt, Form I has an XRPD pattern comprising degree 2θ-reflections (+/−0.2 degrees 2θ) at 6.5°, 20.2°, and 23.5°, and two of the degree 2θ-reflections (+/−0.2 degrees 2θ) at 14.3°, 17.2°, and 19.2°. In some embodiments, Formula I, sodium salt, Form I has an XRPD pattern comprising degree 2θ-reflections (+/−0.2 degrees 2θ) at 6.5°, 14.3°, 17.2°, 19.2°, 20.2°, and 23.5°. In some embodiments, Formula I, sodium salt, Form I has an XRPD pattern comprising any three of the degree 2θ-reflections (+/−0.2 degrees 2θ) at 6.5°, 14.3°, 17.2°, 19.2°, 20.2°, and 23.5°.

In some embodiments, Formula I, sodium salt, Form I has an XRPD pattern comprising degree 2θ-reflections (+/−0.2 degrees 2θ) at 6.5°, 14.3°, 17.2°, 19.2°, 20.2°, and 23.5°, and one, two or three of the degree 2θ-reflections (+/−0.2 degrees 2θ) at 13.0°, 19.8°, and 30.8°. In some embodiments, Formula I, sodium salt, Form I has an XRPD pattern comprising degree 2θ-reflections (+/−0.2 degrees 2θ) at 6.5°, 14.3°, 17.2°, 19.2°, 20.2°, and 23.5°, and one or two of the degree 2θ-reflections (+/−0.2 degrees 2θ) at 13.0°, 19.8°, and 30.8°. In some embodiments, Formula I, sodium salt, Form I has an XRPD pattern comprising degree 2θ-reflections (+/−0.2 degrees 2θ) at 6.5°, 14.3°, 17.2°, 19.2°, 20.2°, and 23.5°, and one of the degree 2θ-reflections (+/−0.2 degrees 2θ) at 13.0°, 19.8°, and 30.8°. In some embodiments, Formula I, sodium salt, Form I has an XRPD pattern comprising degree 2θ-reflections (+/−0.2 degrees 2θ) at 6.5°, 14.3°, 17.2°, 19.2°, 20.2°, and 23.5°, and two of the degree 2θ-reflections (+/−0.2 degrees 2θ) at 13.0°, 19.8°, and 30.8°. In some embodiments, Formula I, sodium salt, Form I has an XRPD pattern comprising degree 2θ-reflections (+/−0.2 degrees 2θ) at 6.5°, 13.0° 14.3°, 17.2°, 19.2°, 19.8°, 20.2°, 23.5°, and 30.8°. In some embodiments, Formula I, sodium salt, Form I has an XRPD pattern comprising any three of the degree 2θ-reflections (+/−0.2 degrees 2θ) at 6.5°, 13.0° 14.3°, 17.2°, 19.2°, 19.8°, 20.2°, 23.5°, and 30.8°.

In some embodiments, Formula I, sodium salt, Form I has an XRPD pattern comprising peaks at:

| Pos. | |
|---|---|
| [°2Th. ± | Rel. Int. |
| 0.2] | [%] |
| 6.4582 | 100.00 |
| 9.1357 | 7.98 |
| 10.9197 | 5.37 |
| 12.9859 | 9.48 |
| 14.3397 | 23.23 |
| 16.6524 | 3.41 |
| 17.1777 | 21.24 |
| 18.2116 | 8.54 |
| 18.7336 | 3.89 |
| 19.2149 | 15.37 |
| 19.7692 | 11.99 |
| 20.2415 | 39.53 |
| 20.9268 | 6.60 |
| 21.8961 | 6.32 |
| 22.5979 | 3.26 |
| 23.1770 | 9.59 |
| 23.4848 | 34.81 |
| 26.4924 | 4.82 |
| 27.2889 | 6.52 |
| 28.4243 | 7.38 |
| 28.8215 | 8.79 |
| 30.1002 | 9.22 |
| 30.7739 | 11.53 |
| 32.8031 | 6.89 |
| 34.1696 | 3.24 |

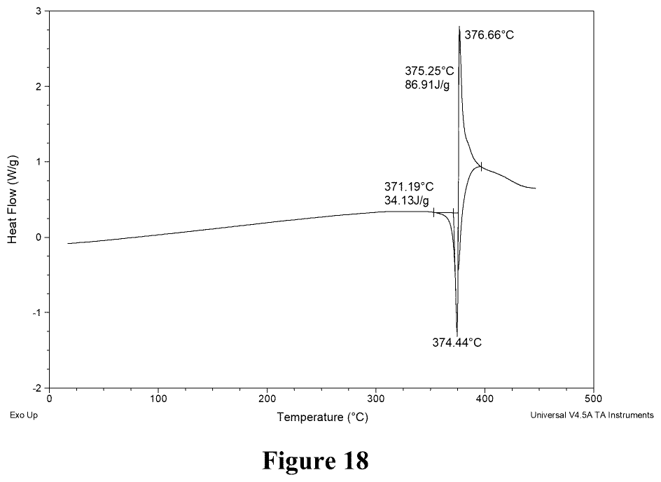

In some embodiments, Formula I, sodium salt, Form I is characterized by a DSC thermogram substantially as shown in FIG. 18.

In some embodiments, Formula I, sodium salt, Form I is characterized by a DSC thermogram having one or both of (i) an endothermic transition at about 371° C. and (ii) an exothermic transition at about 374° C.

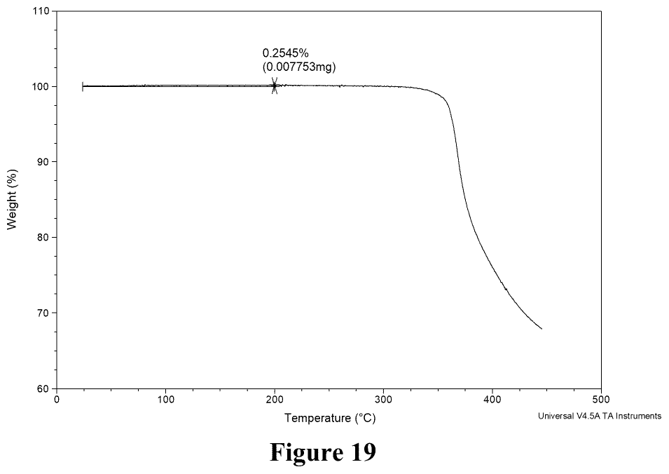

In some embodiments, Formula I, sodium salt, Form I is characterized by a TGA curve substantially as shown in FIG. 19.

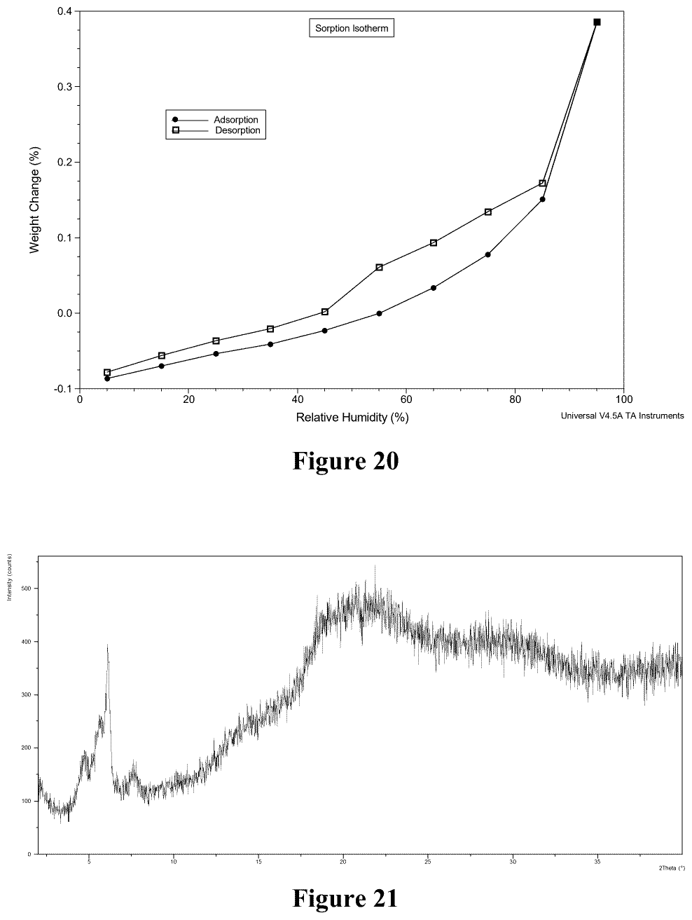

In some embodiments, Formula I, sodium salt, Form I is characterized by a DVS curve substantially as shown in shown in FIG. 20.

In some embodiments, Formula I, sodium salt, Form I is anhydrous. In some embodiments, the Formula I, sodium salt, Form I is unsolvated.

In some embodiments, the compound of Formula I, or a pharmaceutically acceptable salt thereof, is a calcium salt of the compound of Formula I. The calcium salt of the compound of Formula I can be in any morphological form such as, for example, crystalline or amorphous, as well as disordered crystals, liquid crystals, plastic crystals, mesophases, and the like, or any combination thereof. In some embodiments, the calcium salt of the compound of Formula I is crystalline. In some embodiments, the crystalline form of the calcium salt of the compound of Formula I is hydrated. In some embodiments, the calcium salt of the compound of Formula I is a hemi-calcium salt.

In some embodiments, the calcium salt of the compound of Formula I is the Form I (Formula I, calcium salt, Form I). In some embodiments, Formula I, calcium salt, Form I is hydrated. In some embodiments, Formula I, calcium salt, Form I has an XRPD profile substantially as shown in FIG. 21.

In some embodiments, Formula I, calcium salt, Form I has an XRPD pattern comprising degree 2θ-reflections (+/−0.2 degrees 2θ) at 4.8°, 5.6°, 6.1°, and 7.6°. In some embodiments, Formula I, calcium salt, Form I has an XRPD pattern comprising any three of the degree 2θ-reflections (+/−0.2 degrees 2θ) at 4.8°, 5.6°, 6.1°, and 7.6°.

In some embodiments, Formula I, calcium salt, Form I has an XRPD pattern comprising peaks at:

| Pos. | |
|---|---|
| [°2Th. ± | Rel. Int. |
| 0.2] | [%] |
| 4.759 | 32.23 |
| 5.5975 | 52.28 |
| 6.1208 | 100 |
| 7.6264 | 14.89 |

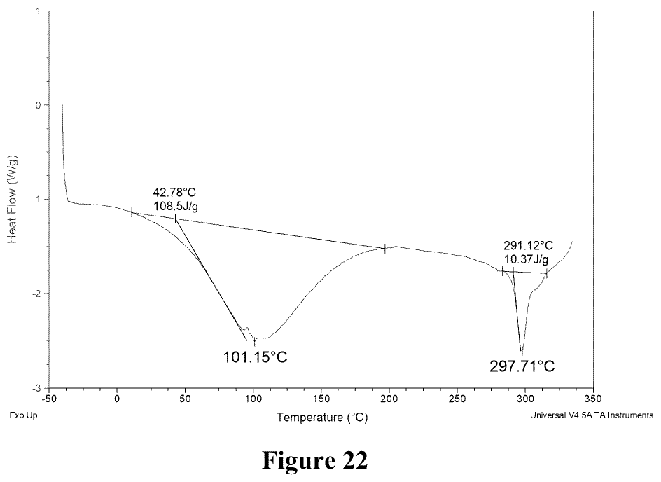

In some embodiments, Formula I, calcium salt, Form I is characterized by a DSC thermogram substantially as shown in FIG. 22.

In some embodiments, Formula I, calcium salt, Form I is characterized by a DSC thermogram having one or both of (i) an endothermic transition at about 43° C. and (ii) an endothermic transition at about 291° C. In some embodiments, Formula I, calcium salt, Form I is characterized by a DSC thermogram having an endothermic transition at about 43° C. and an endothermic transition at about 291° C.

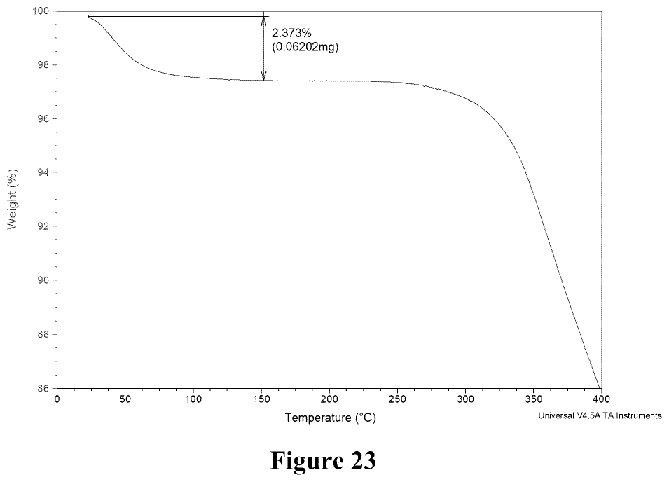

In some embodiments, Formula I, calcium salt, Form I is characterized by a TGA curve substantially as shown in FIG. 23.

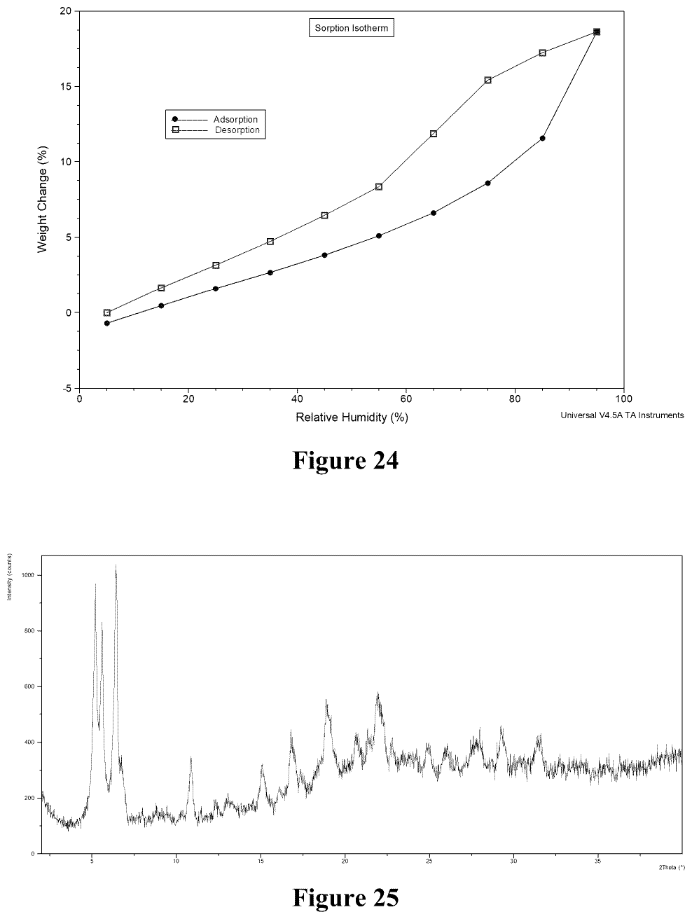

In some embodiments, Formula I, calcium salt, Form I is characterized by a DVS curve substantially as shown in shown in FIG. 24.

In some embodiments, Formula I, calcium salt, Form I is hydrated. In some embodiments, Formula I, calcium salt, Form I is partially or fully hydrated.

In some embodiments, Formula I, calcium salt, Form I is a hemi-calcium salt. In some embodiments, Formula I, calcium salt, Form I is present in a composition comprising a calcium salt of a halide, formate, acetate, triflate, or nitrate. In some embodiments, the calcium salt of a halide is a calcium salt of chloride, such as CaCl⁺. In some embodiments, Formula I, calcium salt, Form I comprises residual CaCl₂.

In some embodiments, Formula I, calcium salt, Form I is present in a composition that also comprises about 0.1 to about 3.0 wt. %, about 0.3 to about 2.5 wt. %, about 0.4 to about 2.0 wt. %, about 0.5 to about 1.0 wt. %, about 0.6 to about 0.9 wt. %, about 0.6 wt %, about 0.7 wt. %, about 0.8 wt %, or about 0.9 wt. % of chloride. In some embodiments, the chloride is in the form of a salt, such as a calcium salt like CaCl⁺ or CaCl₂).

In some embodiments, Formula I, calcium salt, Form I is present in a composition that also comprises the compound of Formula I in free form. In some embodiments, Formula I, calcium salt, Form I is present in a composition that also comprises chloride and the compound of Formula I in free form.

In some embodiments, the calcium salt of the compound of Formula I is the Form II (Formula I, calcium salt, Form II). In some embodiments, Formula I, calcium salt, Form II is hydrated. In some embodiments, Formula I, calcium salt, Form II has an XRPD profile substantially as shown in FIG. 25.

In some embodiments, Formula I, calcium salt, Form II has an XRPD pattern comprising degree 2θ-reflections (+/−0.2 degrees 2θ) at 5.2°, 5.6°, and 6.4°. In some embodiments, Formula I, calcium salt, Form II has an XRPD pattern comprising degree 2θ-reflections (+/−0.2 degrees 2θ) at 5.2°, 5.6°, and 6.4°, and one, two or three of the degree 2θ-reflections (+/−0.2 degrees 2θ) at 10.9°, 18.9°, and 21.9°. In some embodiments, Formula I, calcium salt, Form II has an XRPD pattern comprising degree 2θ-reflections (+/−0.2 degrees 2θ) at 5.2°, 5.6°, and 6.4°, and one or two of the degree 2θ-reflections (+/−0.2 degrees 2θ) at 10.9°, 18.9°, and 21.9°. In some embodiments, Formula I, calcium salt, Form II has an XRPD pattern comprising degree 2θ-reflections (+/−0.2 degrees 2θ) at 5.2°, 5.6°, and 6.4°, and one of the degree 2θ-reflections (+/−0.2 degrees 2θ) at 10.9°, 18.9°, and 21.9°. In some embodiments, Formula I, calcium salt, Form II has an XRPD pattern comprising degree 2θ-reflections (+/−0.2 degrees 2θ) at 5.2°, 5.6°, and 6.4°, and two of the degree 2θ-reflections (+/−0.2 degrees 2θ) at 10.9°, 18.9°, and 21.9°. In some embodiments, Formula I, calcium salt, Form II has an XRPD pattern comprising degree 2θ-reflections (+/−0.2 degrees 2θ) at 5.2°, 5.6°, 6.4°, 10.9°, 18.9°, and 21.9°. In some embodiments, Formula I, calcium salt, Form II has an XRPD pattern comprising any three of the degree 2θ-reflections (+/−0.2 degrees 2θ) at 5.2°, 5.6°, 6.4°, 10.9°, 18.9°, and 21.9°.

In some embodiments, Formula I, calcium salt, Form II has an XRPD pattern comprising degree 2θ-reflections (+/−0.2 degrees 2θ) at 5.2°, 5.6°, 6.4°, 10.9°, 18.9°, and 21.9°, and one, two or three of the degree 2θ-reflections (+/−0.2 degrees 2θ) at 15.1°, 16.8°, and 29.3°. In some embodiments, Formula I, calcium salt, Form II has an XRPD pattern comprising degree 2θ-reflections (+/−0.2 degrees 2θ) at 5.2°, 5.6°, 6.4°, 10.9°, 18.9°, and 21.9°, and one or two of the degree 2θ-reflections (+/−0.2 degrees 2θ) at 15.1°, 16.8°, and 29.3°. In some embodiments, Formula I, calcium salt, Form II has an XRPD pattern comprising degree 2θ-reflections (+/−0.2 degrees 2θ) at 5.2°, 5.6°, 6.4°, 10.9°, 18.9°, and 21.9°, and one of the degree 2θ-reflections (+/−0.2 degrees 2θ) at 15.1°, 16.8°, and 29.3°. In some embodiments, Formula I, calcium salt, Form II has an XRPD pattern comprising degree 2θ-reflections (+/−0.2 degrees 2θ) at 5.2°, 5.6°, 6.4°, 10.9°, 18.9°, and 21.9°, and two of the degree 2θ-reflections (+/−0.2 degrees 2θ) at 15.1°, 16.8°, and 29.3°. In some embodiments, Formula I, calcium salt, Form II has an XRPD pattern comprising degree 2θ-reflections (+/−0.2 degrees 2θ) at 5.2°, 5.6°, 6.4°, 10.9°, 15.1°, 16.8°, 18.9°, 21.9°, and 29.3°. In some embodiments, Formula I, calcium salt, Form II has an XRPD pattern comprising any three of the degree 2θ-reflections (+/−0.2 degrees 2θ) at 5.2°, 5.6°, 6.4°, 10.9°, 15.1°, 16.8°, 18.9°, 21.9°, and 29.3°.

In some embodiments, Formula I, calcium salt, Form II has an XRPD pattern comprising peaks at:

| Pos. | |
|---|---|
| [°2Th. ± | Rel. Int. |
| 0.2] | [%] |
| 5.2027 | 94.25 |
| 5.6215 | 83.25 |
| 6.4645 | 100 |
| 10.8889 | 25.26 |
| 15.0964 | 15.92 |
| 16.8257 | 23.38 |
| 18.9083 | 29.87 |
| 21.9068 | 27.32 |
| 24.9566 | 6.72 |
| 25.9705 | 6.49 |
| 27.8323 | 9.87 |
| 29.2844 | 15.82 |
| 31.4693 | 12.42 |

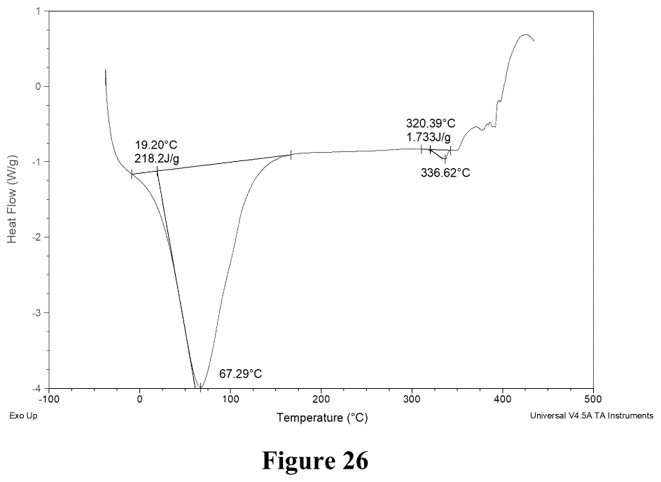

In some embodiments, Formula I, calcium salt, Form II is characterized by a DSC thermogram substantially as shown in FIG. 26.

In some embodiments, Formula I, calcium salt, Form II is characterized by a DSC thermogram having one or both of (i) displays an endothermic transition at about 19° C. and an endothermic transition at about 320° C.

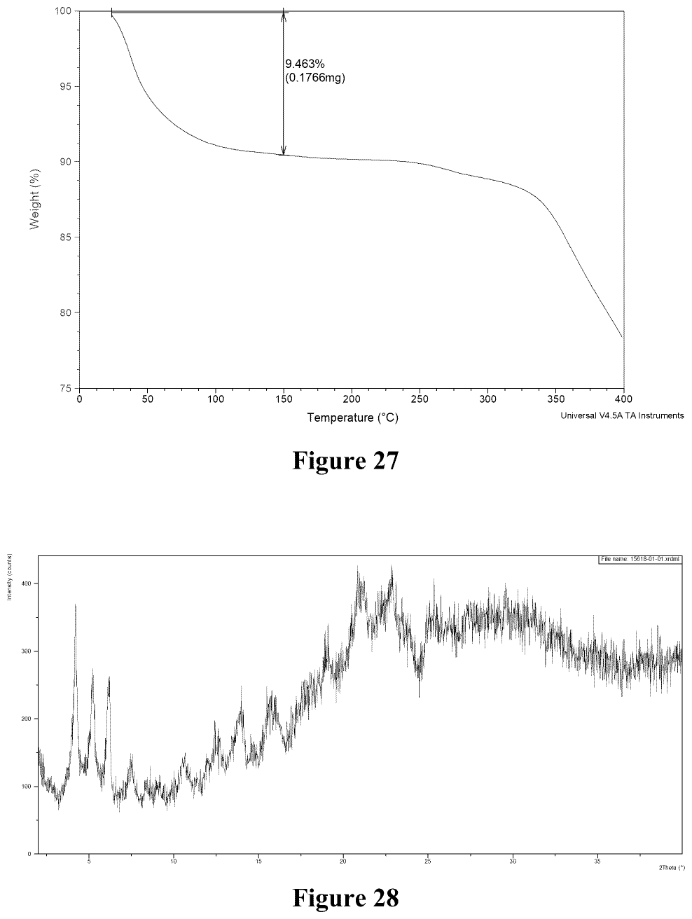

In some embodiments, Formula I, calcium salt, Form II is characterized by a TGA curve substantially as shown in FIG. 27.

In some embodiments, Formula I, calcium salt, Form II is hydrated. In some embodiments, Formula I, calcium salt, Form II is partially or fully hydrated. In some embodiments, Formula I, calcium salt, Form II is fully hydrated.

In some embodiments, the calcium salt of the compound of Formula I is the Form III (Formula I, calcium salt, Form III). In some embodiments, Formula I, calcium salt, Form III is hydrated. In some embodiments, Formula I, calcium salt, Form III has an XRPD profile substantially as shown in FIG. 28.

In some embodiments, Formula I, calcium salt, Form III has an XRPD pattern comprising degree 2θ-reflections (+/−0.2 degrees 2θ) at 4.2°, 5.2°, and 6.2°. In some embodiments, Formula I, calcium salt, Form III has an XRPD pattern comprising degree 2θ-reflections (+/−0.2 degrees 2θ) at 4.2°, 5.2°, and 6.2°, and one, two or three of the degree 2θ-reflections (+/−0.2 degrees 2θ) at 13.9°, 15.7°, and 21.0°. In some embodiments, Formula I, calcium salt, Form III has an XRPD pattern comprising degree 2θ-reflections (+/−0.2 degrees 2θ) at 4.2°, 5.2°, and 6.2°, and one or two of the degree 2θ-reflections (+/−0.2 degrees 2θ) at 13.9°, 15.7°, and 21.0°. In some embodiments, Formula I, calcium salt, Form III has an XRPD pattern comprising degree 2θ-reflections (+/−0.2 degrees 2θ) at 4.2°, 5.2°, and 6.2°, and one of the degree 2θ-reflections (+/−0.2 degrees 2θ) at 13.9°, 15.7°, and 21.0°. In some embodiments, Formula I, calcium salt, Form III has an XRPD pattern comprising degree 2θ-reflections (+/−0.2 degrees 2θ) at 4.2°, 5.2°, and 6.2°, and two of the degree 2θ-reflections (+/−0.2 degrees 2θ) at 13.9°, 15.7°, and 21.0°. In some embodiments, Formula I, calcium salt, Form III has an XRPD pattern comprising degree 2θ-reflections (+/−0.2 degrees 2θ) at 4.2°, 5.2°, 6.2°, 13.9°, 15.7°, and 21.0°. In some embodiments, Formula I, calcium salt, Form III has an XRPD pattern comprising any three of the degree 2θ-reflections (+/−0.2 degrees 2θ) at 4.2°, 5.2°, 6.2°, 13.9°, 15.7°, and 21.0°.

In some embodiments, Formula I, calcium salt, Form III has an XRPD pattern comprising degree 2θ-reflections (+/−0.2 degrees 2θ) at 4.2°, 5.2°, 6.2°, 13.9°, 15.7°, and 21.0°, and one, two or three of the degree 2θ-reflections (+/−0.2 degrees 2θ) at 7.5°, 10.6°, and 12.6°.

In some embodiments, Formula I, calcium salt, Form III has an XRPD pattern comprising degree 2θ-reflections (+/−0.2 degrees 2θ) at 4.2°, 5.2°, 6.2°, 13.9°, 15.7°, and 21.0°, and one or two of the degree 2θ-reflections (+/−0.2 degrees 2θ) at 7.5°, 10.6°, and 12.6°. In some embodiments, Formula I, calcium salt, Form III has an XRPD pattern comprising degree 2θ-reflections (+/−0.2 degrees 2θ) at 4.2°, 5.2°, 6.2°, 13.9°, 15.7°, and 21.0°, and one of the degree 2θ-reflections (+/−0.2 degrees 2θ) at 7.5°, 10.6°, and 12.6°. In some embodiments, Formula I, calcium salt, Form III has an XRPD pattern comprising degree 2θ-reflections (+/−0.2 degrees 2θ) at 4.2°, 5.2°, 6.2°, 13.9°, 15.7°, and 21.0°, and two of the degree 2θ-reflections (+/−0.2 degrees 2θ) at 7.5°, 10.6°, and 12.6°. In some embodiments, Formula I, calcium salt, Form III has an XRPD pattern comprising degree 2θ-reflections (+/−0.2 degrees 2θ) at 4.2°, 5.2°, 6.2°, 7.5°, 10.6°, 12.6°, 13.9°, 15.7°, and 21.0°. In some embodiments, Formula I, calcium salt, Form III has an XRPD pattern comprising any three of the degree 2θ-reflections (+/−0.2 degrees 2θ) at 4.2°, 5.2°, 6.2°, 7.5°, 10.6°, 12.6°, 13.9°, 15.7°, and 21.0°.

In some embodiments, Formula I, calcium salt, Form III has an XRPD pattern comprising peaks at:

| Pos. | |
|---|---|
| [°2Th. ± | Rel. Int. |
| 0.2] | [%] |
| 4.2027 | 100 |
| 5.2494 | 64.58 |
| 6.1747 | 62.82 |
| 7.4813 | 15.12 |
| 10.6013 | 12.69 |
| 12.5542 | 17.46 |
| 13.9203 | 27.71 |
| 15.6797 | 19.8 |
| 21.0148 | 25.12 |

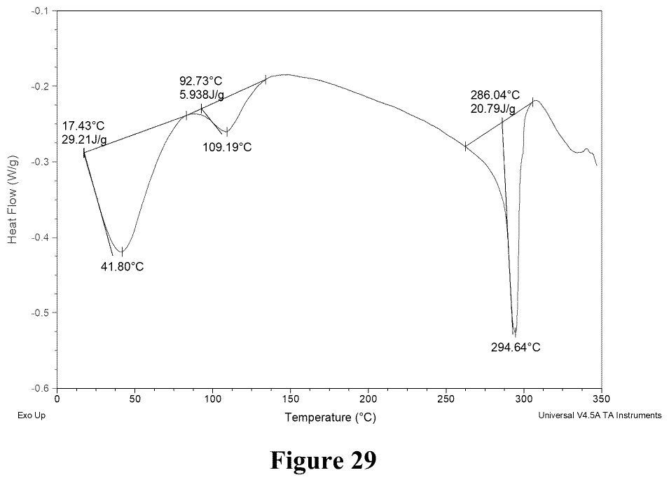

In some embodiments, Formula I, calcium salt, Form III is characterized by a DSC thermogram substantially as shown in FIG. 29.

In some embodiments, Formula I, calcium salt, Form III is characterized by a DSC thermogram having one, two, or three of (i) displays an endothermic transition at about 17° C., (ii) an endothermic transition at about 93° C., and (iii) an endothermic transition at about 286° C.

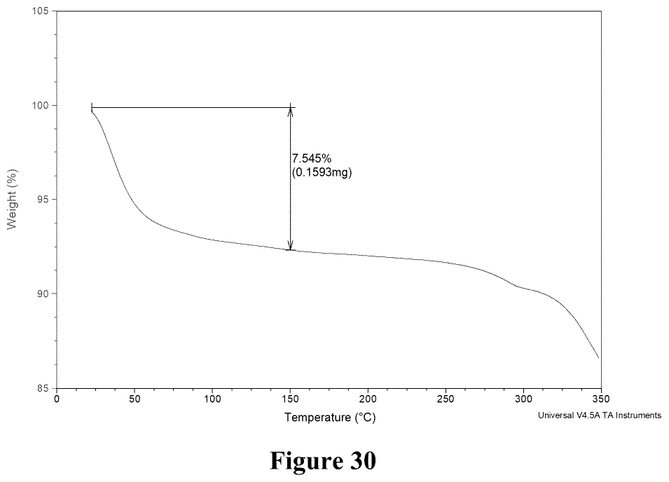

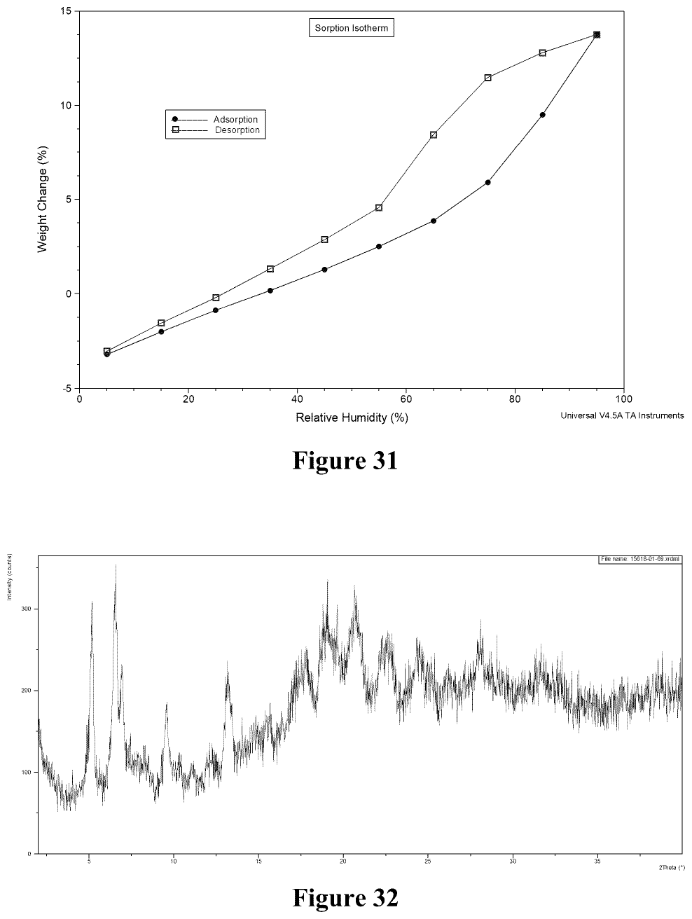

In some embodiments, Formula I, calcium salt, Form III is characterized by a TGA curve substantially as shown in FIG. 30.

In some embodiments, Formula I, calcium salt, Form III is characterized by a DVS curve substantially as shown in FIG. 31.

In some embodiments, Formula I, calcium salt, Form III is hydrated. In some embodiments, Formula I, calcium salt, Form III is partially or fully hydrated.

In some embodiments, the crystalline calcium salt of the compound of Formula I is the Form IV (Formula I, calcium salt, Form IV). In some embodiments, Formula I, calcium salt, Form IV is hydrated. In some embodiments, Formula I, calcium salt, Form IV has an XRPD profile substantially as shown in FIG. 32.

In some embodiments, Formula I, calcium salt, Form IV has an XRPD pattern comprising degree 2θ-reflections (+/−0.2 degrees 2θ) at 5.2°, 6.5°, and 20.7°. In some embodiments, Formula I, calcium salt, Form IV has an XRPD pattern comprising degree 2θ-reflections (+/−0.2 degrees 2θ) at 5.2°, 6.5°, and 20.7°, and one, two or three of the degree 2θ-reflections (+/−0.2 degrees 2θ) at 7.0°, 13.2°, and 18.9°. In some embodiments, Formula I, calcium salt, Form IV has an XRPD pattern comprising degree 2θ-reflections (+/−0.2 degrees 2θ) at 5.2°, 6.5°, and 20.7°, and one or two of the degree 2θ-reflections (+/−0.2 degrees 2θ) at 7.0°, 13.2°, and 18.9°. In some embodiments, Formula I, calcium salt, Form IV has an XRPD pattern comprising degree 2θ-reflections (+/−0.2 degrees 2θ) at 5.2°, 6.5°, and 20.7°, and one of the degree 2θ-reflections (+/−0.2 degrees 2θ) at 7.0°, 13.2°, and 18.9°. In some embodiments, Formula I, calcium salt, Form IV has an XRPD pattern comprising degree 2θ-reflections (+/−0.2 degrees 2θ) at 5.2°, 6.5°, and 20.7°, and two of the degree 2θ-reflections (+/−0.2 degrees 2θ) at 7.0°, 13.2°, and 18.9°. In some embodiments, Formula I, calcium salt, Form IV has an XRPD pattern comprising degree 2θ-reflections (+/−0.2 degrees 2θ) at 5.2°, 6.5°, 7.0°, 13.2°, 18.9°, and 20.7°. In some embodiments, Formula I, calcium salt, Form IV has an XRPD pattern comprising any three of the degree 2θ-reflections (+/−0.2 degrees 2θ) at 5.2°, 6.5°, 7.0°, 13.2°, 18.9°, and 20.7°.

In some embodiments, Formula I, calcium salt, Form IV has an XRPD pattern comprising degree 2θ-reflections (+/−0.2 degrees 2θ) at 5.2°, 6.5°, 7.0°, 13.2°, 18.9°, and 20.7°, and one, two or three of the degree 2θ-reflections (+/−0.2 degrees 2θ) at 9.6°, 17.8°, and 22.9°.

In some embodiments, Formula I, calcium salt, Form IV has an XRPD pattern comprising degree 2θ-reflections (+/−0.2 degrees 2θ) at 5.2°, 6.5°, 7.0°, 13.2°, 18.9°, and 20.7, and one or two of the degree 2θ-reflections (+/−0.2 degrees 2θ) at 9.6°, 17.8°, and 22.9°. In some embodiments, Formula I, calcium salt, Form IV has an XRPD pattern comprising degree 2θ-reflections (+/−0.2 degrees 2θ) at 5.2°, 6.5°, 7.0°, 13.2°, 18.9°, and 20.7°, and one of the degree 2θ-reflections (+/−0.2 degrees 2θ) at 9.6°, 17.8°, and 22.9°. In some embodiments, Formula I, calcium salt, Form IV has an XRPD pattern comprising degree 2θ-reflections (+/−0.2 degrees 2θ) at 5.2°, 6.5°, 7.0°, 13.2°, 18.9°, and 20.7°, and two of the degree 2θ-reflections (+/−0.2 degrees 2θ) at 9.6°, 17.8°, and 22.9°. In some embodiments, Formula I, calcium salt, Form IV has an XRPD pattern comprising degree 2θ-reflections (+/−0.2 degrees 2θ) 5.2°, 6.5°, 7.0°, 9.6°, 13.2°, 17.8°, 18.9°, 20.7°, and 22.9°. In some embodiments, Formula I, calcium salt, Form IV has an XRPD pattern comprising any three of the degree 2θ-reflections (+/−0.2 degrees 2θ) at 5.2°, 6.5°, 7.0°, 9.6°, 13.2°, 17.8°, 18.9°, 20.7°, and 22.9°. In some embodiments, Formula I, calcium salt, Form IV has an XRPD pattern comprising at least three of the degree 2θ-reflections (+/−0.2 degrees 2θ) at 5.2°, 6.5°, 7.0°, 8.4°, 9.6°, 13.2°, 14.7°, 17.8°, 18.9°, 20.7°, and 22.9°. In some embodiments, Formula I, calcium salt, Form IV has an XRPD pattern comprising at least four of the degree 2θ-reflections (+/−0.2 degrees 2θ) at 5.2°, 6.5°, 7.0°, 8.4°, 9.6°, 13.2°, 14.7°, 17.8°, 18.9°, 20.7°, and 22.9°. In some embodiments, Formula I, calcium salt, Form IV has an XRPD pattern comprising at least five of the degree 2θ-reflections (+/−0.2 degrees 2θ) at 5.2°, 6.5°, 7.0°, 8.4°, 9.6°, 13.2°, 14.7°, 17.8°, 18.9°, 20.7°, and 22.9°.In some embodiments, Formula I, calcium salt, Form IV has an XRPD pattern comprising peaks at:

| Pos. | |
|---|---|
| [°2Th. ± | Rel. Int. |
| 0.2] | [%] |
| 5.1906 | 97.5 |
| 6.5315 | 100 |
| 6.9516 | 63.68 |
| 8.4207 | 11.94 |
| 9.5864 | 38.78 |
| 12.1583 | 7.54 |
| 13.1534 | 44.74 |
| 14.7177 | 14.96 |
| 15.6964 | 20.69 |
| 17.8258 | 43.97 |
| 18.895 | 56.04 |
| 20.7192 | 65.87 |
| 22.8522 | 33.95 |
| 24.3624 | 29.15 |
| 26.1189 | 11.88 |
| 28.138 | 24.29 |

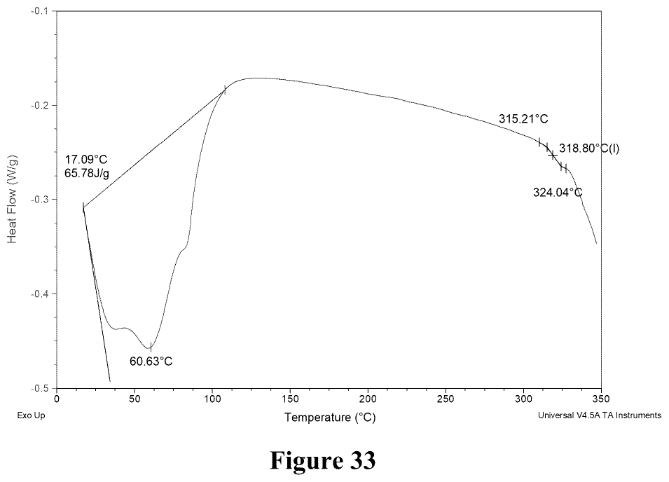

In some embodiments, Formula I, calcium salt, Form IV is characterized by a DSC thermogram substantially as shown in FIG. 33.

In some embodiments, Formula I, calcium salt, Form IV is characterized by a DSC thermogram having one or both of (i) an endothermic transition at about 17° C. and (ii) a baseline shift around 315° C.

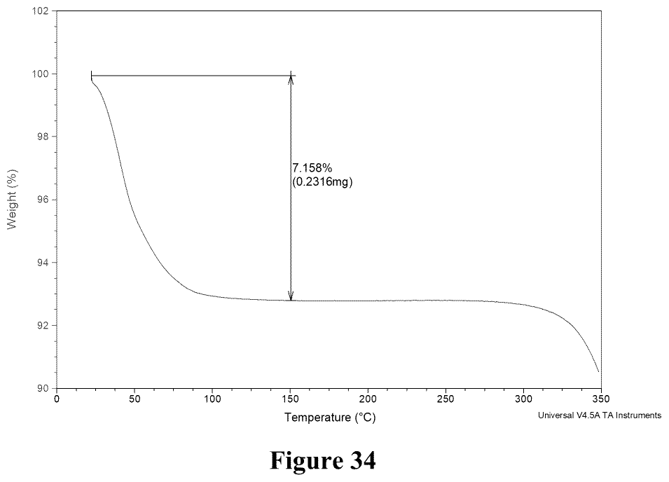

In some embodiments, Formula I, calcium salt, Form IV is characterized by a TGA curve substantially as shown in FIG. 34.

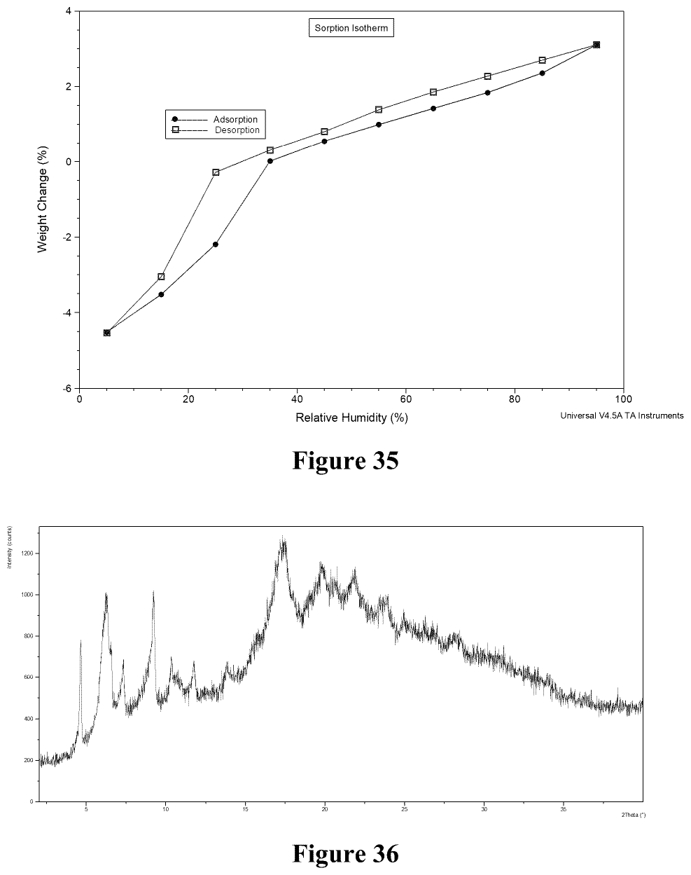

In some embodiments, Formula I, calcium salt, Form IV is characterized by a DVS curve substantially as shown in FIG. 35.

In some embodiments, Formula I, calcium salt, Form IV is hydrated. In some embodiments, Formula I, calcium salt, Form IV is partially or fully hydrated.

In some embodiments, the calcium salt of the compound of Formula I is the Form V (Formula I, calcium salt, Form V). In some embodiments, Formula I, calcium salt, Form V is hydrated. In some embodiments, Formula I, calcium salt, Form V has an XRPD profile substantially as shown in FIG. 36.

In some embodiments, Formula I, calcium salt, Form V has an XRPD pattern comprising degree 2θ-reflections (+/−0.2 degrees 2θ) at 4.7°, 6.3°, and 9.2°. In some embodiments, Formula I, calcium salt, Form V has an XRPD pattern comprising degree 2θ-reflections (+/−0.2 degrees 2θ) at 4.7°, 6.3°, and 9.2°, and one, two or three of the degree 2θ-reflections (+/−0.2 degrees 2θ) at 6.6°, 7.3°, and 17.5°. In some embodiments, Formula I, calcium salt, Form V has an XRPD pattern comprising degree 2θ-reflections (+/−0.2 degrees 2θ) at 4.7°, 6.3°, and 9.2°, and one or two of the degree 2θ-reflections (+/−0.2 degrees 2θ) at 6.6°, 7.3°, and 17.5°. In some embodiments, Formula I, calcium salt, Form V has an XRPD pattern comprising degree 2θ-reflections (+/−0.2 degrees 2θ) at 4.7°, 6.3°, and 9.2°, and one of the degree 2θ-reflections (+/−0.2 degrees 2θ) at 6.6°, 7.3°, and 17.5°. In some embodiments, Formula I, calcium salt, Form V has an XRPD pattern comprising degree 2θ-reflections (+/−0.2 degrees 2θ) at 4.7°, 6.3°, and 9.2°, and two of the degree 2θ-reflections (+/−0.2 degrees 2θ) at 6.6°, 7.3°, and 17.5°. In some embodiments, Formula I, calcium salt, Form V has an XRPD pattern comprising degree 2θ-reflections (+/−0.2 degrees 2θ) at 4.7°, 6.3°, 6.6°, 7.3°, 9.2°, and 17.5°. In some embodiments, Formula I, calcium salt, Form V has an XRPD pattern comprising any three of the degree 2θ-reflections (+/−0.2 degrees 2θ) at 4.7°, 6.3°, 6.6°, 7.3°, 9.2°, and 17.5°.

In some embodiments, Formula I, calcium salt, Form V has an XRPD pattern comprising degree 2θ-reflections (+/−0.2 degrees 2θ) at 4.7°, 6.3°, 6.6°, 7.3°, 9.2°, and 17.5°, and one, two or three of the degree 2θ-reflections (+/−0.2 degrees 2θ) at 10.3°, 19.8°, and 21.9°. In some embodiments, Formula I, calcium salt, Form V has an XRPD pattern comprising degree 2θ-reflections (+/−0.2 degrees 2θ) at 4.7°, 6.3°, 6.6°, 7.3°, 9.2°, and 17.5°, and one or two of the degree 2θ-reflections (+/−0.2 degrees 2θ) at 10.3°, 19.8°, and 21.9°. In some embodiments, Formula I, calcium salt, Form V has an XRPD pattern comprising degree 2θ-reflections (+/−0.2 degrees 2θ) at 4.7°, 6.3°, 6.6°, 7.3°, 9.2°, and 17.5°, and one of the degree 2θ-reflections (+/−0.2 degrees 2θ) at 10.3°, 19.8°, and 21.9°. In some embodiments, Formula I, calcium salt, Form V has an XRPD pattern comprising degree 2θ-reflections (+/−0.2 degrees 2θ) at 4.7°, 6.3°, 6.6°, 7.3°, 9.2°, and 17.5°, and two of the degree 2θ-reflections (+/−0.2 degrees 2θ) at 10.3°, 19.8°, and 21.9°. In some embodiments, Formula I, calcium salt, Form V has an XRPD pattern comprising degree 2θ-reflections (+/−0.2 degrees 2θ) 4.7°, 6.3°, 6.6°, 7.3°, 9.2°, 10.3°, 17.5°, 19.8°, and 21.9°. In some embodiments, Formula I, calcium salt, Form V has an XRPD pattern comprising any three of the degree 2θ-reflections (+/−0.2 degrees 2θ) at 4.7°, 6.3°, 6.6°, 7.3°, 9.2°, 10.3°, 17.5°, 19.8°, and 21.9°.

In some embodiments, Formula I, calcium salt, Form V has an XRPD pattern comprising at least three of the degree 2θ-reflections (+/−0.2 degrees 2θ) at 4.7°, 6.3°, 6.6°, 7.3°, 9.2°, 10.3°, 11.8°, 13.8°, 17.5°, 19.8°, and 21.9°. In some embodiments, Formula I, calcium salt, Form V has an XRPD pattern comprising at least four of the degree 2θ-reflections (+/−0.2 degrees 2θ) at 4.7°, 6.3°, 6.6°, 7.3°, 9.2°, 10.3°, 11.8°, 13.8°, 17.5°, 19.8°, and 21.9°. In some embodiments, Formula I, calcium salt, Form V has an XRPD pattern comprising at least five of the degree 2θ-reflections (+/−0.2 degrees 2θ) at 4.7°, 6.3°, 6.6°, 7.3°, 9.2°, 10.3°, 11.8°, 13.8°, 17.5°, 19.8°, and 21.9°.

In some embodiments, Formula I, calcium salt, Form V has an XRPD pattern comprising peaks at:

| Pos. | |
|---|---|
| [°2Th. ± | Rel. Int. |
| 0.2] | [%] |
| 4.667286 | 85.49 |
| 6.325596 | 100 |
| 6.574366 | 58.89 |
| 7.337722 | 41.98 |
| 9.219071 | 92.48 |
| 10.33942 | 28.4 |
| 11.76972 | 25.41 |
| 13.8499 | 17.35 |
| 17.51082 | 71.54 |
| 19.82631 | 37.1 |
| 21.93564 | 26.21 |
| 23.9084 | 19.19 |
| 28.46511 | 11.11 |

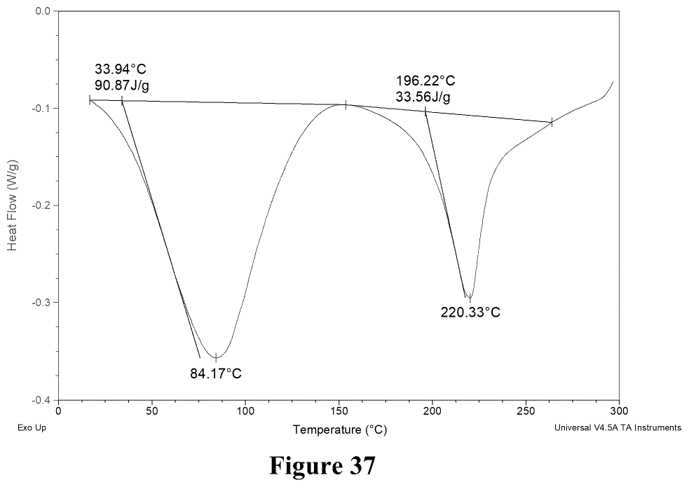

In some embodiments, Formula I, calcium salt, Form V is characterized by a DSC thermogram substantially as shown in FIG. 37.

In some embodiments, Formula I, calcium salt, Form V is characterized by a DSC thermogram having one or both of (i) an endothermic transition at about 34° C. and (ii) an endothermic transition around 196° C.

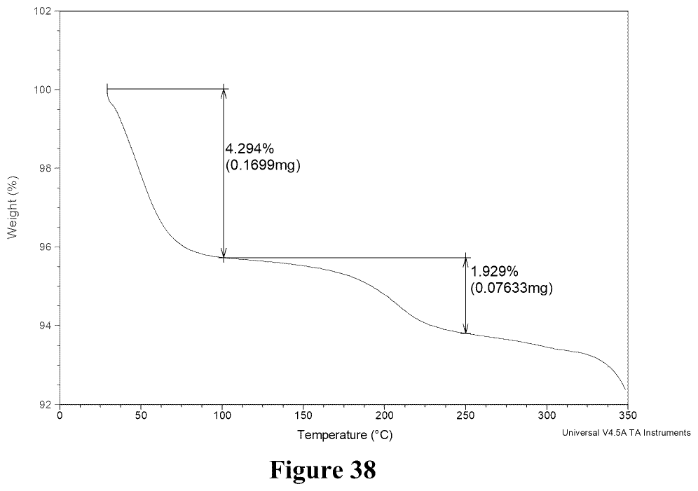

In some embodiments, Formula I, calcium salt, Form V is characterized by a TGA curve substantially as shown in FIG. 38.

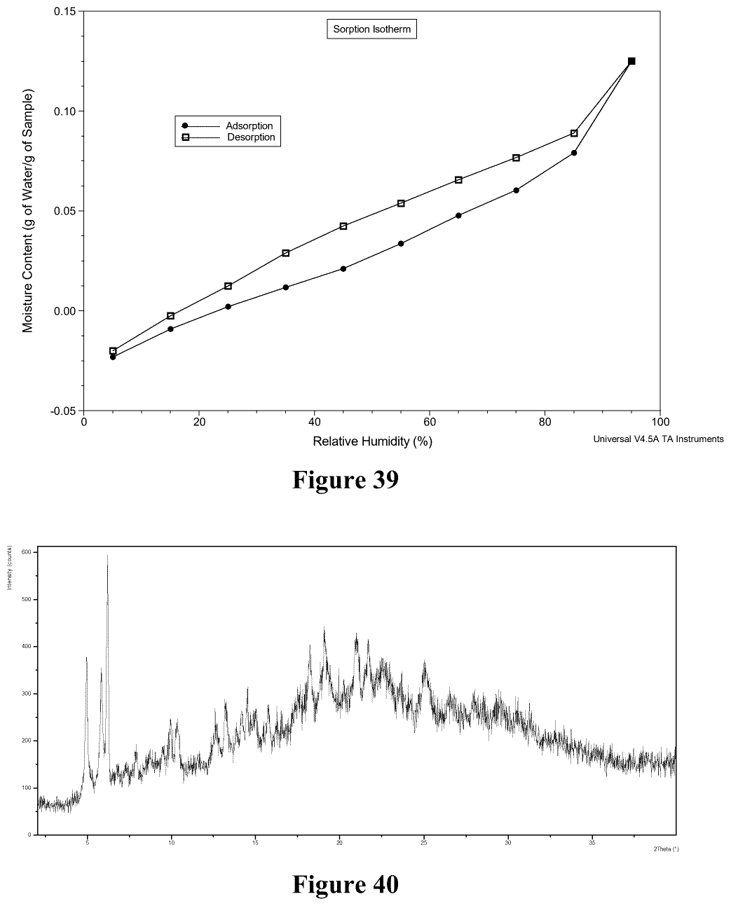

In some embodiments, Formula I, calcium salt, Form V is characterized by a DVS curve substantially as shown in FIG. 39.

In some embodiments, Formula I, calcium salt, Form V is hydrated. In some embodiments, Formula I, calcium salt, Form V is partially or fully hydrated.

In some embodiments, the crystalline calcium salt of the compound of Formula I is the Form VI (Formula I, calcium salt, Form VI). In some embodiments, Formula I, calcium salt, Form VI is hydrated. In some embodiments, Formula I, calcium salt, Form VI has an XRPD profile substantially as shown in FIG. 40.

In some embodiments, Formula I, calcium salt, Form VI has an XRPD pattern comprising degree 2θ-reflections (+/−0.2 degrees 2θ) at 5.0°, 5.8°, and 6.2°. In some embodiments, Formula I, calcium salt, Form VI has an XRPD pattern comprising degree 2θ-reflections (+/−0.2 degrees 2θ) at 5.0°, 5.8°, and 6.2°, and one, two or three of the degree 2θ-reflections (+/−0.2 degrees 2θ) at 18.2°, 19.1°, and 21.0°. In some embodiments, Formula I, calcium salt, Form VI has an XRPD pattern comprising degree 2θ-reflections (+/−0.2 degrees 2θ) at 5.0°, 5.8°, and 6.2°, and one or two of the degree 2θ-reflections (+/−0.2 degrees 2θ) at 18.2°, 19.1°, and 21.0°. In some embodiments, Formula I, calcium salt, Form VI has an XRPD pattern comprising degree 2θ-reflections (+/−0.2 degrees 2θ) at 5.0°, 5.8°, and 6.2°, and one of the degree 2θ-reflections (+/−0.2 degrees 2θ) at 18.2°, 19.1°, and 21.0°. In some embodiments, Formula I, calcium salt, Form VI has an XRPD pattern comprising degree 2θ-reflections (+/−0.2 degrees 2θ) at 5.0°, 5.8°, and 6.2°, and two of the degree 2θ-reflections (+/−0.2 degrees 2θ) at 18.2°, 19.1°, and 21.0°. In some embodiments, Formula I, calcium salt, Form VI has an XRPD pattern comprising degree 2θ-reflections (+/−0.2 degrees 2θ) at 5.0°, 5.8°, 6.2°, 18.2°, 19.1°, and 21.0°. In some embodiments, Formula I, calcium salt, Form VI has an XRPD pattern comprising any three of the degree 2θ-reflections (+/−0.2 degrees 2θ) at 5.0°, 5.8°, 6.2°, 18.2°, 19.1°, and 21.0°.

In some embodiments, Formula I, calcium salt, Form VI has an XRPD pattern comprising degree 2θ-reflections (+/−0.2 degrees 2θ) at 5.0°, 5.8°, 6.2°, 18.2°, 19.1°, and 21.0°, and one, two or three of the degree 2θ-reflections (+/−0.2 degrees 2θ) at 10.0°, 10.3°, and 21.7°.

In some embodiments, Formula I, calcium salt, Form VI has an XRPD pattern comprising degree 2θ-reflections (+/−0.2 degrees 2θ) at 5.0°, 5.8°, 6.2°, 18.2°, 19.1°, and 21.0°, and one or two of the degree 2θ-reflections (+/−0.2 degrees 2θ) at 10.0°, 10.3°, and 21.7°. In some embodiments, Formula I, calcium salt, Form VI has an XRPD pattern comprising degree 2θ-reflections (+/−0.2 degrees 2θ) at 5.0°, 5.8°, 6.2°, 18.2°, 19.1°, and 21.0°, and one of the degree 2θ-reflections (+/−0.2 degrees 2θ) at 10.0°, 10.3°, and 21.7°. In some embodiments, Formula I, calcium salt, Form VI has an XRPD pattern comprising degree 2θ-reflections (+/−0.2 degrees 2θ) at 5.0°, 5.8°, 6.2°, 18.2°, 19.1°, and 21.0°, and two of the degree 2θ-reflections (+/−0.2 degrees 2θ) at 10.0°, 10.3°, and 21.7°. In some embodiments, Formula I, calcium salt, Form VI has an XRPD pattern comprising degree 2θ-reflections (+/−0.2 degrees 2θ) 5.0°, 5.8°, 6.2°, 10.0°, 10.3°, 18.2°, 19.1°, 21.0°, and 21.7°. In some embodiments, Formula I, calcium salt, Form VI has an XRPD pattern comprising any three of the degree 2θ-reflections (+/−0.2 degrees 2θ) at 5.0°, 5.8°, 6.2°, 10.0°, 10.3°, 18.2°, 19.1°, 21.0°, and 21.7°.

In some embodiments, Formula I, calcium salt, Form VI has an XRPD pattern comprising at least three of the degree 2θ-reflections (+/−0.2 degrees 2θ) at 5.0°, 5.8°, 6.2°, 7.9°, 10.0°, 10.3°, 12.6°, 13.2°, 14.5°, 15.8°, 18.2°, 19.1°, 21.0°, and 21.7°. In some embodiments, Formula I, calcium salt, Form VI has an XRPD pattern comprising at least four of the degree 2θ-reflections (+/−0.2 degrees 2θ) at 5.0°, 5.8°, 6.2°, 7.9°, 10.0°, 10.3°, 12.6°, 13.2°, 14.5°, 15.8°, 18.2°, 19.1°, 21.0°, and 21.7°. In some embodiments, Formula I, calcium salt, Form VI has an XRPD pattern comprising at least five of the degree 2θ-reflections (+/−0.2 degrees 2θ) at 5.0°, 5.8°, 6.2°, 7.9°, 10.0°, 10.3°, 12.6°, 13.2°, 14.5°, 15.8°, 18.2°, 19.1°, 21.0°, and 21.7°.

In some embodiments, Formula I, calcium salt, Form VI has an XRPD pattern comprising peaks at:

| Pos. | |
|---|---|
| [°2Th. ± | Rel. Int. |
| 0.2] | [%] |
| 4.97 | 63.7 |
| 5.83 | 53.94 |
| 6.21 | 100 |
| 7.90 | 10.79 |
| 8.65 | 6.8 |
| 9.95 | 22.63 |
| 10.34 | 21.71 |
| 12.61 | 15.78 |
| 13.22 | 19.38 |
| 14.50 | 20.38 |
| 15.77 | 12.66 |
| 18.22 | 29.81 |
| 19.11 | 35.21 |
| 20.98 | 27.28 |
| 21.71 | 26.79 |
| 22.61 | 16.35 |
| 24.99 | 20.77 |
| 26.59 | 8.56 |
| 29.60 | 9.96 |

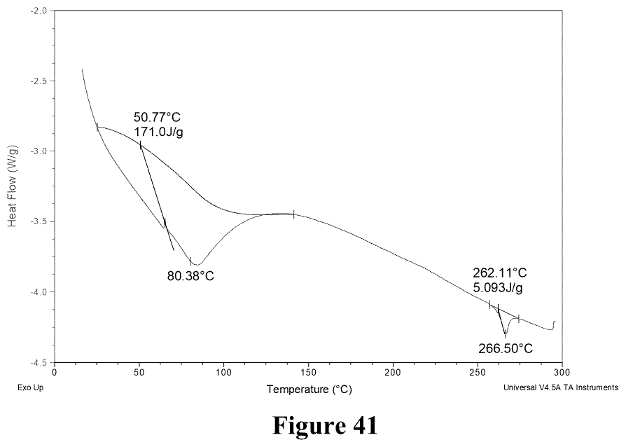

In some embodiments, Formula I, calcium salt, Form VI is characterized by a DSC thermogram substantially as shown in FIG. 41.

In some embodiments, Formula I, calcium salt, Form VI is characterized by a DSC thermogram having one or both of (i) an endothermic transition at about 51° C. and (ii) an endothermic transition around 262° C.

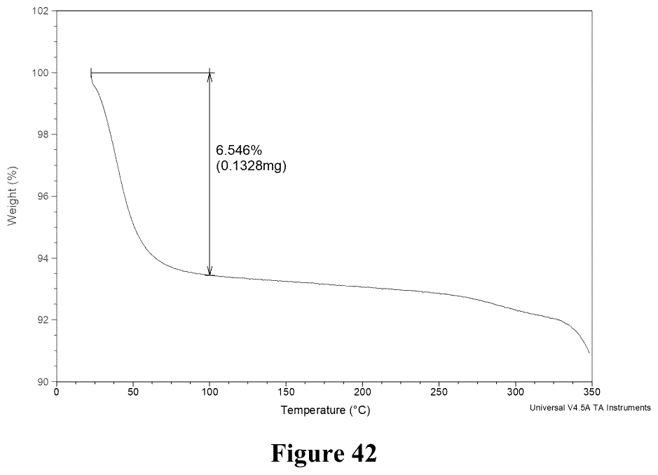

In some embodiments, Formula I, calcium salt, Form VI is characterized by a TGA curve substantially as shown in FIG. 42.

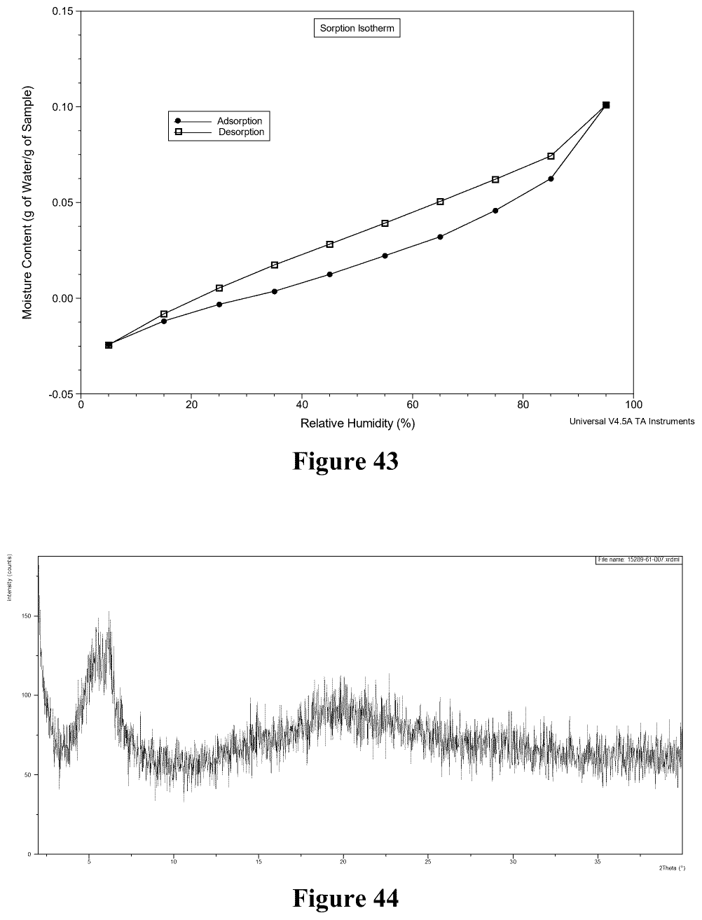

In some embodiments, Formula I, calcium salt, Form VI is characterized by a DVS curve substantially as shown in FIG. 43.

In some embodiments, Formula I, calcium salt, Form VI is hydrated. In some embodiments, Formula I, calcium salt, Form VI is partially or fully hydrated.

In some embodiments, the calcium salt of the compound of Formula I is amorphous (Formula I, calcium salt, amorphous). In some embodiments, Formula I, calcium salt, amorphous has an XRPD profile substantially as shown in FIG. 44.

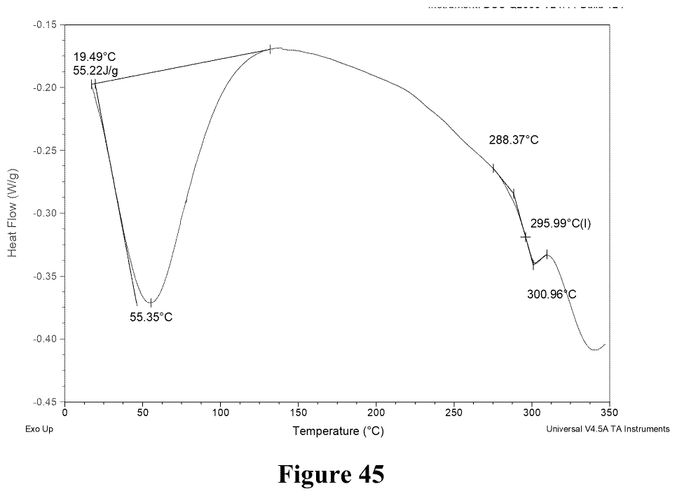

In some embodiments, Formula I, calcium salt, amorphous is characterized by a DSC thermogram substantially as shown in FIG. 45.

In some embodiments, Formula I, calcium salt, amorphous is characterized by a DSC thermogram having a baseline shift around 288° C.

Solid forms of the compound of Formula I, and methods of preparing solid forms thereof, are disclosed in U.S. Patent Application Nos. 63/613,815 and 63/615,780, each of which is incorporated by reference in its entirety.

The compound of Formula I can be present in the pharmaceutical compositions described herein in solvated and/or unsolvated form, and references to “a compound of Formula I” or “a compound of Formula I, or a pharmaceutically acceptable salt thereof” comprise the solvated and unsolvated forms and mixtures thereof. As used herein, and in absence of a specific reference to a particular pharmaceutically acceptable salt and/or solvate of the compound of Formula I, any dosages, whether expressed in, e.g., milligrams or as % by weight, should be taken as referring to the amount of the compound of Formula I, i.e., the amount of:

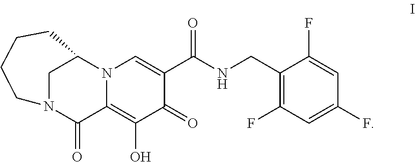 [SMILES: O=C(NCc1c(F)cc(F)cc1F)c1cn2c(c(O)c1=O)C(=O)N1CCCC[C@H]2C1]

Therefore, for example, a reference to “500 mg of the compound of Formula I, or a pharmaceutically acceptable salt thereof” means an amount of the Compound of Formula I, or a pharmaceutically acceptable salt thereof, which provides the same amount as 500 mg of the free acid of the compound of Formula I.

In some embodiments, the compound of Formula I, or pharmaceutically acceptable salt thereof, is micronized. Micronization is the process of reducing the particle size of a bulk solid material (e.g., a pharmaceutical drug substance) to micron or submicron level. Micronization of pharmaceutical bulk solids can be achieved via methods including, for example, milling or grinding.

In some embodiments, the particle size of the compound of Formula I, or pharmaceutically acceptable salt thereof, is controlled by milling, e.g., jet milling (fluid energy milling), bead milling, dry milling spiral milling, or high shear wet milling (HSWM). In an embodiment, the particle size of the compound of Formula I, or pharmaceutically acceptable salt thereof, is controlled by high shear wet milling. High shear wet milling can be either integrated into the crystallization process or performed post crystallization. In some embodiments, the HSWM is performed at a single speed of about 3,000 rpm to about 20,000 rpm; at about 3,000 rpm, about 4,000 rpm, about 5,000 rpm, about 6,000 rpm, about 7,000 rpm, about 8,000 rpm, about 9,000 rpm, about 10,000 rpm, about 11,000 rpm, about 12,000 rpm, about 13,000 rpm, about 14,000 rpm, about 15,000 rpm, or about 16,000 rpm. In an embodiment, the HSWM is performed at a single speed of 4,200 rpm. In an embodiment, the HSWM is performed at a single speed of 5,500 rpm. In an embodiment, the HSWM is performed at a single speed of 8,000 rpm. In an embodiment, the HSWM is performed at a single speed of 12,000 rpm. In an embodiment, the HSWM is performed at a single speed of 16,000 rpm. In some embodiments, the wet-milled granulated particles are passed through a comil upon drying. In some embodiments, the size of the comil screen is about 0.032 inches to about 0.250 inches. In an embodiment, the size of the comil screen is about 0.032 inches. In an embodiment, the size of the comil screen is about 0.250 inches. In some embodiments, the wet-milled granulated particles are dried in a tumble dryer. In some embodiments, the wet-milled granulated particles are dried in an agitated filter/dryer. In some embodiments, the wet-milled granulated particles are dried in a vacuum oven dryer.

In an embodiment, the particle size of the compound of Formula I, or pharmaceutically acceptable salt thereof, is controlled by jet milling. In an embodiment, the particle size of the compound of Formula I, or pharmaceutically acceptable salt thereof, is controlled by loop style jet milling. In an embodiment, the particle size of the compound of Formula I, or pharmaceutically acceptable salt thereof, is controlled by a Model 0202 Jet-O-Mizer (JOM) loop style jet mill system. In an embodiment, the particle size of the compound of Formula I, or pharmaceutically acceptable salt thereof, is controlled by spiral style jet milling. In some embodiments, particles are passed through a sieve after jet milling. In some embodiments, the size of the sieve is 0.355 mm (No. 45).

The particle size distribution of a solid material can be measured using a variety of analytical characterization methods known to those skilled in the art, including, for example, sieving, laser light diffraction, quasi-elastic light scattering, centrifugal sedimentation-optical, electrical resistance zone sensing, microelectrophoresis, light microscopy, and scanning electron microscopy, etc. In some embodiments, the particle size distribution of the compound of Formula I, or pharmaceutically acceptable salt thereof, is measured using light microscopy. In some embodiments, the particle size distribution of the compound of Formula I, or pharmaceutically acceptable salt thereof, is measured using laser light diffraction. The particle size distribution of a solid material can be represented by the d₉₀, d₅₀ and d₁₀ values as defined herein. In some embodiments, the compound of Formula I, or pharmaceutically acceptable salt thereof, has a particle size distribution with a d₉₀ value less than about 120 μm. For example, the compound of Formula I, or pharmaceutically acceptable salt thereof, has a particle size distribution with a d₉₀ value less than about 115 μm, about 110 μm, about 105 μm, about 100 μm, about 95 μm, about 90 μm, about 85 μm, about 80 μm, about 70 μm, about 65 μm, about 60 μm, about 55 μm, about 50 μm, about 45 μm, about 40 μm, about 35 μm, about 30 μm, about 25 μm, about 20 μm, about 15 μm, about 10 μm, about 9 μm, about 8 μm, about 7 μm, about 6 μm, about 5 μm, about 4 μm, about 3 μm, about 2 μm, or about 1 μm. In some embodiments, the compound of Formula I, or pharmaceutically acceptable salt thereof, has a particle size distribution with a d₉₀ value from about 1 μm to about 120 μm, for example, about 1 μm to about 115 μm, about 1 μm to about 110 μm, about 1 μm to about 105 μm, about 1 μm to about 100 μm, about 1 μm to about 95 μm, about 1 μm to about 90 μm, about 1 μm to about 85 μm, about 1 μm to about 80 μm, about 1 μm to about 75 μm, about 1 μm to about 70 μm, about 1 μm to about 65 μm, about 1 μm to about 60 μm, about 1 μm to about 55 μm, about 1 μm to about 50 μm, about 1 μm to about 45 μm, about 1 μm to about 40 μm, about 1 μm to about 35 μm, about 1 μm to about 30 μm, about 1 μm to about 25 μm, about 1 μm to about 20 μm, about 1 μm to about 15 μm, about 1 μm to about 10 μm, 1 μm to about 9 μm, 1 μm to about 8 μm, 1 μm to about 7 μm, 1 μm to about 6 μm, 1 μm to about 5 μm, 1 μm to about 4 μm, 1 μm to about 3 μm, or 1 μm to 2 μm. In some embodiments, the compound of Formula I, or pharmaceutically acceptable salt thereof, has a particle size distribution with a d₉₀ value of ≤about 30 μm. In some embodiments, the compound of Formula I, or pharmaceutically acceptable salt thereof, has a particle size distribution with a d₉₀ value of ≤about 20 μm. In some embodiments, the compound of Formula I, or pharmaceutically acceptable salt thereof, has a particle size distribution with a d₉₀ value of ≤about 15 μm. In some embodiments, the compound of Formula I, or pharmaceutically acceptable salt thereof, has a particle size distribution with a d9o value of ≤about 10 μm. In some embodiments, the compound of Formula I, or pharmaceutically acceptable salt thereof, has a particle size distribution with a d₉₀ value from about 25 μm to about 30 μm. In some embodiments, the compound of Formula I, or pharmaceutically acceptable salt thereof, has a particle size distribution with a d₉₀ value from about 20 μm to about 25 μm. In some embodiments, the compound of Formula I, or pharmaceutically acceptable salt thereof, has a particle size distribution with a d₉₀ value from about 15 μm to about 20 μm. In some embodiments, the compound of Formula I, or pharmaceutically acceptable salt thereof, has a particle size distribution with a d₉₀ value from about 10 μm to about 15 μm. In some embodiments, the compound of Formula I, or pharmaceutically acceptable salt thereof, has a particle size distribution with a d₉₀ value from about 1 μm to about 10 μm. In some embodiments, the compound of Formula I, or pharmaceutically acceptable salt thereof, has a particle size distribution with a d₉₀ value from about 1 μm to about 5 μm. In some embodiments, the compound of Formula I, or pharmaceutically acceptable salt thereof, has a particle size distribution with a d₉₀ value of about 20 μm. In some embodiments, the compound of Formula I, or pharmaceutically acceptable salt thereof, has a particle size distribution with a d₉₀ value of about 19 μm. In some embodiments, the compound of Formula I, or pharmaceutically acceptable salt thereof, has a particle size distribution with a d₉₀ value of about 18 μm. In some embodiments, the compound of Formula I, or pharmaceutically acceptable salt thereof, has a particle size distribution with a d₉₀ value of about 17 μm. In some embodiments, the compound of Formula I, or pharmaceutically acceptable salt thereof, has a particle size distribution with a d₉₀ value of about 16 μm. In some embodiments, the compound of Formula I, or pharmaceutically acceptable salt thereof, has a particle size distribution with a d₉₀ value of about 15 μm. In some embodiments, the compound of Formula I, or pharmaceutically acceptable salt thereof, has a particle size distribution with a d₉₀ value of about 14 μm. In some embodiments, the compound of Formula I, or pharmaceutically acceptable salt thereof, has a particle size distribution with a d₉₀ value of about 13 μm. In some embodiments, the compound of Formula I, or pharmaceutically acceptable salt thereof, has a particle size distribution with a d₉₀ value of about 12 μm. In some embodiments, the compound of Formula I, or pharmaceutically acceptable salt thereof, has a particle size distribution with a d₉₀ value of about 11 μm. In some embodiments, the compound of Formula I, or pharmaceutically acceptable salt thereof, has a particle size distribution with a d₉₀ value of about 10 μm. In some embodiments, the compound of Formula I, or pharmaceutically acceptable salt thereof, has a particle size distribution with a d₉₀ value of about 9 μm. In some embodiments, the compound of Formula I, or pharmaceutically acceptable salt thereof, has a particle size distribution with a d₉₀ value of about 5 μm. In some embodiments, the compound of Formula I, or pharmaceutically acceptable salt thereof, has a particle size distribution with a d₉₀ value of about 4 μm.

In some embodiments, the compound of Formula I, or pharmaceutically acceptable salt thereof, has a particle size distribution with a d₉₀ value from about 1 μm to about 90 μm. In some embodiments, the compound of Formula I, or pharmaceutically acceptable salt thereof, has a particle size distribution with a d₉₀ value from about 15 μm to about 70 μm. In some embodiments, the compound of Formula I, or pharmaceutically acceptable salt thereof, has a particle size distribution with a d₉₀ value from about 1 μm to about 30 μm. In some embodiments, the compound of Formula I, or pharmaceutically acceptable salt thereof, has a particle size distribution with a d₉₀ value from about 5 μm to about 30 μm.

In some embodiments, the compound of Formula I, or pharmaceutically acceptable salt thereof, has a particle size distribution d₉₀ of from about 1 μm to about 30 μm. In some embodiments, the compound of Formula I, or pharmaceutically acceptable salt thereof, has a particle size distribution d₉₀ of from about 1 μm to about 20 μm. In some embodiments, the compound of Formula I, or pharmaceutically acceptable salt thereof, has a particle size distribution d₉₀ of from about 5 μm to about 20 μm. In some embodiments, the compound of Formula I, or pharmaceutically acceptable salt thereof, has a particle size distribution d₉₀ of from about 5 μm to about 15 μm. In some embodiments, the compound of Formula I, or pharmaceutically acceptable salt thereof, has a particle size distribution with a d₉₀ value less than about 20 μm. In some embodiments, the compound of Formula I, or pharmaceutically acceptable salt thereof, has a particle size distribution with a d₉₀ value from about 1 μm to about 10 μm. In some embodiments, the compound of Formula I, or pharmaceutically acceptable salt thereof, has a particle size distribution with a d₉₀ value from about 1 μm to about 5 μm.

In some embodiments, the compound of Formula I, or pharmaceutically acceptable salt thereof, has a particle size distribution d₁₀ of from about 0.1 μm to about 70 μm, about 0.1 μm to about 50 μm, about 0.1 μm to about 20 μm, or about 0.1 μm to about 10 μm. In some embodiments, the compound of Formula I, or pharmaceutically acceptable salt thereof, has a particle size distribution d₁₀ of from about 0.1 μm to about 10 μm. In some embodiments, the compound of Formula I, or pharmaceutically acceptable salt thereof, has a particle size distribution d₁₀ of about 1 μm.

In some embodiments, the compound of Formula I, or pharmaceutically acceptable salt thereof, has a particle size distribution d₅₀ of from about 0.1 μm to about 80 μm, about 0.5 μm to about 50 μm, about 1 μm to about 30 μm, or about 1 μm to about 10 μm. In some embodiments, the compound of Formula I, or pharmaceutically acceptable salt thereof, has a particle size distribution d₅₀ of from about 1 μm to about 10 μm. In some embodiments, the compound of Formula I, or pharmaceutically acceptable salt thereof, has a particle size distribution d₅₀ of from about 1 μm to about 5 μm. In some embodiments, the compound of Formula I, or pharmaceutically acceptable salt thereof, has a particle size distribution d₅₀ of from about 1 μm to about 4 μm.

In some embodiments, the micronized compound of Formula I, or pharmaceutically acceptable salt thereof, is further processed (e.g., milled) after micronization to reduce particle sizes to the submicron level (i.e., nanometer level). In some embodiments, the compound of Formula I, or pharmaceutically acceptable salt thereof, is milled by wet bead milling or high pressure homogenization.

In some embodiments, the compound of Formula I, or pharmaceutically acceptable salt thereof, is milled by wet bead milling.

In some embodiments, the compound of Formula I, or pharmaceutically acceptable salt thereof, is milled by high pressure homogenization.

In some embodiments, the compound of Formula I, or pharmaceutically acceptable salt thereof, has a particle size distribution d₉₀ of less than 1 μm. In some embodiments, the compound of Formula I, or pharmaceutically acceptable salt thereof, has a particle size distribution d₉₀ of from about 100 nm to about 950 nm, about 200 nm to about 900 nm, about 200 nm to about 600 nm, about 300 nm to about 700 nm, about 400 nm to about 800 nm, about 500 nm to about 900 nm, about 200 nm to about 500 nm, about 400 nm to about 900 nm, about 400 nm to about 600 nm, or about 500 nm to about 800 nm.

In some embodiments, the compound of Formula I, or pharmaceutically acceptable salt thereof, has a particle size distribution d₉₀ of from about 200 nm to about 900 nm. In some embodiments, the compound of Formula I, or pharmaceutically acceptable salt thereof, has a particle size distribution d₉₀ of from about 400 nm to about 800 nm.

In some embodiments, the compound of Formula I, or pharmaceutically acceptable salt thereof, has a particle size distribution d₁₀ of from about 1 nm to about 600 nm, about 25 nm to about 500 nm, about 50 nm to about 400 nm, or about 50 nm to about 350 nm. In some embodiments, the compound of Formula I, or pharmaceutically acceptable salt thereof, has a particle size distribution d₁₀ of from about 50 nm to about 350 nm.

In some embodiments, the compound of Formula I, or pharmaceutically acceptable salt thereof, has a particle size distribution d₅₀ of from about 50 nm to about 700 nm, about 75 nm to about 600 nm, about 100 nm to about 550 nm, or about 200 nm to about 500 nm. In some embodiments, the compound of Formula I, or pharmaceutically acceptable salt thereof, has a particle size distribution d₅₀ of from about 200 nm to about 500 nm.

In some embodiments, the compound of Formula I, or pharmaceutically acceptable salt thereof, is processed by a solvent evaporation method (e.g., spray drying, lyophilization, super-critical fluid, co-precipitation, electrospinning). In some embodiments, the compound of Formula I, or a pharmaceutically acceptable salt thereof, is spray dried.

Any amount of the compound of Formula I, or pharmaceutically acceptable salt thereof, can be present in the pharmaceutical compositions described herein. In some embodiments, the amount of the compound of Formula I, or pharmaceutically acceptable salt thereof, is about 5% to about 50% of the total weight of the pharmaceutical composition (wt. %). In some embodiments, the amount of the compound of Formula I, or pharmaceutically acceptable salt thereof, is about 5 to about 45% of the total pharmaceutical composition weight. In some embodiments, the compound of Formula I, or a pharmaceutically acceptable salt thereof, is present at about 15% to about 45% of the total pharmaceutical composition weight.

In some embodiments, the amount of the compound of Formula I, or pharmaceutically acceptable salt thereof, is about 25% to about 45%, about 25% to about 40%, about 30% to about 50%, about 30% to about 45%, about 30% to about 40%, about 35% to about 45%, about 40% to about 45%, about 15% to about 45%, about 15% to about 40%, about 15% to about 35%, about 15% to about 30%, about 15% to about 25% about 15% to about 20%, about 10% to about 40%, or about 10% to about 30% of the total pharmaceutical composition weight. In some embodiments, the compound of Formula I, or a pharmaceutically acceptable salt thereof, is present at about 10 wt. % to about 55 wt. %, about 10 wt. % to about 50 wt. %, about 15 wt. % to about 50 wt. %, or about 15 wt. % to about 45 wt. % of the total pharmaceutical composition. In some embodiments, the compound of Formula I, or a pharmaceutically acceptable salt thereof, is present at about 15% to about 25%, about 25% to about 35%, or about 35% to about 45% of the total pharmaceutical composition weight. In some embodiments, the compound of Formula I, or a pharmaceutically acceptable salt thereof, is present at about 20%, about 33%, or about 38% of the total pharmaceutical composition weight.

In some embodiments, the pharmaceutical composition comprises about 10 wt. % to about 50 wt. % of the compound of Formula I. In some embodiments, the pharmaceutical composition comprises about 10 wt. % to about 50 wt. % of the compound of Formula I and the compound of Formula I has Form I.

In some embodiments, the pharmaceutical composition comprises about 10 wt. % to about 40 wt. % of the compound of Formula I. In some embodiments, the pharmaceutical composition comprises about 10 wt. % to about 40 wt. % of the compound of Formula I and the compound of Formula I has Form I.

In some embodiments, the pharmaceutical composition comprises about 10 wt. % to about 30 wt. % of the compound of Formula I. In some embodiments, the pharmaceutical composition comprises about 10 wt. % to about 30 wt. % of the compound of Formula I and the compound of Formula I has Form I.

In some embodiments, the pharmaceutical composition comprises about 15 wt. % to about 45 wt. % of the compound of Formula I. In some embodiments, the pharmaceutical composition comprises about 15 wt. % to about 45 wt. % of the compound of Formula I and the compound of Formula I has Form I.

In some embodiments, the pharmaceutical composition comprises about 15 wt. % to about 35 wt. % of the compound of Formula I. In some embodiments, the pharmaceutical composition comprises about 15 wt. % to about 35 wt. % of the compound of Formula I and the compound of Formula I has Form I.

In some embodiments, the pharmaceutical composition comprises about 15 wt. % to about 25 wt. % of the compound of Formula I. In some embodiments, the pharmaceutical composition comprises about 15 wt. % to about 25 wt. % of the compound of Formula I and the compound of Formula I has Form I.

In some embodiments, the pharmaceutical composition comprises about 20 wt. % to about 40 wt. % of the compound of Formula I. In some embodiments, the pharmaceutical composition comprises about 20 wt. % to about 40 wt. % of the compound of Formula I and the compound of Formula I has Form I.

In some embodiments, the pharmaceutical composition comprises about 20 wt. % to about 30 wt. % of the compound of Formula I. In some embodiments, the pharmaceutical composition comprises about 20 wt. % to about 30 wt. % of the compound of Formula I and the compound of Formula I has Form I.

In some embodiments, the pharmaceutical composition comprises about 25 wt. % to about 40 wt. % of the compound of Formula I. In some embodiments, the pharmaceutical composition comprises about 25 wt. % to about 40 wt. % of the compound of Formula I and the compound of Formula I has Form I.

In some embodiments, the pharmaceutical composition comprises about 30 wt. % to about 35 wt. % of the compound of Formula I. In some embodiments, the pharmaceutical composition comprises about 30 wt. % to about 35 wt. % of the compound of Formula I and the compound of Formula I has Form I.

In some embodiments, the pharmaceutical composition comprises about 35 wt. % to about 45 wt. % of the compound of Formula I. In some embodiments, the pharmaceutical composition comprises about 35 wt. % to about 45 wt. % of the compound of Formula I and the compound of Formula I has Form I.

In some embodiments, the pharmaceutical composition comprises about 40 wt. % of the compound of Formula I. In some embodiments, the pharmaceutical composition comprises about 40 wt. % of the compound of Formula I and the compound of Formula I has Form I.

In some embodiments, the pharmaceutical composition comprises about 33 wt. % of the compound of Formula I. In some embodiments, the pharmaceutical composition comprises about 33 wt. % of the compound of Formula I and the compound of Formula I has Form I.

In some embodiments, the pharmaceutical composition comprises about 22 wt. % of the compound of Formula I. In some embodiments, the pharmaceutical composition comprises about 22 wt. % of the compound of Formula I and the compound of Formula I has Form I.

In some embodiments, the pharmaceutical composition comprises about 19 wt. % of the compound of Formula I. In some embodiments, the pharmaceutical composition comprises about 19 wt. % of the compound of Formula I and the compound of Formula I has Form I.

In some embodiments, the pharmaceutical composition comprises about 10 wt. % to about 40 wt. % of a sodium salt of the compound of Formula I. In some embodiments, the pharmaceutical composition comprises about 20 wt. % to about 30 wt. % of a sodium salt of the compound of Formula I. In some embodiments, the pharmaceutical composition comprises about 24 wt. % of a sodium salt of the compound of Formula I.

In some embodiments, the pharmaceutical composition comprises about 10 wt. % to about 40 wt. % of a sodium salt of the compound of Formula I and the sodium salt of the compound of Formula I has Form I. In some embodiments, the pharmaceutical composition comprises about 20 wt. % to about 30 wt. % of a sodium salt of the compound of Formula I and the sodium salt of the compound of Formula I has form I. In some embodiments, the pharmaceutical composition comprises about 24 wt. % of a sodium salt of the compound of Formula I and the sodium salt of the compound of Formula I has form I.

In some embodiments, the pharmaceutical composition comprises about 10 wt. % to about 55 wt. % of a calcium salt of the compound of Formula I. In some embodiments, the pharmaceutical composition comprises about 10 wt. % to about 50 wt. % of a calcium salt of the compound of Formula I. In some embodiments, the pharmaceutical composition comprises about 10 wt. % to about 50 wt. % of a calcium salt of the compound of Formula I and the calcium salt of the compound of Formula I has Form I. In some embodiments, the pharmaceutical composition comprises about 10 wt. % to about 40 wt. % of a calcium salt of the compound of Formula I. In some embodiments, the pharmaceutical composition comprises about 10 wt. % to about 40 wt. % of a calcium salt of the compound of Formula I and the calcium salt of the compound of Formula I has Form I. In some embodiments, the pharmaceutical composition comprises about 30 wt. % to about 50 wt. % of a calcium salt of the compound of Formula I. In some embodiments, the pharmaceutical composition comprises about 30 wt. % to about 50 wt. % of a calcium salt of the compound of Formula I and the calcium salt of the compound of Formula I has Form I. In some embodiments, the pharmaceutical composition comprises about 30 wt. % to about 40 wt. % of a calcium salt of the compound of Formula I. In some embodiments, the pharmaceutical composition comprises about 30 wt. % to about 40 wt. % of a calcium salt of the compound of Formula I and the calcium salt of the compound of Formula I has Form I. In some embodiments, the pharmaceutical composition comprises about 38 wt. % of a calcium salt of the compound of Formula I. In some embodiments, the pharmaceutical composition comprises about 38 wt. % of a calcium salt of the compound of Formula I and the calcium salt of the compound of Formula I has Form I. In some embodiments, the pharmaceutical composition comprises about 40 wt. % of a calcium salt of the compound of Formula I. In some embodiments, the pharmaceutical composition comprises about 40 wt. % of a calcium salt of the compound of Formula I and the calcium salt of the compound of Formula I has Form I.

In some embodiments, the pharmaceutical composition comprises about 10 wt. % to about 50 wt. % of a calcium salt of the compound of Formula I and the calcium salt of the compound of Formula I is amorphous. In some embodiments, the pharmaceutical composition comprises about 10 wt. % to about 40 wt. % of a calcium salt of the compound of Formula I and the calcium salt of the compound of Formula I is amorphous. In some embodiments, the pharmaceutical composition comprises about 30 wt. % to about 50 wt. % of a calcium salt of the compound of Formula I and the calcium salt of the compound of Formula I is amorphous. In some embodiments, the pharmaceutical composition comprises about 30 wt. % to about 40 wt. % of a calcium salt of the compound of Formula I and the calcium salt of the compound of Formula I is amorphous. In some embodiments, the pharmaceutical composition comprises about 38 wt. % of a calcium salt of the compound of Formula I and the calcium salt of the compound of Formula I is amorphous. In some embodiments, the pharmaceutical composition comprises about 40 wt. % of a calcium salt of the compound of Formula I and the calcium salt of the compound of Formula I is amorphous.

### III. Pharmaceutical Compositions Comprising the Compound of Formula I

Provided herein are pharmaceutical compositions comprising the compound of Formula I, or a pharmaceutically acceptable salt thereof. The pharmaceutical compositions described herein are generally physically and/or chemically stable. As such the pharmaceutical compositions described herein can be stored at room temperature or refrigerated conditions for an extended period of time without significant degradation and/or change in physical form. In some embodiments, the pharmaceutical compositions can be stored at room temperature for at least one month, for example at least two months, at least three months, at least four months, at least five months, at least six months, at least one year, or at least two years.

The pharmaceutical compositions described herein are suitable for injection. As such, the compound of Formula I, or pharmaceutically acceptable salt thereof, can be formulated as a suspension or solution in a pharmaceutically acceptable liquid vehicle. In some embodiments, the pharmaceutical composition is a suspension formulation. In some embodiments, the suspension formulation is a solid dispersion formulation wherein the compound of Formula I, or pharmaceutically acceptable salt thereof, is dispersed in the pharmaceutically acceptable liquid vehicle. In some embodiments, the pharmaceutical composition is formulated as a microsuspension. In some embodiments, a microsuspension is a dispersion of drug particles typically of between about 1 μm to about 200 μm in diameter. In some embodiments, the pharmaceutical composition is formulated as a nanosuspension. In some embodiments, a nanosuspension is a biphasic, colloidal dispersion of submicron drug particles typically of less than 1 μm in diameter.

In some embodiments, the pharmaceutical compositions described herein are administered parenterally. In some embodiments, the pharmaceutical compositions described herein are administered parenterally via injection. In some embodiments, the pharmaceutical composition is suitable for subcutaneous or intramuscular administration. In some embodiments, the pharmaceutical composition is suitable for subcutaneous administration. In some embodiments, the pharmaceutical composition is suitable for intramuscular administration.

In some embodiments, the pharmaceutical compositions described herein are administered via injection using an injection device. In some embodiments, the injection device is or includes a syringe, which can be employed manually, or as part of a syringe-containing injection device. A wide variety of injection devices can be used, including, but not limited to, a handheld or wearable autoinjector, a handheld or wearable manual injector, an on-body injector, a syrette, a jet injector, or a pen injector, each of which can be reusable or disposable.

In some embodiments, the pharmaceutical compositions provided herein can be administered with a syringe suitable for administration of the compound. In some embodiments, the syringe is disposable. In some embodiments, the syringe is reusable. In some embodiments, the syringe is pre-filled with the pharmaceutical composition. In some embodiments, the syringe is a single-chamber syringe or cartridge. In some embodiments, the syringe is a dual-chamber syringe or cartridge.

In some embodiments, the pharmaceutical compositions provided herein can be administered with an auto-injector comprising a syringe. In some embodiments, the syringe is disposable. In some embodiments, the syringe is reusable. In some embodiments, the syringe is pre-filled with any of the pharmaceutical compositions provided herein.

The pharmaceutical compositions of the present disclosure may be in the form of a sterile injectable preparation, such as a sterile injectable aqueous or organic suspension. This suspension may be formulated according to the known art using those suitable dispersing or wetting agents and suspending agents which have been mentioned herein. The sterile injectable preparation may also be a sterile injectable solution or suspension in a non-toxic parenterally acceptable diluent or solvent. In addition, sterile fixed oils may conventionally be employed as a solvent or suspending medium. In some embodiments, the sterile injectable preparation disclosed herein may also be a sterile injectable solution or suspension prepared from a reconstituted lyophilized powder in a non-toxic parenterally acceptable liquid vehicle.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. In some embodiments, the pharmaceutical composition comprises one or more solubilizing excipients. Examples of solubilizing excipients in a parenteral formulation (e.g., an SC or IM formulation) include, but are not limited to, polyethylene glycol (PEG), methoxy polyethylene glycol (mPEG), N-methylpyrrolidone (NMP), dimethylsulfoxide (DMSO), ethanol, benzyl benzoate, and glycerin.

In some embodiments, the concentration of the compound of Formula I, or a pharmaceutically acceptable salt thereof, in the pharmaceutical composition for injection is from about 100 mg/mL to about 1000 mg/mL, including, e.g., about 200 mg/mL to about 1000 mg/mL, about 300 mg/mL to about 1000 mg/mL, about 100 mg/mL to about 750 mg/mL, about 150 mg/mL to about 600 mg/mL, about 200 mg/mL to about 500 mg/mL, about 300 mg/mL to about 500 mg/mL, or about 400 mg/mL to about 500 mg/mL.

In some embodiments, the concentration of the compound of Formula I, or a pharmaceutically acceptable salt thereof, in the pharmaceutical composition for injection is about 100 mg/mL, about 125 mg/mL, about 150 mg/mL, about 175 mg/mL, about 200 mg/mL, about 225 mg/mL, about 250 mg/mL, about 275 mg/mL, about 300 mg/mL, about 325 mg/mL, about 350 mg/mL, about 375 mg/mL, about 400 mg/mL, about 425 mg/mL, about 450 mg/mL, about 475 mg/mL, about 500 mg/mL, about 525 mg/mL, about 550 mg/mL, about 575 mg/mL, about 600 mg/mL, about 625 mg/mL, about 650 mg/mL, about 675 mg/mL, about 700 mg/mL, about 725 mg/mL, about 750 mg/mL, about 775 mg/mL, about 800 mg/mL, about 825 mg/mL, about 850 mg/mL, about 875 mg/mL, about 900 mg/mL, about 925 mg/mL, about 950 mg/mL, about 975 mg/mL, or about 1000 mg/mL.

In some embodiments, the concentration of the compound of Formula I, or a pharmaceutically acceptable salt thereof, is from about 200 mg/mL to about 500 mg/mL, about 300 mg/mL to about 400 mg/mL, about 200 mg/mL to about 300 mg/mL, about 300 mg/mL to about 400 mg/mL, or about 400 mg/mL to about 500 mg/mL.

In some embodiments, the concentration of the compound of Formula I, or a pharmaceutically acceptable salt thereof, is from about 225 mg/mL to about 400 mg/mL. In some embodiments, the concentration of the compound of Formula I, or a pharmaceutically acceptable salt thereof, is from about 200 mg/mL to about 400 mg/mL.

In some embodiments, the concentration of the compound of Formula I, or a pharmaceutically acceptable salt thereof, is about 200 mg/mL, about 225 mg/mL, about 250 mg/mL, about 275 mg/mL, about 300 mg/mL, about 325 mg/mL, about 350 mg/mL, about 375 mg/mL, about 400 mg/mL, about 425 mg/mL, about 450 mg/mL, about 475 mg/mL, or about 500 mg/mL.

In some embodiments, the concentration of the compound of Formula I, or a pharmaceutically acceptable salt thereof, is about 225 mg/mL, about 250 mg/mL, about 300 mg/mL, about 350 mg/mL, about 400 mg/mL, or about 450 mg/mL.

In some embodiments, the concentration of the compound of Formula I, or a pharmaceutically acceptable salt thereof, is from about 100 mg/mL to about 750 mg/mL. In some embodiments, the concentration of the compound of Formula I, or a pharmaceutically acceptable salt thereof, is from about 150 mg/mL to about 600 mg/mL. In some embodiments, the concentration of the compound of Formula I, or a pharmaceutically acceptable salt thereof, in the pharmaceutical composition for injection is from about 200 mg/mL to about 500 mg/mL.

In some embodiments, the concentration of the compound of Formula I, or a pharmaceutically acceptable salt thereof, in the pharmaceutical composition for injection is from about 200 mg/mL to about 400 mg/mL.

In some embodiments, the concentration of the compound of Formula I, or a pharmaceutically acceptable salt thereof, in the pharmaceutical composition for injection is about 225 mg/mL. In some embodiments, the concentration of the compound of Formula I, or a pharmaceutically acceptable salt thereof, in the pharmaceutical composition for injection is about 350 mg/mL. In some embodiments, the concentration of the compound of Formula I, or a pharmaceutically acceptable salt thereof, in the pharmaceutical composition for injection is about 400 mg/mL.

In some embodiments, the concentration of the compound of Formula I, or a pharmaceutically acceptable salt thereof, in the pharmaceutical composition for injection is from about 400 mg/mL to about 500 mg/mL. In some embodiments, the concentration of the compound of Formula I, or a pharmaceutically acceptable salt thereof, in the pharmaceutical composition for injection is about 400 mg/mL. In some embodiments, the concentration of the compound of Formula I, or a pharmaceutically acceptable salt thereof, in the pharmaceutical composition for injection is about 450 mg/mL. 10248J In some embodiments, the concentration of the compound of Formula I, or a pharmaceutically acceptable salt thereof, in the pharmaceutical composition for injection is from about 200 mg/mL to about 300 mg/mL. In some embodiments, the concentration of the compound of Formula I, or a pharmaceutically acceptable salt thereof, in the pharmaceutical composition for injection is from about 300 mg/mL to about 400 mg/mL. In some embodiments, the concentration of the compound of Formula I, or a pharmaceutically acceptable salt thereof, in the pharmaceutical composition for injection is about 300 mg/mL.

In some embodiments, the concentration of the compound of Formula I, or a pharmaceutically acceptable salt thereof, in the pharmaceutical composition for injection is greater than about 100 mg/mL. In some embodiments, the concentration of the compound of Formula I, or a pharmaceutically acceptable salt thereof, in the pharmaceutical composition for injection is greater than about 200 mg/mL. In some embodiments, the concentration of the compound of Formula I, or a pharmaceutically acceptable salt thereof, in the pharmaceutical composition for injection is greater than about 250 mg/mL. In some embodiments, the concentration of the compound of Formula I, or a pharmaceutically acceptable salt thereof, in the pharmaceutical composition for injection is greater than about 300 mg/mL. In some embodiments, the concentration of the compound of Formula I, or a pharmaceutically acceptable salt thereof, in the pharmaceutical composition for injection is greater than about 350 mg/mL. In some embodiments, the concentration of the compound of Formula I, or a pharmaceutically acceptable salt thereof, in the pharmaceutical composition for injection is greater than about 400 mg/mL.

In some embodiments, the volume of the pharmaceutical composition administered per injection (“injection volume”) is about 0.1 to about 10 mL. In some embodiments, the injection volume is about 0.5 mL to about 6 mL, about 0.1 mL to about 4.5 mL, about 0.1 mL to about 4.0 mL, about 0.1 mL to about 3.5 mL, about 0.1 mL to about 3.0 mL, about 0.1 mL to about 2.5 mL, about 0.1 mL to about 2.0 mL, about 0.1 mL to about 1.5 mL, about 0.1 mL to about 1.0 mL, about 0.1 mL to about 0.5 mL, about 0.5 mL to about 5.0 mL, about 0.5 mL to about 4.5 mL, about 0.5 mL to about 4.0 mL, about 0.5 mL to about 3.5 mL, about 0.5 mL to about 3.0 mL, about 0.5 mL to about 2.5 mL, about 0.5 mL to about 2.0 mL, about 0.5 mL to about 1.5 mL, about 0.5 mL to about 1.0 mL, about 1.0 mL to about 5.0 mL, about 1.0 mL to about 4.5 mL, about 1.0 mL to about 4.0 mL, about 1.0 mL to about 3.5 mL, about 1.0 mL to about 3.0 mL, about 1.0 mL to about 2.5 mL, about 1.0 mL to about 2.0 mL, about 1.0 mL to about 1.5 mL, about 1.5 mL to about 5.0 mL, about 1.5 mL to about 4.5 mL, about 1.5 mL to about 4.0 mL, about 1.5 mL to about 3.5 mL, about 1.5 mL to about 3.0 mL, about 1.5 mL to about 2.5 mL, about 1.5 mL to about 2.0 mL, about 2.0 mL to about 5.0 mL, about 2.0 mL to about 4.5 mL, about 2.0 mL to about 4.0 mL, about 2.0 mL to about 3.5 mL, about 2.0 mL to about 3.0 mL, about 2.0 mL to about 2.5 mL, about 2.5 mL to about 5.0 mL, about 2.5 mL to about 4.5 mL, about 2.5 mL to about 4.0 mL, about 2.5 mL to about 3.5 mL, about 2.5 mL to about 3.0 mL, about 3.0 mL to about 5.0 mL, about 3.0 mL to about 4.5 mL, about 3.0 mL to about 4.0 mL, about 3.0 mL to about 3.5 mL, about 3.5 mL to about 5.0 mL, about 3.5 mL to about 4.5 mL, about 3.5 mL to about 4.0 mL, about 4.0 mL to about 5.0 mL, about 4.0 mL to about 4.5 mL, about 4.5 mL to about 5.0 mL, about 4.5 to about 6.0 mL, about 5.0 to about 6.0 mL, about 5.0 mL to about 5.5 mL, about 5.5 to about 6.0 mL, about 5.0 to about 6.5 mL, about 5.5 to about 6.5 mL, or about 6.0 to about 6.5 mL.

In some embodiments, the injection volume is about 1.5 mL to about 3.5 mL, for example about 1.5 mL, about 1.6 mL, about 1.7 mL, about 1.8 mL, about 1.9 mL, about 2.0 mL, about 2.1 mL, about 2.2 mL, about 2.3 mL, about 2.4 mL, about 2.5 mL, about 2.6 mL, about 2.7 mL, about 2.8 mL, about 2.9 mL, about 3.0 mL, about 3.1 mL, about 3.2 mL, about 3.3 mL, about 3.4 mL, or about 3.5 mL.

In some embodiments, the injection volume is less than or equal to 2 mL. In some embodiments, the injection volume is from about 2 mL to about 10 mL.

In some embodiments, the injection volume is from about 0.5 mL to about 6 mL.

In some embodiments, the injection volume is about 0.5 mL, 1.0 mL, 1.5 mL, 2.0 mL, 2.5 mL, 3.0 mL, 3.5 mL, 4.0 mL, 4.5 mL, 5.0 mL, 5.5 mL, or about 6.0 mL.

In some embodiments, the injection volume is about 0.5 mL to about 2.5 mL. In some embodiments, the volume of the pharmaceutical composition for injection administered is about 0.8 mL to about 1.2 mL. In some embodiments, the injection volume is about 1.0 mL.

In some embodiments, the injection volume is about 1.0 mL to about 3.0 mL. In some embodiments, the injection volume is about 1.8 mL to about 2.2 mL. In some embodiments, the injection volume is about 2.0 mL.

In some embodiments, the injection volume is about 1.5 mL to about 3.5 mL. In some embodiments, the injection volume is about 2.0 mL to about 3.0 mL. In some embodiments, the injection volume is about 2.5 mL.

In some embodiments, the pharmaceutical compositions described herein are evaluated for syringeability and injectability. As defined herein, syringeability refers to the ability of the pharmaceutical composition to pass easily through a hypodermic needle on injection, whereas injectability refers to the performance of the pharmaceutical composition during injection, as measured by parameters such as glide force (measured in Newton, or N) and viscosity (measured in centipoise, or cP), using suitable methods known in the art. In some embodiments, the pharmaceutical composition exhibits a maximum glide force of about 20 N to about 40 N when measured using a 22G ETW (extra thin wall) hypodermic needle (0.5″ length). In some embodiments, the pharmaceutical composition exhibits a maximum glide force of about 20 N to about 35 N when measured using a 22G ETW hypodermic needle (0.5″ length). In some embodiments, the pharmaceutical composition exhibits a maximum glide force of about 24 N to about 30 N when measured using a 22G ETW hypodermic needle (0.5″ length). In some embodiments, the pharmaceutical composition exhibits a maximum glide force of about 20 N to about 40 N when measured using a 20G hypodermic needle (1.5″ length). In some embodiments, the estimated viscosity of the pharmaceutical composition is about 300 cP to about 600 cP. In some embodiments, the estimated viscosity of the pharmaceutical composition is about 300 cP to about 400 cP. In some embodiments, the estimated viscosity of the pharmaceutical composition is about 400 cP to about 500 cP. In some embodiments, the estimated viscosity of the pharmaceutical composition is about 500 cP to about 600 cP.

In some embodiments, the pharmaceutical compositions provided herein provide good tolerability with manageable injection site reactions (ISR). In some embodiments, the pharmaceutical compositions provided herein provide good tolerability with substantially no injection site reactions (ISR). In some embodiments, the pharmaceutical compositions provided herein provide good tolerability with no injection site reactions (ISR). In some embodiments, the pharmaceutical compositions provided herein provide good tolerability with minimal injection site reactions (ISR). An advantage of certain pharmaceutical compositions disclosed herein is that they allow for high concentrations of the compound of Formula I, or pharmaceutically acceptable salt thereof, and therefore can be administered with lower injection volumes.

In some embodiments, the pharmaceutical compositions provided herein provide enhanced plasma concentrations. In some embodiments, the pharmaceutical compositions provided herein provide enhanced pharmacokinetic profiles. In some embodiments, the pharmaceutical compositions provided herein provide enhanced drug release profiles.

### Aqueous Suspensions

In some embodiments, the pharmaceutical composition is an aqueous suspension. As used herein, an “aqueous suspension” refers to a suspension wherein water is present in ≥5% of the total pharmaceutical composition weight (wt. %). In some embodiments, the pharmaceutical composition comprises at least 5 wt. %, at least 10 wt. %, at least 15 wt. %, or at least 50 wt. % water. In some embodiments, the pharmaceutical composition comprises at least 50 wt. % water.

In some embodiments, the pharmaceutical composition comprises less than 50 wt. % water. In some embodiments, the pharmaceutical composition comprises about 5 wt. % to about 90 wt. % water.

In some embodiments, the pharmaceutical composition comprises about 5 wt. % to about 50 wt. % water. In some embodiments, the pharmaceutical composition comprises about 5 wt. % to about 30 wt. % water. In some embodiments, the pharmaceutical composition comprises about 10 wt. % to about 15 wt. %, about 10 wt. % to about 20 wt. %, or about 15 wt. % to about 20 wt. % water. In some embodiments, the pharmaceutical composition comprises about 10 wt. % to about 20 wt. %, about 10 wt. % to about 15 wt. %, or about 15 wt. % to about 20 wt. % water. In some embodiments, the pharmaceutical composition comprises about 16 wt. % water. In some embodiments, the pharmaceutical composition comprises about 13 wt. % water.

In some embodiments, the pharmaceutical composition comprises from about 5 wt. % to about 90 wt. %, about 5 wt. % to about 85 wt. %, about 5 wt. % to about 75 wt. %, about 5 wt. % to about 50 wt. %, about 5 wt. % to about 30 wt. %, about 10 wt. % to about 30 wt. %, about 10 wt. % to about 20 wt. %, about 15 wt. % to about 25 wt. %, about 20 wt. % to about 30 wt. %, or about 15 wt. % to about 20 wt. % water. In some embodiments, the pharmaceutical composition comprises about 5 wt. % to about 95 wt. %, about 15 wt. % to about 90 wt. %, about 25 wt. % to about 90 wt. %, about 50 wt. % to about 95 wt. %, about 50 wt. % to about 90 wt. %, about 60 wt. % to about 80 wt. %, about 50 wt. % to about 70 wt. %, or about 45 wt. % to about 65 wt. % water. In some embodiments, the pharmaceutical composition comprises about 50 wt. % to about 90 wt. %, or about 60 wt. % to about 80 wt. % water.

In some embodiments, the pharmaceutical composition comprises about 55 wt. % to about 85 wt. %, about 50 wt. % to about 80 wt. %, about 55 wt. % to about 65 wt. %, about 45 wt. % to about 65 wt. % water. In some embodiments, the pharmaceutical composition comprises about 59 wt. % water. In some embodiments, the pharmaceutical composition comprises about 56 wt. % water.

In some embodiments, the pharmaceutical composition comprises one or more hydrophilic carriers (e.g., stabilizers). In some embodiments, hydrophilic carriers may increase the surface area of the drug-carrier particles and lead to enhanced drug solubility and dissolution rate. Examples of hydrophilic carriers that may used in the preparation of pharmaceutical formulations include crystalline carriers (e.g., urea and sugars such as D-mannitol, D-fructose, D-maltose), polymeric carriers (e.g., poly(lactic-co-glycolic acid) (PLGA), Povidone (PVP), polyethylene glycol (PEG), polymethacrylates, hydroxypropylmethylcellulose (HPMC), hydroxypropylcellulose (HPC), and sodium carboxymethyl cellulose (CMC)), surfactants (e.g., poloxamers, sodium lauryl sulfate (SLS), Gelucire®, polysorbates (Tween®), vitamin E d-α-tocopheryl polyethylene glycol 1000 succinate (TGPS), polyethylene glycol (15)-hydroxystearate (Kolliphor® HS 15), and polyoxyl-35 castor oil (Kolliphor® ELP)), and mixtures thereof.

In some embodiments, the aqueous suspension comprises a hydrophilic carrier selected from a crystalline carrier, a polymeric carrier, a surfactant, and combinations thereof. In some embodiments, the pharmaceutical composition comprises a polymeric carrier. In some embodiments, the polymeric carrier is selected from polyethylene glycol, povidone, Hypromellose (HPMC), and combinations thereof.

In some embodiments, the aqueous suspension comprises polyethylene glycol. In some embodiments, the polyethylene glycol is selected from PEG 200, PEG 300, PEG 400, PEG 600, PEG 1000, PEG 1500, PEG 3350, PEG 4000, methoxy polyethylene glycol (mPEG), and combinations thereof. In some embodiments, the polyethylene glycol is PEG 300.

In some embodiments, the pharmaceutical composition comprises about 50 wt. % to about 70 wt. % polyethylene glycol, about 60 wt. % to about 70 wt. % polyethylene glycol, or about 50 wt. % to about 55 wt. % polyethylene glycol. In some embodiments, the pharmaceutical composition comprises about 65 wt. % polyethylene glycol. In some embodiments, the pharmaceutical composition comprises about 53 wt. % polyethylene glycol.

In some embodiments, the pharmaceutical composition comprises the compound of Formula I, or pharmaceutically acceptable salt thereof; polyethylene glycol; and water. In some embodiments, the pharmaceutical composition comprises the compound of Formula I, polyethylene glycol, and water. In some embodiments, the pharmaceutical composition comprises the compound of Formula I, polyethylene glycol, and water, wherein the compound of Formula I has Form I. In some embodiments, the pharmaceutical composition comprises the compound of Formula I, polyethylene glycol, and water, wherein the compound of Formula I is micronized. In some embodiments, the pharmaceutical composition comprises the compound of Formula I, polyethylene glycol, and water, wherein the compound of Formula I has a particle size distribution d₉₀ of from about 1 μm to about 90 μm. In some embodiments, the pharmaceutical composition comprises the compound of Formula I, polyethylene glycol, and water, wherein the compound of Formula I has a particle size distribution d₉₀ of from about 5 μm to about 15 μm In some embodiments, the pharmaceutical composition comprises the compound of Formula I, polyethylene glycol, and water, wherein the polyethylene glycol is PEG 300.

In some embodiments, the pharmaceutical composition comprises a mixture comprising polyethylene glycol and water, wherein the ratio of polyethylene glycol and water in the mixture is from about 60:40 to about 90:10 wt. In some embodiments, the pharmaceutical composition comprises a mixture comprising polyethylene glycol and water, wherein the ratio of polyethylene glycol and water in the mixture is from about 60:40 to about 80:20 wt. %. In some embodiments, the pharmaceutical composition comprises a mixture comprising polyethylene glycol and water, wherein the ratio of polyethylene glycol and water in the mixture is about 90:10 wt. %, about 80:20 wt. %, about 70:30 wt. %, or about 60:40 wt. %. In some embodiments, the pharmaceutical composition comprises a mixture comprising polyethylene glycol and water, wherein the ratio of polyethylene glycol and water in the mixture is about 80:20 wt. %. In some embodiments, the pharmaceutical composition comprises a mixture comprising polyethylene glycol and water, wherein the ratio of polyethylene glycol and water in the mixture is about 60:40 wt. %.

In some embodiments, the pharmaceutical composition comprises:
about 15 wt. % to about 50 wt. % of the compound of Formula I;
about 50 wt. % to about 70 wt. % polyethylene glycol; and
about 10 wt. % to about 20 wt. % water.

In some embodiments, the pharmaceutical composition comprises:
about 15 wt. % to about 35 wt. % of the compound of Formula I;
about 50 wt. % to about 70 wt. % polyethylene glycol; and
about 10 wt. % to about 20 wt. % water.

In some embodiments, the pharmaceutical composition comprises:
about 30 wt. % to about 35 wt. % of the compound of Formula I;
about 50 wt. % to about 55 wt. % polyethylene glycol; and
about 10 wt. % to about 15 wt. % water.

In some embodiments, the pharmaceutical composition comprises:
about 33 wt. % of the compound of Formula I;
about 53 wt. % polyethylene glycol; and
about 13 wt. % water.

In some embodiments, the pharmaceutical composition comprises:
about 15 wt. % to about 25 wt. % of the compound of Formula I;
about 60 wt. % to about 70 wt. % polyethylene glycol; and
about 15 wt. % to about 20 wt. % water.

In some embodiments, the pharmaceutical composition comprises:
about 19 wt. % of the compound of Formula I;
about 65 wt. % polyethylene glycol; and
about 16 wt. % water.

In some embodiments, the pharmaceutical composition comprises:
about 10 wt. % to about 40 wt. % of the compound of Formula I; and
about 60 wt. % to about 90 wt. % of a mixture comprising polyethylene glycol and water, wherein the ratio of polyethylene glycol and water in the mixture is from about 60:40 to about 90:10 wt. %.

In some embodiments, the pharmaceutical composition comprises:
about 15 wt. % to about 35 wt. % of the compound of Formula I;
about 50 wt. % to about 65 wt. % of a mixture comprising polyethylene glycol and water; wherein the ratio of polyethylene glycol and water in the mixture is from about 60:40 to about 80:20 wt. %.

In some embodiments, the pharmaceutical composition comprises:
about 30 wt. % to about 35 wt. % of the compound of Formula I; and
about 65 wt. % to about 70 wt. % of a mixture comprising polyethylene glycol and water, wherein the ratio of polyethylene glycol and water in the mixture is about 80:20 wt. %.

In some embodiments, the pharmaceutical composition comprises:
about 15 wt. % to about 25 wt. % of the compound of Formula I; and
about 75 wt. % to about 85 wt. % of mixture comprising polyethylene glycol and water, wherein the ratio of polyethylene glycol and water in the mixture is about 80:20 wt. %.

In some embodiments, the pharmaceutical composition comprises the compound of Formula I, polyethylene glycol, and water, wherein the concentration of the compound of Formula I is from about 200 mg/mL to about 500 mg/mL. In some embodiments, the pharmaceutical composition comprises the compound of Formula I, polyethylene glycol, and water, wherein the concentration of the compound of Formula I is from about 225 mg/mL to about 400 mg/mL. In some embodiments, the pharmaceutical composition comprises the compound of Formula I, polyethylene glycol, and water, wherein the concentration of the compound of Formula I is about 225 mg/mL. In some embodiments, the pharmaceutical composition comprises the compound of Formula I, polyethylene glycol, and water, wherein the concentration of the compound of Formula I is about 400 mg/mL. In some embodiments, the pharmaceutical composition comprises the compound of Formula I, polyethylene glycol, and water, wherein the concentration of the compound of Formula I is about 450 mg/mL.

In some embodiments, the pharmaceutical composition comprises:
about 15 wt. % to about 50 wt. % of the compound of Formula I;
about 40 wt. % to about 70 wt. % polyethylene glycol; and
about 10 wt. % to about 20 wt. % water;
wherein the compound of Formula I is present at a concentration of from about 200 mg/mL to about 500 mg/mL.

In some embodiments, the pharmaceutical composition comprises:
about 15 wt. % to about 50 wt. % of the compound of Formula I;
about 40 wt. % to about 70 wt. % polyethylene glycol; and
about 10 wt. % to about 20 wt. % water;
wherein the compound of Formula I is present at a concentration of about 450 mg/mL.

In some embodiments, the pharmaceutical composition comprises:
about 15 wt. % to about 35 wt. % of the compound of Formula I;
about 50 wt. % to about 70 wt. % polyethylene glycol; and
about 10 wt. % to about 20 wt. % water;
wherein the compound of Formula I is present at a concentration of from about 200 mg/mL to about 500 mg/mL.

In some embodiments, the pharmaceutical composition comprises:
about 30 wt. % to about 35 wt. % of the compound of Formula I;
about 50 wt. % to about 55 wt. % polyethylene glycol; and
about 10 wt. % to about 15 wt. % water;
wherein the compound of Formula I is present at a concentration of from about 200 mg/mL to about 500 mg/mL.

In some embodiments, the pharmaceutical composition comprises:
about 33 wt. % of the compound of Formula I;
about 53 wt. % polyethylene glycol; and
about 13 wt. % water;
wherein the compound of Formula I is present at a concentration of from about 200 mg/mL to about 500 mg/mL.

In some embodiments, the pharmaceutical composition comprises:
about 15 wt. % to about 25 wt. % of the compound of Formula I;
about 60 wt. % to about 70 wt. % polyethylene glycol; and
about 15 wt. % to about 20 wt. % water;
wherein the compound of Formula I is present at a concentration of from about 200 mg/mL to about 500 mg/mL.

In some embodiments, the pharmaceutical composition comprises:
about 19 wt. % of the compound of Formula I;
about 65 wt. % polyethylene glycol; and
about 16 wt. % water;
wherein the compound of Formula I is present at a concentration of from about 200 mg/mL to about 500 mg/mL.

In some embodiments, the pharmaceutical composition comprises:
about 15 wt. % to about 35 wt. % of the compound of Formula I;
about 50 wt. % to about 70 wt. % polyethylene glycol; and
about 10 wt. % to about 20 wt. % water;
wherein the compound of Formula I is present at a concentration of about 400 mg/mL.

In some embodiments, the pharmaceutical composition comprises:
about 30 wt. % to about 35 wt. % of the compound of Formula I;
about 50 wt. % to about 55 wt. % polyethylene glycol; and
about 10 wt. % to about 15 wt. % water;
wherein the compound of Formula I is present at a concentration of about 400 mg/mL.

In some embodiments, the pharmaceutical composition comprises:
about 33 wt. % of the compound of Formula I;
about 53 wt. % polyethylene glycol; and
about 13 wt. % water;
wherein the compound of Formula I is present at a concentration of about 400 mg/mL.

In some embodiments, the pharmaceutical composition comprises:
about 15 wt. % to about 25 wt. % of the compound of Formula I;
about 60 wt. % to about 70 wt. % polyethylene glycol; and
about 15 wt. % to about 20 wt. % water;
wherein the compound of Formula I is present at a concentration of about 400 mg/mL.

In some embodiments, the pharmaceutical composition comprises:
about 19 wt. % of the compound of Formula I;
about 65 wt. % polyethylene glycol; and
about 16 wt. % water;
wherein the compound of Formula I is present at a concentration of about 400 mg/mL.

In some embodiments, the pharmaceutical composition comprises:
about 15 wt. % to about 35 wt. % of the compound of Formula I;
about 50 wt. % to about 70 wt. % polyethylene glycol; and
about 10 wt. % to about 20 wt. % water;
wherein the compound of Formula I is present at a concentration of about 225 mg/mL.

In some embodiments, the pharmaceutical composition comprises:
about 30 wt. % to about 35 wt. % of the compound of Formula I;
about 50 wt. % to about 55 wt. % polyethylene glycol; and
about 10 wt. % to about 15 wt. % water;
wherein the compound of Formula I is present at a concentration of about 225 mg/mL.

In some embodiments, the pharmaceutical composition comprises:
about 33 wt. % of the compound of Formula I;
about 53 wt. % polyethylene glycol; and
about 13 wt. % water;
wherein the compound of Formula I is present at a concentration of about 225 mg/mL.

In some embodiments, the pharmaceutical composition comprises:
about 15 wt. % to about 25 wt. % of the compound of Formula I;
about 60 wt. % to about 70 wt. % polyethylene glycol; and
about 15 wt. % to about 20 wt. % water;
wherein the compound of Formula I is present at a concentration of about 225 mg/mL.

In some embodiments, the pharmaceutical composition comprises:
about 19 wt. % of the compound of Formula I;
about 65 wt. % polyethylene glycol; and
about 16 wt. % water;
wherein the compound of Formula I is present at a concentration of about 225 mg/mL.

In some embodiments, the aqueous suspension comprises a suspending agent. In some embodiments, the suspending agent is selected from methyl cellulose, carboxy methyl cellulose, polyethylene glycol, polypropylene glycol, poly(lactic-co-glycolic acid) (PLGA), glycerin, sodium carboxymethyl cellulose (CMC), hydroxypropyl methylcellulose (also referred to herein as “hypromellose” or “HPMC”), povidone, and any combination thereof.

In some embodiments, the pharmaceutical composition comprises from about 0.01 wt. % to about 10 wt. %, about 0.01 wt. % to about 7 wt. %, about 0.05 to about 5 wt. %, about 0.01 wt. % to about 1 wt. %, about 0.1 wt. % to about 5 wt. %, about 1 wt. % to about 5 wt. %, or about 2 wt. % to about 5 wt. % of the suspending agent. For example, the pharmaceutical composition comprises about 0.1 wt. % to about 4.5 wt. %, 0.1 wt. % to about 4.0 wt. %, 0.1 wt. % to about 3.5 wt. %, 0.1 wt. % to about 3.0 wt. %, 0.1 wt. % to about 2.5 wt. %, 0.1 wt. % to about 2.0 wt. %, 0.1 wt. % to about 1.5 wt. %, 0.1 wt. % to about 1.0 wt. %, 0.5 wt. % to about 5.0 wt. %, 0.5 wt. % to about 4.5 wt. %, 0.5 wt. % to about 4.0 wt. %, 0.5 wt. % to about 3.5 wt. %, 0.5 wt. % to about 3.0 wt. %, 0.5 wt. % to about 2.5 wt. %, 0.5 wt. % to about 2.0 wt. %, 0.5 wt. % to about 1.5 wt. % of the suspending agent.

In some embodiments, the pharmaceutical composition comprises from about 0.01 wt. % to about 7 wt. % of the suspending agent. In some embodiments, the pharmaceutical composition comprises from about 0.01 wt. % to about 1 wt. % of the suspending agent. In some embodiments, the pharmaceutical composition comprises from about 2 wt. % to about 5 wt. % of the suspending agent.

In some embodiments, the suspending agent is selected from Hypromellose (HPMC), povidone K12, sodium CMC, polyethylene glycol, and polyvinyl alcohol.

In some embodiments, the suspending agent is selected from the group consisting of methyl cellulose, carboxy methyl cellulose, hydroxypropyl methylcellulose, sodium CMC, povidone K12, povidone K17, povidone K25, povidone K30, povidone K90, Plasdone K29/32, and any combination thereof. In some embodiments, the suspending agent is selected from the group consisting of hydroxypropyl methylcellulose, povidone K12, povidone K17, povidone K25, Povidone K30, and Povidone K90, Plasdone K29/32 and any combination thereof. In some embodiments, the suspending agent comprises povidone K12. In some embodiments, the suspending agent comprises povidone K12. In some embodiments, the suspending agent comprises hydroxypropyl methylcellulose and povidone K12. In some embodiments, the suspending agent is povidone K12.

In some embodiments, the suspending agent is selected from the group consisting of hydroxypropyl methylcellulose and povidone. In some embodiments, the suspending agent comprises povidone. In some embodiments, the suspending agent comprises hydroxypropyl methylcellulose. In some embodiments, the suspending agent comprises hydroxypropyl methylcellulose and povidone. In some embodiments, the suspending agent is povidone. In some embodiments, the suspending agent is hydroxypropyl methylcellulose.

In some embodiments, the pharmaceutical composition comprises about 0.01 wt. % to about 1 wt. % hydroxypropyl methylcellulose. In some embodiments, the pharmaceutical composition comprises about 0.5 wt. % hydroxypropyl methylcellulose.

In some embodiments, the suspending agent is sodium carboxymethyl cellulose (CMC).

In some embodiments, the suspending agent is polyethylene glycol. In some embodiments, the suspending agent is polyethylene glycol, wherein the polyethylene glycol is selected from PEG 200, PEG 300, PEG 400, PEG 600, PEG 1000, PEG 1500, PEG 3350, PEG 4000, methoxy polyethylene glycol (mPEG), and combinations thereof. In some embodiments, the polyethylene glycol is selected from PEG 300, PEG 3350, PEG 4000, and combinations thereof. In some embodiments, the suspending agent is polyethylene glycol, wherein the polyethylene glycol is PEG 3350. In some embodiments, the pharmaceutical composition comprises about 0.01 wt. % to about 5 wt. % polyethylene glycol.

In some embodiments, the suspending agent is polyvinyl alcohol (PVA).

In some embodiments, the aqueous suspension comprises a buffer. In some embodiments, the buffer is a phosphate buffer. In some embodiments, the buffer comprises sodium phosphate monobasic, sodium phosphate dibasic, and/or sodium chloride. In some embodiments, the buffer comprises sodium phosphate monobasic and sodium phosphate dibasic.

In some embodiments, the buffer is a sodium phosphate buffer. In some embodiments, the pharmaceutical composition comprises about 0.01 wt. % to about 1 wt. % polysorbate. In some embodiments, the buffer is a sodium phosphate buffer comprising sodium phosphate monobasic and sodium phosphate dibasic and is present in the pharmaceutical composition in an amount of about 0.1 wt. % to about 0.3 wt. %. In some embodiments, the amount of the buffer in the pharmaceutical composition is about 0.1 wt. %, about 0.2 wt. %, or about 0.3 wt. %. In some embodiments, the pharmaceutical composition has a phosphate buffer concentration of about 0.1M to about 0.15M. In some embodiments, the amount of buffer in the pharmaceutical composition is the amount of buffer required to maintain a pH of between about 7 and 7.8 (e.g., 7.4).

In some embodiments, the pharmaceutical composition has a pH of from about 7.0 to about 7.8. In some embodiments, the pharmaceutical composition has a pH of about 7.4.

In some embodiments, the aqueous suspension comprises a surfactant (e.g., a wetting agent). In some embodiments, the surfactant is selected from the group consisting of polysorbates (e.g., a Tween), poloxamers, Lecithin, a fatty acid polyethylene glycol ester, an ethoxylated castor oil, sorbitan trioleate, sodium deoxycholate, and vitamin E d-α-tocopheryl polyethylene glycol 1000 succinate (TGPS). In some embodiments, the surfactant is selected from a group consisting of polysorbates, poloxamers, Lecithin, polyethylene glycol (15)-hydroxystearate, polyoxyl-35 castor oil, sorbitan trioleate, sodium deoxycholate, and vitamin E TGPS. In some embodiments, the surfactant is selected from the group consisting of polysorbates, poloxamers, polyethylene glycol (15)-hydroxystearate, polyoxyl-35 castor oil, and vitamin E TGPS.

In some embodiments, the surfactant is a stabilizer. In some embodiments, the aqueous suspension is a nanosuspension comprising a stabilizer. In some embodiments, the surfactant is a stabilizer selected from the group consisting of polysorbates (e.g., a Tween), poloxamers, Lecithin, a fatty acid polyethylene glycol ester, an ethoxylated castor oil, sorbitan trioleate, sodium deoxycholate, DOSS (docusate sodium), and vitamin E d-α-tocopheryl polyethylene glycol 1000 succinate (TGPS).

In some embodiments, the surfactant is selected from a poloxamer, a polysorbate, and combinations thereof. In some embodiments, the surfactant is selected from the group consisting of Tween 20, Tween 80, poloxamer 188, poloxamer 338, poloxamer 407, poloxamer 213, and poloxamer 2930.

In some embodiments, the surfactant comprises polysorbate. In some embodiments, the surfactant is a Tween. In some embodiments, the surfactant is Tween 20, Tween 40, Tween 60, Tween 65, or Tween 80. In some embodiments, the surfactant is Tween 20.

In some embodiments, the surfactant comprises sodium deoxycholate.

In some embodiments, the amount of surfactant in the pharmaceutical composition is from about 0.02 wt. % to about 2.0 wt. %. In some embodiments, the amount of surfactant in the pharmaceutical composition is from about 0.02 wt. % to about 1.50 wt. %, 0.02 wt. % to about 1.00 wt. %, 0.02 wt. % to about 0.50 wt. %, 0.02 wt. % to about 0.25 wt. %, 0.05 wt. % to about 2.00 wt. %, 0.05 wt. % to about 1.50 wt. %, 0.05 wt. % to about 1.00 wt. %, 0.05 wt. % to about 0.50 wt. %, 0.05 wt. % to about 0.25 wt. %, 0.10 wt. % to about 2.00 wt. %, 0.10 wt. % to about 1.50 wt. %, 0.10 wt. % to about 1.00 wt. %, or 0.10 wt. % to about 0.50 wt. %. In some embodiments, the amount of surfactant (e.g., Tween 20) in the pharmaceutical composition is from about 0.1 wt. % to about 1.0 wt. %. In some embodiments, the amount of surfactant (e.g., Tween 20) in the pharmaceutical composition is from about 0.2 wt. % to about 0.6 wt. %. In some embodiments, the amount of surfactant (e.g., Tween 20) in the pharmaceutical composition is about 0.4 wt. %.

In some embodiments, the pharmaceutical composition comprises about 0.01 wt. % to about 5 wt. % polysorbate, or about 0.01 wt. % to about 1 wt. % polysorbate. In some embodiments, the pharmaceutical composition comprises about 0.5 wt. % polysorbate.

In some embodiments, the pharmaceutical composition comprises about 0.01 wt. % to about 5 wt. % sodium deoxycholate, or about 0.01 wt. % to about 1 wt. % sodium deoxycholate.

In some embodiments, the pharmaceutical composition comprises about 0.3 wt. % polysorbate.

In some embodiments, the aqueous suspension comprises a suspending agent, a surfactant, and a buffer. In some embodiments, the aqueous suspension comprises HPMC, a surfactant, and a buffer. In some embodiments, the aqueous suspension comprises HPMC, polysorbate, and a buffer. In some embodiments, the aqueous suspension comprises HPMC, polysorbate, and a phosphate buffer.

In some embodiments, the pharmaceutical composition comprises the compound of Formula I, or pharmaceutically acceptable salt thereof; HPMC; polysorbate; sodium phosphate buffer; and water. In some embodiments, the pharmaceutical composition comprises the compound of Formula I, HPMC, polysorbate, sodium phosphate buffer, and water. In some embodiments, the pharmaceutical composition comprises the compound of Formula I, HPMC, polysorbate, sodium phosphate buffer, and water, wherein the compound of Formula I has Form I. In some embodiments, the pharmaceutical composition comprises the compound of Formula I, HPMC, polysorbate, sodium phosphate buffer, and water, wherein the compound of Formula I is micronized. In some embodiments, the pharmaceutical composition comprises the compound of Formula I, HPMC, polysorbate, sodium phosphate buffer, and water, wherein the compound of Formula I has a particle size distribution d₉₀ of from about 1 μm to about 90 μm. In some embodiments, the pharmaceutical composition comprises the compound of Formula I, HPMC, polysorbate, sodium phosphate buffer, and water, wherein the compound of Formula I has a particle size distribution d₉₀ of from about 5 μm to about 20 μm.

In some embodiments, the pharmaceutical composition comprises:
about 10 wt. % to about 40 wt. % of the compound of Formula I;
about 0.01 wt. % to about 1 wt. % HPMC;
about 0.01 wt. % to about 1 wt. % polysorbate;
about 0.01 wt. % to about 1 wt. % sodium phosphate buffer; and
about 50 wt. % to about 90 wt. % water.

In some embodiments, the pharmaceutical composition comprises:
about 22 wt. % of the compound of Formula I;
about 0.5 wt. % HPMC;
about 0.5 wt. % polysorbate;
about 0.01 wt. % to about 1 wt. % sodium phosphate buffer; and
about 50 wt. % to about 90 wt. % water.

In some embodiments, the pharmaceutical composition comprises a salt of the compound of Formula I, a suspending agent, and a surfactant. In some embodiments, the pharmaceutical composition comprises a salt of the compound of Formula I, a suspending agent, a surfactant, and a buffer.

In some embodiments, the pharmaceutical composition comprises a sodium salt of the compound of Formula I, HPMC, polysorbate, sodium phosphate buffer, and water. In some embodiments, the pharmaceutical composition comprises a sodium salt of the compound of Formula I, HPMC, polysorbate, sodium phosphate buffer, and water, wherein the sodium salt of the compound of Formula I has crystalline Form I. In some embodiments, the pharmaceutical composition comprises a sodium salt of the compound of Formula I, HPMC, polysorbate, sodium phosphate buffer, and water, wherein the sodium salt of the compound of Formula I is micronized. In some embodiments, the pharmaceutical composition comprises a sodium salt of the compound of Formula I, HPMC, polysorbate, sodium phosphate buffer, and water, wherein the sodium salt of the compound of Formula I has a particle size distribution d₉₀ of from about 1 μm to about 90 μm. In some embodiments, the pharmaceutical composition comprises a sodium salt of the compound of Formula I, HPMC, polysorbate, sodium phosphate buffer, and water, wherein the sodium salt of the compound of Formula I has a particle size distribution d₉₀ of from about 1 μm to about 30 μm.

In some embodiments, the pharmaceutical composition comprises:
about 10 wt. % to about 40 wt. % of a sodium salt of the compound of Formula I;
about 0.01 wt. % to about 1 wt. % HPMC;
about 0.01 wt. % to about 1 wt. % polysorbate;
about 0.01 wt. % to about 1 wt. % sodium phosphate buffer; and
about 50 wt. % to about 90 wt. % water.

In some embodiments, the pharmaceutical composition comprises:
about 24 wt. % of the sodium salt of the compound of Formula I;
about 0.5 wt. % HPMC;
about 0.5 wt. % polysorbate;
about 0.01 wt. % to about 1 wt. % sodium phosphate buffer; and
about 50 wt. % to about 90 wt. % water.

In some embodiments, the aqueous suspension comprises polyethylene glycol and a surfactant. In some embodiments, the aqueous suspension comprises polyethylene glycol and polysorbate.

In some embodiments, the pharmaceutical composition comprises the compound of Formula I, or pharmaceutically acceptable salt thereof; polyethylene glycol; polysorbate; and water. In some embodiments, the pharmaceutical composition comprises the compound of Formula I, polyethylene glycol, polysorbate, and water. In some embodiments, the pharmaceutical composition comprises the compound of Formula I, polyethylene glycol, polysorbate, and water, wherein the compound of Formula I has Form I.

In some embodiments, the pharmaceutical composition comprises the compound of Formula I, or pharmaceutically acceptable salt thereof; polyethylene glycol; polysorbate; and water, wherein the pharmaceutical composition is a nanosuspension.

In some embodiments, the pharmaceutical composition comprises the compound of Formula I, polyethylene glycol, polysorbate, and water, wherein the compound of Formula I has a particle size distribution d₉₀ of less than 1 μm. In some embodiments, the pharmaceutical composition comprises the compound of Formula I, polyethylene glycol, polysorbate, and water, wherein the compound of Formula I has a particle size distribution d₉₀ of from about 200 nm to about 900 nm. In some embodiments, the pharmaceutical composition comprises the compound of Formula I, polyethylene glycol, polysorbate, and water, wherein the polyethylene glycol is PEG 3350. In some embodiments, the pharmaceutical composition comprises the compound of Formula I, polyethylene glycol, polysorbate, and water, wherein the polysorbate is Tween 20.

In some embodiments, the pharmaceutical composition comprises:
about 10 wt. % to about 30 wt. % of the compound of Formula I;
about 0.01 wt. % to about 5 wt. % polyethylene glycol;
about 0.01 wt. % to about 5 wt. % polysorbate; and
about 60 wt. % to about 80 wt. % water.

In some embodiments, the aqueous suspension comprises a poloxamer. In general, poloxamers are synthetic non-ionic triblock of linear copolymers having a central hydrophobic chain of polyoxypropylene adjacent to two hydrophilic polypropylene oxide, in certain instances in a 4:2:4 weight ratio. Accordingly, in certain embodiments, the pharmaceutical compositions disclosed herein include a block copolymer comprised of one polyoxypropylene segment and two hydrophilic polypropylene oxide segments. In certain embodiments, the ratio of the polyoxypropylene segment to the two hydrophilic polypropylene oxide segments is 4:2:4 (hydrophilic polypropylene oxide: polyoxypropylene: hydrophilic polypropylene oxide). Poloxamers are generally understood to have the following structure:

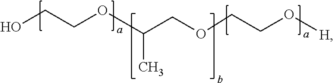

where a and b are integers. For example, a is between about 2 and about 130 and b is between about 15 and about 67. Poloxamer 188, for example, is understood to have a molecular weight from about 7680 to about 9510 Daltons (where a is about 80 and b is about 27). In some instances, poloxamer 188 has an average molecular weight of about 8400 Daltons. Poloxamer 338 has a molecular weight in the range of from about 12700 Da to about 17400 Da (where a is about 141 and b is about 44).

In some embodiments, the poloxamer is selected from poloxamer 188, poloxamer 338, and poloxamer 407. In some embodiments, the poloxamer is poloxamer 338. In some embodiments, the poloxamer is poloxamer 188. In some embodiments, the poloxamer is poloxamer 407.

In some embodiments, the pharmaceutical composition comprises from about 0.1 wt. % to about 10 wt. %, about 1 wt. % to about 7 wt. %, about 0.5 to about 5 wt. %, about 0.1 wt. % to about 5 wt. %, about 1 wt. % to about 5 wt. %, or about 2 wt. % to about 5 wt. % of the poloxamer.

In some embodiments, the pharmaceutical composition comprises from about 1 wt. % to about 7 wt. % of the poloxamer. In some embodiments, the pharmaceutical composition comprises from about 2 wt. % to about 5 wt. % of the poloxamer. In some embodiments, the pharmaceutical composition comprises about 2 wt. % of the poloxamer.

In some embodiments, the pharmaceutical composition comprises about 1 wt. % to about 5 wt. % of poloxamer 338. In some embodiments, the pharmaceutical composition comprises about 1 wt. % to about 3 wt. % of poloxamer 338. In some embodiments, the pharmaceutical composition comprises about 3 wt. % of poloxamer 338.

In some embodiments, the pharmaceutical composition comprises about 1 wt. % to about 5 wt. % of poloxamer 407. In some embodiments, the pharmaceutical composition comprises about 1 wt. % to about 3 wt. % of poloxamer 407. In some embodiments, the pharmaceutical composition comprises about 2 wt. % of poloxamer 407. In some embodiments, the pharmaceutical composition comprises about 3 wt. % of poloxamer 407.

In some embodiments, the aqueous suspension comprises a tonicity agent. As used herein, “tonicity agent” refers to any pharmaceutically acceptably excipient capable of modifying the tonicity of the suspension (i.e., the osmotic pressure gradient). In some embodiments, the tonicity agent is selected from saline, glycerin, mannitol, dextrose, trehalose, and a salt solution (e.g., sodium chloride solution). In some embodiments, the tonicity agent is mannitol.

In some embodiments, the pharmaceutical composition comprises about 1 wt. % to about 5 wt. %, or about 1 wt. % to about 3 wt. % mannitol. In some embodiments, the pharmaceutical composition comprises about 2 wt. % mannitol. In some embodiments, the pharmaceutical composition comprises about 2.5 wt. % mannitol.

In some embodiments, the aqueous suspension comprises a poloxamer, and further comprises a tonicity agent.

In some embodiments, the aqueous suspension comprises a poloxamer, and further comprises one or more additional surfactants. In some embodiments, the aqueous suspension comprises a poloxamer and a polysorbate.

In some embodiments, the pharmaceutical composition comprises a compound of Formula I, or pharmaceutically acceptable salt thereof; a poloxamer; and water.

In some embodiments, the pharmaceutical composition comprises a calcium salt of the compound of Formula I, a poloxamer, and water.

In some embodiments, the pharmaceutical composition comprises a calcium salt of the compound of Formula I, poloxamer, and water, wherein the calcium salt of the compound of Formula I is micronized. In some embodiments, the pharmaceutical composition comprises a calcium salt of the compound of Formula I, poloxamer, and water, wherein the calcium salt of the compound of Formula I has a particle size distribution d₉₀ of from about 1 μm to about 90 μm. In some embodiments, the pharmaceutical composition comprises a calcium salt of the compound of Formula I, poloxamer, and water, wherein the calcium salt of the compound of Formula I has a particle size distribution d₉₀ of from about 1 μm to about 20 μm. In some embodiments, the pharmaceutical composition comprises a calcium salt of the compound of Formula I, poloxamer, and water, wherein the calcium salt of the compound of Formula I has Form I. In some embodiments, the pharmaceutical composition comprises a calcium salt of the compound of Formula I, poloxamer, and water, wherein the calcium salt of the compound of Formula I is amorphous.

In some embodiments, the pharmaceutical composition comprises:
about 30 wt. % to about 40 wt. % of a calcium salt of the compound of Formula I;
about 1 wt. % to about 5 wt. % of the poloxamer; and
about 55 wt. % to about 65 wt. % water.

In some embodiments, the pharmaceutical composition comprises a calcium salt of the compound of Formula I, poloxamer 338, and water.

In some embodiments, the pharmaceutical composition comprises:
about 30 wt. % to about 40 wt. % of a calcium salt of the compound of Formula I;
about 1 wt. % to about 5 wt. % of poloxamer 338; and
about 55 wt. % to about 65 wt. % water.

In some embodiments, the pharmaceutical composition comprises:
about 38 wt. % of a calcium salt of the compound of Formula I;
about 3 wt. % of poloxamer 338; and
about 59 wt. % water.

In some embodiments, the pharmaceutical composition comprises a calcium salt of the compound of Formula I, or pharmaceutically acceptable salt thereof; a poloxamer; a tonicity agent; and water. In some embodiments, the pharmaceutical composition comprises a calcium salt of the compound of Formula I, a poloxamer, a tonicity agent, and water. In some embodiments, the pharmaceutical composition comprises a calcium salt of the compound of Formula I, poloxamer 338, mannitol, and water.

In some embodiments, the pharmaceutical composition comprises:
about 30 wt. % to about 50 wt. % of a calcium salt of the compound of Formula I;
about 1 wt. % to about 5 wt. % of poloxamer 338;
about 1 wt. % to about 5 wt. % mannitol; and
about 45 wt. % to about 65 wt. % water.

In some embodiments, the pharmaceutical composition comprises:
about 40 wt. % of a calcium salt of the compound of Formula I;
about 3 wt. % of poloxamer 338;
about 2 wt. % mannitol; and
about 55 wt. % water.

In some embodiments, the pharmaceutical composition comprises a compound of Formula I, a poloxamer, and water. In some embodiments, the pharmaceutical composition comprises a compound of Formula I, a poloxamer, and water, wherein the compound of Formula I has Form I. In some embodiments, the pharmaceutical composition comprises a compound of Formula I, a poloxamer, and water, wherein the compound of Formula I has a particle size distribution d₉₀ of from about 1 μm to about 90 μm. In some embodiments, the pharmaceutical composition comprises a compound of Formula I, a poloxamer, and water, wherein the compound of Formula I has a particle size distribution d₉₀ of from about 1 μm to about 90 μm. In some embodiments, the pharmaceutical composition comprises a compound of Formula I, a poloxamer, and water, wherein the compound of Formula I has a particle size distribution d₉₀ of from about 5 μm to about 15 μm.

In some embodiments, the pharmaceutical composition comprises a compound of Formula I, a poloxamer, and water, wherein the pharmaceutical composition is a nanosuspension. In some embodiments, the pharmaceutical composition comprises a compound of Formula I, a poloxamer, and water, wherein the pharmaceutical composition is a nanosuspension and wherein the compound of Formula I has Form I. In some embodiments, the pharmaceutical composition comprises a compound of Formula I, a poloxamer, and water, wherein the compound of Formula I has a particle size distribution d₉₀ of less than 1 μm. In some embodiments, the pharmaceutical composition comprises a compound of Formula I, a poloxamer, and water, wherein the compound of Formula I has a particle size distribution d₉₀ of from about 200 nm to about 900 nm.

In some embodiments, the compound of Formula I has a particle size distribution d₁₀ of about 50 nm to about 350 nm. In some embodiments, the compound of Formula I has a particle size distribution d₁₀ of about 200 nm to about 500 nm. In some embodiments, the compound of Formula I has a particle size distribution d₉₀ of about 400 nm to about 800 nm. In some embodiments, the compound of Formula I has a particle size distribution d₁₀ of about 50 nm to about 350 nm, d₅₀ of about 200 nm to about 500 nm, and d₉₀ of about 400 nm to about 800 nm.

In some embodiments, the pharmaceutical composition comprises:
about 15 wt. % to about 45 wt. % of the compound of Formula I;
about 1 wt. % to about 5 wt. % of the poloxamer; and
about 55 wt. % to about 85 wt. % water.

In some embodiments, the pharmaceutical composition comprises a compound of Formula I, poloxamer 407, and water. In some embodiments, the pharmaceutical composition comprises a compound of Formula I, a poloxamer, a tonicity agent, and water. In some embodiments, the pharmaceutical composition comprises a compound of Formula I, poloxamer 407, mannitol, and water.

In some embodiments, the pharmaceutical composition comprises:
about 35 wt. % to about 45 wt. % of the compound of Formula I;
about 1 wt. % to about 5 wt. % of poloxamer 407;
about 1 wt. % to about 5 wt. % mannitol; and
about 55 wt. % to about 65 wt. % water.

In some embodiments, the pharmaceutical composition comprises:
about 20 wt. % to about 40 wt. % of the compound of Formula I;
about 1 wt. % to about 3 wt. % of poloxamer 407;
about 1 wt. % mannitol to about 3 wt. % mannitol; and
about 50 wt. % water to about 80 wt. % water.

In some embodiments, the pharmaceutical composition comprises:
about 40 wt. % of the compound of Formula I;
about 2 wt. % of poloxamer 407;
about 2 wt. % mannitol; and
about 56 wt. % water.

In some embodiments, the pharmaceutical composition comprises a compound of Formula I, or a pharmaceutically acceptable salt thereof; a poloxamer; a surfactant; a tonicity agent; and water. In some embodiments, the pharmaceutical composition comprises a compound of Formula I; a poloxamer; sodium deoxycholate; mannitol; and water. In some embodiments, the pharmaceutical composition comprises a compound of Formula I, Form I; poloxamer 407; sodium deoxycholate; mannitol; and water.

In some embodiments, the pharmaceutical composition comprises:
about 20 wt. % to about 50 wt. % of the compound of Formula I;
about 0.01 wt. % to about 5 wt. % poloxamer;
about 0.01 wt. % to about 5 wt. % sodium deoxycholate
about 1 wt. % to about 5 wt. % mannitol; and
about 50 wt. % to about 80 wt. % water.

In some embodiments, the pharmaceutical composition comprises:
about 20 wt. % to about 50 wt. % of the compound of Formula I;
about 1 wt. % to about 5 wt. % poloxamer 407;
about 0.01 wt. % to about 5 wt. % sodium deoxycholate
about 1 wt. % to about 5 wt. % mannitol; and
about 50 wt. % to about 80 wt. % water.

In some embodiments, the pharmaceutical composition comprises:
about 25 wt. % to about 35 wt. % of the compound of Formula I;
about 1 wt. % to about 5 wt. % poloxamer 407;
about 0.01 wt. % to about 1 wt. % sodium deoxycholate
about 1 wt. % to about 5 wt. % mannitol; and
about 55 wt. % to about 75 wt. % water.

In some embodiments, the pharmaceutical composition comprises:
about 30 wt. % of the compound of Formula I;
about 3 wt. % poloxamer 407;
about 0.3 wt. % sodium deoxycholate
about 2.0 to about 2.5 wt. % mannitol; and
about 64.2 wt. % to about 64.7 wt. % water.

In some embodiments, the pharmaceutical composition comprises:
about 30 wt. % of the compound of Formula I;
about 3 wt. % poloxamer 407;
about 0.3 wt. % sodium deoxycholate
about 2.5 wt. % mannitol; and
about 64.2 wt. % water.

In some embodiments, the pharmaceutical composition comprises a compound of Formula I, a poloxamer, a surfactant, a tonicity agent, and water, wherein the compound of Formula I has a particle size distribution d₉₀ of less than 1 μm. In some embodiments, the pharmaceutical composition comprises a compound of Formula I, a poloxamer, sodium deoxycholate, mannitol, and water, wherein the compound of Formula I has a particle size distribution d₉₀ of from about 200 nm to about 800 nm.

In some embodiments, the pharmaceutical composition comprises a compound of Formula I, a poloxamer, sodium deoxycholate, mannitol, and water, wherein the compound of Formula I has a particle size distribution d₁₀ of from about 100 nm to about 500 nm, or about 300 nm to 350 nm.

In some embodiments, the pharmaceutical composition comprises a compound of Formula I, a poloxamer, sodium deoxycholate, mannitol, and water, wherein the compound of Formula I has a particle size distribution d₅₀ of from about 300 nm to about 700 nm, or about 450 nm to about 500 nm.

In some embodiments, the pharmaceutical composition comprises a compound of Formula I, a poloxamer, sodium deoxycholate, mannitol, and water, wherein the compound of Formula I has a particle size distribution d₉₀ of from about 500 nm to about 900 nm, about 500 nm to about 800 nm, about 600 nm to about 800 nm, or about 725 nm to about 775 nm.

In some embodiments, the pharmaceutical composition comprises a compound of Formula I, a poloxamer, sodium deoxycholate, mannitol, and water, wherein the compound of Formula I has a particle size distribution d₁₀ of about 100 nm to about 500 nm, d₅₀ of about 300 nm to about 700 nm, and d₉₀ of about 500 nm to about 800 nm.

In some embodiments, the aqueous suspension comprises a pharmaceutically acceptable salt solution. Examples of pharmaceutically acceptable salt solutions include but are not limited to Ringer's solution and isotonic sodium chloride solution.

### Organic Suspensions

In some embodiments, the pharmaceutical composition is an organic suspension. As used herein, an “organic suspension” refers to a suspension wherein water is present in <5% of the total pharmaceutical composition weight (wt. %). In some embodiments, the pharmaceutical composition comprises less than 2 wt. % water. In some embodiments, the pharmaceutical composition comprises less than 1 wt. % water. In some embodiments, the pharmaceutical composition comprises essentially no water. In some embodiments, the pharmaceutical composition does not comprise any water.

In some embodiments, the organic suspension comprises a pharmaceutically acceptable oil, a pharmaceutically acceptable high-boiling liquid, or a combination thereof.

Examples of pharmaceutically acceptable oils include but are not limited to beeswax, fatty acids (e.g., oleic acid), soy fatty acids, vitamin E (e.g., vitamin E d-α-tocopheryl polyethylene glycol 1000 succinate (TPGS)), corn oil mono-di-triglycerides, medium chain C8-10 monoglycerides and diglycerides, propylene glycol dicaprylate/dicaprate, glyceryl monolinoleate, glyceryl monooleate, triglycerides (including both long-chain triglycerides and medium-chain triglycerides), polyoxylglycerides (e.g., oleoyl polyoxyl-6 glycerides), an ethoxylated castor oil (e.g., polyoxyl-35 castor oil (Kolliphor® ELP)), and a polyethylene glycol hydroxystearate (e.g., polyethylene glycol (15)-hydroxystearate (Kolliphor® HS 15)). The term “triglyceride” refers to an oil comprising a glycerol molecule and three fatty acid chains, and encompasses the naturally-derived and semi-synthetic/synthetic oils, for example castor oil, cottonseed oil, sesame oil, linseed oil, safflower oil, peanut oil, soybean oil, coconut oil, olive oil, corn oil, almond oil, poppyseed oil, sunflower oil, almond oil, vegetable oil, and mixtures thereof. Triglycerides also encompass: tricaprylin, caprylic/capric triglyceride (e.g., Miglyol 810, Miglyol 812 N (Miglyol 812), CAPTEX 355, and the like), caprylic/capric/linoleic triglyceride, caprylic/capric/succinic triglyceride, propylene glycol dicaprylate/dicaprate (e.g., Miglyol 840 and CAPTEX 200, and the like), glycerol triacetate (triacetin), glyceryl stearates, and the like, including mixtures thereof. The term “medium chain” fatty acid refers to a fatty acid having 6-12 carbons, while “short chain” fatty acids are ones with fewer than 6 carbons.

The fatty acids in triglycerides can be branched or unbranched, saturated or unsaturated, and can be of the same or different lengths within each triglyceride molecule. In some embodiments, the pharmaceutically acceptable oil comprises synthetic mono- or diglycerides.

Examples of pharmaceutically acceptable high-boiling liquids include but are not limited to N-methylpyrrolidone, N-butylpyrrolidone, dimethylsulfoxide, 1,3-butanediol, propylene glycol, benzyl benzoate, glycerol, and combinations thereof. In some embodiments, the pharmaceutical acceptable high-boiling liquid is a pharmaceutically acceptable organic solvent with a boiling point of greater than about 150° C., greater than about 175° C., or greater than about 185° C.

In some embodiments, the pharmaceutical composition comprises N-methylpyrrolidone and dimethylsulfoxide. In some embodiments, the N-methylpyrrolidone and dimethylsulfoxide are present in a 1:1 ratio.

In some embodiments, the pharmaceutically acceptable oil or pharmaceutically acceptable high-boiling liquid is selected from sesame oil, a medium chain triglyceride, castor oil, polyethylene glycol, propylene glycol, benzyl benzoate, glycerol, oleic acid, a polyoxylglyceride, dimethylsulfoxide, N-methylpyrrolidone, or a combination thereof.

In some embodiments, the pharmaceutically acceptable oil or a pharmaceutically acceptable high-boiling liquid is present in the composition in an amount of about 60% to about 90% by weight. In some embodiments, the pharmaceutically acceptable oil or a pharmaceutically acceptable high-boiling liquid is present in the composition in an amount of about 60% to about 70%, about 65% to about 75%, about 70% to about 80%, about 75% to about 85%, or about 80% to about 90% by weight. In some embodiments, the pharmaceutically acceptable oil or a pharmaceutically acceptable high-boiling liquid is present in the composition in an amount of about 60% to about 75% by weight or about 65% to about 70% by weight.

In some embodiments, the pharmaceutical composition comprises a compound of Formula I, or pharmaceutically acceptable salt thereof, and a pharmaceutically acceptable oil.

The oil absorption (half-life rate) after subcutaneous injection in different animals is different for different oily vehicles (see, e.g., Larsen, S. W. et al. The AAPS Journal, 2009; 11: 762). For example, the half-life rate for sesame oil is reportedly longer than that of Miglyol. This slower absorption rate of certain oily vehicles may allow formation of a depot at the injection site, thereby extending the release of a drug into systemic circulation.

In some embodiments, the pharmaceutically acceptable oil is sesame oil, a medium chain triglyceride, or a combination thereof.

In some embodiments, the pharmaceutical composition comprises sesame oil. In some embodiments, the pharmaceutical composition comprises about 60 wt. % to about 90 wt. %, about 60 wt. % to about 75 wt. %, or about 65 wt. % to about 70 wt. % sesame oil. In some embodiments, the pharmaceutical composition comprises sesame oil. In some embodiments, the pharmaceutical composition comprises about 67 wt. % sesame oil.

In some embodiments, the pharmaceutical composition comprises the compound of Formula I, or pharmaceutically acceptable salt thereof, and sesame oil. In some embodiments, the pharmaceutical composition comprises the compound of Formula I and sesame oil. In some embodiments, the pharmaceutical composition comprises the compound of Formula I and sesame oil, wherein the compound of Formula I has Form I. In some embodiments, the pharmaceutical composition comprises the compound of Formula I and sesame oil, wherein the compound of Formula I the compound of Formula I is micronized. In some embodiments, the pharmaceutical composition comprises the compound of Formula I and sesame oil, wherein the compound of Formula I has a particle size distribution d₉₀ of from about 1 μm to about 90 μm.

In some embodiments, the pharmaceutical composition comprises the compound of Formula I and sesame oil, wherein the compound of Formula I has a particle size distribution d₉₀ of from about 5 μm to about 15 μm.

In some embodiments, the pharmaceutical composition comprises:
about 10 wt. % to about 50 wt. % of the compound of Formula I; and
about 50 wt. % to about 90 wt. % sesame oil.

In some embodiments, the pharmaceutical composition comprises:
about 10 wt. % to about 40 wt. % of the compound of Formula I; and
about 60 wt. % to about 90 wt. % sesame oil.

In some embodiments, the pharmaceutical composition comprises:
about 25 wt. % to about 40 wt. % of the compound of Formula I; and
about 60 wt. % to about 75 wt. % sesame oil.

In some embodiments, the pharmaceutical composition comprises:
about 30 wt. % to about 35 wt. % of the compound of Formula I; and
about 65 wt. % to about 70 wt. % sesame oil.

In some embodiments, the pharmaceutical composition comprises:
about 33 wt. % of the compound of Formula I; and
about 67 wt. % sesame oil.

In some embodiments, the pharmaceutical composition comprises the compound of Formula I and sesame oil, wherein the concentration of the compound of Formula I is from about 200 mg/mL to about 500 mg/mL. In some embodiments, the pharmaceutical composition comprises the compound of Formula I and sesame oil, wherein the concentration of the compound of Formula I is from about 300 mg/mL to about 400 mg/mL. In some embodiments, the pharmaceutical composition comprises the compound of Formula I and sesame oil, wherein the concentration of the compound of Formula I about 350 mg/mL.

In some embodiments, the pharmaceutical composition comprises:
about 10 wt. % to about 40 wt. % of the compound of Formula I; and
about 60 wt. % to about 90 wt. % sesame oil;
wherein the compound of Formula I is present at a concentration of about 200 mg/mL to about 500 mg/mL.

In some embodiments, the pharmaceutical composition comprises:
about 25 wt. % to about 40 wt. % of the compound of Formula I; and
about 60 wt. % to about 75 wt. % sesame oil;
wherein the compound of Formula I is present at a concentration of about 200 mg/mL to about 500 mg/mL.

In some embodiments, the pharmaceutical composition comprises:
about 30 wt. % to about 35 wt. % of the compound of Formula I; and
about 65 wt. % to about 70 wt. % sesame oil;
wherein the compound of Formula I is present at a concentration of about 200 mg/mL to about 500 mg/mL.

In some embodiments, the pharmaceutical composition comprises:
about 33 wt. % of the compound of Formula I; and
about 67 wt. % sesame oil;
wherein the compound of Formula I is present at a concentration of about 200 mg/mL to about 500 mg/mL.

In some embodiments, the pharmaceutical composition comprises:
about 10 wt. % to about 40 wt. % of the compound of Formula I; and
about 60 wt. % to about 90 wt. % sesame oil;
wherein the compound of Formula I is present at a concentration of about 300 mg/mL to about 400 mg/mL.

In some embodiments, the pharmaceutical composition comprises:
about 25 wt. % to about 40 wt. % of the compound of Formula I; and
about 60 wt. % to about 75 wt. % sesame oil;
wherein the compound of Formula I is present at a concentration of about 300 mg/mL to about 400 mg/mL.

In some embodiments, the pharmaceutical composition comprises:
about 30 wt. % to about 35 wt. % of the compound of Formula I; and
about 65 wt. % to about 70 wt. % sesame oil;
wherein the compound of Formula I is present at a concentration of about 300 mg/mL to about 400 mg/mL.

In some embodiments, the pharmaceutical composition comprises:
about 33 wt. % of the compound of Formula I; and
about 67 wt. % sesame oil;
wherein the compound of Formula I is present at a concentration of about 300 mg/mL to about 400 mg/mL.

In some embodiments, the pharmaceutical composition comprises:
about 10 wt. % to about 40 wt. % of the compound of Formula I; and
about 60 wt. % to about 90 wt. % sesame oil;
wherein the compound of Formula I is present at a concentration of about 350 mg/mL.

In some embodiments, the pharmaceutical composition comprises:
about 25 wt. % to about 40 wt. % of the compound of Formula I; and
about 60 wt. % to about 75 wt. % sesame oil;
wherein the compound of Formula I is present at a concentration of about 350 mg/mL.

In some embodiments, the pharmaceutical composition comprises:
about 30 wt. % to about 35 wt. % of the compound of Formula I; and
about 65 wt. % to about 70 wt. % sesame oil;
wherein the compound of Formula I is present at a concentration of about 350 mg/mL.

In some embodiments, the pharmaceutical composition comprises:
about 33 wt. % of the compound of Formula I; and
about 67 wt. % sesame oil;
wherein the compound of Formula I is present at a concentration of about 350 mg/mL.

In some embodiments, the pharmaceutical composition comprises a pharmaceutically acceptable medium chain triglyceride (MCT). In some embodiments, the pharmaceutical composition comprises a compound of Formula I, or a pharmaceutically acceptable salt thereof, and a medium chain triglyceride comprising one or more C6-C7 fatty acid chains. In some embodiments, the pharmaceutical composition comprises a compound of Formula I, or a pharmaceutically acceptable salt thereof, and a medium chain triglyceride comprising one or more C8-C9 fatty acid chains. In some embodiments, the pharmaceutical composition comprises a compound of Formula I, or a pharmaceutically acceptable salt thereof, and a medium chain triglyceride comprising one or more C10-C12 fatty acid chains. In some embodiments, the pharmaceutical composition comprises a compound of Formula I, or a pharmaceutically acceptable salt thereof, and a medium chain triglyceride comprising one or more C6-C8 fatty acid chains. In some embodiments, the pharmaceutical composition comprises a compound of Formula I, or a pharmaceutically acceptable salt thereof, and a medium chain triglyceride comprising one or more C8-C12 fatty acid chains. In some embodiments, the pharmaceutical composition comprises a compound of Formula I, or a pharmaceutically acceptable salt thereof, and a medium chain triglyceride comprising one or more C6 fatty acid chains. In some embodiments, the pharmaceutical composition comprises a compound of Formula I, or a pharmaceutically acceptable salt thereof, and a medium chain triglyceride comprising one or more C7 fatty acid chains. In some embodiments, the pharmaceutical composition comprises a compound of Formula I, or a pharmaceutically acceptable salt thereof, and a medium chain triglyceride comprising one or more C8 fatty acid chains. In some embodiments, the pharmaceutical composition comprises a compound of Formula I, or a pharmaceutically acceptable salt thereof, and a medium chain triglyceride comprising one or more C9 fatty acid chains. In some embodiments, the pharmaceutical composition comprises a compound of Formula I, or a pharmaceutically acceptable salt thereof, and a medium chain triglyceride comprising one or more C10 fatty acid chains. In some embodiments, the pharmaceutical composition comprises a compound of Formula I, or a pharmaceutically acceptable salt thereof, and a medium chain triglyceride comprising one or more C1I fatty acid chains. In some embodiments, the pharmaceutical composition comprises a compound of Formula I, or a pharmaceutically acceptable salt thereof, and a medium chain triglyceride comprising one or more C12 fatty acid chains.

In some embodiments, the pharmaceutical composition comprises a compound of Formula I, or a pharmaceutically acceptable salt thereof, and a medium chain triglyceride, wherein the medium chain triglyceride is a triglyceride ester of saturated coconut/palmkernel oil derived caprylic and capric fatty acids and plant derived glycerol (e.g., Miglyol 812 N).

In some embodiments, the pharmaceutical composition comprises a propylene glycol diester. In some embodiments, the pharmaceutical composition comprises Miglyol 840.

In some embodiments, the pharmaceutical composition comprises Miglyol 810, Miglyol 812 N, Miglyol 840, or mixtures thereof. In some embodiments, the pharmaceutical composition comprises a medium chain triglyceride which is Miglyol 812 N. In some embodiments, the pharmaceutical composition comprises about 60 wt. % to about 90 wt. % Miglyol 812 N.

In some embodiments, the pharmaceutical composition comprises the compound of Formula I, or pharmaceutically acceptable salt thereof, and a medium chain triglyceride.

In some embodiments, the pharmaceutical composition comprises the compound of Formula I and a medium chain triglyceride. In some embodiments, the pharmaceutical composition comprises the compound of Formula I and a medium chain triglyceride, wherein the compound of Formula I has Form I. In some embodiments, the pharmaceutical composition comprises the compound of Formula I and a medium chain triglyceride, wherein the compound of Formula I is micronized. In some embodiments, the pharmaceutical composition comprises the compound of Formula I and a medium chain triglyceride, wherein the compound of Formula I has a particle size distribution d₉₀ of from about 1 μm to about 90 μm. In some embodiments, the pharmaceutical composition comprises the compound of Formula I and a medium chain triglyceride, wherein the compound of Formula I has a particle size distribution d₉₀ of from about 5 μm to about 15 μm.

In some embodiments, the pharmaceutical composition comprises the compound of Formula I and Miglyol 812 N. In some embodiments, the pharmaceutical composition comprises the compound of Formula I and Miglyol 812 N, wherein the compound of Formula I has Form I.

In some embodiments, the pharmaceutical composition comprises the compound of Formula I and Miglyol 812 N, wherein the compound of Formula I is micronized. In some embodiments, the pharmaceutical composition comprises the compound of Formula I and Miglyol 812N, wherein the compound of Formula I has a particle size distribution d₉₀ of from about 1 μm to about 90 μm. In some embodiments, the pharmaceutical composition comprises the compound of Formula I and Miglyol 812 N, wherein the compound of Formula I has a particle size distribution d₉₀ of from about 5 μm to about 15 μm.

In some embodiments, the pharmaceutical composition comprises:
about 10 wt. % to about 40 wt. % of the compound of Formula I; and
about 60 wt. % to about 90 wt. % medium chain triglyceride.

In some embodiments, the pharmaceutical composition comprises:
about 10 wt. % to about 40 wt. % of the compound of Formula I; and
about 60 wt. % to about 90 wt. % medium chain triglyceride.
wherein the compound of Formula I is present at a concentration of about 200 mg/mL to about 500 mg/mL.

In some embodiments, the pharmaceutical composition comprises:
about 10 wt. % to about 40 wt. % of the compound of Formula I; and
about 60 wt. % to about 90 wt. % medium chain triglyceride.
wherein the compound of Formula I is present at a concentration of about 250 mg/mL to about 400 mg/mL.

In some embodiments, the pharmaceutical composition comprises:
about 10 wt. % to about 40 wt. % of the compound of Formula I; and
about 60 wt. % to about 90 wt. % medium chain triglyceride.
wherein the compound of Formula I is present at a concentration of about 300 mg/mL.

In some embodiments, the pharmaceutical composition comprises:
about 10 wt. % to about 40 wt. % of the compound of Formula I; and
about 60 wt. % to about 90 wt. % medium chain triglyceride.
wherein the compound of Formula I is present at a concentration of about 350 mg/mL.

In some embodiments, the pharmaceutical composition comprises:
about 10 wt. % to about 40 wt. % of the compound of Formula I; and
about 60 wt. % to about 90 wt. % medium chain triglyceride.
wherein the compound of Formula I is present at a concentration of about 400 mg/mL.

In some embodiments, the pharmaceutical composition comprises:
about 10 wt. % to about 40 wt. % of the compound of Formula I; and
about 60 wt. % to about 90 wt. % medium chain triglyceride.

In some embodiments, the pharmaceutical composition comprises:
about 10 wt. % to about 40 wt. % of the compound of Formula I; and
about 60 wt. % to about 90 wt. % Miglyol 812 N.

In some embodiments, the pharmaceutical composition comprises:
about 10 wt. % to about 40 wt. % of the compound of Formula I; and
about 60 wt. % to about 90 wt. % Miglyol 812 N.
wherein the compound of Formula I is present at a concentration of about 200 mg/mL to about 500 mg/mL.

In some embodiments, the pharmaceutical composition comprises:
about 10 wt. % to about 40 wt. % of the compound of Formula I; and
about 60 wt. % to about 90 wt. % Miglyol 812 N.
wherein the compound of Formula I is present at a concentration of about 250 mg/mL to about 400 mg/mL.

In some embodiments, the pharmaceutical composition comprises:
about 10 wt. % to about 40 wt. % of the compound of Formula I; and
about 60 wt. % to about 90 wt. % Miglyol 812 N.
wherein the compound of Formula I is present at a concentration of about 300 mg/mL.

In some embodiments, the pharmaceutical composition comprises:
about 10 wt. % to about 40 wt. % of the compound of Formula I; and
about 60 wt. % to about 90 wt. % Miglyol 812 N.
wherein the compound of Formula I is present at a concentration of about 350 mg/mL.

In some embodiments, the pharmaceutical composition comprises:
about 10 wt. % to about 40 wt. % of the compound of Formula I; and
about 60 wt. % to about 90 wt. % Miglyol 812 N.
wherein the compound of Formula I is present at a concentration of about 400 mg/mL.

In some embodiments, the pharmaceutical composition comprises:
about 10 wt. % to about 40 wt. % of the compound of Formula I; and
about 60 wt. % to about 90 wt. % Miglyol 812 N.
wherein the compound of Formula I is present at a concentration of about 450 mg/mL.

In some embodiments, the pharmaceutical composition comprises a salt of the compound of Formula I and a medium chain triglyceride.

In some embodiments, the pharmaceutical composition comprises a sodium salt of the compound of Formula I and a medium chain triglyceride. In some embodiments, the pharmaceutical composition comprises a sodium salt of the compound of Formula I and a medium chain triglyceride, wherein the sodium salt of the compound of Formula I has crystalline Form I. In some embodiments, the pharmaceutical composition comprises a sodium salt of the compound of Formula I and a medium chain triglyceride, wherein the sodium salt of the compound of Formula I is micronized. In some embodiments, the pharmaceutical composition comprises a sodium salt of the compound of Formula I and a medium chain triglyceride, wherein the sodium salt of the compound of Formula I has a particle size distribution d₉₀ of from about 1 μm to about 90 μm. In some embodiments, the pharmaceutical composition comprises a sodium salt of the compound of Formula I and a medium chain triglyceride, wherein the sodium salt of the compound of Formula I has a particle size distribution d₉₀ of from about 5 μm to about 15 μm.

In some embodiments, the pharmaceutical composition comprises:
about 10 wt. % to about 40 wt. % of a sodium salt of the compound of Formula I; and
about 60 wt. % to about 90 wt. % medium chain triglyceride.

In some embodiments, the pharmaceutical composition comprises:
about 10 wt. % to about 40 wt. % of a sodium salt of the compound of Formula I; and
about 60 wt. % to about 90 wt. % medium chain triglyceride;
wherein the sodium salt of the compound of Formula I is present at a concentration of from about 200 mg/mL to about 500 mg/mL.

In some embodiments, the pharmaceutical composition comprises:
about 10 wt. % to about 40 wt. % of a sodium salt of the compound of Formula I; and
about 60 wt. % to about 90 wt. % medium chain triglyceride;
wherein the sodium salt of the compound of Formula I is present at a concentration of about 400 mg/mL.

In some embodiments, the pharmaceutical composition comprises a sodium salt of the compound of Formula I and Miglyol 812 N. In some embodiments, the pharmaceutical composition comprises a sodium salt of the compound of Formula I and Miglyol 812 N, wherein the sodium salt of the compound of Formula I has crystalline Form I. In some embodiments, the pharmaceutical composition comprises a sodium salt of the compound of Formula I and Miglyol 812 N, wherein the sodium salt of the compound of Formula I is micronized. In some embodiments, the pharmaceutical composition comprises a sodium salt of the compound of Formula I and Miglyol 812 N, wherein the sodium salt of the compound of Formula I has a particle size distribution d₉₀ of from about 1 μm to about 90 μm. In some embodiments, the pharmaceutical composition comprises a sodium salt of the compound of Formula I and Miglyol 812 N, wherein the sodium salt of the compound of Formula I has a particle size distribution d₉o of from about 5 μm to about 15 μm.

In some embodiments, the pharmaceutical composition comprises:
about 10 wt. % to about 40 wt. % of a sodium salt of the compound of Formula I; and
about 60 wt. % to about 90 wt. % Miglyol 812 N.

In some embodiments, the pharmaceutical composition comprises:
about 10 wt. % to about 40 wt. % of a sodium salt of the compound of Formula I; and
about 60 wt. % to about 90 wt. % Miglyol 812 N;
wherein the sodium salt of the compound of Formula I is present at a concentration of from about 200 mg/mL to about 500 mg/mL.

In some embodiments, the pharmaceutical composition comprises:
about 10 wt. % to about 40 wt. % of a sodium salt of the compound of Formula I; and
about 60 wt. % to about 90 wt. % Miglyol 812 N;
wherein the sodium salt of the compound of Formula I is present at a concentration of about 400 mg/mL.

In some embodiments, the pharmaceutical composition comprises a calcium salt of the compound of Formula I and a medium chain triglyceride. In some embodiments, the pharmaceutical composition comprises a calcium salt of the compound of Formula I and a medium chain triglyceride, wherein the calcium salt of compound of Formula I has Form I. In some embodiments, the pharmaceutical composition comprises a calcium salt of the compound of Formula I and a medium chain triglyceride, wherein the calcium salt of compound of Formula I is amorphous. In some embodiments, the pharmaceutical composition comprises a calcium salt of the compound of Formula I and a medium chain triglyceride, wherein the calcium salt of the compound of Formula I is micronized. In some embodiments, the pharmaceutical composition comprises a calcium salt of the compound of Formula I and a medium chain triglyceride, wherein the calcium salt of the compound of Formula I has a particle size distribution d₉₀ of from about 1 μm to about 90 μm. In some embodiments, the pharmaceutical composition comprises a calcium salt of the compound of Formula I and a medium chain triglyceride, wherein the calcium salt of the compound of Formula I has a particle size distribution d₉₀ of from about 1 μm to about 30 μm. In some embodiments, the pharmaceutical composition comprises a calcium salt of the compound of Formula I and a medium chain triglyceride, wherein the calcium salt of the compound of Formula I has a particle size distribution d₉₀ of from about 1 μm to about 10 μm. In some embodiments, the pharmaceutical composition comprises a calcium salt of the compound of Formula I and a medium chain triglyceride, wherein the calcium salt of the compound of Formula I has a particle size distribution d₉₀ of from about 1 μm to about 5 μm.

In some embodiments, the pharmaceutical composition comprises:
about 10 wt. % to about 55 wt. % of a calcium salt of the compound of Formula I; and
about 45 wt. % to about 90 wt. % medium chain triglyceride.

In some embodiments, the pharmaceutical composition comprises:
about 10 wt. % to about 55 wt. % of a calcium salt of the compound of Formula I; and
about 35 wt. % to about 90 wt. % medium chain triglyceride;
wherein the calcium salt of the compound of Formula I is present at a concentration of from about 200 mg/mL to about 500 mg/mL.

In some embodiments, the pharmaceutical composition comprises:
about 10 wt. % to about 40 wt. % of a calcium salt of the compound of Formula I; and
about 60 wt. % to about 90 wt. % medium chain triglyceride.

In some embodiments, the pharmaceutical composition comprises:
about 10 wt. % to about 40 wt. % of a calcium salt of the compound of Formula I; and
about 60 wt. % to about 90 wt. % medium chain triglyceride;
wherein the calcium salt of the compound of Formula I is present at a concentration of from about 200 mg/mL to about 500 mg/mL.

In some embodiments, the pharmaceutical composition comprises:
about 10 wt. % to about 40 wt. % of a sodium salt of the compound of Formula I; and
about 60 wt. % to about 90 wt. % medium chain triglyceride;
wherein the sodium salt of the compound of Formula I is present at a concentration of from about 400 mg/mL to about 500 mg/mL.

In some embodiments, the pharmaceutical composition comprises a calcium salt of the compound of Formula I and Miglyol 812 N. In some embodiments, the pharmaceutical composition comprises a calcium salt of the compound of Formula I and Miglyol 812 N, wherein the calcium salt of compound of Formula I has Form I. In some embodiments, the pharmaceutical composition comprises a calcium salt of the compound of Formula I and Miglyol 812 N, wherein the calcium salt of compound of Formula I is amorphous. In some embodiments, the pharmaceutical composition comprises a calcium salt of the compound of Formula I and Miglyol 812 N, wherein the calcium salt of the compound of Formula I is micronized. In some embodiments, the pharmaceutical composition comprises a calcium salt of the compound of Formula I and Miglyol 812 N, wherein the calcium salt of the compound of Formula I has a particle size distribution d₉₀ of from about 1 μm to about 90 μm. In some embodiments, the pharmaceutical composition comprises a calcium salt of the compound of Formula I and Miglyol 812 N, wherein the calcium salt of the compound of Formula I has a particle size distribution d₉₀ of from about 1 μm to about 30 μm. In some embodiments, the pharmaceutical composition comprises a calcium salt of the compound of Formula I and Miglyol 812 N, wherein the calcium salt of the compound of Formula I has a particle size distribution d₉₀ of from about 1 μm to about 10 μm. In some embodiments, the pharmaceutical composition comprises a calcium salt of the compound of Formula I and Miglyol 812 N, wherein the calcium salt of the compound of Formula I has a particle size distribution d₉₀ of from about 1 μm to about 5 μm.

In some embodiments, the pharmaceutical composition comprises:
about 10 wt. % to about 55 wt. % of a calcium salt of the compound of Formula I; and
about 35 wt. % to about 90 wt. % Miglyol 812 N.

In some embodiments, the pharmaceutical composition comprises:
about 10 wt. % to about 55 wt. % of a calcium salt of the compound of Formula I; and
about 35 wt. % to about 90 wt. % Miglyol 812 N;
wherein the calcium salt of the compound of Formula I is present at a concentration of from about 200 mg/mL to about 500 mg/mL.

In some embodiments, the pharmaceutical composition comprises:
about 10 wt. % to about 40 wt. % of a calcium salt of the compound of Formula I; and
about 60 wt. % to about 90 wt. % Miglyol 812 N.

In some embodiments, the pharmaceutical composition comprises:
about 10 wt. % to about 40 wt. % of a calcium salt of the compound of Formula I; and
about 60 wt. % to about 90 wt. % Miglyol 812 N;
wherein the calcium salt of the compound of Formula I is present at a concentration of from about 200 mg/mL to about 500 mg/mL.

In some embodiments, the pharmaceutical composition comprises:
about 10 wt. % to about 40 wt. % of a sodium salt of the compound of Formula I; and
about 60 wt. % to about 90 wt. % Miglyol 812 N;
wherein the sodium salt of the compound of Formula I is present at a concentration of from about 400 mg/mL to about 500 mg/mL.

In some embodiments, the pharmaceutical composition comprising a pharmaceutically acceptable oil, a pharmaceutically acceptable high-boiling liquid, or a combination thereof further comprises a surfactant. In some embodiments, the pharmaceutical composition comprising a pharmaceutically acceptable oil, a pharmaceutically acceptable high-boiling liquid, or a combination thereof further comprises a surfactant selected from a poloxamer, a polysorbate, and combinations thereof.

Any of the pharmaceutical compositions disclosed herein and their individual components (the compound of Formula I, or a pharmaceutically acceptable salt thereof, and one or more components of the liquid vehicle) can be sterilized to remove endotoxin and/or bioburden using one or more suitable methods known in the art, for example, by autoclaving, dry heating, Gamma radiation, infrared radiation, ultraviolet (UV) radiation, processing with high-velocity electrons (E-beam), or sterile filtration. In some embodiments, the pharmaceutical compositions are sterilized using gamma radiation. In some embodiments, the pharmaceutical compositions are sterilized using sterile filtration. In some embodiments, the one or more pharmaceutically acceptable excipients are sterilized using gamma radiation. In some embodiments, the pharmaceutically acceptable excipients are sterilized using sterile filtration. In some embodiments, the liquid vehicle is sterilized using gamma radiation. In some embodiments, the liquid vehicle is sterilized using sterile filtration.

Any of the pharmaceutical compositions disclosed herein can be administered by injection. In some embodiments, the pharmaceutical composition is for subcutaneous (SC) or intramuscular (IM) administration. Site reactions to injections and infusions are common adverse events (AE), which are any unfavorable and unintended sign, symptom, or disease temporally associated with the use of a medical treatment or procedure regardless of whether it is considered related to the medical treatment or procedure. Injection site reactions (ISR) associated with SC or IM administration of a pharmaceutical composition can be measured by monitoring the following parameters: injection site pain or tenderness, injection site erythema or redness, injection site induration or swelling, and injection site pruritus, on a scale of Grade 1 (mild) to Grade 4 (potentially life-threatening) based on the severity of the symptoms (Division of AIDS (DAIDS) Table for Grading the Severity of Adult and Pediatric Adverse Events, Corrected Version 2.1, July 2017, page 24).

Any of the pharmaceutical compositions provided herein can be for use in a method for treating or preventing an HIV infection. Accordingly, provided herein is a method for treating or preventing an HIV infection in a human, comprising administering to the human a therapeutically effective amount of any one of the disclosed pharmaceutical compositions.

In some embodiments, the pharmaceutical composition is administered not more frequently than once every four weeks (Q4W) or once per month. In some embodiments, the pharmaceutical composition is administered once every four weeks (Q4W) or once per month. In some embodiments, the pharmaceutical composition is administered once every eight weeks (Q8W) or every two months. In some embodiments, the pharmaceutical composition is administered once every twelve weeks (Q12W) or every three months.

In some embodiments, the pharmaceutical composition is administered once per month (Q1M). In some embodiments, the pharmaceutical composition is administered every two months (Q2M). In some embodiments, the pharmaceutical composition is administered once every three months (Q3M). In some embodiments, the pharmaceutical composition is administered once every four months (Q4M). In some embodiments, the pharmaceutical composition is administered once every five months (Q5M). In some embodiments, the pharmaceutical composition is administered once every six months (Q6M).

In some embodiments, the therapeutically effective amount of the compound of Formula I is the amount of the pharmaceutical composition comprising from about 100 mg to about 2500 mg of the compound of Formula I, or a pharmaceutically acceptable salt thereof. In some embodiments, the pharmaceutical composition comprises of from about 200 mg to about 1000 mg of the compound of Formula I, or a pharmaceutically acceptable salt thereof. In some embodiments, the pharmaceutical composition comprises of from about 200 mg to about 400 mg of the compound of Formula I, or a pharmaceutically acceptable salt thereof. In some embodiments, the pharmaceutical composition comprises of from about 400 mg to about 600 mg of the compound of Formula I, or a pharmaceutically acceptable salt thereof. In some embodiments, the pharmaceutical composition comprises of from about 600 mg to about 800 mg of the compound of Formula I, or a pharmaceutically acceptable salt thereof. In some embodiments, the pharmaceutical composition comprises of from about 800 mg to about 1000 mg of the compound of Formula I, or a pharmaceutically acceptable salt thereof. In some embodiments, the pharmaceutical composition comprises about 850 mg of the compound of Formula I, or a pharmaceutically acceptable salt thereof.

In some embodiments, the pharmaceutical compositions further comprise one or more additional therapeutic agents (e.g., one, two, three, or four additional therapeutic agents).

In some embodiments, an additional agent is an anti-inflammatory agent. In some embodiments, the anti-inflammatory agent is a steroid, for example a corticosteroid. In some embodiments, the anti-inflammatory agent is dexamethasone.

In some embodiments, the additional agent is a pharmacokinetic enhancer. In some embodiments, the additional agent is hyaluronidase. In some embodiments, the additional agent is hyaluronidase and the hyaluronidase enhances pharmacokinetics and/or increases tolerability of the pharmaceutical composition (e.g., provides improved, manageable, minimal or no injection site reactions).

In some embodiments, the additional therapeutic agents are anti-HIV agents independently selected from HIV capsid inhibitors, HIV protease inhibiting compounds, HIV nonnucleoside inhibitors of reverse transcriptase, HIV nucleoside inhibitors of reverse transcriptase, HIV nucleoside reverse transcriptase translocation inhibitors, HIV integrase inhibitors, and CCR5 inhibitors.

In some embodiments, the pharmaceutical compositions do not comprise an additional therapeutic agent. In other words, the compound of Formula I, or pharmaceutically acceptable salt thereof, is the sole therapeutic agent.

In some embodiments, the pharmaceutical compositions do not comprise an additional anti-HIV agent. In other words, the compound of Formula I, or pharmaceutically acceptable salt thereof, is the sole anti-HIV agent.

### IV. Methods of Use

Provided herein are methods for treating or preventing an HIV infection in a human, comprising administering to the human by injection a therapeutically effective amount of a compound of Formula I, or a pharmaceutically acceptable salt thereof. The compound of Formula I, or a pharmaceutically acceptable salt thereof, administered by injection can be formulated as a pharmaceutical composition described herein. Such pharmaceutical compositions (i.e., pharmaceutical formulations) can be long-acting meaning that the pharmaceutical formulations provided herein can maintain a therapeutically effective amount of the compound of Formula I, or a pharmaceutically acceptable salt thereof, in the plasma and/or the PBMCs of the human. In some embodiments, the long-acting formulations have at least a one month duration of action. The long-acting formulations can be administered to the human less frequently than, e.g., immediate release or short-acting forms.

Accordingly, provided herein is a method for treating or preventing an HIV infection in a human, comprising administering to the human by injection a therapeutically effective amount of any of the pharmaceutical compositions provided herein.

Also provided herein is the use of any of the pharmaceutical compositions provided herein for treating or preventing an HIV infection.

The pharmaceutical compositions may be administered to a subject in accordance with an effective dosing regimen for a desired period of time or duration. In some embodiments of the methods provided herein, the pharmaceutical composition is administered once every 1 week to about 6 months. In some embodiments, the pharmaceutical composition is administered at a frequency of about 1 week to about 5 months, about 1 week to about 4 months, about 1 week to about 3 months, about 1 week to about 2 months, about 1 week to about 1 month, about 1 week to about 4 weeks, about 1 week to about 3 weeks, about 1 week to about 2 weeks, about 2 weeks to 6 months, about 2 weeks to about 5 months, about 2 weeks to about 4 months, about 2 weeks to about 3 months, about 2 weeks to about 2 months, about 2 weeks to about 1 month, about 2 weeks to about 4 weeks, about 2 weeks to about 3 weeks, about 3 weeks to about 6 months, about 3 weeks to about 5 months, about 3 weeks to about 4 months, about 3 weeks to about 3 months, about 3 weeks to about 2 months, about 3 weeks to about 1 month, about 3 weeks to about 4 weeks, about 4 weeks to about 6 months, about 4 weeks to about 5 months, about 4 weeks to about 4 months, about 4 weeks to about 3 months, about 4 weeks to about 2 months, about 4 weeks to about 1 month, about 1 month to about 6 months, about 1 month to about 5 months, about 1 month to about 4 months, about 1 month to about 3 months, about 1 month to about 3 months, about 2 months to about 6 months, about 2 months to about 5 months, about 2 months to about 4 months, about 2 months to about 3 months, about 2 months to about 3 months, about 3 months to about 6 months, about 3 months to about 5 months, about 3 months to about 4 months, about 4 months to about 6 months, about 3 months to about 5 months, or about 5 months to about 6 months.

In some embodiments, the pharmaceutical composition is administered once every 28 days. In some embodiments, the pharmaceutical composition is administered once every 1 month. In some embodiments the pharmaceutical composition is administered at a frequency of once every month or less. In some embodiments, the pharmaceutical composition is administered at a frequency of once every two months or less. In some embodiments, the pharmaceutical composition is administered at a frequency of once every three months or less.

In some embodiments of the methods provided herein, the pharmaceutical composition is administered not more frequently than once every four weeks (Q4W) or once per month.

In some embodiments, the pharmaceutical composition is administered once every four weeks (Q4W). In some embodiments, the pharmaceutical composition is administered once every five weeks (Q5W). In some embodiments, the pharmaceutical composition is administered once every six weeks (Q6W). In some embodiments, the pharmaceutical composition is administered once every seven weeks (Q7W). In some embodiments, the pharmaceutical composition is administered once every eight weeks (Q8W). In some embodiments, the pharmaceutical composition is administered once every nine weeks (Q9W). In some embodiments, the pharmaceutical composition is administered once every ten weeks (Q10W). In some embodiments, the pharmaceutical composition is administered once every eleven weeks (Q11W). In some embodiments, the pharmaceutical composition is administered once every twelve weeks (Q12W). In some embodiments, the pharmaceutical composition is administered once every fourteen weeks (Q14W). In some embodiments, the pharmaceutical composition is administered once every sixteen weeks (Q16W).

In some embodiments, the pharmaceutical composition is administered once per month (Q1M). In some embodiments, the pharmaceutical composition is administered every two months (Q2M). In some embodiments, the pharmaceutical composition is administered once every three months (Q3M). In some embodiments, the pharmaceutical composition is administered once every four months (Q4M). In some embodiments, the pharmaceutical composition is administered once every five months (Q5M). In some embodiments, the pharmaceutical composition is administered once every six months (Q6M).

In some embodiments, the pharmaceutical composition is administered in a dose from about 100 mg to about 2500 mg of the compound of Formula I, or a pharmaceutically acceptable salt thereof, wherein the dose amount is based on the free acid form. In some embodiments, the pharmaceutical composition is administered in a dose from about 100 mg to about 2500 mg, about 100 mg to about 2400 mg, about 100 mg to about 2300 mg, about 100 mg to about 2200 mg, about 100 mg to about 2100 mg, about 100 mg to about 2000 mg, about 100 mg to about 1900 mg. about 100 mg to about 1800 mg, about 100 mg to about 1700 mg, about 100 mg to about 1600 mg, about 100 mg to about 1500 mg, about 100 mg to about 1400 mg, about 100 mg to about 1300 mg, about 100 mg to about 1200 mg, about 100 mg to about 1100 mg, about 100 mg to about 1000 mg, about 100 mg to about 900 mg, about 100 mg to about 800 mg, about 100 mg to about 700 mg, about 100 mg to about 600 mg, about 100 mg to about 500 mg, about 100 mg to about 400 mg, about 100 mg to about 300 mg, or about 100 mg to about 200 mg of the compound of Formula I, or a pharmaceutically acceptable salt thereof, wherein the dose amount is based on the free acid form.

In some embodiments, the pharmaceutical composition is administered in a dose from about 200 mg to about 2500 mg of the compound of Formula I, or a pharmaceutically acceptable salt thereof, wherein the dose amount is based on the free acid form. In some embodiments, the pharmaceutical composition is administered in a dose from about 200 mg to about 2000 mg of the compound of Formula I, or a pharmaceutically acceptable salt thereof, wherein the dose amount is based on the free acid form. In some embodiments, the pharmaceutical composition is administered in a dose from about 200 mg to about 1500 mg of the compound of Formula I, or a pharmaceutically acceptable salt thereof, wherein the dose amount is based on the free acid form. In some embodiments, the pharmaceutical composition is administered in a dose from about 200 mg to about 1000 mg of the compound of Formula I, or a pharmaceutically acceptable salt thereof, wherein the dose amount is based on the free acid form. In some embodiments, the pharmaceutical composition is administered in a dose from about 200 mg to about 400 mg of compound of Formula I, or a pharmaceutically acceptable salt thereof, wherein the dose amount is based on the free acid form. In some embodiments, the pharmaceutical composition is administered in a dose from about 400 mg to about 600 mg of compound of Formula I, or a pharmaceutically acceptable salt thereof, wherein the dose amount is based on the free acid form. In some embodiments, the pharmaceutical composition is administered in a dose from about 600 mg to about 800 mg of compound of Formula I, or a pharmaceutically acceptable salt thereof, wherein the dose amount is based on the free acid form. In some embodiments, the pharmaceutical composition is administered in a dose from about 800 mg to about 1000 mg of compound of Formula I, or a pharmaceutically acceptable salt thereof, wherein the dose amount is based on the free acid form. In some embodiments, the pharmaceutical composition is administered in a dose from about 1000 mg to about 1200 mg of compound of Formula I, or a pharmaceutically acceptable salt thereof, wherein the dose amount is based on the free acid form.

In some embodiments, the pharmaceutical composition is administered in a dose from about 500 mg to about 1000 mg of the compound of Formula I, or a pharmaceutically acceptable salt thereof, wherein the dose amount is based on the free acid form. In some embodiments, the pharmaceutical composition is administered in a dose from about 600 mg to about 1000 mg of the compound of Formula I, or a pharmaceutically acceptable salt thereof, wherein the dose amount is based on the free acid form. In some embodiments, the pharmaceutical composition is administered in a dose from about 750 mg to about 1000 mg of the compound of Formula I, or a pharmaceutically acceptable salt thereof, wherein the dose amount is based on the free acid form.

In some embodiments, the pharmaceutical composition is administered in a dose of about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, about 1000 mg, about 1025 mg, about 1050 mg, about 1075 mg, about 1100 mg, about 1125 mg, about 1150 mg, about 1175 mg, about 1200 mg, about 1225 mg, about 1250 mg, about 1275 mg, about 1300 mg, about 1325 mg, about 1350 mg, about 1375 mg, about 1400 mg, about 1425 mg, about 1450 mg, about 1475 mg, about 1500 mg, about 1525 mg, about 1550 mg, about 1575 mg, about 1600 mg, about 1625 mg, about 1650 mg, about 1675 mg, about 1700 mg, about 1725 mg, about 1750 mg, about 1775 mg, about 1800 mg, about 1825 mg, about 1850 mg, about 1875 mg, about 1900 mg, about 1925 mg, about 1950 mg, about 1975 mg, about 2000 mg, about 2025 mg, about 2050 mg, about 2075 mg, about 2100 mg, about 2125, about 2150 mg, about 2175 mg, about 2200 mg, about 2225 mg, about 2250 mg, about 2275 mg, about 2300 mg, about 2325 mg, about 2350 mg, about 2375 mg, about 2400 mg, about 2425 mg, about 2450 mg, about 2475 mg, or about 2500 mg of the compound of Formula I, or a pharmaceutically acceptable salt thereof, wherein the dose amount is based on the free acid form.

In some embodiments, the pharmaceutical composition is administered in a dose of about 850 mg of the compound of Formula I, or a pharmaceutically acceptable salt thereof, wherein the dose amount is based on the free acid form.

In some embodiments, the pharmaceutical composition is administered once every three months at a dose of about 500 mg to 1000 mg of the compound of Formula I, or a pharmaceutically acceptable salt thereof, wherein the dose amount is based on the free acid form. In some embodiments, the pharmaceutical composition is administered once every three months at a dose of about 700 mg to 1200 mg of the compound of Formula I, or a pharmaceutically acceptable salt thereof, wherein the dose amount is based on the free acid form. In some embodiments, the pharmaceutical composition is administered once every three months at a dose of about 850 mg of the compound of Formula I, or a pharmaceutically acceptable salt thereof, wherein the dose amount is based on the free acid form.

In some embodiments of the methods provided herein, the human is infected with HIV.

In some embodiments of the methods provided herein, the human has an HIV-1 RNA copy number of less than about 50 copies/mL (i.e., is virologically suppressed).

In some embodiments, the HIV infection is HIV-1 infection characterized by HIV-1 mutant resistance to one or more antiviral medications. In some embodiments, the HIV infection is an HIV-1 infection characterized by HIV-1 mutant resistance to two or more antiretroviral medications. In some embodiments, the HIV infection is an HIV-1 infection characterized by HIV-1 mutant resistance to three or more antiretroviral medications.

In some embodiments, the HIV-1 mutant is resistant to a protease inhibitor (PI), a nucleoside or nucleotide reverse transcriptase inhibitor (NRTI), a non-nucleoside or non-nucleotide reverse transcriptase inhibitor (NNRTI), or an integrase strand transfer inhibitor (INSTI). In certain embodiments, the HIV-1 mutant resistant to a protease inhibitor is selected from I50V, I84V/L90M, G48V/V82A/L90M, and G48V/V82S. In certain embodiments, the HIV-1 mutant resistant to a nucleoside or nucleotide reverse transcriptase inhibitor is selected from K65R, M184V, and 6TAMs. In certain embodiments, the HIV-1 mutant resistant to a non-nucleoside or non-nucleotide reverse transcriptase inhibitor is selected from K103N, Y181C, Y188L, L100I/K103N, and K103N/Y181C. In certain embodiments, the HIV-1 mutant resistant to a integrase strand transfer inhibitor is selected from Y143R, E138K/Q148K, G140S/Q148R, E92Q/N155H, N155H/Q148R, and R263K/M50I.

In some embodiments, the human (or “patient”) is infected with HIV-1 resistant to at least one antiretroviral medication. In some embodiments, the patient is infected with multidrug resistant HIV-1 which is resistant to at least one antiretroviral medication from each of two different classes of antiretroviral medications. In some embodiments, the patient is infected with multidrug resistant HIV-1 which is resistant to at least one antiretroviral medication from each of three different classes of antiretroviral medications. In some embodiments, the different classes of antiretroviral medications are selected from a nucleoside or nucleotide reverse transcriptase inhibitor (NRTI), a non-nucleoside or non-nucleotide reverse transcriptase inhibitor (NNRTI), a protease inhibitor (PI), and an integrase strand transfer inhibitor (INSTI).

In some embodiments, the NRTI is selected from emtricitabine, lamivudine (3TC), zidovudine (azidothymidine (AZT)), didanosine (ddI), dideoxyinosine, tenofovir, tenofovir alafenamide, tenofovir alafenamide hemifumarate, tenofovir disoproxil fumarate, stavudine (d4T), zalcitabine (dideoxycytidine, ddC), and abacavir.

In some embodiments, the NNRTI is selected from efavirenz, etravirine, rilpivirine, nevirapine, and delavirdine.

In some embodiments, the PI is selected from amprenavir, atazanavir, darunavir, fosamprenavir, indinavir, lopinavir, nelfinavir, ritonavir, saquinavir, and tipranavir.

In some embodiments, the INSTI is selected from raltegravir, elvitegravir, and dolutegravir.

In some embodiments of the method, the patient had been previously treated with at least one antiretroviral medication for at least 3 months, at least 6 months, at least 9 months, or at least 12 months.

In some embodiments, the patient failed a prior HIV treatment regimen including administration of at least one antiretroviral medication. In certain embodiments, the prior treatment regimen included administration of at least one antiretroviral medication from each of two different classes of antiretroviral medications. In certain embodiments, the prior treatment regimen included administration of at least one antiretroviral medication from each of three different classes of antiretroviral medications. In some embodiments, the different classes of antiretroviral medications are selected from a nucleoside reverse transcriptase inhibitor (NRTI), a non-nucleoside reverse transcriptase inhibitor (NNRTI), a protease inhibitor (PI), and an integrase strand transfer inhibitor (INSTI).

In some embodiments of the methods provided herein, the compound of Formula I, or a pharmaceutically acceptable salt thereof, is administered as a long-acting injectable pharmaceutical composition (e.g., a solution or suspension formulation).

In some embodiments of the methods provided herein, the compound of Formula I, or a pharmaceutically acceptable salt thereof, is administered as a solution formulation. In some embodiments, the solution formulation comprises N-methylpyrrolidone, N-butylpyrrolidone, dimethylsulfoxide, or any combination thereof. In some embodiments, the solution formulation comprises N-methylpyrrolidone.

In some embodiments of the methods provided herein, the compound of Formula I, or a pharmaceutically acceptable salt thereof, is administered as a suspension formulation.

In some embodiments of the methods provided herein, the compound of Formula I, or a pharmaceutically acceptable salt thereof, is administered as an organic suspension formulation.

In some embodiments of the methods provided herein, the compound of Formula I, or a pharmaceutically acceptable salt thereof, is administered as an aqueous suspension formulation.

In some embodiments of the methods provided herein, the pharmaceutical composition comprising the compound of Formula I, or a pharmaceutically acceptable salt thereof, is administered subcutaneously.

In some embodiments of the methods provided herein, the pharmaceutical composition comprising the compound of Formula I, or a pharmaceutically acceptable salt thereof, is administered intramuscularly.

In some embodiments of the methods provided herein, the compound of Formula I, or a pharmaceutically acceptable salt thereof, is administered in combination with one or more additional therapeutic agents (e.g., one, two, three, or four additional therapeutic agents).

In certain embodiments, the present description provides a method for treating an HIV infection, comprising administering to a patient in need thereof a therapeutically effective amount of a composition described herein, in combination with a therapeutically effective amount of one or more additional therapeutic agents.

Combination or co-administration of the compound of Formula I, or a pharmaceutically acceptable salt thereof, with one or more additional therapeutic agents generally refers to simultaneous or sequential administration of the compound of Formula I, or a pharmaceutically acceptable salt thereof, and one or more additional therapeutic agents, such that therapeutically effective amount of the compound of Formula I, or a pharmaceutically acceptable salt thereof, and the one or more additional therapeutic agents are both present in the body of the patient. When administered sequentially, the combination may be administered in two or more administrations.

Co-administration includes administration of unit dosages of the compounds disclosed herein before or after administration of unit dosages of one or more additional therapeutic agents. For example, the compound disclosed herein may be administered within seconds, minutes, or hours of the administration of the one or more additional therapeutic agents. In some embodiments, a unit dose of a compound disclosed herein is administered first, followed within seconds or minutes by administration of a unit dose of one or more additional therapeutic agents.

Alternatively, a unit dose of one or more additional therapeutic agents is administered first, followed by administration of a unit dose of a compound disclosed herein within seconds or minutes. In other embodiments, a unit dose of a compound disclosed herein is administered first, followed, after a period of hours (e.g., 1-12 hours), by administration of a unit dose of one or more additional therapeutic agents. In yet other embodiments, a unit dose of one or more additional therapeutic agents is administered first, followed, after a period of hours (e.g., 1-12 hours), by administration of a unit dose of a compound disclosed herein.

In some embodiments, the additional therapeutic agent may be an anti-HIV agent. In some instances, the additional therapeutic agent can be HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, HIV entry inhibitors, HIV maturation inhibitors, HIV capsid inhibitors, HIV Tat or Rev inhibitors, immunomodulators, immunotherapeutic agents, antibody-drug conjugates, gene modifiers, gene editors (such as CRISPR/Cas9, zinc finger nucleases, homing nucleases, synthetic nucleases, TALENs), cell therapies (such as chimeric antigen receptor T-cell, CAR-T, and engineered T-cell receptors, TCR-T, autologous T-cell therapies, engineered B cells), latency reversing agents, immune-based therapies, phosphatidylinositol 3-kinase (PI3K) inhibitors, HIV antibodies, bispecific antibodies and “antibody-like” therapeutic proteins, HIV p17 matrix protein inhibitors, IL-13 antagonists, peptidyl-prolyl cis-trans isomerase A modulators, protein disulfide isomerase inhibitors, complement C5a receptor antagonists, DNA methyltransferase inhibitor, Fatty acid synthase inhibitor, HIV vif gene modulators, Vif dimerization antagonists, HIV-1 viral infectivity factor inhibitors, HIV-1 Nef modulators, TNF alpha ligand inhibitors, HIV Nef inhibitors, Hck tyrosine kinase modulators, mixed lineage kinase-3 (MLK-3) inhibitors, HIV-1 splicing inhibitors, integrin antagonists, nucleoprotein inhibitors, splicing factor modulators, COMM domain containing protein 1 modulators, HIV ribonuclease H inhibitors, IFN antagonists, retrocyclin modulators, CDK-4 inhibitors, CDK-6 inhibitors, CDK-9 inhibitors, dendritic ICAM-3 grabbing nonintegrin 1 inhibitors, HIV GAG protein inhibitors, HIV POL protein inhibitors, Complement Factor H modulators, ubiquitin ligase inhibitors, deoxycytidine kinase inhibitors, cyclin dependent kinase inhibitors, proprotein convertase PC9 stimulators, ATP dependent RNA helicase DDX3X inhibitors, reverse transcriptase priming complex inhibitors, G6PD and NADH-oxidase inhibitors, mTOR complex 1 inhibitors, mTOR complex 2 inhibitors, P-Glycoprotein modulators, TAT protein inhibitors, Prolylendopeptidase inhibitors, Phospholipase A2 inhibitors, pharmacokinetic enhancers, HIV gene therapy, HIV vaccines, and combinations thereof.

In some embodiments, the additional therapeutic agent is selected from the group consisting of combination drug products for HIV, other drugs for treating HIV, HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors, latency reversing agents, capsid inhibitors, immune-based therapies, PI3K inhibitors, HIV antibodies, and bispecific antibodies, and “antibody-like” therapeutic proteins, and combinations thereof.

In some embodiments, the additional therapeutic agents are each independently selected from HIV capsid inhibitors, HIV protease inhibiting compounds, HIV nonnucleoside inhibitors of reverse transcriptase, HIV nucleoside inhibitors of reverse transcriptase, HIV nucleotide inhibitors of reverse transcriptase, HIV integrase inhibitors, and CCR5 inhibitors.

### HIV Combination Drug Products

Examples of combination drug products include, but are not limited to, ATRIPLA® (efavirenz, tenofovir disoproxil fumarate, and emtricitabine); COMPLERA® (EVIPLERA®; rilpivirine, tenofovir disoproxil fumarate, and emtricitabine); STRIBILD® (elvitegravir, cobicistat, tenofovir disoproxil fumarate, and emtricitabine); TRUVADA® (tenofovir disoproxil fumarate and emtricitabine; TDF+FTC); DESCOVY® (tenofovir alafenamide and emtricitabine); ODEFSEY® (tenofovir alafenamide, emtricitabine, and rilpivirine); GENVOYA® (tenofovir alafenamide, emtricitabine, cobicistat, and elvitegravir); darunavir, tenofovir alafenamide hemifumarate, emtricitabine, and cobicistat; efavirenz, lamivudine, and tenofovir disoproxil fumarate; lamivudine and tenofovir disoproxil fumarate; tenofovir and lamivudine; tenofovir alafenamide and emtricitabine; tenofovir alafenamide hemifumarate and emtricitabine; tenofovir alafenamide hemifumarate, emtricitabine, and rilpivirine; tenofovir alafenamide hemifumarate, emtricitabine, cobicistat, and elvitegravir; tenofovir analog; COMBIVIR® (zidovudine and lamivudine; AZT+3TC); EPZICOM® (KIVEXA®; abacavir sulfate and lamivudine; ABC+3TC); KALETRA® (ALUVIA®; lopinavir and ritonavir); TRIUMEQ® (dolutegravir, abacavir, and lamivudine); BIKTARVY® (bictegravir+emtricitabine+tenofovir alafenamide), DOVATO® (dolutegravir+lamivudine), TRIZIVIR® (abacavir sulfate, zidovudine, and lamivudine; ABC+AZT+3TC); atazanavir and cobicistat; atazanavir sulfate and cobicistat; atazanavir sulfate and ritonavir; darunavir and cobicistat; dolutegravir and rilpivirine; dolutegravir and rilpivirine hydrochloride; dolutegravir, abacavir sulfate, and lamivudine; lamivudine, nevirapine, and zidovudine; raltegravir and lamivudine; doravirine, lamivudine, and tenofovir disoproxil fumarate; doravirine, lamivudine, and tenofovir disoproxil; dolutegravir+lamivudine, lamivudine+abacavir+zidovudine, lamivudine+abacavir, lamivudine+tenofovir disoproxil fumarate, lamivudine+zidovudine+nevirapine, lopinavir+ritonavir, lopinavir+ritonavir+abacavir+lamivudine, lopinavir+ritonavir+zidovudine+lamivudine, tenofovir+lamivudine, and tenofovir disoproxil fumarate+emtricitabine+rilpivirine hydrochloride, lopinavir, ritonavir, zidovudine, lopinavir+ritonavir+abacavir+lamivudine, lamivudine, cabotegravir+rilpivirine, 3-BNC117+albuvirtide, elpida (elsulfavirine, VM-1500), and VM-1500A, lenacapavir+islatravir (oral, injectable), and dual-target HIV-1 reverse transcriptase/nucleocapsid protein 7 inhibitors.

### Other HIV Drugs

Examples of other drugs for treating HIV include, but are not limited to, aspernigrin C, acemannan, alisporivir, BanLec, deferiprone, Gamimune, metenkefalin, naltrexone, Prolastin, REP 9, RPI-MN, VSSP, Hlviral, SB-728-T, 1,5-dicaffeoylquinic acid, rHIV7-shl-TAR-CCR5RZ, AAV-eCD4-Ig gene therapy, MazF gene therapy, BlockAide, bevirimat derivatives, ABBV-382, ABX-464, AG-1105, APH-0812, APH0202, bryostatin-1, bryostatin analogs, BIT-225, BRII-732, BRII-778, CYT-107, CS-TATI-1, fluoro-beta-D-arabinose nucleic acid (FANA)-modified antisense oligonucleotides, FX-101, griffithsin, GSK-3739937, GSK-3739937 (long-acting), HGTV-43, HPH-116, HS-10234, hydroxychloroquine, IMB-10035, IMO-3100, IND-02, JL-18008, LADAVRU, MK-1376, MK-2048, MK-4250, MK-8507, MK-8558, MK-8591 (islatravir), NOV-205, OB-002H, ODE-Bn-TFV, PA-1050040 (PA-040), PC-707, PGN-007, QF-036, 5-648414, SCY-635, SB-9200, SCB-719, TR-452, TEV-90110, TEV-90112, TEV-90111, TEV-90113, RN-18, DIACC-1010, Fasnall, Immuglo, 2-CLIPS peptide, HRF-4467, thrombospondin analogs, TBL-1004HI, VG-1177, xl-081, AVI-CO-004, rfhSP-D, [18F]-MC-225, URMC-099-C, RES-529, Verdinexor, IMC-M113V, IML-106, antiviral fc conjugate (AVC), WP-1096, WP-1097, Gammora, ISR-CO48, ISR-48, ISR-49, MK-8527, cannabinoids, ENOB-HV-32, HiviCide-I, T-1144, VIR-576, nipamovir, Covimro, and ABBV-1882.

### HIV Protease Inhibitors

Examples of HIV protease inhibitors include, but are not limited to, amprenavir, atazanavir, brecanavir, darunavir, fosamprenavir, fosamprenavir calcium, indinavir, indinavir sulfate, lopinavir, nelfinavir, nelfinavir mesylate, ritonavir, saquinavir, saquinavir mesylate, tipranavir, ASC-09+ritonavir, AEBL-2, DG-17, GS-1156, TMB-657 (PPL-100), T-169, BL-008, MK-8122, TMB-607, GRL-02031, and TMC-310911.

Additional examples of HIV protease inhibitors are described, e.g., in U.S. Pat. No. 10,294,234, and U.S. Patent Application Publication Nos. US2020030327 and US2019210978.

### HIV Gag Protein Inhibitors

Examples of HIV Gag protein inhibitors include, but are not limited to, HRF-10071.

### HIV Ribonuclease H Inhibitors

Examples of HIV ribonuclease H inhibitors include, but are not limited to, NSC-727447.

### HIV Nef Inhibitors

Examples of HIV Nef inhibitors include, but are not limited to, FP-1.

### HIV Reverse Transcriptase Inhibitors

Examples of HIV non-nucleoside or non-nucleotide inhibitors of reverse transcriptase include, but are not limited to, dapivirine, delavirdine, delavirdine mesylate, doravirine, efavirenz, etravirine, lentinan, nevirapine, rilpivirine, ACC-007, ACC-008, AIC-292, F-18, KM-023, PC-1005, M1-TFV, M2-TFV, VM-1500A-LAI, PF-3450074, elsulfavirine (sustained release oral, HIV infection), doravirine+islatravir (fixed dose combination/oral tablet formulation, HIV-1 infection), elsulfavirine (long acting injectable nanosuspension, HIV infection), and elsulfavirine (VM-1500).

Examples of HIV nucleoside or nucleotide inhibitors of reverse transcriptase include, but are not limited to, adefovir, adefovir dipivoxil, azvudine, emtricitabine, tenofovir, tenofovir alafenamide, tenofovir alafenamide fumarate, tenofovir alafenamide hemifumarate, tenofovir disoproxil, tenofovir disoproxil fumarate, tenofovir octadecyloxyethyl ester (AGX-1009), tenofovir disoproxil hemifumarate, VIDEX® and VIDEX EC® (didanosine, ddl), abacavir, abacavir sulfate, alovudine, apricitabine, censavudine, didanosine, elvucitabine, festinavir, fosalvudine tidoxil, CMX-157, dapivirine, doravirine, etravirine, OCR-5753, tenofovir disoproxil orotate, fozivudine tidoxil, lamivudine, phosphazid, stavudine, zalcitabine, zidovudine, rovafovir etalafenamide (GS-9131), GS-9148, MK-8504, islatravir, MK-8583, VM-2500, and KP-1461.

Additional examples of HIV nucleoside or nucleotide inhibitors of reverse transcriptase include, but are not limited to, those described in patent publications US2007049754, US2016250215, US2016237062, US2016251347, US2002119443, US2013065856, US2013090473, US2014221356, and WO04096286.

### HIV Integrase Inhibitors

Examples of HIV integrase inhibitors include, but are not limited to, elvitegravir, elvitegravir (extended-release microcapsules), curcumin, derivatives of curcumin, chicoric acid, derivatives of chicoric acid, 3,5-dicaffeoylquinic acid, derivatives of 3,5-dicaffeoylquinic acid, aurintricarboxylic acid, derivatives of aurintricarboxylic acid, caffeic acid phenethyl ester, derivatives of caffeic acid phenethyl ester, tyrphostin, derivatives of tyrphostin, quercetin, derivatives of quercetin, raltegravir, PEGylated raltegravir, dolutegravir, JTK-351, bictegravir, AVX-15567, cabotegravir (long acting injectable), diketo quinolin-4-1 derivatives, integrase-LEDGF inhibitor, ledgins, M-522, M-532, MK-0536, NSC-310217, NSC-371056, NSC-48240, NSC-642710, NSC-699171, NSC-699172, NSC-699173, NSC-699174, stilbenedisulfonic acid, T169, STP-0404, VM-3500, XVIR-110, and ACC-017.

Examples of HIV non-catalytic site, or allosteric, integrase inhibitors (NCINI) include, but are not limited to, CX-05045, CX-05168, and CX-14442.

Additional examples of HIV capsid inhibitors include, but are not limited to, those described in U.S. Patent Application Publication Nos. US2014221356 and US2016016973.

### HIV Viral Infectivity Factor Inhibitors

Examples of HIV viral infectivity factor inhibitors include, but are not limited to, 2-amino-N-(2-methoxyphenyl)-6-((4-nitrophenyl)thio)benzamide derivatives, and Irino-L.

### HIV Entry Inhibitors

Examples of HIV entry (fusion) inhibitors include, but are not limited to, AAR-501, LBT-5001, cenicriviroc, CCR5 inhibitors, gp41 inhibitors, CD4 attachment inhibitors, gp120 inhibitors, gp160 inhibitors, and CXCR4 inhibitors.

Examples of CCR5 inhibitors include, but are not limited to, aplaviroc, vicriviroc, maraviroc, maraviroc (long acting injectable nanoemulsion), cenicriviroc, leronlimab (PRO-140), adaptavir (RAP-101), nifeviroc (TD-0232), anti-GP120/CD4 or CCR5 bispecific antibodies, B-07, MB-66, polypeptide C25P, TD-0680, thioraviroc and vMIP (Haimipu).

Examples of gp41 inhibitors include, but are not limited to, albuvirtide, enfuvirtide, griffithsin (gp41/gp120/gp160 inhibitor), BMS-986197, enfuvirtide biobetter, enfuvirtide biosimilar, HIV-1 fusion inhibitors (P26-Bapc), ITV-1, ITV-2, ITV-3, ITV-4, CPT-31, Cl3hmAb, lipuvirtide, PIE-12 trimer and sifuvirtide.

Examples of CD4 attachment inhibitors include, but are not limited to, ibalizumab and CADA analogs

Examples of gp120 inhibitors include, but are not limited to, anti-HIV microbicide, Radha-108 (receptol) 3B3-PE38, BMS818251, BanLec, bentonite-based nanomedicine, fostemsavir tromethamine, IQP-0831, VVX-004, and BMS-663068.

Examples of gp160 inhibitors include, but are not limited to, fangchinoline.

Examples of CXCR4 inhibitors include, but are not limited to, plerixafor, ALT-1188, N15 peptide, and vMIP (Haimipu).

### HIV Maturation Inhibitors

Examples of HIV maturation inhibitors include, but are not limited to, BMS-955176, GSK-3640254 and GSK-2838232.

### Latency Reversing Agents

Examples of latency reversing agents include, but are not limited to, toll-like receptor (TLR) agonists (including TLR7 agonists, e.g., GS-9620, TLR8 agonists, and TLR9 agonists), histone deacetylase (HDAC) inhibitors, proteasome inhibitors such as velcade, protein kinase C (PKC) activators, Smyd2 inhibitors, BET-bromodomain 4 (BRD4) inhibitors (such as ZL-0580, apabetalone), ionomycin, IAP antagonists (inhibitor of apoptosis proteins, such as APG-1387, LBW-242), SMAC mimetics (including TL32711, LCL161, GDC-0917, HGS1029, AT-406, Debio-1143), PMA, SAHA (suberanilohydroxamic acid, or suberoyl, anilide, and hydroxamic acid), NIZ-985, IL-15 modulating antibodies (including IL-15, IL-15 fusion proteins, and IL-15 receptor agonists), JQ1, disulfiram, amphotericin B, and ubiquitin inhibitors such as largazole analogs, APH-0812, and GSK-343. Examples of PKC activators include, but are not limited to, indolactam, prostratin, ingenol B, and DAG-lactones.

Additional examples of TLR7 agonists include, but are not limited to, those described in U.S. Patent Application Publication No. US2010143301.

Additional examples of TLR8 agonists include, but are not limited to, those described in U.S. Patent Application Publication No. US2017071944.

### Histone Deacetylase (HDAC) Inhibitors

In some embodiments, the agents as described herein are combined with an inhibitor of a histone deacetylase, e.g., histone deacetylase 1, histone deacetylase 9 (HDAC9, HD7, HD7b, HD9, HDAC, HDAC7, HDAC7B, HDAC9B, HDAC9FL, HDRP, MITR; Gene ID: 9734). Examples of HDAC inhibitors include without limitation, abexinostat, ACY-241, AR-42, BEBT-908, belinostat, CKD-581, CS-055 (HBI-8000), CT-101, CUDC-907 (fimepinostat), entinostat, givinostat, mocetinostat, panobinostat, pracinostat, quisinostat (JNJ-26481585), resminostat, ricolinostat, romidepsin, SHP-141, TMB-ADC, valproic acid (VAL-001), vorinostat, tinostamustine, remetinostat, and entinostat.

### Capsid Inhibitors

Examples of capsid inhibitors include, but are not limited to, capsid polymerization inhibitors or capsid disrupting compounds, HIV nucleocapsid p7 (NCp7) inhibitors such as azodicarbonamide, HIV p24 capsid protein inhibitors, lenacapavir (GS-6207), GS-CA1, AVI-621, AVI-101, AVI-201, AVI-301, and AVI-CAN1-15 series, PF-3450074, HIV-1 capsid inhibitors (HIV-1 infection, Shandong University), and compounds described in (GSK WO2019/087016).

Additional examples of capsid inhibitors include, but not limited to, those described in U.S. Patent Application Publication Nos. US2018051005 and US2016108030.

### Cytochrome P450 3 Inhibitors

Examples of Cytochrome P450 3 inhibitors include, but are not limited to, those described in U.S. Pat. No. 7,939,553.

### RNA Polymerase Modulators

Examples of RNA polymerase modulators include, but are not limited to, those described in U.S. Pat. Nos. 10,065,958 and 8,008,264.

### Immune Checkpoint Modulators

In various embodiments, the agents as described herein, are combined with one or more blockers or inhibitors of inhibitory immune checkpoint proteins or receptors and/or with one or more stimulators, activators or agonists of one or more stimulatory immune checkpoint proteins or receptors. Blockade or inhibition of inhibitory immune checkpoints can positively regulate T-cell or NK cell activation and prevent immune escape of infected cells. Activation or stimulation of stimulatory immune check points can augment the effect of immune checkpoint inhibitors in infective therapeutics. In various embodiments, the immune checkpoint proteins or receptors regulate T cell responses (e.g., reviewed in Xu et al., J Exp Clin Cancer Res. (2018) 37:110). In various embodiments, the immune checkpoint proteins or receptors regulate NK cell responses (e.g., reviewed in Davis et al., Semin Immunol. (2017) 31:64-75 and Chiossone et al., Nat Rev Immunol. (2018) 18(11):671-688).

Examples of immune checkpoint proteins or receptors include without limitation CD27, CD70; CD40, CD40LG; CD47, CD48 (SLAMF2), transmembrane and immunoglobulin domain containing 2 (TMIGD2, CD28H), CD84 (LY9B, SLAMF5), CD96, CD160, MS4A1 (CD20), CD244 (SLAMF4); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); natural killer cell cytotoxicity receptor 3 ligand 1 (NCR3LG1, B7H6); HERV-H LTR-associating 2 (HHLA2, B7H7); inducible T cell co-stimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF8 (CD30), TNFSF8 (CD30L); TNFRSF1OA (CD261, DR4, TRAILR1), TNFRSF9 (CD137), TNFSF9 (CD137L); TNFRSF1OB (CD262, DR5, TRAILR2), TNFRSF1O (TRAIL); TNFRSF14 (HVEM, CD270), TNFSF14 (HVEML); CD272 (B and T lymphocyte associated (BTLA)); TNFRSF17 (BCMA, CD269), TNFSF13B (BAFF); TNFRSF18 (GITR), TNFSF18 (GITRL); MHC class I polypeptide-related sequence A (MICA); MHC class I polypeptide-related sequence B (MICB); CD274 (CD274, PDL1, PD-L1); programmed cell death 1 (PDCD1, PD1, PD-1); cytotoxic T-lymphocyte associated protein 4 (CTLA4, CD152); CD80 (B7-1), CD28; nectin cell adhesion molecule 2 (NECTIN2, CD112); CD226 (DNAM-1); Poliovirus receptor (PVR) cell adhesion molecule (PVR, CD155); PVR related immunoglobulin domain containing (PVRIG, CDI12R); T cell immunoreceptor with Ig and ITIM domains (TIGIT); T cell immunoglobulin and mucin domain containing 4 (TIMD4; TIM4); hepatitis A virus cellular receptor 2 (HAVCR2, TIMD3, TIM3); galectin 9 (LGALS9); lymphocyte activating 3 (LAG3, CD223); signaling lymphocytic activation molecule family member 1 (SLAMFI, SLAM, CD150); lymphocyte antigen 9 (LY9, CD229, SLAMF3); SLAM family member 6 (SLAMF6, CD352); SLAM family member 7 (SLAMF7, CD319); UL16 binding protein 1 (ULBPI); UL16 binding protein 2 (ULBP2); UL16 binding protein 3 (ULBP3); retinoic acid early transcript 1E (RAETIE; ULBP4); retinoic acid early transcript 1G (RAETIG; ULBP5); retinoic acid early transcript 1L (RAETIL; ULBP6); lymphocyte activating 3 (CD223); killer cell immunoglobulin like receptor, three Ig domains and long cytoplasmic tail 1 (KIR, CD158E1); killer cell lectin like receptor C1 (KLRC1, NKG2 Å, CD159 Å); killer cell lectin like receptor K1 (KLRK1, NKG2D, CD314); killer cell lectin like receptor C2 (KLRC2, CD159c, NKG2C); killer cell lectin like receptor C3 (KLRC3, NKG2E); killer cell lectin like receptor C4 (KLRC4, NKG2F); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); killer cell immunoglobulin like receptor, three Ig domains and long cytoplasmic tail 1 (KIR3DL1); killer cell lectin like receptor D1 (KLRD1); SLAM family member 7 (SLAMF7); and Hematopoietic Progenitor Kinase 1 (HPK1, MAP4K1).

In various embodiments, the agents described herein are combined with one or more blockers or inhibitors of one or more T-cell inhibitory immune checkpoint proteins or receptors. Illustrative T-cell inhibitory immune checkpoint proteins or receptors include without limitation CD274 (CD274, PDL1, PD-L1); programmed cell death 1 ligand 2 (PDCDILG2, PD-L2, CD273); programmed cell death 1 (PDCD1, PD1, PD-1); cytotoxic T-lymphocyte associated protein 4 (CTLA4, CD152); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSFi1, VSIG3); TNFRSF14 (HVEM, CD270), TNFSFi4 (HVEML); CD272 (B and T lymphocyte associated (BTLA)); PVR related immunoglobulin domain containing (PVRIG, CDI12R); T cell immunoreceptor with Ig and ITIM domains (TIGIT); lymphocyte activating 3 (LAG3, CD223); hepatitis A virus cellular receptor 2 (HAVCR2, TIMD3, TIM3); galectin 9 (LGALS9); killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); and killer cell immunoglobulin like receptor, three Ig domains and long cytoplasmic tail 1 (KIR3DL1). In various embodiments, the agents, as described herein, are combined with one or more agonist or activators of one or more T-cell stimulatory immune checkpoint proteins or receptors. Illustrative T-cell stimulatory immune checkpoint proteins or receptors include without limitation CD27, CD70; CD40, CD40LG; inducible T cell costimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF9 (CD137), TNFSF9 (CD137L); TNFRSF18 (GITR), TNFSFi8 (GITRL); CD80 (B7-1), CD28; nectin cell adhesion molecule 2 (NECTIN2, CD 112); CD226 (DNAM-1); CD244 (2B4, SLAMF4), Poliovirus receptor (PVR) cell adhesion molecule (PVR, CD155). See, e.g., Xu et al., J Exp Clin Cancer Res. (2018) 37:110.

In various embodiments, the agents as described herein, are combined with one or more blockers or inhibitors of one or more NK-cell inhibitory immune checkpoint proteins or receptors. Illustrative NK-cell inhibitory immune checkpoint proteins or receptors include without limitation killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); killer cell immunoglobulin like receptor, three Ig domains and long cytoplasmic tail 1 (KIR3DL1); killer cell lectin like receptor C1 (KLRC1, NKG2A, CD159A); and killer cell lectin like receptor D1 (KLRD1, CD94). In various embodiments, the agents as described herein, are combined with one or more agonist or activators of one or more NK-cell stimulatory immune checkpoint proteins or receptors. Illustrative NK-cell stimulatory immune checkpoint proteins or receptors include without limitation CD16, CD226 (DNAM-1); CD244 (2B4, SLAMF4); killer cell lectin like receptor K1 (KLRK1, NKG2D, CD314); SLAM family member 7 (SLAMF7). See, e.g., Davis et al., Semin Immunol. (2017) 31:64-75; Fang et al., Semin Immunol. (2017) 31:37-54; and Chiossone et al., Nat Rev Immunol. (2018) 18(11):671-688.

In some embodiments, the one or more immune checkpoint inhibitors comprises a proteinaceous (e.g., antibody or fragment thereof, or antibody mimetic) inhibitor of PD-L1 (CD274), PD-1 (PDCD1) or CTLA4. In some embodiments, the one or more immune checkpoint inhibitors comprises a small organic molecule inhibitor of PD-L1 (CD274), PD-1 (PDCD1) or CTLA4. In some embodiments, the small molecule inhibitor of CD274 or PDCD1 is selected from the group consisting of GS-4224, GS-4416, INCB086550 and MAX10181. In some embodiments, the small molecule inhibitor of CTLA4 comprises BPI-002.

Examples of inhibitors of CTLA4 that can be co-administered include without limitation ipilimumab, tremelimumab, BMS-986218, AGEN1181, AGEN1884, BMS-986249, MK-1308, REGN-4659, ADU-1604, CS-1002, BCD-145, APL-509, JS-007, BA-3071, ONC-392, AGEN-2041, JHL-1155, KN-044, CG-0161, ATOR-1144, PBI-5D3H5, BPI-002, as well as multi-specific inhibitors FPT-155 (CTLA4/PD-L1/CD28), PF-06936308 (PD-1/CTLA4), MGD-019 (PD-1/CTLA4), KN-046 (PD-1/CTLA4), MEDI-5752 (CTLA4/PD-1), XmAb-20717 (PD-1/CTLA4), and AK-104 (CTLA4/PD-1).

Examples of inhibitors of PD-L1 (CD274) or PD-1 (PDCD1) that can be co-administered include without limitation pembrolizumab, nivolumab, cemiplimab, pidilizumab, AMP-224, MEDI0680 (AMP-514), spartalizumab, atezolizumab, avelumab, durvalumab, BMS-936559, CK-301, PF-06801591, BGB-A317 (tislelizumab), GLS-010 (WBP-3055), AK-103 (HX-008), AK-105, CS-1003, HLX-10, MGA-012, BI-754091, AGEN-2034, JS-001 (toripalimab), JNJ-63723283, genolimzumab (CBT-501), LZM-009, BCD-100, LY-3300054, SHR-1201, SHR-1210 (camrelizumab), Sym-021, ABBV-181(budigalimab), PD1-PIK, BAT-1306, (MSB0010718C), CX-072, CBT-502, TSR-042 (dostarlimab), MSB-2311, JTX-4014, BGB-A333, SHR-1316, CS-1001 (WBP-3155, KN-035, IBI-308 (sintilimab), HLX-20, KL-A167, STI-A1014, STI-A1015 (IMC-001), BCD-135, FAZ-053, TQB-2450, MDX1105-01, GS-4224, GS-4416, INCB086550, MAX10181, as well as multi-specific inhibitors FPT-155 (CTLA4/PD-L1/CD28), PF-06936308 (PD-1/CTLA4), MGD-013 (PD-1/LAG-3), FS-118 (LAG-3/PD-L1) MGD-019 (PD-1/CTLA4), KN-046 (PD-1/CTLA4), MEDI-5752 (CTLA4/PD-1), RO-7121661 (PD-1/TIM-3), XmAb-20717 (PD-1/CTLA4), AK-104 (CTLA4/PD-1), M7824 (PD-L1/TGFD-EC domain), CA-170 (PD-L1/VISTA), CDX-527 (CD27/PD-L1), LY-3415244 (TIM3/PDL1), and INBRX-105 (4-1BB/PDL1).

In various embodiments, the agents as described herein are combined with anti-TIGIT antibodies, such as BMS-986207, RG-6058, and AGEN-1307.

### TNF Receptor Superfamily (TNFRSF) Member Agonists or Activators

In various embodiments, the agents as described herein are combined with an agonist of one or more TNF receptor superfamily (TNFRSF) members, e.g., an agonist of one or more of TNFRSF1A (NCBI Gene ID: 7132), TNFRSF1B (NCBI Gene ID: 7133), TNFRSF4 (OX40, CD134; NCBI Gene ID: 7293), TNFRSF5 (CD40; NCBI Gene ID: 958), TNFRSF6 (FAS, NCBI Gene ID: 355), TNFRSF7 (CD27, NCBI Gene ID: 939), TNFRSF8 (CD30, NCBI Gene ID: 943), TNFRSF9 (4-1BB, CD137, NCBI Gene ID: 3604), TNFRSF10A (CD261, DR4, TRAILR1, NCBI Gene ID: 8797), TNFRSF10B (CD262, DR5, TRAILR2, NCBI Gene ID: 8795), TNFRSF10C (CD263, TRAILR3, NCBI Gene ID: 8794), TNFRSF10D (CD264, TRAILR4, NCBI Gene ID: 8793), TNFRSF11A (CD265, RANK, NCBI Gene ID: 8792), TNFRSF11B (NCBI Gene ID: 4982), TNFRSF12A (CD266, NCBI Gene ID: 51330), TNFRSF13B (CD267, NCBI Gene ID: 23495), TNFRSF13C (CD268, NCBI Gene ID: 115650), TNFRSF16 (NGFR, CD271, NCBI Gene ID: 4804), TNFRSF17 (BCMA, CD269, NCBI Gene ID: 608), TNFRSF18 (GITR, CD357, NCBI Gene ID: 8784), TNFRSF19 (NCBI Gene ID: 55504), TNFRSF21 (CD358, DR6, NCBI Gene ID: 27242), and TNFRSF25 (DR3, NCBI Gene ID: 8718).

Examples of anti-TNFRSF4 (OX40) antibodies that can be co-administered include without limitation, MEDI6469, MEDI6383, MEDI0562 (tavolixizumab), MOXR0916, PF-04518600, RG-7888, GSK-3174998, INCAGN1949, BMS-986178, GBR-8383, ABBV-368, and those described in WO2016179517, WO2017096179, WO2017096182, WO2017096281, and WO2018089628.

Examples of anti-TNFRSF5 (CD40) antibodies that can be co-administered include without limitation RG7876, SEA-CD40, APX-005M and ABBV-428.

In some embodiments, the anti-TNFRSF7 (CD27) antibody varlilumab (CDX-1127) is co-administered.

Examples of anti-TNFRSF9 (4-1BB, CD137) antibodies that can be co-administered include without limitation urelumab, utomilumab (PF-05082566), AGEN2373 and ADG-106.

Examples of anti-TNFRSF18 (GITR) antibodies that can be co-administered include without limitation, MEDI1873, FPA-154, INCAGN-1876, TRX-518, BMS-986156, MK-1248, GWN-323, and those described in WO2017096179, WO2017096276, WO2017096189, and WO2018089628. In some embodiments, an antibody, or fragment thereof, co-targeting TNFRSF4 (OX40) and TNFRSF18 (GITR) is co-administered. Such antibodies are described, e.g., in WO2017096179 and WO2018089628.

### Bi- and Tri-Specific Natural Killer (NK)-Cell Engagers

In various embodiments, the crystalline forms, salts and co-crystals as described herein, are combined with a bi-specific NK-cell engager (BiKE) or a tri-specific NK-cell engager (TriKE) (e.g., not having an Fc) or bi-specific antibody (e.g., having an Fc) against an NK cell activating receptor, e.g., CD16 Å, C-type lectin receptors (CD94/NKG2C, NKG2D, NKG2E/H and NKG2F), natural cytotoxicity receptors (NKp30, NKp44 and NKp46), killer cell C-type lectin-like receptor (NKp65, NKp80), Fc receptor FcγR (which mediates antibody-dependent cell cytotoxicity), SLAM family receptors (e.g., 2B4, SLAM6 and SLAM7), killer cell immunoglobulin-like receptors (KIR) (KIR-2DS and KIR-3DS), DNAM-1 and CD137 (41BB). As appropriate, the anti-CD16 binding bi-specific molecules may or may not have an Fc. Illustrative bi-specific NK-cell engagers that can be co-administered target CD16 and one or more HIV-associated antigens as described herein. BiKEs and TriKEs are described, e.g., in Felices et al., Methods Mol Biol. (2016) 1441:333-346; Fang et al., Semin Immunol. (2017) 31:37-54. Examples of trispecific NK cell engagers (TRiKE) include, but are not limited to, OXS-3550, HIV-TriKE, and CD16-IL-15-B7H3 TriKe.

### Indoleamine-Pyrrole-2,3-Dioxygenase (IDO1) Inhibitors

In various embodiments, the crystalline forms, salts and co-crystals as described herein are combined with an inhibitor of indoleamine 2,3-dioxygenase 1 (IDO1; NCBI Gene ID: 3620). Examples of IDO1 inhibitors include without limitation, BLV-0801, epacadostat, F-001287, GBV-1012, GBV-1028, GDC-0919, indoximod, NKTR-218, NLG-919-based vaccine, PF-06840003, pyranonaphthoquinone derivatives (SN-35837), resminostat, SBLK-200802, BMS-986205, shIDO-ST, EOS-200271, KHK-2455, and LY-3381916.

### Toll-Like Receptor (TLR) Agonists

In various embodiments, the crystalline forms, salts and co-crystals as described herein are combined with an agonist of a toll-like receptor (TLR), e.g., an agonist of TLR1 (NCBI Gene ID: 7096), TLR2 (NCBI Gene ID: 7097), TLR3 (NCBI Gene ID: 7098), TLR4 (NCBI Gene ID: 7099), TLR5 (NCBI Gene ID: 7100), TLR6 (NCBI Gene ID: 10333), TLR7 (NCBI Gene ID: 51284), TLR8 (NCBI Gene ID: 51311), TLR9 (NCBI Gene ID: 54106), and/or TLR10 (NCBI Gene ID: 81793). Example TLR7 agonists that can be co-administered include without limitation AL-034, DSP-0509, GS-9620 (vesatolimod), vesatolimod analog, LHC-165, TMX-101 (imiquimod), GSK-2245035, resiquimod, DSR-6434, DSP-3025, IMO-4200, MCT-465, MEDI-9197, 3M-051, SB-9922, 3M-052, Limtop, TMX-30X, TMX-202, RG-7863, RG-7854, RG-7795, and the compounds disclosed in US20100143301 (Gilead Sciences), US20110098248 (Gilead Sciences), and US20090047249 (Gilead Sciences), US20140045849 (Janssen), US20140073642 (Janssen), WO2014/056953 (Janssen), WO2014/076221 (Janssen), WO2014/128189 (Janssen), US20140350031 (Janssen), WO2014/023813 (Janssen), US20080234251 (Array Biopharma), US20080306050 (Array Biopharma), US20100029585 (Ventirx Pharma), US20110092485 (Ventirx Pharma), US20110118235 (Ventirx Pharma), US20120082658 (Ventirx Pharma), US20120219615 (Ventirx Pharma), US20140066432 (Ventirx Pharma), US20140088085 (Ventirx Pharma), US20140275167 (Novira Therapeutics), and US20130251673 (Novira Therapeutics). TLR7/TLR8 agonists include without limitation NKTR-262, telratolimod and BDB-001. TLR8 agonists include without limitation E-6887, IMO-4200, IMO-8400, IMO-9200, MCT-465, MEDI-9197, motolimod, resiquimod, GS-9688, VTX-1463, VTX-763, 3M-051, 3M-052, and the compounds disclosed in US20140045849 (Janssen), US20140073642 (Janssen), WO2014/056953 (Janssen), WO2014/076221 (Janssen), WO2014/128189 (Janssen), US20140350031 (Janssen), WO2014/023813 (Janssen), US20080234251 (Array Biopharma), US20080306050 (Array Biopharma), US20100029585 (Ventirx Pharma), US20110092485 (Ventirx Pharma), US20110118235 (Ventirx Pharma), US20120082658 (Ventirx Pharma), US20120219615 (Ventirx Pharma), US20140066432 (Ventirx Pharma), US20140088085 (Ventirx Pharma), US20140275167 (Novira Therapeutics), and US20130251673 (Novira Therapeutics). TLR9 agonists include without limitation AST-008, cobitolimod, CMP-001, IMO-2055, IMO-2125, 5-540956, litenimod, MGN-1601, BB-001, BB-006, IMO-3100, IMO-8400, IR-103, IMO-9200, agatolimod, DIMS-9054, DV-1079, DV-1179, AZD-1419, lefitolimod (MGN-1703), CYT-003, CYT-003-QbG10, tilsotolimod and PUL-042. Examples of TLR3 agonist include rintatolimod, poly-ICLC, RIBOXXON®, Apoxxim, RIBOXXIM®, IPH-33, MCT-465, MCT-475, and ND-1.1. TLR4 agonists include, but are not limited to, G-100 and GSK-1795091.

### CDK Inhibitors or Antagonists

In some embodiments, the crystalline forms, salts and co-crystals described herein are combined with an inhibitor or antagonist of CDK. In some embodiments, the CDK inhibitor or antagonist is selected from the group consisting of VS2-370.

### STING Agonists, RIG-I and NOD2 Modulators

In some embodiments, the crystalline forms, salts and co-crystals described herein are combined with a stimulator of interferon genes (STING). In some embodiments, the STING receptor agonist or activator is selected from the group consisting of ADU-S100 (MIW-815), SB-11285, MK-1454, SR-8291, AdVCA0848, GSK-532, SYN-STING, MSA-1, SR-8291, STING agonist (latent HIV), 5,6-dimethylxanthenone-4-acetic acid (DMXAA), cyclic-GAMP (cGAMP) and cyclic-di-AMP. In some embodiments, the agents described herein are combined with a RIG-I modulator such as RGT-100, or NOD2 modulator, such as SB-9200, and IR-103.

### LAG-3 and TIM-3 Inhibitors

In certain embodiments, the crystalline forms, salts and co-crystals as described herein are combined with an anti-TIM-3 antibody, such as TSR-022, LY-3321367, MBG-453, INCAGN-2390.

In certain embodiments, the crystalline forms, salts and co-crystals herein are combined with an anti LAG-3 (Lymphocyte-activation) antibody, such as relatlimab (ONO-4482), LAG-525, MK-4280, REGN-3767, INCAGN2385.

### Interleukin Agonists

In certain embodiments, the crystalline forms, salts and co-crystals described herein are combined with an interleukin agonist, such as IL-2, IL-7, IL-15, IL-10, IL-12 agonists; examples of IL-2 agonists such as proleukin (aldesleukin, IL-2); BC-IL (Cel-Sci), pegylated IL-2 (e.g., NKTR-214); modified variants of IL-2 (e.g., THOR-707), bempegaldesleukin, AIC-284, ALKS-4230, CUI-101, Neo-2/15; examples of IL-15 agonists, such as ALT-803, NKTR-255, and hetIL-15, interleukin-15/Fc fusion protein, AM-0015, NIZ-985, SO-C101, IL-15 Synthorin (pegylated 11-15), P-22339, and a IL-15-PD-1 fusion protein N-809; examples of IL-7 include without limitation CYT-107.

Examples of additional immune-based therapies that can be combined with the crystalline forms, salts and co-crystals of this disclosure include, but are not limited to, interferon alfa, interferon alfa-2b, interferon alfa-n3, pegylated interferon alfa, interferon gamma; FLT3 agonists such as CDX-301, GS-3583, gepon, normferon, peginterferon alfa-2a, peginterferon alfa-2b, and RPI-MN.

Phosphatidylinositol 3-kinase (PI3K) Inhibitors

Examples of PI3K inhibitors include, but are not limited to, idelalisib, alpelisib, buparlisib, CAI orotate, copanlisib, duvelisib, gedatolisib, neratinib, panulisib, perifosine, pictilisib, pilaralisib, puquitinib mesylate, rigosertib, rigosertib sodium, sonolisib, taselisib, AMG-319, AZD-8186, BAY-1082439, CLR-1401, CLR-457, CUDC-907, DS-7423, EN-3342, GSK-2126458, GSK-2269577, GSK-2636771, INCB-040093, LY-3023414, MLN-1117, PQR-309, RG-7666, RP-6530, RV-1729, SAR-245409, SAR-260301, SF-1126, TGR-1202, UCB-5857, VS-5584, XL-765, and ZSTK-474.

### Alpha-4/Beta-7 Antagonists

Examples of Integrin alpha-4/beta-7 antagonists include, but are not limited to, PTG-100, TRK-170, abrilumab, etrolizumab, carotegrast methyl, and vedolizumab.

### HPK1 Inhibitors

Examples of HPK1 inhibitors include, but are not limited to, ZYF-0272, and ZYF-0057.

### HIV Targeting Antibodies

Examples of HIV antibodies, bispecific antibodies, and “antibody-like” therapeutic proteins include, but are not limited to, DARTs®, DUOBODIES®, BITES®, XmAbs®, TandAbs®, Fab derivatives, bNAbs (broadly neutralizing HIV-1 antibodies), TMB-360, TMB-370, and those targeting HIV gp120 or gp41, antibody-Recruiting Molecules targeting HIV, anti-CD63 monoclonal antibodies, anti-GB virus C antibodies, anti-GP120/CD4, gp120 bispecific monoclonal antibody, CCR5 bispecific antibodies, anti-Nef single domain antibodies, anti-Rev antibody, camelid derived anti-CD18 antibodies, camelid-derived anti-ICAM-1 antibodies, DCVax-001, gp140 targeted antibodies, gp41-based HIV therapeutic antibodies, human recombinant mAbs (PGT-121), PGT121.414.LS, ibalizumab, ibalizumab (second generation), Immuglo, MB-66, clone 3 human monoclonal antibody targeting KLIC (HIV infection), GS-9721, BG-HIV, VRC-HIVMAB091-00-AB.

Various bNAbs may be used. Examples include, but are not limited to, those described in U.S. Pat. Nos. 8,673,307, 9,493,549, 9,783,594, 10,239,935, US2018371086, US2020223907, WO2014/063059, WO2012/158948, WO2015/117008, and PCT/US2015/41272, and WO2017/096221, including antibodies 12A12, 12A21, NIH45-46, bANC131, 8ANC134, 1B2530, INC9, 8ANC195. 8ANC196, 10-259, 10-303, 10-410, 10-847, 10-996, 10-1074, 10-1121, 10-1130, 10-1146, 10-1341, 10-1369, and 10-1074GM. Additional examples include those described in Klein et al., Nature, 492(7427): 118-22 (2012), Horwitz et al., Proc Natl Acad Sci USA, 110(41): 16538-43 (2013), Scheid et al., Science, 333: 1633-1637 (2011), Scheid et al., Nature, 458:636-640 (2009), Eroshkin et al, Nucleic Acids Res., 42 (Database issue):Dl 133-9 (2014), Mascola et al., Immunol Rev., 254(1):225-44 (2013), such as 2F5, 4E10, M66.6, CAP206-CH12, 10E81 (all of which bind the MPER of gp41); PG9, PG16, CHO1-04 (all of which bind V1V2-glycan), 2G12 (which binds to outer domain glycan); b12, HJ16, CH103-106, VRCO1-03, VRC-PG04, 04b, VRC-CH30-34, 3BNC62, 3BNC89, 3BNC91, 3BNC95, 3BNC104, 3BNC176, and 8ANC131 (all of which bind to the CD4 binding site).

Additional broadly neutralizing antibodies that can be used as a second therapeutic agent in a combination therapy are described, e.g., in U.S. Pat. Nos. 8,673,307; 9,493,549; 9,783,594; and WO 2012/154312; WO2012/158948; WO 2013/086533; WO 2013/142324; WO2014/063059; WO 2014/089152, WO 2015/048462; WO 2015/103549; WO 2015/117008; WO2016/014484; WO 2016/154003; WO 2016/196975; WO 2016/149710; WO2017/096221; WO 2017/133639; WO 2017/133640, which are hereby incorporated herein by reference in their entireties for all purposes. Additional examples include, but are not limited to, those described in Sajadi et al., Cell. (2018) 173(7):1783-1795; Sajadi et al., J Infect Dis. (2016) 213(1):156-64; Klein et al., Nature, 492(7427): 118-22 (2012), Horwitz et al., Proc Natl Acad Sci USA, 110(41): 16538-43 (2013), Scheid et al., Science, 333: 1633-1637 (2011), Scheid et al., Nature, 458:636-640 (2009), Eroshkin et al., Nucleic Acids Res., 42 (Database issue):Dl 133-9 (2014), Mascola et al., Immunol Rev., 254(1):225-44 (2013), such as 2F5, 4E10, M66.6, CAP206-CH12, 10E8, 10E8v4, 10E8-5R-100cF, DH511.11P, 7b2, 10-1074, and LN01 (all of which bind the MPER of gp41).

Examples of additional antibodies include, but are not limited to, bavituximab, UB-421, BF520.1, BiIA-SG, CHO1, CH59, C2F5, C4E10, C2F5+C2G12+C4E10, CAP256V2LS, 3BNC117, 3BNC117-LS, 3BNC60, DH270.1, DH270.6, D1D2, 10-1074-LS, Cl3hmAb, GS-9722 (elipovimab), DH411-2, BG18, GS-9721, GS-9723, PGT145, PGT121, PGT-121.60, PGT-121.66, PGT122, PGT-123, PGT-124, PGT-125, PGT-126, PGT-151, PGT-130, PGT-133, PGT-134, PGT-135, PGT-128, PGT-136, PGT-137, PGT-138, PGT-139, MDX010 (ipilimumab), DH511, DH511-2, N6, N6LS, N49P6, N49P7, N49P7.1, N49P9, N49P11, N60P1.1, N60P25.1, N60P2.1, N60P31.1, N60P22, NIH 45-46, PGC14, PGG14, PGT-142, PGT-143, PGT-144, PGDM1400, PGDM12, PGDM21, PCDN-33 Å, 2Dm2m, 4Dm2m, 6Dm2m, PGDM1400, MDX010 (ipilimumab), VRC01, VRC-01-LS, A32, 7B2, 10E8, VRC-07-523, VRC07-523LS, VRC24, VRC41.01, 10E8VLS, 3810109, 10E8v4, IMC-HIV, iMabm36, eCD4-Ig, IOMA, CAP256-VRC26.25, DRVIA7,VRC-HIVMAB080-00-AB, VRC-HIVMAB060-00-AB, P2G12, VRC07, 354BG8, 354BG18, 354BG42, 354BG33, 354BG129, 354BG188, 354BG411, 354BG426, VRC29.03, CAP256, CAP256-VRC26.08, CAP256-VRC26.09, CAP256-VRC26.25, PCT64-24E and VRC38.01, PGT-151, CAP248-2B, 35022, ACS202, VRC34 and VRC34.01, 10E8, 10E8v4, 10E8-5R-100cF, 4E10, DH511.11P, 2F5, 7b2, and LN01.

Examples of HIV bispecific and trispecific antibodies include without limitation MGD014, B12BiTe, BiIA-SG, TMB-bispecific, SAR-441236, VRC-01/PGDM-1400/10E8v4, 10E8.4/iMab, 10E8v4/PGT121-VRC01.

Examples of in vivo delivered bNAbs include without limitation AAV8-VRC07; mRNA encoding anti-HIV antibody VRC01; and engineered B-cells encoding 3BNC117 (Hartweger et al., J. Exp. Med. 2019, 1301).

### Pharmacokinetic Enhancers

Examples of pharmacokinetic enhancers include, but are not limited to, cobicistat, ritonavir, and hyaluronidase.

### Additional Therapeutic Agents

Examples of additional therapeutic agents include, but are not limited to, the compounds disclosed in WO 2004/096286 (Gilead Sciences), WO 2006/015261 (Gilead Sciences), WO 2006/110157 (Gilead Sciences), WO 2012/003497 (Gilead Sciences), WO 2012/003498 (Gilead Sciences), WO 2012/145728 (Gilead Sciences), WO 2013/006738 (Gilead Sciences), WO 2013/159064 (Gilead Sciences), WO 2014/100323 (Gilead Sciences), US 2013/0165489 (University of Pennsylvania), US 2014/0221378 (Japan Tobacco), US 2014/0221380 (Japan Tobacco), WO 2009/062285 (Boehringer Ingelheim), WO 2010/130034 (Boehringer Ingelheim), WO 2013/006792 (Pharma Resources), US 20140221356 (Gilead Sciences), US 20100143301 (Gilead Sciences) and WO 2013/091096 (Boehringer Ingelheim).

### HIV Vaccines

Examples of HIV vaccines include, but are not limited to, peptide vaccines, recombinant subunit protein vaccines, live vector vaccines, DNA vaccines, HIV MAG DNA vaccine, CD4-derived peptide vaccines, vaccine combinations, adenoviral vector vaccines (an adenoviral vector such as Ad5, Ad26 or Ad35), simian adenovirus (chimpanzee, gorilla, rhesus i.e. rhAd), adeno-associated virus vector vaccines, Chimpanzee adenoviral vaccines (e.g., ChAdOXI, ChAd68, ChAd3, ChAd63, ChAd83, ChAd155, ChAd157, Pan5, Pan6, Pan7, Pan9), Coxsackieviruses based vaccines, enteric virus based vaccines, Gorilla adenovirus vaccines, lentiviral vector based vaccine, arenavirus vaccines (such as LCMV, Pichinde), bi-segmented or tri-segmented arenavirus based vaccine, trimer-based HIV-1 vaccine, measles virus based vaccine, flavivirus vector based vaccines, tobacco mosaic virus vector based vaccine, Varicella-zoster virus based vaccine, Human parainfluenza virus 3 (PIV3) based vaccines, poxvirus based vaccine (modified vaccinia virus Ankara (MVA), orthopoxvirus-derived NYVAC, and avipoxvirus-derived ALVAC (canarypox virus) strains); fowlpox virus based vaccine, rhabdovirus-based vaccines, such as VSV and marabavirus; recombinant human CMV (rhCMV) based vaccine, alphavirus-based vaccines, such as semliki forest virus, venezuelan equine encephalitis virus and sindbis virus; (see Lauer, Clinical and Vaccine Immunology, 2017, DOI: 10.1128/CVI.00298-16); LNP formulated mRNA based therapeutic vaccines; LNP-formulated self-replicating RNA/self-amplifying RNA vaccines.

Examples of vaccines include: AAVLP-HIV vaccine, AE-298p, anti-CD40.Env-gp140 vaccine, Ad4-EnvC150, BG505 SOSIP.664 gp140 adjuvanted vaccine, BG505 SOSIP.GT1.1 gp140 adjuvanted vaccine, ChAdOx1.tHIVconsvl vaccine, CMV-MVA triplex vaccine, ChAdOx1.HTI, Chimigen HIV vaccine, ConM SOSIP.v7 gp140, ALVAC HIV (vCP1521), AIDSVAX B/E (gp120), monomeric gp120 HIV-1 subtype C vaccine, MPER-656 liposome subunit vaccine, Remune, ITV-1, Contre Vir, Ad5-ENVA-48, DCVax-001 (CDX-2401), Vacc-4×, Vacc-C5, VAC-3S, multiclade DNA recombinant adenovirus-5 (rAd5), rAd5 gag-pol env A/B/C vaccine, Pennvax-G, Pennvax-GP, Pennvax-G/MVA-CMDR, HIV-TriMix-mRNA vaccine, HIV-LAMP-vax, Ad35, Ad35-GRIN, NAcGM3/VSSP ISA-51, poly-ICLC adjuvanted vaccines, TatImmune, GTU-multiHIV (FIT-06), ChAdV63.HIVconsv, gp140[delta]V2.TV1+MF-59, rVSVIN HIV-1 gag vaccine, SeV-EnvF, SeV-Gag vaccine, AT-20, DNK-4, ad35-Grin/ENV, TBC-M4, HIVAX, HIVAX-2, N123-VRC-34.01 inducing epitope-based HIV vaccine, NYVAC-HIV-PT1, NYVAC-HIV-PT4, DNA-HIV-PT123, rAAV1-PG9DP, GOVX-B11, GOVX-B21, GOVX-C55, TVI-HIV-1, Ad-4 (Ad4-env Clade C+Ad4-mGag), Paxvax, EN41-UGR7C, EN41-FPA2, ENOB-HV-11, ENOB-HV-12, PreVaxTat, AE-H, MYM-V101, CombiHIVvac, ADVAX, MYM-V201, MVA-CMDR, MagaVax, DNA-Ad5 gag/pol/nef/nev (HVTN505), MVATG-17401, ETV-01, CDX-1401, DNA and Sev vectors vaccine expressing SCaVII, rcAD26.MOS1.HIV-Env, Ad26.Mod.HIV vaccine, Ad26.Mod.HIV+MVA mosaic vaccine+gp140, AGS-004, AVX-101, AVX-201, PEP-6409, SAV-001, ThV-01, TL-01, TUTI-16, VGX-3300, VIR-1111, IHV-001, and virus-like particle vaccines such as pseudovirion vaccine, CombiVICHvac, LFn-p24 B/C fusion vaccine, GTU-based DNA vaccine, HIV gag/pol/nef/env DNA vaccine, anti-TAT HIV vaccine, conjugate polypeptides vaccine, dendritic-cell vaccines (such as DermaVir), gag-based DNA vaccine, GI-2010, gp41 HIV-1 vaccine, HIV vaccine (PIKA adjuvant), i-key/MHC class II epitope hybrid peptide vaccines, ITV-2, ITV-3, ITV-4, LIPO-5, multiclade Env vaccine, MVA vaccine, Pennvax-GP, pp71-deficient HCMV vector HIV gag vaccine, rgp160 HIV vaccine, RNActive HIV vaccine, SCB-703, Tat Oyi vaccine, TBC-M4, UBI HIV gp120, Vacc-4×+romidepsin, variant gp120 polypeptide vaccine, rAd5 gag-pol env A/B/C vaccine, DNA.HTI and MVA.HTI, VRC-HIVDNA016-00-VP+VRC-HIVADV014-00-VP, INO-6145, JNJ-9220, gp145 C.6980; eOD-GT8 60mer based vaccine, PD-201401, env (A, B, C, A/E)/gag (C) DNA Vaccine, gp120 (A,B,C,A/E) protein vaccine, PDPHV-201401, Ad4-EnvCN54, EnvSeq-1 Envs HIV-1 vaccine (GLA-SE adjuvanted), HIV p24gag prime-boost plasmid DNA vaccine, HIV-1 iglbl2 neutralizing VRC-01 antibody-stimulating anti-CD4 vaccine, arenavirus vector-based vaccines (Vaxwave, TheraT), MVA-BN HIV-1 vaccine regimen, mRNA based prophylactic vaccines, VPI-211, multimeric HIV gp120 vaccine (Fred Hutchinson cancer center), TBL-1203HI, CH505 TF chTrimer, CD40.HIVRI.Env vaccine, Drep-HIV-PT-1, mRNA-1644, and mRNA-1574.

### Birth Control (Contraceptive) Combination Therapy

In certain embodiments, the agents described herein are combined with a birth control or contraceptive regimen. Therapeutic agents used for birth control (contraceptive) that can be combined with an agent of this disclosure include without limitation cyproterone acetate, desogestrel, dienogest, drospirenone, estradiol valerate, ethinyl Estradiol, ethynodiol, etonogestrel, levomefolate, levonorgestrel, lynestrenol, medroxyprogesterone acetate, mestranol, mifepristone, misoprostol, nomegestrol acetate, norelgestromin, norethindrone, noretynodrel, norgestimate, ormeloxifene, segestersone acetate, ulipristal acetate, and any combinations thereof.

In a particular embodiment, a compound disclosed herein, or a pharmaceutically acceptable salt thereof, is combined with one, two, three, or four additional therapeutic agents selected from ATRIPLA® (efavirenz, tenofovir disoproxil fumarate, and emtricitabine); COMPLERA® (EVIPLERA®; rilpivirine, tenofovir disoproxil fumarate, and emtricitabine); STRIBILD® (elvitegravir, cobicistat, tenofovir disoproxil fumarate, and emtricitabine); TRUVADA® (tenofovir disoproxil fumarate and emtricitabine; TDF+FTC); DESCOVY® (tenofovir alafenamide and emtricitabine); ODEFSEY® (tenofovir alafenamide, emtricitabine, and rilpivirine); GENVOYA® (tenofovir alafenamide, emtricitabine, cobicistat, and elvitegravir); BIKTARVY® (bictegravir+emtricitabine+tenofovir alafenamide), adefovir; adefovir dipivoxil; cobicistat; emtricitabine; tenofovir; tenofovir alafenamide and elvitegravir; tenofovir alafenamide+elvitegravir (rectal formulation, HIV infection); tenofovir disoproxil; tenofovir disoproxil fumarate; tenofovir alafenamide; tenofovir alafenamide hemifumarate; TRIUMEQ® (dolutegravir, abacavir, and lamivudine); dolutegravir, abacavir sulfate, and lamivudine; raltegravir; PEGylated raltegravir; raltegravir and lamivudine; lamivudine+lopinavir+ritonavir+abacavir; maraviroc; tenofovir+emtricitabine+maraviroc, enfuvirtide; ALUVIA® (KALETRA®; lopinavir and ritonavir); COMBIVIR® (zidovudine and lamivudine; AZT+3TC); EPZICOM® (LIVEXA®; abacavir sulfate and lamivudine; ABC+3TC); TRIZIVIR® (abacavir sulfate, zidovudine, and lamivudine; ABC+AZT+3TC); rilpivirine; rilpivirine hydrochloride; atazanavir sulfate and cobicistat; atazanavir and cobicistat; darunavir and cobicistat; atazanavir; atazanavir sulfate; dolutegravir; elvitegravir; ritonavir; atazanavir sulfate and ritonavir; darunavir; lamivudine; prolastin; fosamprenavir; fosamprenavir calcium efavirenz; etravirine; nelfinavir; nelfinavir mesylate; interferon; didanosine; stavudine; indinavir; indinavir sulfate; tenofovir and lamivudine; zidovudine; nevirapine; saquinavir; saquinavir mesylate; aldesleukin; zalcitabine; tipranavir; amprenavir; delavirdine; delavirdine mesylate; Radha-108 (receptol); lamivudine and tenofovir disoproxil fumarate; efavirenz, lamivudine, and tenofovir disoproxil fumarate; phosphazid; lamivudine, nevirapine, and zidovudine; abacavir; and abacavir sulfate.

In some embodiments, the crystalline forms, salts and co-crystals disclosed herein, or a pharmaceutical composition thereof, is combined with an HIV nucleoside or nucleotide inhibitor of reverse transcriptase and an HIV non-nucleoside inhibitor of reverse transcriptase. In another specific embodiment, an agent disclosed herein, or a pharmaceutical composition thereof, is combined with an HIV nucleoside or nucleotide inhibitor of reverse transcriptase, and an HIV protease inhibiting compound. In an additional embodiment, an agent disclosed herein, or a pharmaceutical composition thereof, is combined with an HIV nucleoside or nucleotide inhibitor of reverse transcriptase, an HIV non-nucleoside inhibitor of reverse transcriptase, and a pharmacokinetic enhancer. In certain embodiments, an agent disclosed herein, or a pharmaceutical composition thereof, is combined with at least one HIV nucleoside inhibitor of reverse transcriptase, an integrase inhibitor, and a pharmacokinetic enhancer. In another embodiment, an agent disclosed herein, or a pharmaceutical composition thereof, is combined with two HIV nucleoside or nucleotide inhibitors of reverse transcriptase.

In another embodiment, the crystalline forms, salts and co-crystals disclosed herein is combined with a first additional therapeutic agent chosen from dolutegravir, cabotegravir, islatravir, darunavir, bictegravir, elsulfavirine, rilpivirine, and lenacapavir and a second additional therapeutic agent chosen from emtricitabine and lamivudine.

In some embodiments, the crystalline forms, salts and co-crystals disclosed herein are combined with a first additional therapeutic agent (a contraceptive) selected from the group consisting of cyproterone acetate, desogestrel, dienogest, drospirenone, estradiol valerate, ethinyl Estradiol, ethynodiol, etonogestrel, levomefolate, levonorgestrel, lynestrenol, medroxyprogesterone acetate, mestranol, mifepristone, misoprostol, nomegestrol acetate, norelgestromin, norethindrone, noretynodrel, norgestimate, ormeloxifene, segestersone acetate, ulipristal acetate, and any combinations thereof.

### Gene Therapy and Cell Therapy

In certain embodiments, the crystalline forms, salts and co-crystals described herein are combined with a gene or cell therapy regimen. Gene therapy and cell therapy include without limitation the genetic modification to silence a gene; genetic approaches to directly kill the infected cells; the infusion of immune cells designed to replace most of the patient's own immune system to enhance the immune response to infected cells, or activate the patient's own immune system to kill infected cells, or find and kill the infected cells; genetic approaches to modify cellular activity to further alter endogenous immune responsiveness against the infection. Examples of cell therapy include without limitation LB-1903, ENOB-HV-01, ENOB-HV-21, ENOB-HV-31, GOVX-BO1, HSPCs overexpressing ALDH1 (LV-800, HIV infection), AGT103-T, and SupTI cell based therapy. Examples of dendritic cell therapy include without limitation AGS-004. CCR5 gene editing agents include without limitation SB-728T, SB-728-HSPC. CCR5 gene inhibitors include without limitation Cal-1, and lentivirus vector CCR5 shRNA/TRIM5alpha/TAR decoy-transduced autologous CD34-positive hematopoietic progenitor cells (HIV infection/HIV-related lymphoma). In some embodiments, C34-CCR5/C34-CXCR4 expressing CD4-positive T-cells are co-administered with one or more multi-specific antigen binding molecules. In some embodiments, the agents described herein are co-administered with AGT-103-transduced autologous T-cell therapy or AAV-eCD4-Ig gene therapy.

### Gene Editors

In certain embodiments, the crystalline forms, salts and co-crystals disclosed herein are combined with a gene editor, e.g., an HIV targeted gene editor. In various embodiments, the genome editing system can be selected from the group consisting of: a CRISPR/Cas9 complex, a zinc finger nuclease complex, a TALEN complex, a homing endonucleases complex, and a meganuclease complex. An illustrative HIV targeting CRISPR/Cas9 system includes without limitation EBT-101.

### CAR-T Cell Therapy

In some embodiments, the crystalline forms, salts and co-crystals disclosed herein can be co-administered with a population of immune effector cells engineered to express a chimeric antigen receptor (CAR), wherein the CAR comprises an HIV antigen binding domain. The HIV antigen include an HIV envelope protein or a portion thereof, gp120 or a portion thereof, a CD4 binding site on gp120, the CD4-induced binding site on gp120, N glycan on gp120, the V2 of gp120, the membrane proximal region on gp41. The immune effector cell is a T-cell or an NK cell. In some embodiments, the T-cell is a CD4+ T-cell, a CD8+ T-cell, or a combination thereof. Cells can be autologous or allogeneic. Examples of HIV CAR-T include A-1801, A-1902, convertible CAR-T, VC-CAR-T, CMV-N6-CART, anti-HIV duoCAR-T, anti-CD4 CART-cell therapy, CD4 CAR+C34-CXCR4+CCR5 ZFN T-cells, dual anti-CD4 CART-T cell therapy (CD4 CAR+C34-CXCR4 T-cells), anti-CD4 MicAbody antibody+anti-MicAbody CAR T-cell therapy (iNKG2D CAR, HIV infection), GP-120 CAR-T therapy, autologous hematopoietic stem cells genetically engineered to express a CD4 CAR and the C46 peptide.

### TCR T-Cell Therapy

In certain embodiments, the crystalline forms, salts and co-crystals disclosed herein are combined with a population of TCR-T-cells. TCR-T-cells are engineered to target HIV derived peptides present on the surface of virus-infected cells, for example, ImmTAV.

### B-Cell Therapy

In certain embodiments, the crystalline forms, salts and co-crystals disclosed herein are combined with a population of B cells genetically modified to express broadly neutralizing antibodies, such as 3BNC117 (Hartweger et al., J. Exp. Med. 2019, 1301, Moffett et al., Sci. Immunol. 4, eaax0644 (2019) 17 May 2019.

The crystalline form, salt or co-crystal disclosed herein may be combined with one, two, three, or four additional therapeutic agents in any dosage amount of the crystalline form, salt or co-crystal (e.g., from 1 mg to 1000 mg of compound).

In one embodiment, kits comprising crystalline form, salt or co-crystal disclosed herein, or a pharmaceutically acceptable salt thereof, in combination with one or more (e.g., one, two, three, one or two, or one to three) additional therapeutic agents are provided.

In one embodiment, the additional therapeutic agent or agents of the kit is an anti-HIV agent, selected from HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, HIV entry inhibitors, HIV maturation inhibitors, immunomodulators, immunotherapeutic agents, antibody-drug conjugates, gene modifiers, gene editors (such as CRISPR/Cas9, zinc finger nucleases, homing nucleases, synthetic nucleases, TALENs), cell therapies (such as chimeric antigen receptor T-cell, CAR-T, and engineered T cell receptors, TCR-T, autologous T cell therapies), compounds that target the HIV capsid, latency reversing agents, HIV bNAbs, immune-based therapies, phosphatidylinositol 3-kinase (PI3K) inhibitors, HIV antibodies, broadly neutralizing HIV antibodies, bispecific antibodies and “antibody-like” therapeutic proteins, HIV p17 matrix protein inhibitors, IL-13 antagonists, peptidyl-prolyl cis-trans isomerase A modulators, protein disulfide isomerase inhibitors, complement C5a receptor antagonists, DNA methyltransferase inhibitor, HIV vif gene modulators, Vif dimerization antagonists, HIV viral infectivity factor inhibitors, TAT protein inhibitors, HIV Nef modulators, Hck tyrosine kinase modulators, mixed lineage kinase-3 (MLK-3) inhibitors, HIV splicing inhibitors, Rev protein inhibitors, integrin antagonists, nucleoprotein inhibitors, splicing factor modulators, COMM domain containing protein 1 modulators, HIV ribonuclease H inhibitors, retrocyclin modulators, CDK-9 inhibitors, dendritic ICAM-3 grabbing nonintegrin 1 inhibitors, HIV GAG protein inhibitors, HIV POL protein inhibitors, Complement Factor H modulators, ubiquitin ligase inhibitors, deoxycytidine kinase inhibitors, cyclin dependent kinase inhibitors, proprotein convertase PC9 stimulators, ATP dependent RNA helicase DDX3X inhibitors, reverse transcriptase priming complex inhibitors, G6PD and NADH-oxidase inhibitors, pharmacokinetic enhancers, HIV gene therapy, HIV vaccines, and combinations thereof.

In some embodiments, the additional therapeutic agent or agents of the kit are selected from combination drugs for HIV, other drugs for treating HIV, HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors, latency reversing agents, capsid inhibitors, immune-based therapies, PI3K inhibitors, HIV antibodies, and bispecific antibodies, and “antibody-like” therapeutic proteins, and combinations thereof.

In a specific embodiment, the kit includes a compound disclosed herein, or a pharmaceutically acceptable salt thereof, and an HIV nucleoside or nucleotide inhibitor of reverse transcriptase. In a specific embodiment, the kit includes a compound disclosed herein, or a pharmaceutically acceptable salt thereof, and an HIV nucleoside or nucleotide inhibitor of reverse transcriptase and an HIV non-nucleoside inhibitor of reverse transcriptase. In another specific embodiment, the kit includes a compound disclosed herein, or a pharmaceutically acceptable salt thereof, and an HIV nucleoside or nucleotide inhibitor of reverse transcriptase, and an HIV protease inhibiting compound. In an additional embodiment, the kit includes a compound disclosed herein, or a pharmaceutically acceptable salt thereof, an HIV nucleoside or nucleotide inhibitor of reverse transcriptase, an HIV non-nucleoside inhibitor of reverse transcriptase, and a pharmacokinetic enhancer. In certain embodiments, the kit includes a compound disclosed herein, or a pharmaceutically acceptable salt thereof, at least one HIV nucleoside inhibitor of reverse transcriptase, an integrase inhibitor, and a pharmacokinetic enhancer. In another embodiment, the kit includes a compound disclosed herein, or a pharmaceutically acceptable salt thereof, and two HIV nucleoside or nucleotide inhibitors of reverse transcriptase. In a specific embodiment, the kit includes a compound disclosed herein, or a pharmaceutically acceptable salt thereof, an HIV nucleoside or nucleotide inhibitor of reverse transcriptase and an HIV capsid inhibitor. In a specific embodiment, the kit includes a compound disclosed herein, or a pharmaceutically acceptable salt thereof, an HIV nucleoside inhibitor of reverse transcriptase and an HIV capsid inhibitor. In a specific embodiment, the kit includes a compound disclosed herein, or a pharmaceutically acceptable salt thereof, and an HIV capsid inhibitor. In a specific embodiment, the kit includes a compound disclosed herein, or a pharmaceutically acceptable salt thereof, and one, two, three or four HIV bNAbs. In a specific embodiment, the kit includes a compound disclosed herein, or a pharmaceutically acceptable salt thereof, one, two, three or four HIV bNAbs and an HIV capsid inhibitor. In a specific embodiment, the kit includes a compound disclosed herein, or a pharmaceutically acceptable salt thereof, one, two, three or four HIV bNAbs, an HIV capsid inhibitor, and an HIV nucleoside inhibitor of reverse transcriptase.

### HIV Long Acting Therapy

Examples of drugs that are being developed as long acting regimens include, but are not limited to, cabotegravir, rilpivirine, any integrase LA, VM-1500 LAI, maraviroc (LAI), tenofovir implant, islatravir implant, doravirine, raltegravir, and long acting dolutegravir.

### Uses

Also provided herein are uses of any of the disclosed pharmaceutical compositions for treating or preventing an HIV infection.

Also provided herein are any of the pharmaceutical compositions provided herein for use in a method for treating or preventing HIV infection.

Also provided herein are any of the pharmaceutical compositions provided herein for use in a method for treating or preventing HIV infection, comprising administering to the human the pharmaceutical composition by injection.

Also provided herein are uses of any of the pharmaceutical compositions provided herein in the manufacture of a medicament for treating or preventing an HIV infection in a human, comprising administering to the human the pharmaceutical composition by injection.

### V. Methods of Preparation

Also provided herein are methods of preparing the pharmaceutical compositions described herein. Pharmaceutical suspension formulations can be prepared using methods known to those skilled in the art, for example, using the kneading method, a melting method (hot-melt extrusion, melt agglomeration), a solvent evaporation method (spray drying, lyophilization, super-critical fluid, co-precipitation, electrospinning), or a melt evaporation method. Depending upon the liquid vehicle and the method used in the preparation of the suspension formulation, the drug can exist as amorphous solids, crystalline solids, or other solid forms dispersed within the formulation. Examples of analytical characterization of suspension formulations include X-ray powder diffraction analysis (XRPD), differential-scanning calorimetry (DSC), thermogravimetric analysis (TGA), particle size analysis (PSD), scanning electron microscopy (SEM), and dissolution testing.

In some embodiments, provided herein is a method of preparing a pharmaceutical composition disclosed herein, comprising mixing the compound of Formula I, or a pharmaceutically acceptable salt thereof, with a liquid vehicle to form a suspension suitable for injection.

In some embodiments, the method comprises vortexing the suspension one or more times. In some embodiments, the method further comprises sonicating the suspension to break up any agglomerates of the compound of Formula I, or pharmaceutically acceptable salt thereof.

In some embodiments, the method comprises preparing a microsuspension. In some embodiments of the methods disclosed herein, the compound of Formula I, or a pharmaceutically acceptable salt thereof, is micronized. In some embodiments, the compound of Formula I, or pharmaceutically acceptable salt thereof, is micronized by milling as described herein, e.g., jet milling (fluid energy milling), bead milling, dry milling spiral milling, or high shear wet milling (HSWM). In some embodiments, the compound of Formula I, or pharmaceutically acceptable salt thereof, is micronized by jet milling.

In some embodiments of the methods disclosed herein, the compound of Formula I, or pharmaceutically acceptable salt thereof, has a particle size distribution d₉₀ of from about 1 μm to about 90 μm. In some embodiments of the methods disclosed herein, the compound of Formula I, or pharmaceutically acceptable salt thereof, has a particle size distribution d₉₀ of from about 1 μm to about 20 μm. In some embodiments of the methods disclosed herein, the compound of Formula I, or pharmaceutically acceptable salt thereof, has a particle size distribution d₉₀ of from about 5 μm to about 15 μm.

In some embodiments, the method comprises preparing a nanosuspension. In some embodiments, the method of preparing a nanosuspension comprises:
mixing the compound of Formula I, or a pharmaceutically acceptable salt thereof, with a liquid vehicle to form a pre-suspension; and
milling the pre-suspension to form a nanosuspension suitable for injection.

In some embodiments, the pre-suspension is milled by wet bead milling or high shear homogenization. In some embodiments, the compound of Formula I, or pharmaceutically acceptable salt thereof, is milled by wet bead milling. In some embodiments, the wet bead milling comprises grinding the pre-suspension with zirconium oxide beads to reduce the particle size distribution. In some embodiments, the pre-suspension is milled by high shear homogenization. In some embodiments, the high shear homogenization comprises utilizing pressure to pass the pre-suspension through a narrow orifice, thereby causing high shear and cavitation energy to reduce the particle size distribution.

In some embodiments, the high shear homogenization is performed at a single speed of about 3,000 rpm to about 20,000 rpm; at about 3,000 rpm, about 4,000 rpm, about 5,000 rpm, about 6,000 rpm, about 7,000 rpm, about 8,000 rpm, about 9,000 rpm, about 10,000 rpm, about 11,000 rpm, about 12,000 rpm, about 13,000 rpm, about 14,000 rpm, about 15,000 rpm, or about 16,000 rpm. In an embodiment, the high shear homogenization is performed at a single speed of 12,000 rpm.

In some embodiments of the methods disclosed herein, the compound of Formula I, or pharmaceutically acceptable salt thereof, has a particle size distribution d₉₀ of less than 1 μm. In some embodiments of the methods disclosed herein, the compound of Formula I, or pharmaceutically acceptable salt thereof, has a particle size distribution d₉₀ of from about 200 nm to about 900 nm.

In some embodiments, one or more excipients (e.g., stabilizers) are added to the nanosuspension after the pre-suspension is milled. In some embodiments, the method of preparing a nanosuspension comprises:
mixing the compound of Formula I, or a pharmaceutically acceptable salt thereof, with a liquid vehicle to form a pre-suspension; and
milling the pre-suspension to form a nanosuspension; and
adding one or more excipients to the nanosuspension to form a nanosuspension suitable for injection.

In some embodiments, the method further comprises diluting the suspension. In some embodiments, the method of preparing a nanosuspension further comprises diluting the nanosuspension to a target concentration. In some embodiments, the method of preparing a nanosuspension comprises mixing the compound of Formula I, or a pharmaceutically acceptable salt thereof, with a liquid vehicle to form a pre-suspension; milling the pre-suspension to form a second pre-suspension; and diluting the second pre-suspension to form a nanosuspension suitable for injection.

In some embodiments, the method of preparing a nanosuspension comprises diluting the nanosuspension (e.g., the second pre-suspension) to a concentration of about 200 mg/mL to about 500 mg/mL of the compound of Formula I, or pharmaceutically acceptable salt thereof. In some embodiments, the method of preparing a nanosuspension comprises diluting the nanosuspension (e.g., the second pre-suspension) to a concentration of about 200 mg/mL to about 400 mg/mL of the compound of Formula I, or pharmaceutically acceptable salt thereof. In some embodiments, the method of preparing a nanosuspension comprises diluting the nanosuspension (e.g., the second pre-suspension) to a concentration of about 200 mg/mL to about 300 mg/mL of the compound of Formula I, or pharmaceutically acceptable salt thereof. In some embodiments, the method of preparing a nanosuspension comprises diluting the nanosuspension (e.g., the second pre-suspension) to a concentration of about 300 mg/mL to about 400 mg/mL of the compound of Formula I, or pharmaceutically acceptable salt thereof. In some embodiments, the method of preparing a nanosuspension comprises diluting the nanosuspension (e.g., the second pre-suspension) to a concentration of about 400 mg/mL to about 500 mg/mL of the compound of Formula I, or pharmaceutically acceptable salt thereof.

In some embodiments, the nanosuspension is prepared utilizing precipitation technologies. In some embodiments, the method of preparing a nanosuspension comprises:
mixing the compound of Formula I, or a pharmaceutically acceptable salt thereof, with a solvent to form a solvent phase;
mixing one or more excipients with water to form a water phase;
emulsifying the solvent phase in the water phase using high pressure homogenization;
removing the solvent to form a first nanosuspension; and
concentrating the first nanosuspension to form a nanosuspension suitable for injection.

In some embodiments, the water phase comprises one or more excipients selected from PEG 300, PEG 400, PEG 3350, PEG 6000, poloxamer 188, poloxamer 338, poloxamer 407, PVA, PVP, Tween 20, Tween 80, sodium deoxycholate, polyethylene glycol (15)-hydroxystearate (Kolliphor® HS 15), and polyoxyl-35 castor oil (Kolliphor® ELP).

The first nanosuspension can be concentrated to achieve a concentration suitable for injection. In some embodiments, the concentration of the first nanosuspension comprises cross flow filtration of the first nanosuspension. In some embodiments, the cross flow filtration comprises a filter having a molecular weight cut off of about 50 kDa to about 1000 kDa, about 100 kDa to about 800 kDa, about 200 kDa to about 700 kDa, about 400 kDa to about 600 kDa, or about 500 kDa. In some embodiments, the cross flow filtration comprises a polyethersulfone (PES) membrane.

In some embodiments, the method of preparing a nanosuspension comprises concentrating the first nanosuspension to a concentration of about 200 mg/mL to about 500 mg/mL of the compound of Formula I, or pharmaceutically acceptable salt thereof. In some embodiments, the method of preparing a nanosuspension comprises concentrating the first nanosuspension to a concentration of about 200 mg/mL to about 400 mg/mL of the compound of Formula I, or pharmaceutically acceptable salt thereof. In some embodiments, the method of preparing a nanosuspension comprises concentrating the first nanosuspension to a concentration of about 200 mg/mL to about 300 mg/mL of the compound of Formula I, or pharmaceutically acceptable salt thereof (e.g., 250 mg/mL). In some embodiments, the method of preparing a nanosuspension comprises concentrating the first nanosuspension to a concentration of about 300 mg/mL to about 400 mg/mL of the compound of Formula I, or pharmaceutically acceptable salt thereof. In some embodiments, the method of preparing a nanosuspension comprises concentrating the first nanosuspension to a concentration of about 400 mg/mL to about 500 mg/mL of the compound of Formula I, or pharmaceutically acceptable salt thereof.

In some embodiments, the compound of Formula I, or a pharmaceutically acceptable salt thereof, is spray dried. In some embodiments, the spray dried compound of Formula I, or pharmaceutically acceptable salt thereof, has a particle size distribution d₉₀ of from about 1 μm to about 90 μm.

In some embodiments, the compound of Formula I, or a pharmaceutically acceptable salt thereof, is hot melt extruded with a hydrophilic carrier. In some embodiments, the hydrophilic carrier is PLGA.

In some embodiments, the method further comprises preparing the liquid vehicle. In some embodiments, preparing the liquid vehicle comprises homogenizing the liquid vehicle (i.e., thoroughly mixing the components of the liquid vehicle to form a homogenous solution).

Also provided herein are pharmaceutical compositions prepared by any of the methods disclosed herein.

### VI. Kits

The pharmaceutical compositions provided herein can be formed from components provided by a kit. In one aspect, provided herein is a kit comprising the components of a pharmaceutical composition described herein in a suitable packaging. In some embodiments, the kit further comprises instructions for making and using the pharmaceutical compositions.

Accordingly, provided herein is a kit comprising:
a first container comprising a compound of Formula I, or pharmaceutically acceptable salt thereof, in solid form; and
a second container comprising a liquid vehicle.

In some embodiments, the kit comprises:
a first container comprising a compound of Formula I, or pharmaceutically acceptable salt thereof; and
a second container comprising a liquid vehicle; and
instructions for combining the contents of the first and second containers to prepare a pharmaceutical composition.

The second container can include any of the liquid vehicles described herein. In some embodiments, the liquid vehicle is an aqueous suspending vehicle. In some embodiments, the liquid vehicle is an organic suspending vehicle.

In some embodiments, provided herein is a kit comprising:
a first container comprising a compound of Formula I, or pharmaceutically acceptable salt thereof; and
a second container comprising an aqueous suspending vehicle comprising a hydrophilic carrier and water.

In some embodiments, provided herein is a kit comprising:
a first container comprising a compound of Formula I; and
a second container comprising an aqueous suspending vehicle comprising a hydrophilic carrier and water.

In some embodiments, the aqueous suspending vehicle comprises polyethylene glycol and water. In some embodiments, the aqueous suspending vehicle comprises about 70 wt. % to about 90 wt. % polyethylene glycol; and about 10 wt. % to about 30 wt. % water. In some embodiments, the aqueous suspending vehicle comprises about 80 wt. % polyethylene glycol, and about 20 wt. % water. In some embodiments, the polyethylene glycol in the aqueous suspending vehicle is PEG 300.

In some embodiments, the organic suspending vehicle comprises a pharmaceutically acceptable oil. In some embodiments, the pharmaceutically acceptable oil is castor oil, cottonseed oil, sesame oil, linseed oil, safflower oil, peanut oil, soybean oil, coconut oil, olive oil, corn oil, almond oil, poppyseed oil, sunflower oil, almond oil, vegetable oil, and mixtures thereof. In some embodiments, the pharmaceutically acceptable oil is selected from tricaprylin, caprylic/capric triglyceride (e.g., Miglyol 810, Miglyol 812, CAPTEX 355, and the like), caprylic/capric/linoleic triglyceride, caprylic/capric/succinic triglyceride, propylene glycol dicaprylate/dicaprate (e.g., Miglyol 840 and CAPTEX 200, and the like), glycerol triacetate (triacetin), glyceryl stearates, and the like, including mixtures thereof.

In some embodiments, provided herein is a kit comprising:
a first container comprising a compound of Formula I, or pharmaceutically acceptable salt thereof; and
a second container comprising a pharmaceutically acceptable oil.

In some embodiments, provided herein is a kit comprising:
a first container comprising a compound of Formula I; and
a second container comprising pharmaceutically acceptable oil.

In some embodiments, the second container comprises a pharmaceutically acceptable oil, wherein the pharmaceutically acceptable oil comprises sesame oil. In some embodiments, the second container comprises a pharmaceutically acceptable oil, wherein the pharmaceutically acceptable oil comprises a medium chain triglyceride. In some embodiments, the second container comprises a pharmaceutically acceptable oil, wherein the pharmaceutically acceptable oil comprises a medium chain triglyceride which is Miglyol 812 N.

In some embodiments, the first container comprises a compound of Formula I free acid. In some embodiments, the first container comprises a compound of Formula I, wherein the compound of Formula I is crystalline Form I.

In some embodiments, the first container comprises a calcium salt of the compound of Formula I. In some embodiments, the calcium salt of the compound of Formula I is a hemi-calcium salt. In some embodiments, the calcium salt of the compound of Formula I has Form I. In some embodiments, the calcium salt of the compound of Formula I is amorphous.

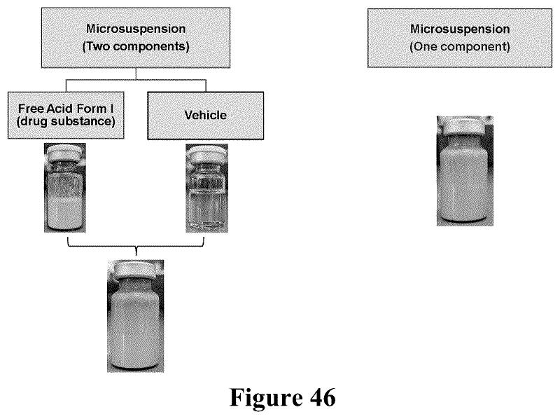

FIG. 46 shows an example kit comprising a first container comprising a compound of Formula I and a second container comprising a liquid vehicle. A pharmaceutical composition (e.g., a microsuspension) can be prepared from the provided components.

Optionally associated with such containers can be a notice in the form prescribed by a governmental agency regulating the manufacture, use or sale of pharmaceuticals, which notice reflects approval by the agency for the manufacture, use or sale for human administration. The kits may be in unit dosage forms, bulk packages (e.g., multi-dose packages) or sub-unit doses. Kits may also include multiple unit doses of the compounds and instructions for use and be packaged in quantities sufficient for storage and use in pharmacies (e.g., hospital pharmacies and compounding pharmacies).

In some embodiments, the kits disclosed herein are provided with suitable packaging for use in the methods described herein. Suitable packaging is known in the art and includes, for example, vials, vessels, ampules, bottles, jars, flexible packaging, prefilled syringes, empty disposable syringes, autoinjectors, needles, and the like. The packaging and components may further be sterilized and/or sealed.

The invention will be described in greater detail by way of specific examples. The following examples are offered for illustrative purposes, and are not intended to limit the invention in any manner. Those of skill in the art will readily recognize a variety of noncritical parameters which can be changed or modified to yield essentially the same results.

### EXAMPLES

### General Methods

X-ray powder diffraction (XRPD) analysis was conducted on a diffractometer (PANalytical XPERT-PRO, PANalytical B. V., Almelo, Netherlands) using copper radiation (Cu Kα, λ=1.541874 Å). Samples were spread evenly on a zero background sample plate. The generator was operated at a voltage of 45 kV and amperage of 40 mA. Slits were Soller 0.02 rad, antiscatter 1.0°, and divergence. Scans were performed from 2 to 40° 2θ with a 0.0167 step size. Data analysis was performed using X'Pert Data Viewer V1.2d (PANalytical B.V., Almelo, Netherlands). X-ray powder diffraction analysis was also conducted on a diffractometer (Rigaku MiniFlex, Rigaku Corporation, Beijing, China) using copper radiation (Cu Kα, λ=1.541874 Å). Samples were spread evenly on a zero background sample plate. The generator was operated at a voltage of 40 kV and amperage of 15 mA. Scans were performed from 2 to 40° 2θ with a 0.050 degree step size and a speed of 2 degrees/minute. Data analysis was also performed using X'Pert Data Viewer V1.2d (PANalytical B.V., Almelo, Netherlands).

Differential Scanning Calorimetry (DSC) was run on a Q2000 (TA Instruments, New Castle, DE) by loading 1-10 mg of material into a crimped or open Tzero standard aluminum pan and heating the sample at 10° C./min from 20 to 300° C. or above. The sample and reference pans were under a 50 mL/min nitrogen purge. Data analysis was completed using Universal Analysis 2000 Version 4.5A (TA Instruments, New Castle, DE).

Thermogravimetric analysis (TGA) was used to evaluate sample weight loss as a function of temperature on either a Q5000 or Q500 (TA Instruments, New Castle, DE), by loading 1-10 mg of material onto a weigh pan and heating the sample to 350° C. or above at a rate of 10° C./min. The sample and reference pans were under a 60 mL/min and 40 mL/min nitrogen purge, respectively. Data analysis was completed using Universal Analysis 2000 Version 4.5A (TA Instruments, New Castle, DE).

Hygroscopicity was studied using dynamic vapor sorption (DVS, TA Q5000 SA, TA Instruments, New Castle, DE or DVS, DVS Intrinsic, Surface Measurement Systems, London, UK). A sample (1-20 mg) was placed in an aluminum DVS pan and loaded on the sample side of the twin pan balance. The water sorption and desorption were studied as a function of relative humidity (RH) at 25° C. In 10% RH increments, the relative humidity was increased from 5% RH to 95% RH and then decreased back to 5%. Each relative humidity increment had an equilibration time of 180 minutes, unless weight change % was less than 0.002% in 30 minutes.

Data analysis was performed using Universal Analysis 2000 Version 4.7A (TA Instruments, New Castle, DE) for TA DVS runs and Microsoft Excel for SMS DVS runs.

### Example 1. Compound of Formula I, Form I

### Example 1a

Compound of Formula I, Form I is an unsolvated phase. It was isolated when amorphous compound of Formula I was slurried separately in the following solvent systems at room temperature for about 14 days: Acetone, MeCN, EtOH, EtOAc, IPA, IPOAc, MeTHF, toluene, THF, and water.

### Example 1b

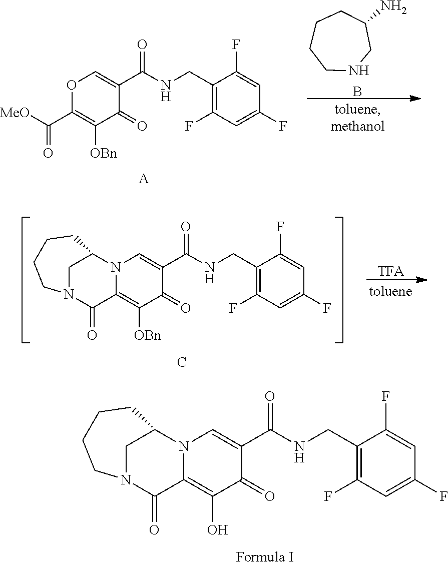

Compound of Formula I, Form I was also isolated when methyl 3-(benzyloxy)-4-oxo-5-((2,4,6-trifluorobenzyl)carbamoyl)-4H-pyran-2-carboxylate (A) (1.0 equiv, scaling factor), toluene (5.4 volumes), (S)-azepan-3-amine (B) (1.1 equiv), and methanol (0.6 volumes) were charged to a reactor. The mixture was agitated at about 55° C. until the reaction was deemed complete. The mixture was then concentrated under reduced pressure. Toluene (4.0 volumes) was charged, and the mixture was concentrated again under reduced pressure. Toluene (4.0 volumes) and trifluoroacetic acid (5.0 equiv) were then charged, and the mixture was agitated at about 55° C. until the reaction was deemed complete. The mixture was then concentrated under reduced pressure. Toluene (4.0 volumes) was charged, and the mixture was again concentrated under reduced pressure. 2-Propanol (2.0 volumes) was charged, and the mixture was agitated at about 55° C. for about 1 hour. Additional 2-propanol (10 volumes) was then charged over about 1 hour, then the mixture was cooled to about 25° C. and agitated for about 16 hours. The slurry was filtered and resulting cake rinsed with 2-propanol (3.0 volumes), rinsed twice with ethanol (3.0 volumes), then dried to afford the compound of Formula I, Form I.

### Example 1c

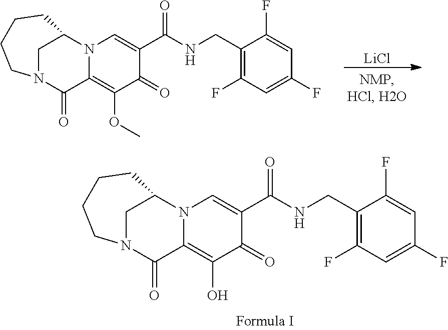

Compound of Formula I, Form I was also isolated when (7S)-12-methoxy-1,11-dioxo-N-(2,4,6-trifluorobenzyl)-1,4,5,6,7,11-hexahydro-3H-2,7-methanopyrido[1,2-a][1,4]diazonine-10-carboxamide (D) (1.00 equiv, scaling factor), lithium chloride (3.0 equiv), and N-methyl-2-pyrolidine (3.0 volumes) were charged to a reactor. The mixture was heated to about 100° C. and agitated until the reaction was deemed complete. The reaction mixture was then charged dropwise to another reactor containing 1 M hydrochloric acid (3.0 volumes) at about 2° C. The slurry was then agitated at about 25° C. for about 16 hours. The slurry was then filtered and rinsed twice with water (3.0 volumes), then rinsed twice with 2-propanol (3.0 volumes), and finally dried to afford the compound of Formula I, Form I.

### Example 1d

Compound of Formula I, Form I was also isolated when methyl 3-(benzyloxy)-4-oxo-5-((2,4,6-trifluorobenzyl)carbamoyl)-4H-pyran-2-carboxylate (A) (1.00 equiv, scaling factor), (S)-azepan-3-amine (B) (1.1 equiv.), sodium bicarbonate (2.15 equiv.), and methanol (6.0 volumes) to a reactor. The mixture was heated to about 60° C. and agitated until the reaction was deemed complete. Ethyl acetate (10.0 volumes) and saturated aqueous ammonium chloride (5.0 volumes) were charged and the mixture agitated for about 15 minutes. The phases were allowed to separate, and the lower layer was discarded. Water (5.0 volumes) was charged, and the mixture agitated for about 15 minutes. The phases were allowed to separate, and the lower layer was discarded. The mixture was then concentrated under reduced pressure. Toluene (4.0 volumes) was charged, and the mixture was concentrated again under reduced pressure.

Toluene (4.0 volumes) and trifluoroacetic acid (4.0 volumes) was charged. The mixture was heated to about 60° C. and agitated until the reaction was deemed complete. The mixture was concentrated under reduced pressure. Ethyl acetate (2.0 volumes) was charged, and the mixture was seeded with Form I at room temperature. Heptane (2.0 volumes) was then charged, and the mixture agitated for about 30 minutes. Additional heptane (4.0 volumes) was charged, and the mixture agitated for about 12 h. The slurry was then filter and rinsed with ethyl acetate (1.0 volume), and dried to afford compound of Formula I, Form I.

### Example 1e

The compound of Formula I, Form I was also isolated when the compound of Formula I, sodium salt Form I (1.00 equiv, scaling factor) and ethanol (10.0 volumes) were charged to a reactor. Glacial acetic acid (2.0 equiv) was charged over about 1 h during which the mixture was agitated and subjected to high sheer wet milling at about 15,000 rpm with recirculation. Agitation and wet-milling was continued for about 30 minutes and the mixture was further agitated for about 3 hours. The slurry was then filtered, rinsed twice with ethanol (1.0 volumes), and dried at about 50° C. for about 20 hours.

The Compound of Formula I, Form I XRPD pattern is shown in Error! Reference source not found. and is characterized by Tier 1 reflections at 17.8, 22.0, 25.4° 20, but also Tier 2 at 15.6, 23.1, 29.2° 20, and Tier 3 at 11.1, 12.6, 17.3° 20. A list of 2-theta peaks is provided below:

Peak Table

| Pos. | Rel. Int. |
|---|---|
| [°2Th.] | [%] |
| 10.2051 | 17.91 |
| 11.0588 | 34.41 |
| 12.6118 | 31.59 |
| 14.5193 | 17.32 |
| 15.6463 | 41.62 |
| 17.2528 | 38.69 |
| 17.8322 | 100.00 |
| 19.6384 | 14.92 |
| 20.7623 | 16.29 |
| 21.9574 | 96.32 |
| 23.1489 | 44.24 |
| 24.2050 | 21.41 |
| 25.0185 | 40.07 |
| 25.3612 | 84.53 |
| 26.2356 | 11.77 |
| 27.7314 | 10.03 |
| 28.6533 | 34.12 |
| 29.1593 | 49.06 |
| 30.5358 | 20.39 |
| 34.8604 | 22.44 |
| 38.5013 | 12.61 |

The DSC curve is shown in FIG. 2 Error! Reference source not found. and displays one endothermic transition at about 246° C. The TGA curve is shown in FIG. 3 Error! Reference source not found. and indicates that the phase is unsolvated. The dynamic vapor sorption curve is shown in FIG. 4 Error! Reference source not found. and the data indicate that the form absorbs about 0.097% of water up to 95% RH at 25° C. The material was found to not have changed form post experiment.

Single crystal data was collected on the compound of Formula I, Form I and the data are summarized in Table 1 Error! Reference source not found. and FIG. 5 Error! Reference source not found.

The crystal system is monoclinic and the space group is P2₁. The cell parameters and calculated volume are: a=8.94841(12) Å, b=8.58582(16) Å, c=12.59306(18) Å, α=90°, β=103.2526(13)°, γ=90°, V=941.75(3) Å³. The molecular weight is 421.37 g mol⁻¹ with Z=2, resulting in a calculated density of 1.486 g cm⁻³.

TABLE 2
Crystal Data and Data Collection Parameters for Formula I, Form I

| | |
|---|---|
| Empirical formula | C₂₀H₁₈F₃N₃O₄ |
| Formula weight (g mol⁻¹) | 421.37 |
| Temperature (K) | 300.1(2) |
| Wavelength (Å) | 1.54184 |
| Crystal system | monoclinic |
| Space group | P2₁ |
| Unit cell parameters | |
| a = 8.94841(12) Å | α = 90° |
| b = 8.58582(16) Å | β = 103.2526(13)° |
| c = 12.59306(18) Å | γ = 90° |
| Unit cell volume (Å³) | 941.75(3) |
| Cell formula units, Z | 2 |
| Calculated density (g cm⁻³) | 1.486 |
| Absorption coefficient (mm⁻¹) | 1.070 |
| F(000) | 436 |
| Crystal size (mm³) | 0.21 × 0.08 × 0.02 |
| Reflections used for cell measurement | 6168 |
| θ range for cell measurement | 5.0740°-77.2490° |
| Total reflections collected | 9769 |
| Index ranges | −10 ≤ h ≤ 11; −10 ≤ k ≤ 9; −15 ≤ / ≤ 15 |
| θ range for data collection | 0ₘᵢₙ = 5.078°, 0ₘₐₓ = 77.513° |
| Completeness to θₘₐₓ | 97.8% |
| Completeness to θ_{full} = 67.684° | 99.8% |
| Absorption correction | multi-scan |
| Transmission coefficient range | 0.891-1.000 |
| Refinement method | full matrix least-squares on F² |
| Independent reflections | 3339 [Rᵢₙₜ = 0.0233, R_{σ} = 0.0230] |
| Reflections [I > 2σ(I)] | 3062 |
| Reflections/restraints/parameters | 3339/1/343 |
| Goodness-of-fit on F² | S = 1.05 |
| Final residuals [I > 2σ(I)] | R = 0.0303, R_{w} = 0.0811 |
| Final residuals [all reflections] | R = 0.0331, R_{w} = 0.0835 |
| Largest diff. peak and hole (e Å⁻³) | 0.104, −0.149 |
| Max/mean shift/standard uncertainty | 0.000/0.000 |
| Absolute structure determination | Flack |
| | parameter: 0.00(8) |
| | Hooft parameter: |
| | 0.03(7) |
| | Friedel coverage: 66.4% |

### Example 2. Compound of Formula I, Form II

Compound of Formula I, Form II is a metastable methanol solvated phase. It was isolated when compound of Formula I, Form I was slurried in methanol for about 60 days and identified by x-ray of the wet cake. Upon drying compound of Formula I, Form II under ambient conditions, it was found to convert to compound of Formula I, Form III.

The XRPD pattern for the compound of Formula I, Form II is shown in FIG. 6 Error! Reference source not found. and is characterized by Tier 1 reflections at 9.5, 10.9, 21.2° 20, but also Tier 2 at 14.7, 17.8, 18.9° 20, and Tier 3 at 6.3, 28.0, 28.7° 20. A list of 2-theta peaks is provided below:

Peak Table

| Pos. | |
|---|---|
| [°2Th.] | Rel. Int. |
| 5.4610 | 17.91 |
| 6.3322 | 25.41 |
| 8.9092 | 23.95 |
| 9.4542 | 100.00 |
| 10.9298 | 69.05 |
| 13.2035 | 4.15 |
| 14.7383 | 62.27 |
| 15.6982 | 15.85 |
| 16.3292 | 15.33 |
| 17.2677 | 16.89 |
| 17.7594 | 48.53 |
| 18.0435 | 39.97 |
| 18.8815 | 55.12 |
| 19.4545 | 19.10 |
| 21.2189 | 64.10 |
| 23.1999 | 32.03 |
| 23.9297 | 32.13 |
| 25.7520 | 21.42 |
| 27.0226 | 14.90 |
| 27.4848 | 12.72 |
| 28.0375 | 43.04 |
| 28.6660 | 40.58 |
| 29.4663 | 20.64 |
| 30.1798 | 8.24 |
| 30.6728 | 18.29 |

### Example 3. Compound of Formula I, Form III

### Example 3a

Compound of Formula I, Form III is a methanol solvated phase. It was isolated when amorphous compound of Formula I was slurried in MeOH at room temperature for about 14 days and isolated as a dry cake.

### Example 3b

Compound of Formula I, Form III was also made when Form II was dried under ambient conditions.

### Example 3c

Compound of Formula I, Form III was also isolated when compound of Formula I, sodium salt, Form I (1.00 equiv, scaling factor), methanol (5.0 volumes), and ethyl acetate (5.0 volumes) were charged to a reactor. The mixture was heated to about 40° C. and glacial acetic acid (1.6 equiv) was charged. The mixture was then heated to about 60° C., agitated for about 30 minutes, and then cooled to about 20° C. over about 2 hours. The slurry was agitated for about 16 hours at about 20° C. The slurry was then filtered, rinsed with 50% methanol in ethyl acetate (2.0 volumes), and dried at about 50° C. for about 24 h to afford Compound of Formula I, Form III.

The compound of Formula I, Form III XRPD pattern is shown in FIG. 7 and is characterized by Tier 1 reflections at 15.8, 17.3, 26.4° 20, but also Tier 2 at 14.9, 18.1, 21.8° 2θ, and Tier 3 at 5.8, 9.7, 10.5° 20. A list of 2-theta peaks is provided below:

Peak Table

| Pos. | Rel. Int. |
|---|---|
| [°2Th.] | [%] |
| 5.7681 | 24.93 |
| 9.7205 | 22.56 |
| 10.5053 | 40.02 |
| 11.5440 | 29.19 |
| 14.9183 | 53.73 |
| 15.8074 | 64.25 |
| 17.2791 | 100.00 |
| 18.0835 | 51.41 |
| 19.4465 | 36.71 |
| 21.7763 | 55.41 |
| 23.7336 | 8.60 |
| 24.7317 | 5.43 |
| 26.3864 | 88.10 |
| 29.4755 | 23.36 |
| 30.2145 | 37.76 |
| 30.7309 | 18.01 |

The DSC curve is shown in FIG. 8 and displays an endothermic transition at about 156° C., an endothermic transition at about 180° C., an exothermic transition at about 186° C., and an endothermic transition at about 247° C. The TGA curve is shown in FIG. 9 and indicates that the phase is solvated. The dynamic vapor sorption curve is shown in FIG. 10 and the data indicate that the form absorbs about 0.87% of water up to 95% RH at 25° C. The material was found to not have changed form post experiment.

### Example 4. Compound of Formula I, Form IV

Compound of Formula I, Form IV is a hydrated phase. It was isolated when 500 μL of a DMSO stock solution (˜50 mg/mL, compound of Formula I) was added to either 50 mL of 0.1 wt/v % poloxamer 188 in simulated gastric fluid, 0.1 wt/v % poloxamer 338 in simulated gastric fluid, 0.1 wt/v % tocopheryl polyethylene glycol succinate in simulated gastric fluid, or 0.1 wt/v % tween 80 in simulated gastric fluid and then stirred at room temperature for around 24 hours.

The compound of Formula I, Form IV XRPD pattern is shown in FIG. 11 and is characterized by Tier 1 reflections at 4.1, 12.3, 16.4° 2θ, but also Tier 2 at 13.7, 23.4, 25.4° 2θ, and Tier 3 at 8.1, 18.9, 27.4° 2θ. A list of 2-theta peaks is provided below:

Peak Table

| Pos. | Rel. Int. |
|---|---|
| [°2Th.] | [%] |
| 4.0905 | 61.21 |
| 8.1684 | 25.79 |
| 12.2546 | 85.48 |
| 13.7183 | 59.27 |
| 15.5882 | 25.92 |
| 16.3620 | 100.00 |
| 17.4922 | 23.39 |
| 18.4025 | 6.79 |
| 18.9112 | 34.07 |
| 21.3775 | 18.03 |
| 22.1915 | 9.88 |
| 23.3990 | 54.83 |
| 24.6191 | 18.60 |
| 25.4026 | 52.81 |
| 27.4476 | 29.32 |
| 28.4581 | 15.08 |
| 28.8361 | 16.20 |
| 31.4865 | 14.90 |
| 33.0815 | 17.71 |
| 35.1201 | 13.63 |

The DSC curve is shown in FIG. 12 and displays an endothermic transition at about 33° C., an exothermic transition at about 86° C., and an endothermic transition at about 244° C. The TGA curve is shown in FIG. 13 and indicates that the phase is hydrated. The dynamic vapor sorption curve is shown in Error! Reference source not found. and the data indicate that the form absorbs about 3.5% of water up to 95% RH at 25° C. The material was found to not have changed form post experiment.

### Example 5. Compound of Formula I, Amorphous

Compound of Formula I, amorphous was isolated when approximately 20 g of compound of Formula I, Form I was dissolved in dichloromethane (˜250 ml) at room temperature and the solution was spray dried to yield a powder. The inlet temperature for the spray drier was about 50° C., while the outlet temperature as about 35° C.

The XRPD pattern for the compound of Formula I, amorphous is shown in FIG. 15 and is characterized by a broad hump. The DSC curve is shown in FIG. 16 indicating a glass transition around 110° C.

### Example 6. Compound of Formula I, Sodium Salt, Form I

### Example 6a

Sodium salt of the compound of Formula I is an unsolvated phase. It was isolated when about 1000 mg of the compound of Formula I, Form I was placed in a vial with −10 mL of EtOH. Next, about 96 mg of solid NaOH was added along with about 1 mL of water and the sample was sonicated for about 60 minutes to yield a precipitate. Next, about 10 mL of EtOH was added and the sample was stirred at room temperature for about 3 days then filtered and air dried.

### Example 6b

Sodium salt of the compound of Formula I was also isolated when compound of Formula I, Form I (1.0 equiv, scaling factor) and ethanol (10 volumes) were charged to a reactor. The mixture was heated to about 75° C. and 25 wt % sodium hydroxide in water (1.1 equiv) was charged to the reactor, followed by rinsing forward with water (0.2 volumes). The reaction mixture was then agitated for about 30 minutes, cooled to about 2θ° C. over about 4 hours, and then agitated for about 18 hours. The slurry was filtered, rinsed twice with 95% ethanol in water (2.0 volumes), and dried at about 50° C.

The sodium salt XRPD pattern is shown in FIG. 17 and is characterized by Tier 1 reflections at 6.5, 20.2, 23.5° 2θ, but also Tier 2 at 14.3, 17.2, 19.2° 2θ, and Tier 3 at 13.0, 19.8, 30.8° 2θ. A list of 2-theta peaks is provided below:

Peak Table

| Pos. | Rel. Int. |
|---|---|
| [°2Th.] | [%] |
| 6.4582 | 100.00 |
| 9.1357 | 7.98 |
| 10.9197 | 5.37 |
| 12.9859 | 9.48 |
| 14.3397 | 23.23 |
| 16.6524 | 3.41 |
| 17.1777 | 21.24 |
| 18.2116 | 8.54 |
| 18.7336 | 3.89 |
| 19.2149 | 15.37 |
| 19.7692 | 11.99 |
| 20.2415 | 39.53 |
| 20.9268 | 6.60 |
| 21.8961 | 6.32 |
| 22.5979 | 3.26 |
| 23.1770 | 9.59 |
| 23.4848 | 34.81 |
| 26.4924 | 4.82 |
| 27.2889 | 6.52 |
| 28.4243 | 7.38 |
| 28.8215 | 8.79 |
| 30.1002 | 9.22 |
| 30.7739 | 11.53 |
| 32.8031 | 6.89 |
| 34.1696 | 3.24 |

The DSC curve is shown in FIG. 18 and displays an endothermic transition at about 371° C. and an exothermic transition at about 374° C. The TGA curve is shown in FIG. 19 and indicates that the phase is unsolvated. The dynamic vapor sorption curve is shown in FIG. 20 and the data indicate that the form absorbs about 0.48% of water up to 95% RH at 25° C. The material was found to not have changed form post experiment.

### Example 7. Compound of Formula I, Calcium Salt, Form I

Compound of Formula I, calcium salt, Form I is a hydrated phase. It was isolated when around 1 equiv. of Compound of Formula I, Form I, around 6 volumes of ethanol, and around 2.5 volumes of water were charged to a reactor and stirred. Next around 1.05 equiv. of 50% w/v aq. potassium hydroxide was then charged and the mixture was agitated for about 24 hours at room temperature, then polish filtered into a clean reactor. Then around 0.2 volumes of heptane were added, followed by around 0.5 equiv. of a 1 M aq. calcium chloride solution charged over about 6 hours. The mixture was agitated for about 1 hour, then the slurry was then filtered, and the solids rinsed with around 5.0 volumes of water twice.

The Compound of Formula I, calcium salt, Form I XRPD pattern is shown in FIG. 21 and is characterized by reflections at 6.1, 5.6, 4.8, and 7.6° 2θ. A list of 2-theta peaks is provided below:

Peak Table

| Pos. | Rel. Int. |
|---|---|
| [°2Th.] | [%] |
| 4.759 | 32.23 |
| 5.5975 | 52.28 |
| 6.1208 | 100 |
| 7.6264 | 14.89 |

The DSC curve is shown in FIG. 22 and displays an endothermic transition at about 43° C. and an endothermic transition at about 291° C. The TGA curve is shown in FIG. 23 and indicates that the phase is hydrated. The dynamic vapor sorption curve is shown in FIG. 24 and the data indicate that the form absorbs about −18% of water up to 95% RH at 25° C.

### Example 8. Compound of Formula I, Calcium Salt, Form II

Compound of Formula I, calcium salt, Form II is a hydrated phase. It was isolated when compound of Formula I, calcium salt, Form I was slurred around 4 day at room temperature in either 9:1 THF:water (v/v) or 8:2 THF:water (v/v).

The Compound of Formula I, calcium salt, Form II XRPD pattern is shown in FIG. 25 Error! Reference source not found. and is characterized by Tier 1 reflections at 6.4, 5.2, 5.6° 2θ, but also Tier 2 at 18.9, 21.9, 10.9° 2θ, and Tier 3 at 16.8, 15.1, 29.3° 2θ. A list of 2-theta peaks is provided below:

Peak Table

| Pos. | Rel. Int. |
|---|---|
| [°2Th.] | [%] |
| 5.2027 | 94.25 |
| 5.6215 | 83.25 |
| 6.4645 | 100 |
| 10.8889 | 25.26 |
| 15.0964 | 15.92 |
| 16.8257 | 23.38 |
| 18.9083 | 29.87 |
| 21.9068 | 27.32 |
| 24.9566 | 6.72 |
| 25.9705 | 6.49 |
| 27.8323 | 9.87 |
| 29.2844 | 15.82 |
| 31.4693 | 12.42 |

The DSC curve is shown in FIG. 26 and displays an endothermic transition at about 19° C., and an endothermic transition at about 320° C. The TGA curve is shown in FIG. 27 and indicates that the phase is hydrated.

### Example 9. Compound of Formula I, Calcium Salt, Form III

Compound of Formula I, calcium salt, Form III is a hydrated phase. It was isolated when compound of Formula I, calcium salt, Form I was slurred around 4 day at room temperature in either 9:1 IPA:water (v/v), 8:2 IPA:water (v/v), or 7:3 IPA:water (v/v). Alternatively, it was isolated when Form I was slurred around 14 day at room temperature in either acetone, 6:4 MeOH:water (v/v), 5:5 MeOH:water (v/v), 3:7 EtOH:water (v/v), 2:8 EtOH:water (v/v), or 1:9 IPA:water (v/v).

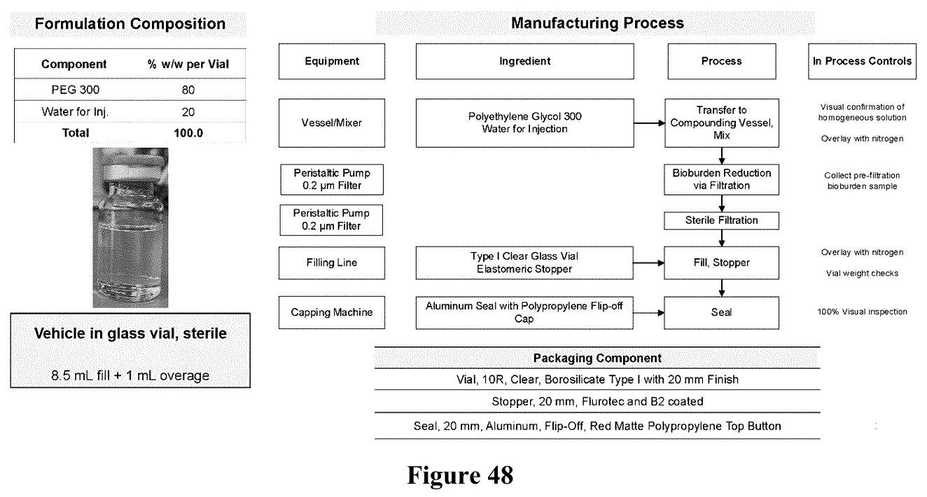

The compound of Formula I, calcium salt, Form III XRPD pattern is shown in FIG. 48 Error! Reference source not found. and is characterized by Tier 1 reflections at 4.2, 5.2, 6.2° 2θ, but also Tier 2 at 13.9, 21.0, 15.7° 2θ, and Tier 3 at 12.6, 7.5, 10.6° 2θ.

Peak Table

| Pos. | Rel. Int. |
|---|---|
| [°2Th.] | [%] |
| 4.2027 | 100 |
| 5.2494 | 64.58 |
| 6.1747 | 62.82 |
| 7.4813 | 15.12 |
| 10.6013 | 12.69 |
| 12.5542 | 17.46 |
| 13.9203 | 27.71 |
| 15.6797 | 19.8 |
| 21.0148 | 25.12 |

The DSC curve is shown in FIG. 29 and displays an endothermic transition at about 17° C., an endothermic transition at about 93° C., and an endothermic transition at about 286° C. The TGA curve is shown in FIG. 30 and indicates that the phase is hydrated. The dynamic vapor sorption curve is shown in FIG. 31 and the data indicate that the form absorbs about ˜14% of water up to 95% RH at 25° C.

### Example 10. Compound of Formula I, Calcium Salt, Form IV

Compound of Formula I, calcium salt, Form IV is a hydrated phase. It was isolated when around 1 equiv. of compound of Formula I Form I, around 6 volumes of ethanol, and around 2.5 volumes of water were charged to a reactor and stirred. Next around 1.05 equiv. of 50% w/v aq. potassium hydroxide was then charged and the mixture was agitated for about 24 hours at room temperature, then polish filtered into a clean reactor. Then around 0.2 volumes of Heptane were added, followed by around 0.5 equiv. of a 1 M aq. calcium chloride solution charged over about 6 hours. The mixture was agitated for about 1 hour, then the slurry was then filtered, and the mother liquors retained and held at room temperature for around 2 weeks to yield a solid that was filtered and dried.

The compound of Formula I, calcium salt, Form IV XRPD pattern is shown in FIG. 32 and is characterized by Tier 1 reflections at 6.5, 5.2, 20.7° 2θ, but also Tier 2 at 7.0, 18.9, 13.2° 2θ, and Tier 3 at 17.8, 9.6, 22.9° 2θ.

Peak Table

| Pos. | Rel. Int. |
|---|---|
| [°2Th.] | [%] |
| 5.1906 | 97.5 |
| 6.5315 | 100 |
| 6.9516 | 63.68 |
| 8.4207 | 11.94 |
| 9.5864 | 38.78 |
| 12.1583 | 7.54 |
| 13.1534 | 44.74 |
| 14.7177 | 14.96 |
| 15.6964 | 20.69 |
| 17.8258 | 43.97 |
| 18.895 | 56.04 |
| 20.7192 | 65.87 |
| 22.8522 | 33.95 |
| 24.3624 | 29.15 |
| 26.1189 | 11.88 |
| 28.138 | 24.29 |

The DSC curve is shown in FIG. 33 and displays an endothermic transition at about 17° C., and a baseline shift around 315° C. The TGA curve is shown in FIG. 34 and indicates that the phase is hydrated. The dynamic vapor sorption curve is shown in FIG. 35 and the data indicate that the form absorbs about −3% of water up to 95% RH at 25° C.

### Example 11. Compound of Formula I, Calcium Salt, Form V

Compound of Formula I, calcium salt, Form V is a hydrated phase. It was isolated when to a reactor was charged around 1 equiv. of compound of Formula I, Form I, around 6 volumes of methanol, and around 3 volumes of ammonium buffer (3 M NH₄OH, 1 M NH₄Cl). The mixture was agitated at about 25° C. for about 10 minutes then a 1 M solution of CaCl₂ (0.5 equiv) was added over about 3 h. The resulting mixture was aged for about 18 h then filtered, and the cake washed twice with 3 volumes of water. The wet-cake was dried under vacuum at about 50° C.

The compound of Formula I, calcium salt, Form V XRPD pattern is shown in FIG. 36 and is characterized by Tier 1 reflections at 6.3, 9.2, 4.7° 2θ, but also Tier 2 at 17.5, 6.6, 7.3° 2θ, and Tier 3 at 19.8, 10.3, 21.9° 2θ.

Peak Table

| Pos. | Rel. Int. |
|---|---|
| [°2Th.] | [%] |
| 4.667286 | 85.49 |
| 6.325596 | 100 |
| 6.574366 | 58.89 |
| 7.337722 | 41.98 |
| 9.219071 | 92.48 |
| 10.33942 | 28.4 |
| 11.76972 | 25.41 |
| 13.8499 | 17.35 |
| 17.51082 | 71.54 |
| 19.82631 | 37.1 |
| 21.93564 | 26.21 |
| 23.9084 | 19.19 |
| 28.46511 | 11.11 |

The DSC curve is shown in FIG. 37 and displays an endothermic transition at about 34° C., and an endothermic transition at about 1962° C. The TGA curve is shown in FIG. 38 and indicates that the phase is hydrated. The dynamic vapor sorption curve is shown in FIG. 39 and the data indicate that the form absorbs about −0.13% of water up to 95% RH at 25° C.

### Example 12. Compound of Formula I, Calcium Salt, Form VI

Compound of Formula I, calcium salt, Form VI is a hydrated phase. It was isolated when Formula I, calcium salt, Form V was slurried at room temperature in MeOH for about 14 days.

The compound of Formula I, calcium salt, Form VI XRPD pattern is shown in FIG. 40 and is characterized by Tier 1 reflections at 6.2, 5.0, 5.8° 2θ, but also Tier 2 at 19.1, 18.2, 21.0° 2θ, and Tier 3 at 21.7, 10.0, 10.3° 2θ.

Peak Table

| Pos. | Rel. Int. |
|---|---|
| [°2Th.] | [%] |
| 4.97 | 63.7 |
| 5.83 | 53.94 |
| 6.21 | 100 |
| 7.90 | 10.79 |
| 8.65 | 6.8 |
| 9.95 | 22.63 |
| 10.34 | 21.71 |
| 12.61 | 15.78 |
| 13.22 | 19.38 |
| 14.50 | 20.38 |
| 15.77 | 12.66 |
| 18.22 | 29.81 |
| 19.11 | 35.21 |
| 20.98 | 27.28 |
| 21.71 | 26.79 |
| 22.61 | 16.35 |
| 24.99 | 20.77 |
| 26.59 | 8.56 |
| 29.60 | 9.96 |

The DSC curve is shown in FIG. 41 and displays an endothermic transition at about 51° C., and an endothermic transition at about 262° C. The TGA curve is shown in FIG. 42 and indicates that the phase is hydrated. The dynamic vapor sorption curve is shown in FIG. 43 and the data indicate that the form absorbs about −0.1% of water up to 95% RH at 25° C.

### Example 13. Compound of Formula I, Calcium Salt, Amorphous

### Example 13a

Compound of Formula I, calcium salt, amorphous was isolated when approximately 2 grams potassium salt was placed in a vial with about 20 mL of 1:1 EtOH:water (v/v) to yield a solution. Then around 500 mg of calcium chloride was added to yield a precipitate and the sample then stirred at RT for 3 nights then solids isolated. Alternatively, it was made when around 1.00 equiv. of Compound of Formula I, Form I and around 5.0 volumes of ethanol were charged to a reactor and stirred at room temperature. Next around 1.2 equiv. of 50% w/v aq. potassium hydroxide was charged, followed by around 3.5 volumes of water. The mixture was agitated for about 2 hours at room temperature, then polish filtered into a clean reactor. Then around 0.5 equiv. of 1 M aq. calcium chloride solution was charged over about 19 hours while agitating. The slurry was then filtered, the solids rinsed with around 10.0 volumes of water three times, and then dried. Its XRPD pattern is shown in FIG. 44 and is characterized by broad humps. The DSC curve is shown in FIG. 45 indicating a baseline shift around 288° C.

### Example 13b

The compound of Formula I, calcium salt, amorphous was also made when to a reactor was charged the compound of Formula I, Form I (1.00 equiv), calcium hydroxide (0.5 equiv), methanol (4 volumes), tetrahydrofuran (4.0 volumes), and triethylamine (2.0 equiv). The mixture was agitated at about 25° C. then water (1.0 volume) was charged, and the resulting mixture aged for about 12 hours. The reaction stream was then polish filtered into a clean reactor and water (9.0 volumes) was charged over about 2 hours, and the resulting slurry aged for about 12 hours. The slurry was then filtered, and the cake washed twice with 3 volumes of 2θ:80 methanol/water containing 1 vol % triethylamine. The wet-cake was dried under vacuum at 60° C.

### Example 13c

The compound of Formula I, calcium salt, amorphous was also made when to a reactor was charged the compound of Formula I, Form I (1.00 equiv, scaling factor), calcium oxide (0.5 equiv), and methanol (5 volumes). The mixture was heated to about 65° C. and aged for about 12 hours. To the mixture was charged tetrahydrofuran (5 volumes), triethylamine (2.0 equiv), and water (1.0 volume) and the resulting mixture aged for about 1 hour. The reaction stream was then polish filtered into a clean reactor and water (9.0 volumes) was charged over about 2 hours, and the resulting slurry aged for about 12 hours. The slurry was then filtered, and the cake washed twice with 3 volumes of 2θ:80 methanol/water containing 1 vol % triethylamine. The wet-cake was dried under vacuum at 60° C.

### Example 14. Preparation of Components for Suspension Formulations

### Example 14a: Preparation of Compound of Formula I, Form I

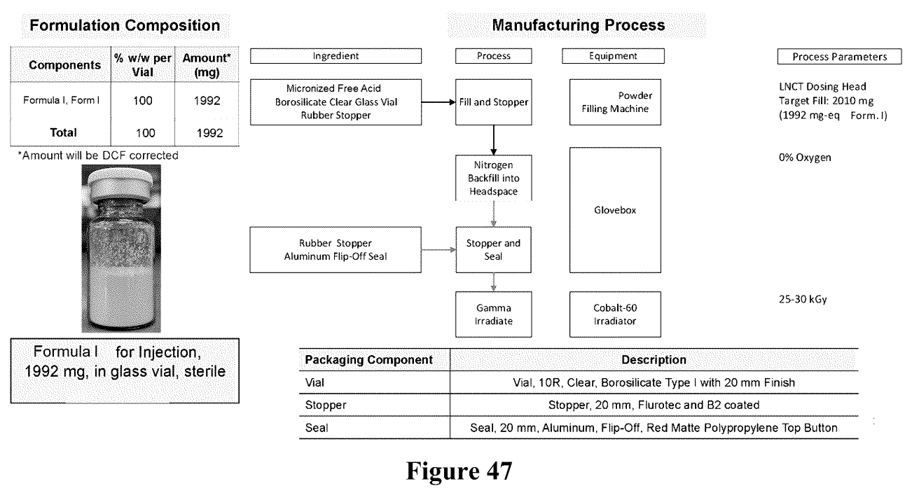

FIG. 47 shows an example manufacturing process to provide a compound of Formula I, Form I to be utilized in pharmaceutical compositions for injection. The compound of Formula I, Form I was micronized and placed in a borosilicate clear glass vial with a rubber stopper. The vial was nitrogen backfilled into the headspace, stoppered, sealed with an aluminum flip-off seal, and sterilized with gamma radiation.

### Example 14b: Preparation of PEG300/Water Liquid Vehicle

FIG. 48 shows an example manufacturing process to provide a liquid vehicle comprising PEG300 (80 wt. %) and water for injection (20 wt. %). PEG300 and water for injection were transferred to a compounding vessel and mixed. The mixture was subject to bioburden reduction via filtration, followed by sterile filtration. The mixture was places in a glass vial, stoppered with an elastomeric stopper, and sealed with an aluminum seal having a polypropylene flip-off cap.

### Example 14c: Preparation of a Sesame Oil Liquid Vehicle

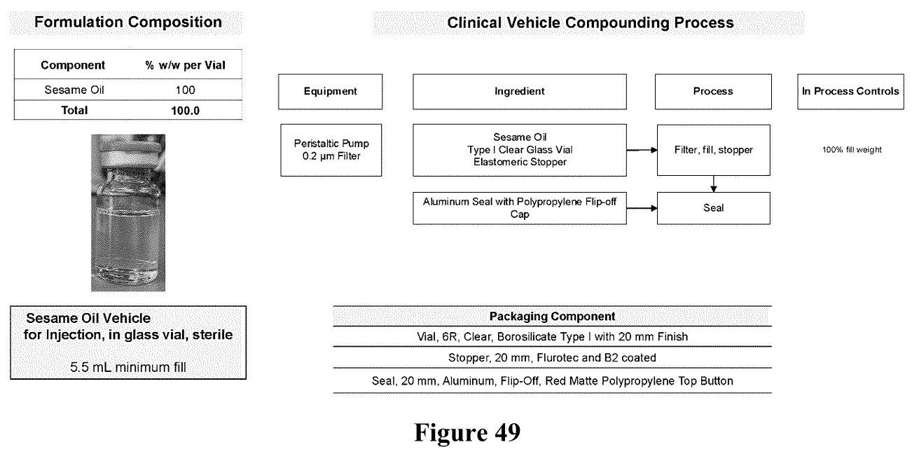

FIG. 49 shows an example manufacturing process to provide a liquid vehicle comprising sesame oil. Sesame oil was transferred to a compounding vessel and subject to bioburden reduction via filtration, followed by sterile filtration. The vehicle was places in a glass vial, stoppered with an elastomeric stopper, and sealed with an aluminum seal having a polypropylene flip-off cap.

### Example 15. Preparation of Microsuspension Formulations

### Example 15a: PEG/Water formulations (Two-Component)

Example suspension formulations comprise polyethylene glycol and water. PEG 300 (80% w/w) and water for injection (20% w/w) were added to a vessel and homogenized. For smaller scale reactions, a stir bar was utilized to homogenize the mixture. For larger scale reactions, an overhead mixer or large stir bar was utilized to homogenize the mixture.

The compound of Formula I, or pharmaceutically acceptable salt thereof, (e.g., jetmilled compound of Formula I) was added to a vial. The desired amount of homogenous PEG/water (80/20 w/w) vehicle was added to the vial to achieve the target concentration (e.g., 225 mg/mL to 450 mg/mL). The vial was vortexed to fully wet the compound of Formula I, or pharmaceutically acceptable salt thereof. The vial was again vortexed prior to withdrawal of the suspension to ensure a homogeneous suspension was withdrawn.

### Example 15b: Oil-Based Formulations (Two-Component)

The compound of Formula I, or pharmaceutically acceptable salt thereof, (e.g., jetmilled compound of Formula I) was added to a vial. The desired amount of oil-based vehicle (e.g., pharmaceutically acceptable oil) was added to the vial to achieve the target concentration (e.g., 225 mg/mL to 400 mg/mL). The vial was vortexed to fully wet the compound of Formula I, or pharmaceutically acceptable salt thereof. The vial was again vortexed prior to withdrawal of the suspension to ensure a homogeneous suspension was withdrawn.

### Example 15c: PEG/Water Formulations (One-Component)

PEG 300 (80% w/w) and water for injection (20% w/w) were compounded as described in Example 14b. The compound of Formula I, or pharmaceutically acceptable salt thereof (e.g., jetmilled compound of Formula I) was added to achieve the target concentration (e.g., 400 mg/mL) while stirring the compounded vehicle. The suspension was confirmed to be homogenous, and then filled into vials.

### Example 15d: Oil-Based Formulations (One-Component)

Oil-based vehicle (e.g., sesame oil or Miglyol 812 N) was added to a compounding vessel. The compound of Formula I, or pharmaceutically acceptable salt thereof (e.g., jetmilled compound of Formula I) was added to achieve the target concentration (e.g., 350 mg/mL) while stirring the compounded vehicle. The suspension was confirmed to be homogenous, and then filled into vials.

### Example 16. Microsuspension Formulations

Example suspension formulations were prepared according to procedures described in Example 15 with the amounts and components listed in Tables 3-6.

### (I) PEG/Water Suspensions

TABLE 3
Sample PEG/Water suspensions

Compound of Formula I
concentration
(mg/mL)

| | |
|---|---|
| 400 | 225 |

| | |
|---|---|
| Component | % w/w |

| | | |
|---|---|---|
| Compound of Formula I | 33.29 | 19.12 |
| PEG 300 | 53.37 | 64.70 |
| Water for injection | 13.34 | 16.18 |
| Total | 100.00 | 100.00 |

Other suspensions of the compound of Formula I in 80/20 (w/w %) PEG/WET were prepared with a concentration of 450 mg/mL compound of Formula I.

### (II) Sesame Oil Suspensions

TABLE 4
Sample sesame oil suspension

| | Compound of Formula I |
|---|---|
| | concentration (mg/mL) |
| | 350 |
| Component | % w/w |
| Compound of Formula I | 33.27 |
| Sesame oil | 66.73 |
| Total | 100.00 |

### (III) Triglyceride Suspensions

Suspensions of the compound of Formula I, Form I were achieved in a triglyceride based media, e.g., Miglyol 812 N, a triglyceride ester of saturated coconut/palm kernel oil derived caprylic and capric fatty acids and plant derived glycerol (60/40 caprylic acid (C8:0)/capric acid (C1c:0). Sample formulations were prepared at a concentration of 300 mg/mL and 400 mg/mL compound of Formula I, Form I.

TABLE 5
Sample Miglyol 812N formulations

| | | | Compound |
|---|---|---|---|
| | | | of Formula I |
| | | Formulation | concentration |
| Sample | Compound | Vehicle | (mg/mL) |
| III-A | Compound of Formula I, | 100% Miglyol | 300, 350, 400, |
| | Form I | 812N | 450, or 800 |
| III-B | Compound of Formula I, | 100% Miglyol | 300 or 400 |
| | sodium salt, Form I | 812N | |
| III-C | Compound of Formula I, | 100% Miglyol | 400 |
| | calcium salt, Form I | 812N | |
| III-D | Compound of Formula I, | 100% Miglyol | 400 |
| | calcium salt, amorphous | 812N | |

### (IV) Aqueous Suspensions

Suspensions of the compound of Formula I and pharmaceutically acceptable salts thereof were achieved in other aqueous liquid vehicles. Sample formulations are listed in Table 6.

TABLE 6
Sample aqueous formulations

| | | | Compound of |
|---|---|---|---|
| | | | Formula I |
| | | Formulation | concentration |
| Sample | Compound | Vehicle | (mg/mL) |
| IV-A | Compound of | 0.5% HPMC E4M, 0.5% | 230-250 |
| | Formula I, | Tween 20, 99% PBS (pH | |
| | Form I | 7.4) | |
| IV-B | Compound of | 0.5% HPMC E4M, 0.5% | 250 |
| | Formula I, | Tween 20, 99% PBS (pH | |
| | sodium salt | 7.4) | |
| IV-C | Compound of | P338 (3 wt. % of total | 378 |
| | Formula I, | formulation) | |
| | calcium salt, | WFI (59 wt. % of total | |
| | amorphous | formulation) | |
| IV-D | Compound of | 91.67% WFI | 400 |
| | Formula I, | 5% P338 | |
| | calcium salt, | 3.33% Mannitol | |
| | amorphous | | |
| IV-F | Compound of | P407 (2 wt. % of total | 475 |
| | Formula I, | formulation) | |
| | Form I | Mannitol (2 wt. % of total | |
| | | formulation) | |
| | | WFI | |

### Example 17: Preparation of Nanosuspension Formulations

The liquid vehicle was prepared by weighing and adding water for injection (WFI) to a vessel. Stabilizer(s)/stabilizing excipient(s) (e.g., suspending agents, surfactants) were added to the vessel. The mixture was homogenized and confirmed to be a solution.

The compound of Formula I (e.g., jetmilled compound of Formula I) was added to the vessel containing the liquid vehicle to achieve the target concentration (e.g., 20% to 40% w/w compound of Formula I).

The suspension was homogenized using a stir bar or overhead mixture to form a “pre-suspension.” The pre-suspension was processed using wet bead milling or high pressure homogenization to achieve the desired particle size distribution (PSD). In the wet bead milling process, zirconium oxide beads were utilized in a chamber to grind the compound of Formula I suspension into the desired PSD range. In the high pressure homogenization process, pressure was utilized to force the suspension through a narrow orifice, causing high shear and cavitation energy to reduce the PSD.

The concentration of the final suspension (second pre-suspension) was measured and diluted to target concentration (e.g., 200 mg/mL to 400 mg/mL compound of Formula I).

The tonicity of the formulation was optionally adjusted by adding a tonicity agent (e.g., mannitol) to the pre-suspension. The tonicity agent could alternatively be added to the final suspension (second-presuspension).

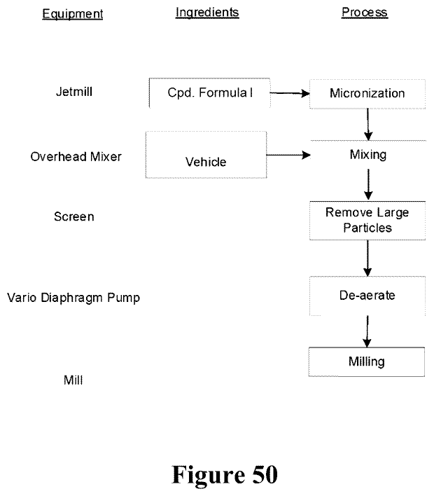

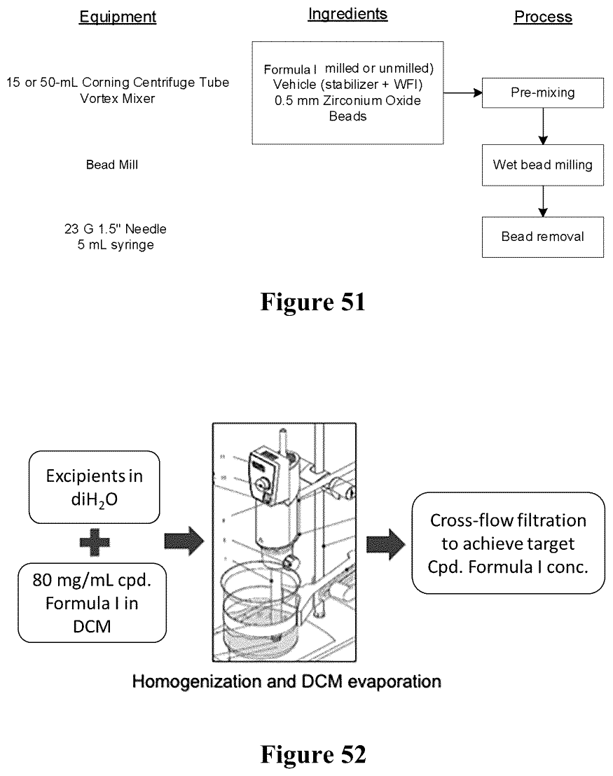

FIGS. 50 and 51 depict example processes for preparing a nanosuspension formulation using wet bead milling.

### Example 18: Nanosuspension Formulations

Nanosuspensions of the compound of Formula I were achieved using the procedure described in Example 17. Sample formulations are provided in Table 7.

TABLE 7
Sample nanosuspension formulations

| | | Formulation |
|---|---|---|
| Sample | Compound | Vehicle |
| V-A | Compound of Formula I, | PEG 3350 (2% w/w) |
| | Form I (20% w/w) | Tween 20 (2% w/w) |
| | | WFI (76% w/w) |
| V-B | Compound of Formula I, Form I | P407 (2% w/w) |
| | (20-40% w/w, 200-500 mg/mL) | Mannitol (2% w/w) |
| | | WFI |
| V-C | Compound of Formula I, | P407 (3% w/w) |
| | Form I (30% w/w) | Sodium deoxycholate (0.3% |
| | | w/w) |
| | | Mannitol (2% w/w) |
| | | WFI (64.7% w/w) |
| V-D | Compound of Formula I, | P407 (3% w/w) |
| | Form I (30% w/w) | Sodium deoxycholate (0.3% |
| | | w/w) |
| | | Mannitol (2.5% w/w) |
| | | WFI (64.2% w/w) |

% w/w in Table 7 refers to the weight percent of the total formulation

Formula V-C was prepared at a 2θ0 mL batch size. Formula V-D was prepared at a 2 kg batch size (approx. 1600 mL output nanosuspension). Additional characterization data for these formulations are provided in Table 8.

TABLE 8
Characterization of Nanosuspensions

| | | |
|---|---|---|
| Formulation | V-C | V-D |

| Concentration (mg/mL) | 333 | | 333 | |
|---|---|---|---|---|
| % AN | 99.73 | | 99.76 | |
| PSD (Laser Diffraction) | d₁₀ 316 | nm | d₁₀ 317 | nm |
| | d₅₀ 472 | nm | d₅₀ 476 | nm |
| | d₉₀ 740 | nm | d₉₀ 754 | nm |
| DLS | Z-avg 244 | nm | Z-avg 368 | nm |

| | |
|---|---|
| PDI 0.166 | PDI 0.177 |

| | | |
|---|---|---|
| Zeta potential (mV) | −13.4 | −12.6 |
| pH | 7.45 | 7.43 |

7.33

| | | |
|---|---|---|
| Osmolality (mOsm/kg) | 246 | 336 |

| | | 311 |
|---|---|---|
| | | 300 |
| XRD | Compound of | Compound of |
| | Formula I, Form I | Formula I, Form I |

### Example 19: Preparation of Nanosuspension Formulations

The compound of Formula I (e.g., jetmilled compound of Formula I) was dissolved in a solvent phase (80 mg/mL compound of Formula I in dichloromethane (DCM)). The water phase was prepared, including 25 mg/mL Tween 20 and 50 mg/mL PVA in 40% PEG 300 in water (500 mL).

The solvent phase was emulsified in the water phase using an Ultraturrax (high shear homogenizer) at 12,000 rpm for 20 minutes. The dichloromethane was evaporated. The resulting concentration of the compound of Formula I was 8 mg/mL.

The nanosuspension was concentrated by cross flow filtration (CFF) using 500 kDA PES membrane 32 fold, which resulted in a concentration of the compound of Formula I of 250 mg/mL. Tween 20 and PVA concentrations did not change during CFF.

FIG. 52 depicts an example process for preparing a nanosuspension formulation using high shear homogenization and concentration by CFF.

All references, including publications, patents, and patent documents are incorporated by reference herein, as though individually incorporated by reference. The present disclosure provides reference to various embodiments and techniques. However, it should be understood that many variations and modifications may be made while remaining within the spirit and scope of the present disclosure.

1. A pharmaceutical composition comprising a compound of Formula I: 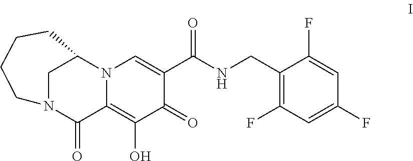 [SMILES: O=C(NCc1c(F)cc(F)cc1F)c1cn2c(c(O)c1=O)C(=O)N1CCCC[C@H]2C1]
or a pharmaceutically acceptable salt thereof, and a pharmaceutically acceptable liquid vehicle;
wherein the pharmaceutical composition is a suspension formulation.

2. The pharmaceutical composition of claim 1, wherein the pharmaceutical composition is suitable for administration via injection.

3.-5. (canceled)

6. The pharmaceutical composition of claim 1, wherein the compound of Formula I, or a pharmaceutically acceptable salt thereof, is present at a concentration of about 200 mg/mL to about 500 mg/mL, about 300 mg/mL to about 400 mg/mL, about 200 mg/mL to about 300 mg/mL, or about 400 mg/mL to about 500 mg/mL.

7.-12. (canceled)

13. The pharmaceutical composition of claim 1, wherein the compound of Formula I, or a pharmaceutically acceptable salt thereof, is present at about 10 wt. % to about 55 wt. %, about 10 wt. % to about 50 wt. %, about 15 wt. % to about 50 wt. %, or about 15% to about 45% of the total pharmaceutical composition weight.

14.-24. (canceled)

25. The pharmaceutical composition of claim 1, comprising the compound of Formula I.

26. The pharmaceutical composition of claim 25, wherein the compound of Formula I is crystalline.

27. The pharmaceutical composition of claim 26, wherein the compound of Formula I is crystalline Form I, wherein the crystalline Form I is characterized by an X-ray powder diffraction (XRPD) pattern comprising ° 2-θ peaks (±0.2° 2-θ) at 17.8°, 22.0°, and 25.4°.

28.-38. (canceled)

39. The pharmaceutical composition of claim 1, comprising a pharmaceutically acceptable salt of the compound of Formula I.

40.-55. (canceled)

56. The pharmaceutical composition of claim 1, which is an aqueous suspension, wherein the aqueous suspension comprises about 5 wt. % to about 90 wt. % water.

57.-67. (canceled)

68. The pharmaceutical composition of claim 1 comprising:
about 10 wt. % to about 40 wt. % of the compound of Formula I; and
about 60 wt. % to about 90 wt. % of a mixture comprising polyethylene glycol and water, wherein the ratio of polyethylene glycol and water in the mixture is from about 60:40 to about 90:10 wt. %.

69.-153. (canceled)

154. The pharmaceutical composition of claim 1, which is an organic suspension, wherein the organic suspension comprises a pharmaceutically acceptable oil, a pharmaceutically acceptable high-boiling liquid, or a combination thereof.

155. (canceled)

156. The pharmaceutical composition of claim 154, wherein the pharmaceutically acceptable oil or pharmaceutically acceptable high-boiling liquid is selected from sesame oil, a medium chain triglyceride, castor oil, polyethylene glycol, propylene glycol, benzyl benzoate, glycerol, oleic acid, a polyoxylglyceride, polyethylene glycol hydroxystearate, an ethoxylated castor oil, dimethylsulfoxide, N-methylpyrrolidone, or a combination thereof.

157. The pharmaceutical composition of claim 154, wherein the pharmaceutically acceptable oil is sesame oil, a medium chain triglyceride, or a combination thereof.

158. The pharmaceutical composition of claim 154, wherein the pharmaceutically acceptable oil or a pharmaceutically acceptable high-boiling liquid is present in the composition in an amount of about 60% to about 90% by weight.

159.-161. (canceled)

162. The pharmaceutical composition of claim 154, comprising sesame oil.

163. The pharmaceutical composition of claim 162, comprising:
about 10 wt. % to about 40 wt. % of the compound of Formula I; and
about 60 wt. % to about 90 wt. % sesame oil.

164. The pharmaceutical composition of claim 162, comprising:
about 10 wt. % to about 40 wt. % of the compound of Formula I; and
about 60 wt. % to about 90 wt. % sesame oil;
wherein the compound of Formula I is present at a concentration of about 200 mg/mL to about 500 mg/mL.

165.-192. (canceled)

193. A method for treating or preventing an HIV infection in a human, comprising administering to the human by injection a therapeutically effective amount of the pharmaceutical composition of claim 1.

194.-256. (canceled)

257. A method of preparing a pharmaceutical composition according to claim 1, comprising mixing the compound of Formula I, or a pharmaceutically acceptable salt thereof, with a liquid vehicle to form a suspension suitable for injection.

258.-273. (canceled)

274. A kit comprising:
i) a first container comprising a compound of Formula I, or a pharmaceutically acceptable salt thereof;
ii) a second container comprising a liquid vehicle; and
iii) instructions for combining the contents of the first and second containers to prepare a pharmaceutical composition of claim 1.

275.-292. (canceled)
